(19) United States
(12) Patent Application Publication
(10) Pub. No.: US 2021/0220408 A1
(43) Pub. Date: Jul. 22, 2021

(54) ARYL HYDROCARBON RECEPTOR ANTAGONISTS AND METHODS OF USE

(71) Applicant: Magenta Therapeutics Inc., Cambridge, MA (US)

(72) Inventors: Anthony BOITANO, Waban, MA (US); Michael COOKE, Boston, MA (US); Kevin A. GONCALVES, Boston, MA (US); Megan HOBAN, Medford, MA (US)

(21) Appl. No.: 17/273,510

(22) PCT Filed: Sep. 4, 2019
(86) PCT No.: PCT/US2019/049502

§ 371 Date: Mar. 4, 2021

Related U.S. Application Data
(60) Provisional application No. 62/882,838, filed on Aug. 5, 2019.
(60) Provisional application No. 62/726,884, filed on Sep. 4, 2018.

(51) Int. Cl. A61K 35/28; C07D 295/135; C07D 261/08; C07D 405/12; C07D 249/06; C07D 487/04; C07C 255/60; C07D 498/04; C07D 513/04; C07D 237/20; C07D 231/56; C07D 237/24; C07D 249/12; C07D 209/44; C07D 277/28; C07D 275/04; C07C 233/81; C07D 213/75; C07D 207/34; C07D 495/04; C07D 215/38; C07D 417/12; C07D 235/18; C07D 307/91; C07D 495/14; C07D 471/04; C07D 471/14; C07D 409/12; C07D 413/12; C07D 213/82; C07D 249/04; C07D 417/04; C07D 401/14; A61K 31/5375; A61K 31/277; A61K 31/42; A61K 31/443; A61K 31/519; A61K 31/4192; A61K 31/437; A61K 31/501; A61K 31/50; A61K 31/416; A61K 31/429; A61K 31/4196; A61K 31/4035; A61K 31/426; A61K 31/428; A61K 31/167; A61K 31/4162; A61K 31/44; A61K 31/415; A61K 31/47; A61K 31/427; A61K 31/454; A61K 31/4184; A61K 31/343; A61K 31/4365; A61K 31/381; A61K 31/4155; A61K 31/4436; A61K 31/423; A61K 31/55; A61K 31/421; A61K 31/425; A61K 31/4412; A61K 31/505; A61K 31/422; A61K 31/444; A61K 31/4545; A61K 31/4439; A61K 31/4704; A61K 31/4709; A61K 31/5377; A61K 31/366; C12N 5/0789; G01N 33/50; A61K 45/06

(52) U.S. Cl. CPC A61K 35/28; C07D 295/135; C07D 261/08; C07D 405/12; C07D 249/06; C07D 487/04; C07C 255/60; C07D 498/04; C07D 513/04; C07D 237/20; C07D 231/56; C07D 237/24; C07D 249/12; C07D 209/44; C07D 277/28; C07D 275/04; C07C 233/81; C07D 213/75; C07D 207/34; C07D 495/04; C07D 215/38; C07D 417/12; C07D 235/18; C07D 307/91; C07D 495/14; C07D 471/04; C07D 471/14; C07D 409/12; C07D 413/12; C07D 213/82; C07D 249/04; C07D 417/04; C07D 401/14; A61K 31/5375; A61K 31/277; A61K 31/42; A61K 31/443; A61K 31/519; A61K 31/4192; A61K 31/437; A61K 31/501; A61K 31/50; A61K 31/416; A61K 31/429; A61K 31/4196; A61K 31/4035; A61K 31/426; A61K 31/428; A61K 31/167; A61K 31/4162; A61K 31/44; A61K 31/415; A61K 31/47; A61K 31/427; A61K 31/454; A61K 31/4184; A61K 31/343; A61K 31/4365; A61K 31/381; A61K 31/4155; A61K 31/4436; A61K 31/423; A61K 31/55; A61K 31/421; A61K 31/425; A61K 31/4412; A61K 31/505; A61K 31/422; A61K 31/444; A61K 31/4545; A61K 31/4439; A61K 31/4704; A61K 31/4709; A61K 31/5377; A61K 31/366; C12N 5/0647; G01N 33/5073; A61K 45/06; C12N 2501/999; C12N 2501/145; C12N 2501/2306; C12N 2501/125; C12N 2501/26

(57) ABSTRACT

The disclosure relates to aryl hydrocarbon receptor antagonists as well as methods of modulating aryl hydrocarbon receptor activity and expanding hematopoietic stem cells by culturing hematopoietic stem or progenitor cells in the presence of these agents. Additionally, the disclosure provides methods of treating various pathologies, such as cancer, by administration of these aryl hydrocarbon receptor antagonists. Additionally, the disclosure provides methods of treating various pathologies in a patient by administration of expanded hematopoietic stem cells. The disclosure further provides kits containing aryl hydrocarbon receptor antagonists that can be used for the expansion of hematopoietic stem cells. The disclosure further relates to pharmaceutical compositions comprising the compounds and methods of treating or preventing a disease in which aryl hydrocarbon receptor plays a role.

### RELATED APPLICATIONS

This application claims priority to U.S. Application Nos. 62/882,838, filed Aug. 5, 2019, and 62/726,884, filed Sep. 4, 2018, the entire contents of each of which are incorporated herein by reference.

### FIELD

The present disclosure relates to aryl hydrocarbon receptor antagonists useful, for example, for ex vivo expansion and maintenance of hematopoietic stem cells, methods of treating or preventing a disease in which aryl hydrocarbon receptor plays a role, as well as methods of treating various hematopoietic pathologies by administration of the expanded hematopoietic stem cells and treatment of various pathologies, such as cancer, by administration of the arylhydrocarbon receptor antagonist.

### BACKGROUND

While hematopoietic stem cells have significant therapeutic potential, a limitation that has hindered their clinical use has been the difficulty associated with obtaining sufficient numbers of these cells. In particular, hematopoietic stem cells rapidly differentiate during ex vivo culture limiting the use of hematopoietic stem cells (HSCs) as a therapeutic modality by the loss of multi-potency.

Cancer remains one of the most deadly threats to human health. In the U.S., cancer affects nearly 1.3 million new patients each year, and is the second leading cause of death after heart disease, accounting for approximately 1 in 4 deaths. It is also predicted that cancer may surpass cardiovascular diseases as the number one cause of death within the next decade. Solid tumors are responsible for many of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making timely detection and treatment extremely difficult.

There is currently a need for novel agents that modulate and hydrocarbon receptor activity. There is currently a need for compositions and methods for the ex vivo maintenance, propagation, and expansion of HSCs that preserve the multi-potency and hematopoietic functionality of such cells, such as compounds that modulate and hydrocarbon receptor activity. There is currently a need for novel agents for use in therapeutic compositions and methods thereof for inhibiting cancer cell proliferation and tumor cell invasion and metastasis, such as compounds that modulate aryl hydrocarbon receptor activity.

### SUMMARY

The present, disclosure features aryl hydrocarbon receptor antagonists as well as methods of expanding hematopoietic stem cells by culturing hematopoietic stem ceils in the presence of such agents. Additionally described herein are kits containing aryl hydrocarbon receptor antagonists that can be used for the expansion of hematopoietic stem ceils. Additionally, the disclosure provides methods of treating various hematopoietic pathologies in a patient by administration of expanded hematopoietic stem cells. The patient may be suffering, for example, from a hemoglobinopathy or another disease of a cell in the hematopoietic lineage, and is thus in need of hematopoietic stem cell transplantation. As described herein, hematopoietic stem cells are capable of differentiating into a multitude of cell types in the hematopoietic family, and can be administered to a patient, in order to populate or reconstitute a blood cell type that is deficient in the patient. The disclosure thus provides methods of treating a variety of hematopoietic conditions, such as hematologic malignancy, sickle cell anemia, thalassemia, Fanconi anemia, Wiskott-Aldrich syndrome, adenosine deaminase deficiency-severe combined immunodeficiency, metachromatic leukodystrophy, Diamond-Blackfan anemia and Schwachman-Diamond syndrome, human immunodeficiency virus infection, and acquired immune deficiency syndrome, among others.

In a first aspect the disclosure features an aryl hydrocarbon receptor (AHR) modulator compound represented by Formula (I) or a salt thereof

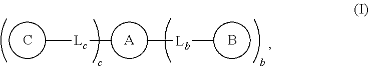

wherein:

A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;

b is 0 or 1;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *-—NR_{bb}C(O)NR_{bb}—**, *C(O) —**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O) —**, *—NR_{bb}—R_{ba}—(O)—**, *—O—R_{ba}—NR_{bb-}**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and A and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

c is 0 or 1;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *NR_{cb}*—R_{ca}—**, *—C(O)—**, *—SO₂—**, *—N═CR_{cb}—**, *—CR_{cb}═N—**, *—C(O)NR_{cb}**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C6 alkyl;

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl;

when c is 1, b is 1; and

when b is 0 and c is 0, A is an optionally substituted tricyclic ring selected from 14-membered aryl and 12- to 14-membered saturated or unsaturated heterocycle comprising 1-3 heteroatoms selected from N, O and S.

In some embodiments, b is 1 and c is 0.

In some embodiments, A is an optionally substituted monocyclic ring selected from the group consisting of benzene, pyridine, thiazole, piperazine, pyrimidine.1,2,3-triazole, pyrazole, furan, isoxazole, 4H-pyridazine, thiophene, oxazole, and 2H-pyridine.

In some embodiments, A is an optionally substituted monocyclic ring selected from the group consisting of:

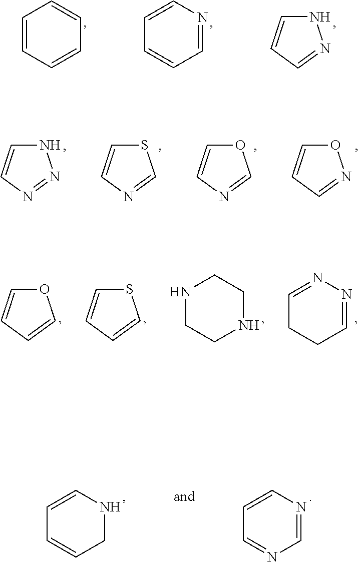

In some embodiments, A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, and [1,2,4]triazolo[1,5-a]pyrimidine.

In some embodiments, A is an optionally substituted bicyclic ring selected from the group consisting of:

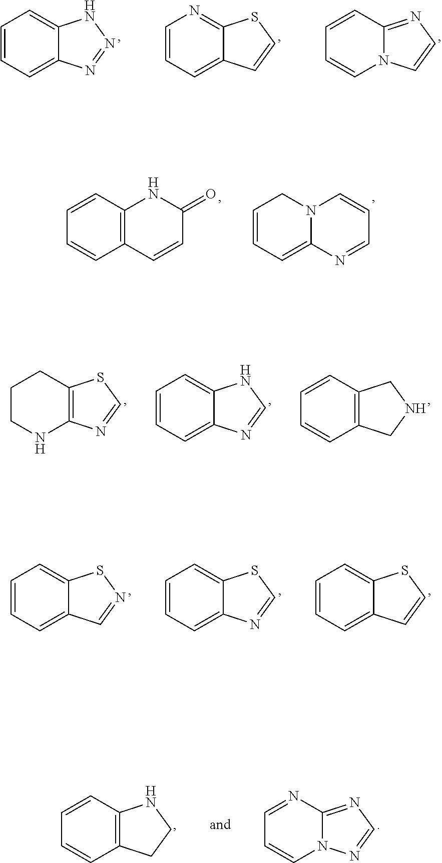

In some embodiments, A is an optionally substituted tricyclic ring selected from the group consisting of 4H-pyrido[1,2-a]thieno[2,3-d]pyrimidine, 2,4-dihydrothiochromeno[4,3-c]pyrazole, 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-]quinolone.

In some embodiments, A is an optionally substituted tricyclic ring selected from the group consisting of

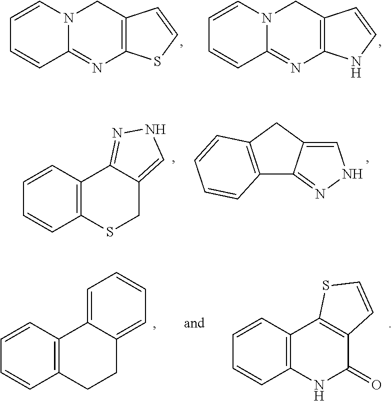

In some embodiments, A is an optionally substituted tricyclic 13-membered ring comprising 2 heteroatoms selected from the group consisting of nitrogen and sulfur.

In some embodiments, B is an optionally substituted monocyclic ring selected from the group consisting of benzene, pyridine, pyrazole, thiophene, 1,2,3-triazole, pyrimidine, pyrrole, imidazole, pyrazine, pyrrolidine, 2,3-dihydropyrrole, 2,3-dihydrothiazole, 1,2,3,4-tetrahydropyridine, 1,2,3,6-tetrahydropyridine, isoxazole, and 1,3,4-oxadiazole.

In some embodiments, B is an optionally substituted monocyclic ring selected from the group consisting of:

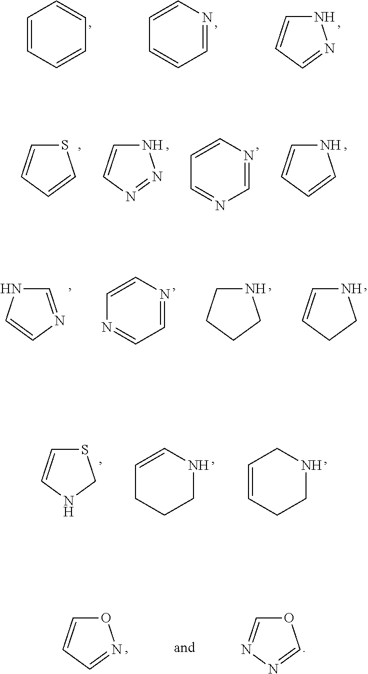

In some embodiments, B is an optionally substituted bicyclic ring selected from the group consisting of quinolone, benzo[d]imidazole, benzo[d]oxazole, indoline, thieno[2,3-d]pyrimidine, benzo[d]isothiazole, indole, naphthalene, and benzofuran.

In some embodiments, B is an optionally substituted monocyclic ring selected from the group consisting of:

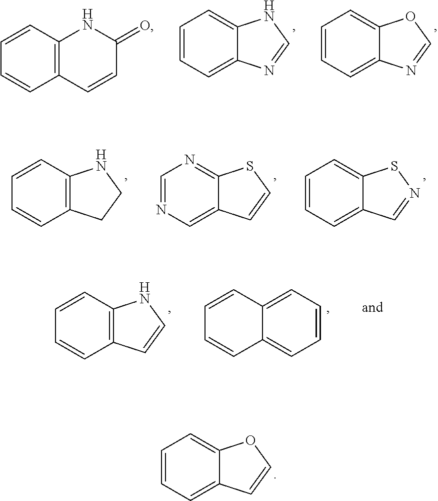

In some embodiments, B is an optionally substituted tricyclic dibenzo[b,d]furan.

In some embodiments, B is an optionally substituted

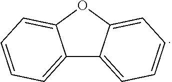 [SMILES: c1ccc2c(c1)oc1ccccc12]

In some embodiments, C is an optionally substituted monocyclic ring selected from the group consisting of benzene, isoxazole, pyridazine, thiazole, 1,3,4-oxadiazole, pyridine, pyrazole, pyrrole, thiophene, pyrimidine, morpholine, furan, and piperidine.

In some embodiments, C is an optionally substituted monocyclic ring selected from the group consisting of:

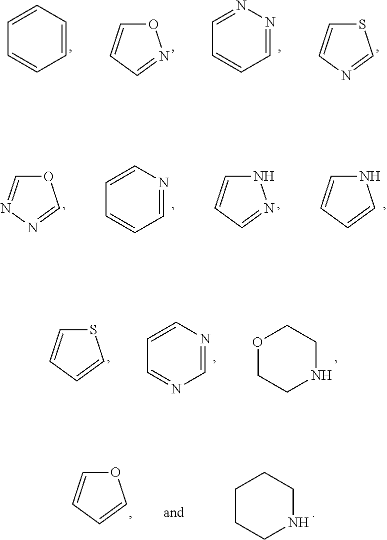

In some embodiments, C is an optionally substituted benzene. some embodiments, C is an optionally substituted

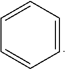

In some embodiments, C is an optionally substituted bicyclic ring selected from the group consisting of benzo[d]oxazole, imidazo[1,2-a]pyridine, quinazoline, indole, 1,2,3,4-tetrahydronaphthalene, benzo[d] imidazole and benzo[d] thiazole.

In some embodiments, C is an optionally substituted bicyclic ring selected from the group consisting of:

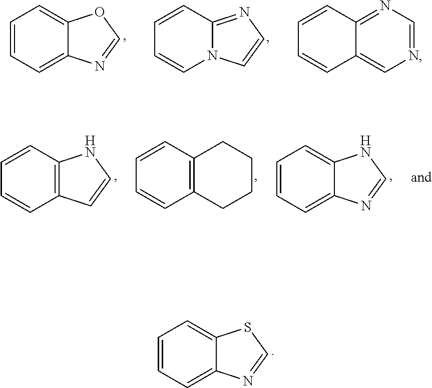

In some embodiments, L_{b} is a covalent bond, *—O—**, *—NH—**, *—NHC(O)NH—**, *—C(O)—**, *—SO₂—**, *=N—**, *—C(O)—N=**, *—OCH₂—**, *—C(O)NH—**, *—NR_{bb}C(O)—**, *—NH(CH₂)₂O—**, *—NH—R_{ba}—**, *—R_{ba}—NR_{bb}—**, *—SCH₂—**, *—SO₂CH₂—**, *—NH—N═CR_{bb}—**, *—C(O)NH—N═CH—**, *—CH₂C(O)NH—**, *—NHC(O)CH₂NH—**, *—NHC(O)OCH₂—**, or *—CH₂N(CH₃)CH₂C(O)NHC(O)NH—**.

In some embodiments, L_{b} is a covalent bond or *—C(O)NH—**.

In some embodiments, L_{b} is a covalent bond.

In some embodiments, L_{b} is *—C(O)NH—**.

In some embodiments, L_{c} is a covalent bond, *—NH—**, C₁-C₃ alkyl, *—C(O)—**, *—N═CH₂—**, *—C(O)NH—**, *—SO₂—**, *—SCH₂—**, or *—OCH₂—**.

In some embodiments, L_{c} is a covalent bond.

In some embodiments, A is optionally substituted with one or more of: —CF₃, —OCF₃, —CN, —NO₂, —N(R)₂, —OR, —SR—C(O)N(R)₂, —S(O)₂N(R)₂, —NRS(O)₂R, halo, oxo, ═NOR—NROH, C₃-C₆ cycloalkyl, —S(CH₂)F, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)F, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, B is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)N(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, C is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the disclosure features a compound represented by Formula (Ia) or a salt thereof

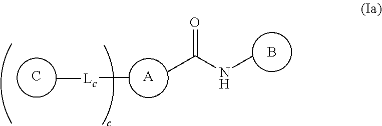

wherein

A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

c is 0 or 1;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—*, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;

each R_{cb} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (Ia) and A is an optionally substituted monocyclic ring selected from the group consisting of benzene, pyridine, thiazole, 1,2,3-triazole, pyrazole, furan, isoxazole, 4H-pyridazine, thiophene, oxazole, 2H-pyridine, thizaole, pyrrole, and pyridinone.

In some embodiments, the compound is represented by Formula (Ia) and A is an optionally substituted monocyclic ring selected from the group consisting of benzene, pyridine, thiazole, 1,2,3-triazole, pyrazole, furan, isoxazole, 4H-pyridazine, thiophene, oxazole, and 2H-pyridine.

In some embodiments, the compound is represented by Formula (Ia) and A is an optionally substituted monocyclic ring selected from the group consisting of:

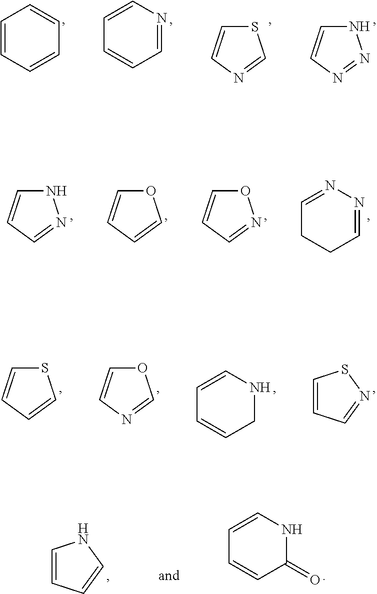

In some embodiments, the compound is represented by Formula (Ia) and A is an optionally substituted monocyclic ring selected from the group consisting of:

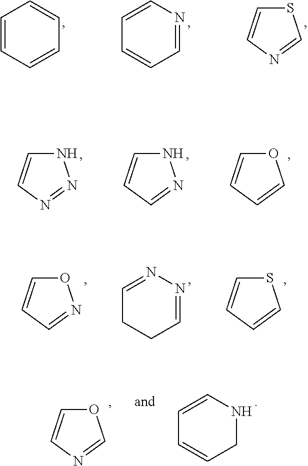

In some embodiments, the compound is represented by Formula (Ia) and A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, [1,2,4]triazolo[1,5-a]pyrimidine, naphthalene, thieno[3,2-d]imidazole, imidazo[1,5-a]pyridine, thieneo[3,2-d]pyrazole, indole, 2,3-dihydro-1H-indene, 5,6-dihydro-4H-cyclopenta[b]thiophene, and 2,3-dihydrobenzofuran.

In some embodiments, the compound is represented by Formula (Ia) and A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, and [1,2,4]triazolo[1,5-a]pyrimidine.

In some embodiments, the compound is represented by Formula (Ia) and A is an optionally substituted bicyclic ring selected from the group consisting of:

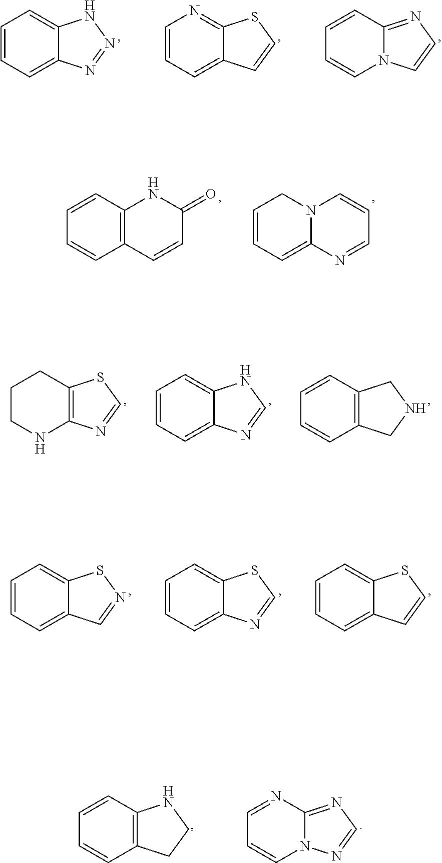

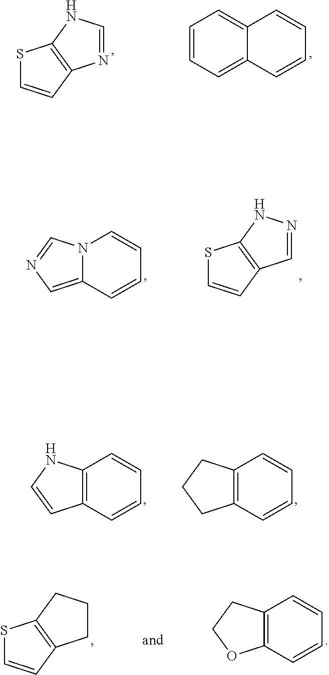

In some embodiments, the compound is represented by Formula (Ia) and A is an optionally substituted bicyclic ring selected from the group consisting of:

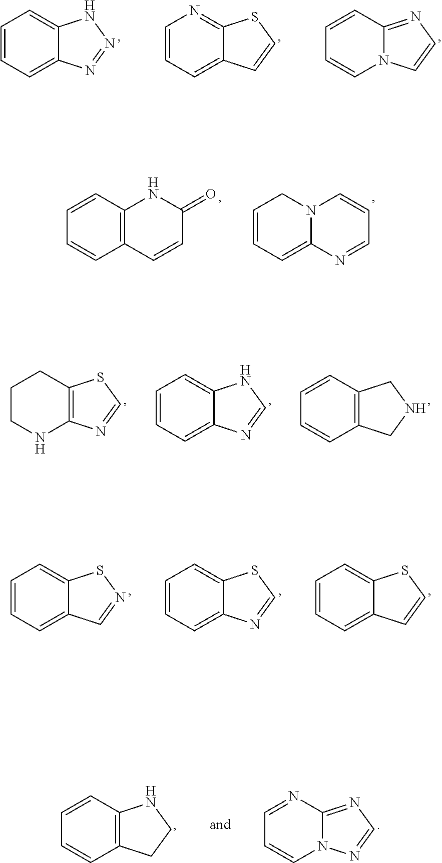

In some embodiments, the compound is represented by Formula (Ia) and A is an optionally substituted tricyclic ring selected from the group consisting of 4H-pyrido[1,2-a]thieno[2,3-d]pyrimidine, 4H-pyrido[1,2-a]pyrrolo[2,3-d]pyrimidine, 2,4-dihydrothiochromeno[4,3-c]pyrazole, 3H-benz[e]indole, and 6,7,8,9=tetrahydrothieno[2,3-c]isoquinoline.

In some embodiments, the compound is represented by Formula (Ia) and A is an optionally substituted tricyclic ring selected from the group consisting of 4H-pyrido[1,2-a]thieno[2,3-d]pyrimidine, 4H-pyrido[1,2-a]pyrrolo[2,3-d]pyrimidine, and 2,4-dihydrothiochromeno[4,3-c]pyrazole.

In some embodiments, the compound is represented by Formula (Ia) and A is an optionally substituted tricyclic ring selected from the group consisting of:

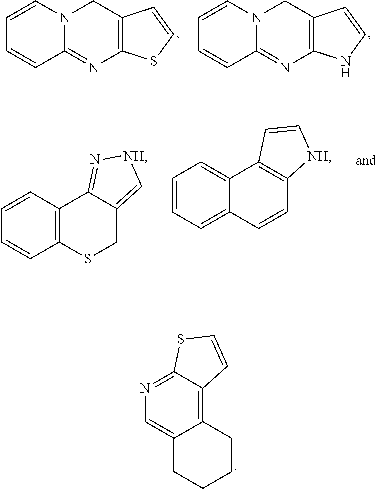

In some embodiments, the compound is represented by Formula (Ia) and A is an optionally substituted tricyclic ring selected from the oup consisting of:

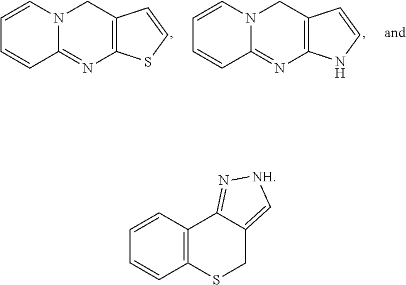

In some embodiments, the compound is represented by Formula (Ia) and B is an optionally substituted monocyclic ring selected from the group consisting of benzene, pyridine, pyrazole, thiophene, pyrimidine, thiazole, isoxazole, imidazole, 1,2,4-triazole, 1,3,4-triazole, pyridine-2-one, and pyran-2-one.

In some embodiments, the compound is represented by Formula (Ia) and B is an optionally substituted monocyclic ring selected from the group consisting of benzene, pyridine, pyrazole, and thiophene.

In some embodiments, the compound is represented by Formula (Ia) and B is an optionally substituted monocyclic ring selected from the group consisting of:

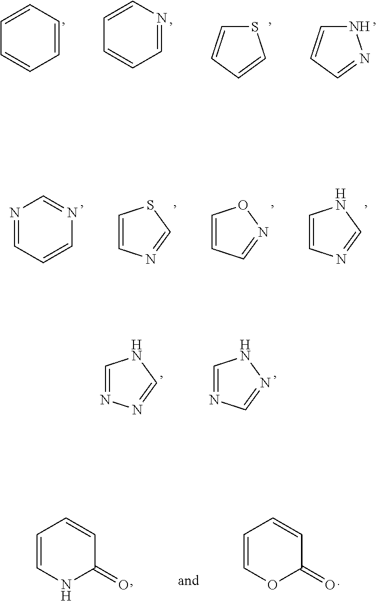

In some embodiments, the compound is represented by Formula (Ia) and B is an optionally substituted monocyclic ring selected from the group consisting of:

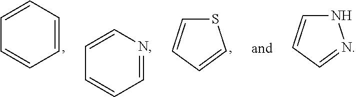

In some embodiments, the compound is represented by Formula (Ia) and B is an optionally substituted bicyclic ring selected from the group consisting of indoline, quinolone, benzo[d]imidazole, benzo[d]oxazole, benzo[b]thiophene, benzo[d]thiazole, naphthalene, quinolone, 4H-chromen-4-one, 5,6-dihydro-4H-cyclopenta[b]thiophene, 4,5,6,7-tetrahydrobenzo[b]thiophene, and 7,8-2H-1-quinoline-2,5(6H)-dione.

In some embodiments, the compound is represented by Formula (Ia) and B is an optionally substituted bicyclic ring selected from the group consisting of indoline, quinolone, benzo[d]imidazole, and benzo[d]oxazole.

In some embodiments, the compound is represented by Formula (Ia) and B is an optionally substituted bicyclic ring selected from the group consisting of:

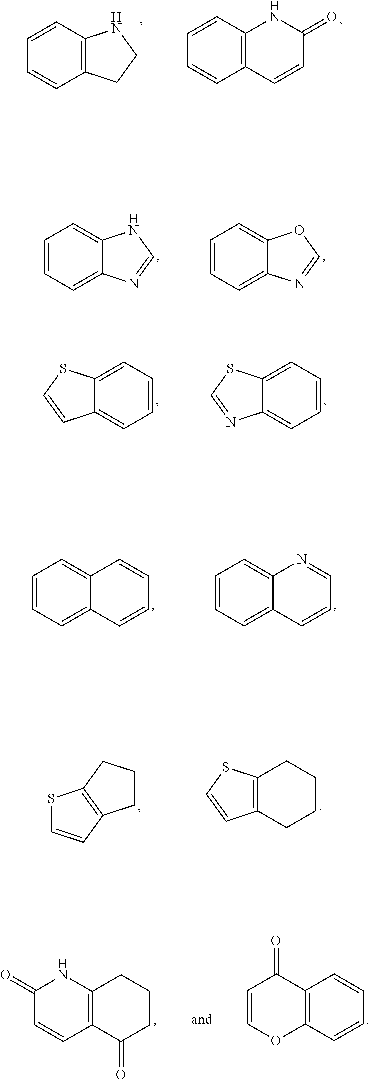

In some embodiments, the compound is represented by Formula (Ia) and B is an optionally substituted bicyclic ring selected from the group consisting of:

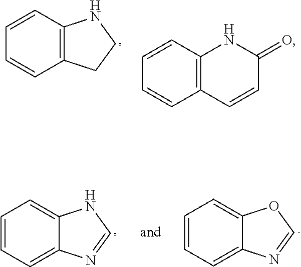

In some embodiments, the compound is represented by Formula (Ia) and B is an optionally substituted dibenzo[b,d]furan.

In some embodiments, the compound is represented by Formula (Ia) and B is an optionally substituted

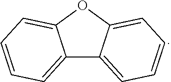 [SMILES: c1ccc2c(c1)oc1ccccc12]

In some embodiments, the compound is represented by Formula (Ia) and L is selected from the group consisting of a covalent bond, *—NH—**, and C₁-C₃ alkyl.

In some embodiments, the compound is represented by Formula (Ia) and C is an optionally substituted monocyclic ring selected from the group consisting of benzene, isoxazole, pyridazine, thiazole, pyrazole, imidazole, pyrimidine, pyridine, morpholine, and imidazolidine-2,4-dione.

In some embodiments, the compound is represented by Formula (Ia) and C is an optionally substituted monocyclic ring selected from the group consisting of benzene, isoxazole, pyridazine, and thiazole.

In some embodiments, the compound is represented by Formula (Ia) and C is an optionally substituted monocyclic ring selected from the group consisting of:

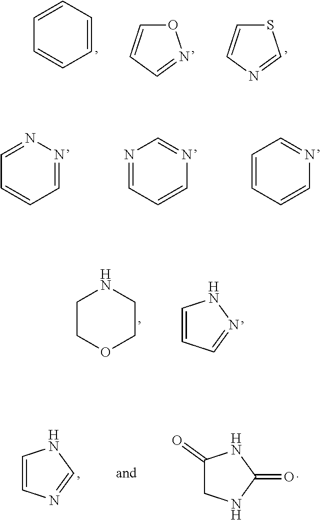

In some embodiments, the compound is represented by Formula (Ia) and C is an optionally substituted monocyclic ring selected from the group consisting of:

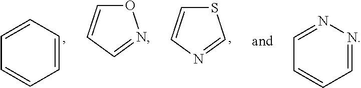

In some embodiments, the compound is represented by Formula (Ia) and C is an optionally substituted benzo[d]oxazole.

In some embodiments, the compound is represented by Formula (Ia) and C is an optionally substituted

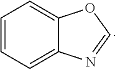

In some embodiments, the compound is represented by Formula (Ia) and A, B, or both A and B is an optionally substituted benzene.

In some embodiments, the compound is represented by Formula (Ia) and A, B, or both A and B is an optionally substituted

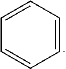

In some embodiments, the compound is represented by Formula (Ia) and A or B is an optionally substituted thiophene.

In some embodiments, the compound is represented by Formula(Ia) and A or B is an optionally substituted

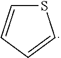

In some embodiments, the compound is represented by Formula (Ia) and c is 0.

In some embodiments, the compound represented by Formula (Ia) is a compound or salt thereof of Table 1 below.

In some embodiments, the compound is a compound or a salt thereof of Table 1 below:

TABLE 1
AHR antagonists

| Compd. | |
|---|---|
| No. | Structure |
| 1 | 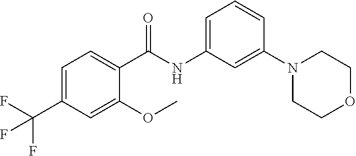 [SMILES: COc1cc(C(F)(F)F)ccc1C(=O)Nc1cccc(N2CCOCC2)c1] |
| 2 | 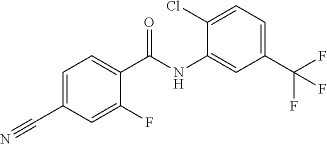 [SMILES: N#Cc1ccc(C(=O)Nc2cc(C(F)(F)F)ccc2Cl)c(F)c1] |
| 3 | 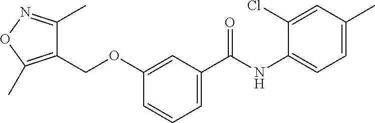 [SMILES: Cc1ccc(NC(=O)c2cccc(OCc3c(C)noc3C)c2)c(Cl)c1] |
| 4 | 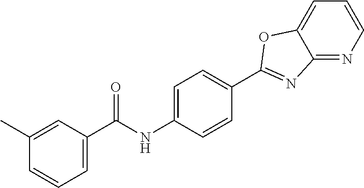 [SMILES: Cc1cccc(C(=O)Nc2ccc(-c3nc4ncccc4o3)cc2)c1] |
| 5 | 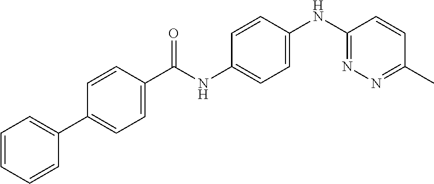 [SMILES: Cc1ccc(Nc2ccc(NC(=O)c3ccc(-c4ccccc4)cc3)cc2)nn1] |
| 6 | 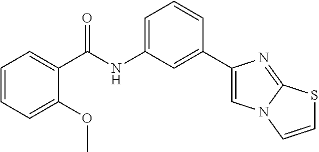 [SMILES: COc1ccccc1C(=O)Nc1cccc(-c2cn3ccsc3n2)c1] |
| 7 | 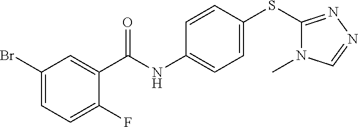 [SMILES: Cn1cnnc1Sc1ccc(NC(=O)c2cc(Br)ccc2F)cc1] |
| 8 | 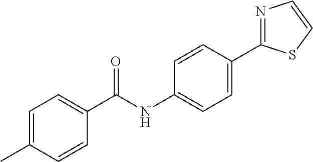 [SMILES: Cc1ccc(C(=O)Nc2ccc(-c3nccs3)cc2)cc1] |
| 9 | 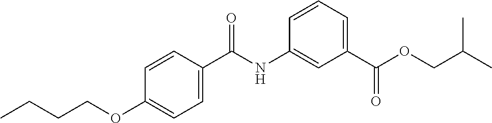 [SMILES: CCCCOc1ccc(C(=O)Nc2cccc(C(=O)OCC(C)C)c2)cc1] |
| 10 | 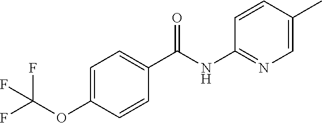 [SMILES: Cc1ccc(NC(=O)c2ccc(OC(F)(F)F)cc2)nc1] |
| 11 | 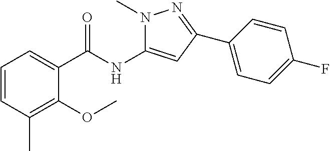 [SMILES: COc1c(C)cccc1C(=O)Nc1cc(-c2ccc(F)cc2)nn1C] |
| 12 | 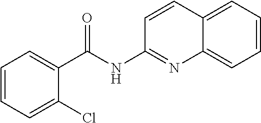 [SMILES: O=C(Nc1ccc2ccccc2n1)c1ccccc1Cl] |
| 13 | 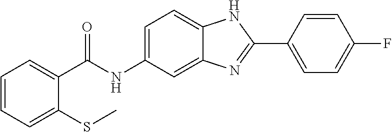 [SMILES: CSc1ccccc1C(=O)Nc1ccc2[nH]c(-c3ccc(F)cc3)nc2c1] |
| 14 | 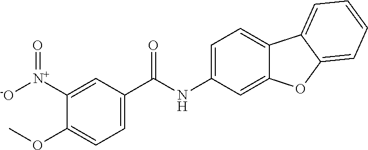 |
| 15 | 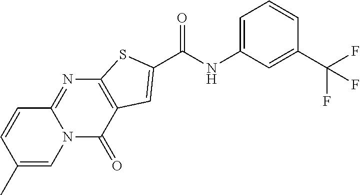 [SMILES: Cc1ccc2nc3sc(C(=O)Nc4cccc(C(F)(F)F)c4)cc3c(=O)n2c1] |
| 16 | 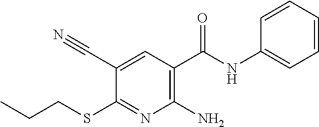 [SMILES: CCCSc1nc(N)c(C(=O)Nc2ccccc2)cc1C#N] |
| 17 | 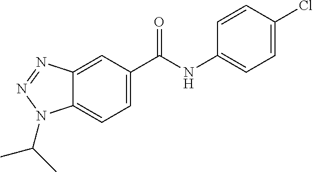 [SMILES: CC(C)n1nnc2cc(C(=O)Nc3ccc(Cl)cc3)ccc21] |
| 18 | 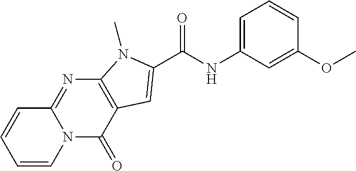 [SMILES: COc1cccc(NC(=O)c2cc3c(=O)n4ccccc4nc3n2C)c1] |
| 19 | 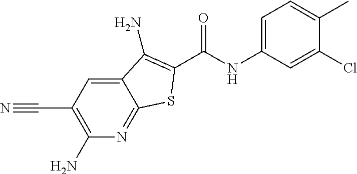 [SMILES: Cc1ccc(NC(=O)c2sc3nc(N)c(C#N)cc3c2N)cc1Cl] |
| 20 | 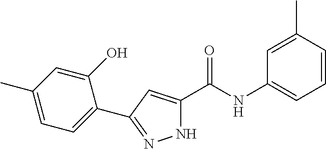 [SMILES: Cc1cccc(NC(=O)c2cc(-c3ccc(C)cc3O)n[nH]2)c1] |
| 21 | 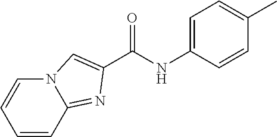 [SMILES: Cc1ccc(NC(=O)c2cn3ccccc3n2)cc1] |
| 22 | 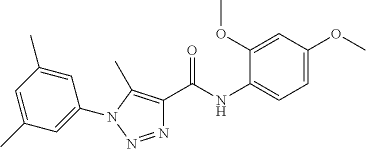 |
| 23 | 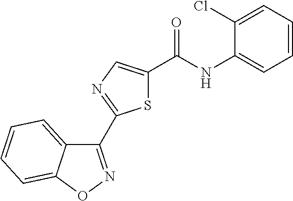 [SMILES: O=C(Nc1ccccc1Cl)c1cnc(-c2noc3ccccc23)s1] |
| 24 | 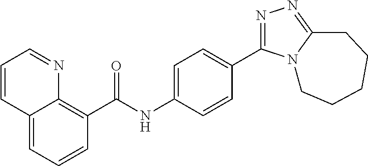 [SMILES: O=C(Nc1ccc(-c2nnc3n2CCCCC3)cc1)c1cccc2cccnc12] |
| 25 | 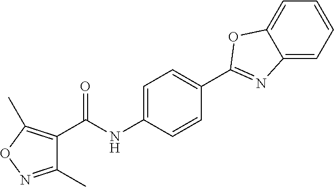 [SMILES: Cc1noc(C)c1C(=O)Nc1ccc(-c2nc3ccccc3o2)cc1] |
| 26 | 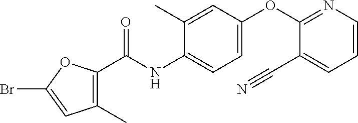 [SMILES: Cc1cc(Oc2ncccc2C#N)ccc1NC(=O)c1oc(Br)cc1C] |
| 27 | 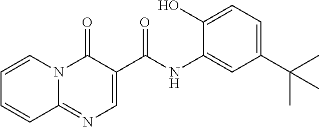 [SMILES: CC(C)(C)c1ccc(O)c(NC(=O)c2cnc3ccccn3c2=O)c1] |
| 28 | 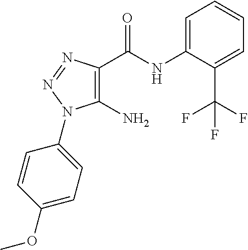 [SMILES: COc1ccc(-n2nnc(C(=O)Nc3ccccc3C(F)(F)F)c2N)cc1] |
| 29 | 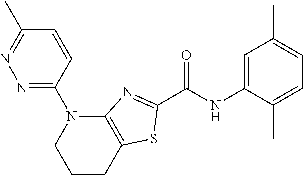 [SMILES: Cc1ccc(C)c(NC(=O)c2nc3c(s2)CCCN3c2ccc(C)nn2)c1] |
| 30 | 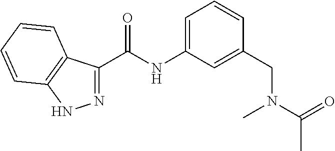 [SMILES: CC(=O)N(C)Cc1cccc(NC(=O)c2n[nH]c3ccccc23)c1] |
| 31 | 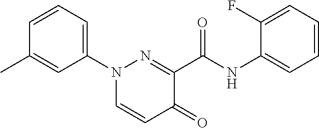 [SMILES: Cc1cccc(-n2ccc(=O)c(C(=O)Nc3ccccc3F)n2)c1] |
| 32 | 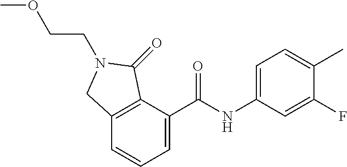 [SMILES: COCCN1Cc2cccc(C(=O)Nc3ccc(C)c(F)c3)c2C1=O] |
| 33 | 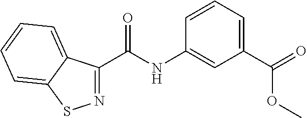 [SMILES: COC(=O)c1cccc(NC(=O)c2nsc3ccccc23)c1] |
| 34 | 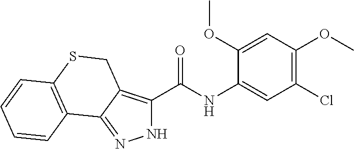 [SMILES: COc1cc(OC)c(NC(=O)c2[nH]nc3c2CSc2ccccc2-3)cc1Cl] |
| 35 | 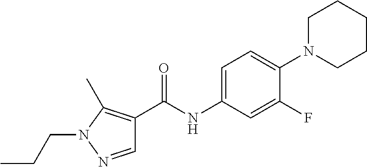 [SMILES: CCCn1ncc(C(=O)Nc2ccc(N3CCCCC3)c(F)c2)c1C] |
| 36 | 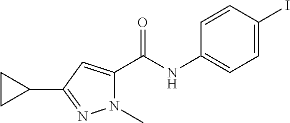 [SMILES: Cn1nc(C2CC2)cc1C(=O)Nc1ccc(I)cc1] |
| 37 | 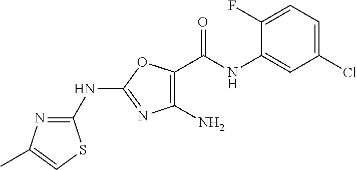 [SMILES: Cc1csc(Nc2nc(N)c(C(=O)Nc3cc(Cl)ccc3F)o2)n1] |
| 38 | 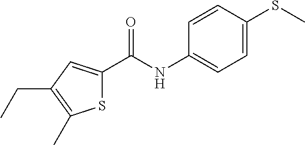 [SMILES: CCc1cc(C(=O)Nc2ccc(SC)cc2)sc1C] |
| 39 | 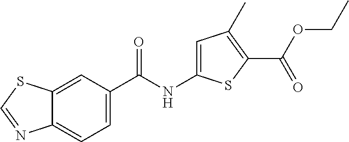 [SMILES: CCOC(=O)c1sc(NC(=O)c2ccc3ncsc3c2)cc1C] |
| 40 | 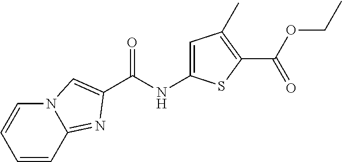 [SMILES: CCOC(=O)c1sc(NC(=O)c2cn3ccccc3n2)cc1C] |
| 41 | 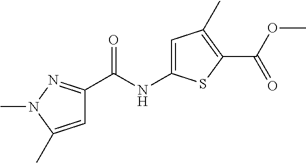 |
| 42 | 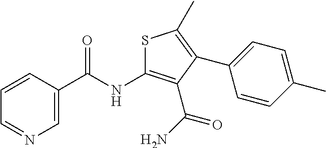 [SMILES: Cc1ccc(-c2c(C)sc(NC(=O)c3cccnc3)c2C(N)=O)cc1] |
| 43 | 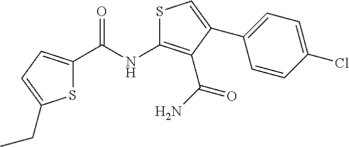 [SMILES: CCc1ccc(C(=O)Nc2scc(-c3ccc(Cl)cc3)c2C(N)=O)s1] |
| 44 | 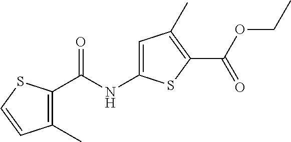 [SMILES: CCOC(=O)c1sc(NC(=O)c2sccc2C)cc1C] |
| 45 | 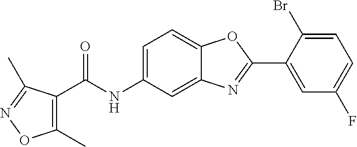 [SMILES: Cc1noc(C)c1C(=O)Nc1ccc2oc(-c3cc(F)ccc3Br)nc2c1] |
| 46 | 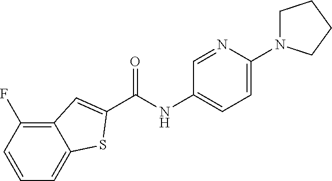 [SMILES: O=C(Nc1ccc(N2CCCC2)nc1)c1cc2c(F)cccc2s1] |
| 47 | 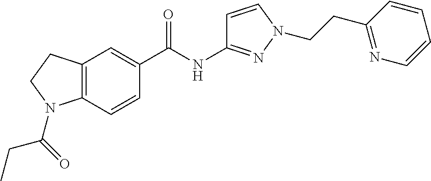 [SMILES: CCC(=O)N1CCc2cc(C(=O)Nc3ccn(CCc4ccccn4)n3)ccc21] |
| 48 | 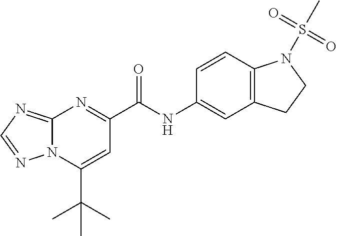 [SMILES: CC(C)(C)c1cc(C(=O)Nc2ccc3c(c2)CCN3S(C)(=O)=O)nc2ncnn12] |
| 49 | 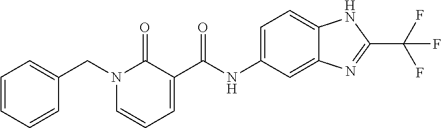 [SMILES: O=C(Nc1ccc2[nH]c(C(F)(F)F)nc2c1)c1cccn(Cc2ccccc2)c1=O] |

In some embodiments, the compound represented by Formula (Ia) is a compound or salt thereof of Table 1A below.

In some embodiments, the compound is a compound or a salt thereof of Table 1A below:

TABLE 1A
AHR antagonists

| Compd. | |
|---|---|
| No. | Structure |
| 1A | 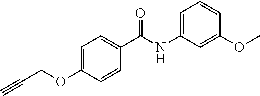 [SMILES: C#CCOc1ccc(C(=O)Nc2cccc(OC)c2)cc1] |
| 2A | 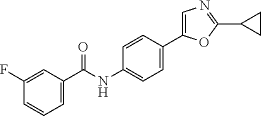 [SMILES: O=C(Nc1ccc(-c2cnc(C3CC3)o2)cc1)c1cccc(F)c1] |
| 3A | 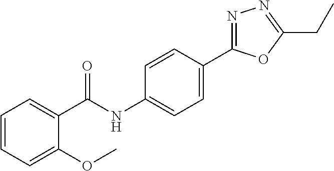 [SMILES: CCc1nnc(-c2ccc(NC(=O)c3ccccc3OC)cc2)o1] |
| 4A | 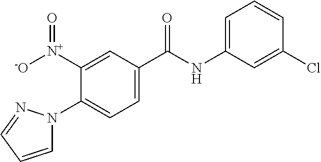 |
| 5A | 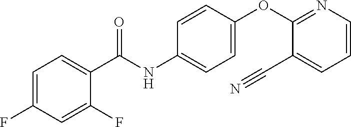 [SMILES: N#Cc1cccnc1Oc1ccc(NC(=O)c2ccc(F)cc2F)cc1] |
| 6A | 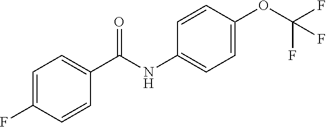 [SMILES: O=C(Nc1ccc(OC(F)(F)F)cc1)c1ccc(F)cc1] |
| 7A | 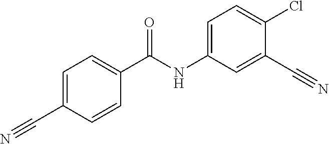 [SMILES: N#Cc1ccc(C(=O)Nc2ccc(Cl)c(C#N)c2)cc1] |
| 8A | 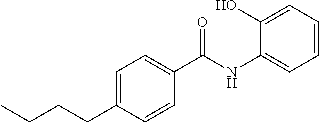 [SMILES: CCCCc1ccc(C(=O)Nc2ccccc2O)cc1] |
| 9A | 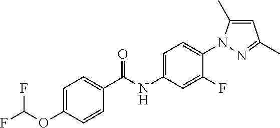 [SMILES: Cc1cc(C)n(-c2ccc(NC(=O)c3ccc(OC(F)F)cc3)cc2F)n1] |
| 10A | 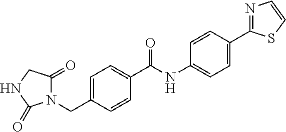 [SMILES: O=C(Nc1ccc(-c2nccs2)cc1)c1ccc(CN2C(=O)CNC2=O)cc1] |
| 11A | 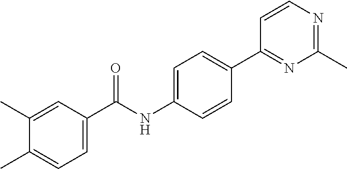 |
| 12A | 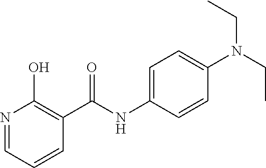 [SMILES: CCN(CC)c1ccc(NC(=O)c2cccnc2O)cc1] |
| 13A | 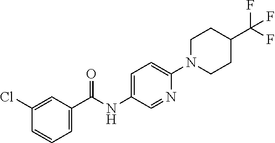 [SMILES: O=C(Nc1ccc(N2CCC(C(F)(F)F)CC2)nc1)c1cccc(Cl)c1] |
| 14A | 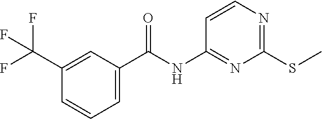 [SMILES: CSc1nccc(NC(=O)c2cccc(C(F)(F)F)c2)n1] |
| 15A | 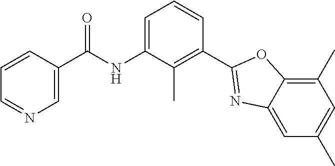 [SMILES: Cc1cc(C)c2oc(-c3cccc(NC(=O)c4cccnc4)c3C)nc2c1] |
| 16A | 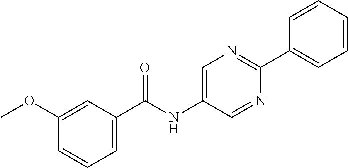 [SMILES: COc1cccc(C(=O)Nc2cnc(-c3ccccc3)nc2)c1] |
| 17A | 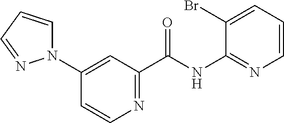 [SMILES: O=C(Nc1ncccc1Br)c1cc(-n2cccn2)ccn1] |
| 18A | 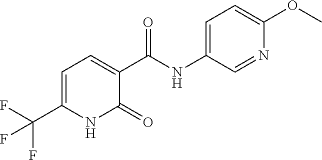 [SMILES: COc1ccc(NC(=O)c2ccc(C(F)(F)F)[nH]c2=O)cn1] |
| 19A | 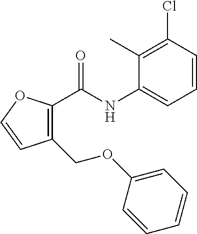 [SMILES: Cc1c(Cl)cccc1NC(=O)c1occc1COc1ccccc1] |
| 20A | 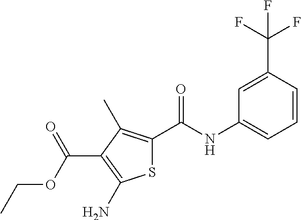 [SMILES: CCOC(=O)c1c(N)sc(C(=O)Nc2cccc(C(F)(F)F)c2)c1C] |
| 21A | 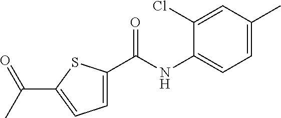 [SMILES: CC(=O)c1ccc(C(=O)Nc2ccc(C)cc2Cl)s1] |
| 22A | 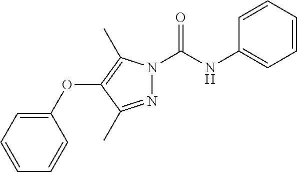 [SMILES: Cc1nn(C(=O)Nc2ccccc2)c(C)c1Oc1ccccc1] |
| 23A | 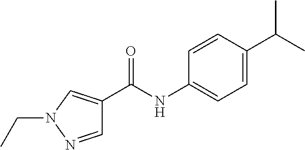 [SMILES: CCn1cc(C(=O)Nc2ccc(C(C)C)cc2)cn1] |
| 24A | 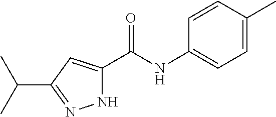 [SMILES: Cc1ccc(NC(=O)c2cc(C(C)C)n[nH]2)cc1] |
| 25A | 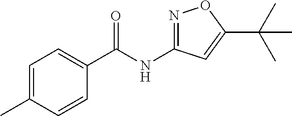 [SMILES: Cc1ccc(C(=O)Nc2cc(C(C)(C)C)on2)cc1] |
| 26A | 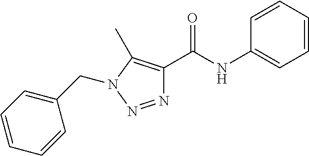 [SMILES: Cc1c(C(=O)Nc2ccccc2)nnn1Cc1ccccc1] |
| 27A | 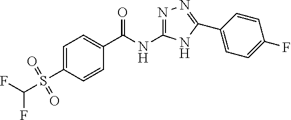 [SMILES: O=C(Nc1nnc(-c2ccc(F)cc2)[nH]1)c1ccc(S(=O)(=O)C(F)F)cc1] |
| 28A | 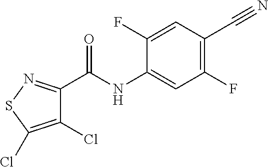 [SMILES: N#Cc1cc(F)c(NC(=O)c2nsc(Cl)c2Cl)cc1F] |
| 29A | 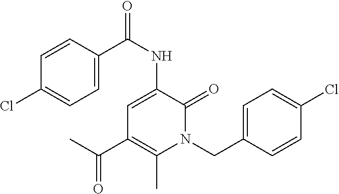 [SMILES: CC(=O)c1cc(NC(=O)c2ccc(Cl)cc2)c(=O)n(Cc2ccc(Cl)cc2)c1C] |
| 30A | 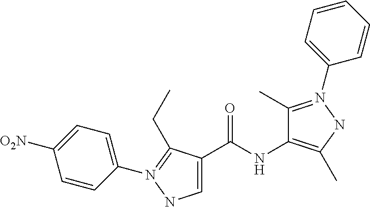 |
| 31A | 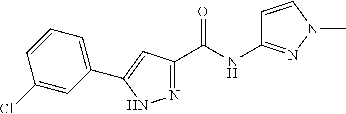 [SMILES: Cn1ccc(NC(=O)c2cc(-c3cccc(Cl)c3)[nH]n2)n1] |
| 32A | 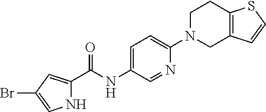 [SMILES: O=C(Nc1ccc(N2CCc3sccc3C2)nc1)c1cc(Br)c[nH]1] |
| 33A | 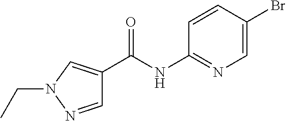 [SMILES: CCn1cc(C(=O)Nc2ccc(Br)cn2)cn1] |
| 34A | 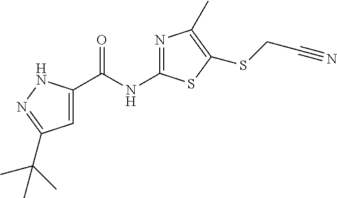 [SMILES: Cc1nc(NC(=O)c2cc(C(C)(C)C)n[nH]2)sc1SCC#N] |
| 35A | 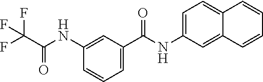 [SMILES: O=C(Nc1ccc2ccccc2c1)c1cccc(NC(=O)C(F)(F)F)c1] |
| 36A | 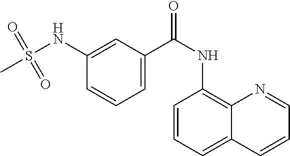 [SMILES: CS(=O)(=O)Nc1cccc(C(=O)Nc2cccc3cccnc23)c1] |
| 37A | 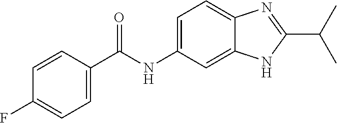 [SMILES: CC(C)c1nc2ccc(NC(=O)c3ccc(F)cc3)cc2[nH]1] |
| 38A | 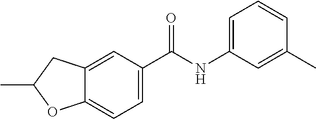 |
| 39A | 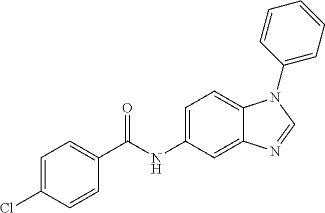 [SMILES: O=C(Nc1ccc2c(c1)ncn2-c1ccccc1)c1ccc(Cl)cc1] |
| 40A | 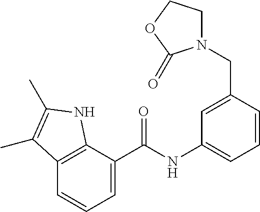 [SMILES: Cc1[nH]c2c(C(=O)Nc3cccc(CN4CCOC4=O)c3)cccc2c1C] |
| 41A | 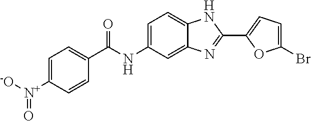 |
| 42A | 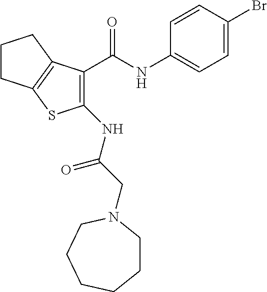 [SMILES: O=C(CN1CCCCCC1)Nc1sc2c(c1C(=O)Nc1ccc(Br)cc1)CCC2] |
| 43A | 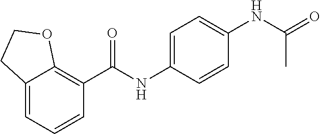 [SMILES: CC(=O)Nc1ccc(NC(=O)c2cccc3c2OCC3)cc1] |
| 44A | 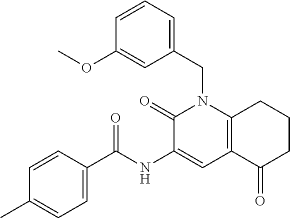 [SMILES: COc1cccc(Cn2c3c(cc(NC(=O)c4ccc(C)cc4)c2=O)C(=O)CCC3)c1] |
| 45A | 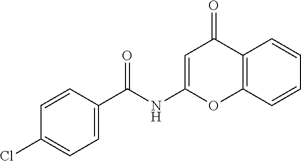 [SMILES: O=C(Nc1cc(=O)c2ccccc2o1)c1ccc(Cl)cc1] |
| 46A | 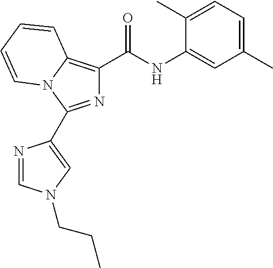 [SMILES: CCCn1cnc(-c2nc(C(=O)Nc3cc(C)ccc3C)c3ccccn23)c1] |
| 47A | 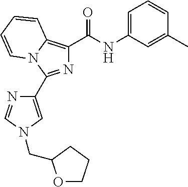 [SMILES: Cc1cccc(NC(=O)c2nc(-c3cn(CC4CCCO4)cn3)n3ccccc23)c1] |
| 48A | 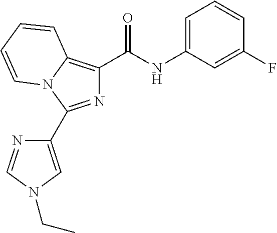 [SMILES: CCn1cnc(-c2nc(C(=O)Nc3cccc(F)c3)c3ccccn23)c1] |
| 49A | 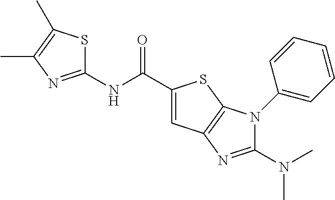 [SMILES: Cc1nc(NC(=O)c2cc3nc(N(C)C)n(-c4ccccc4)c3s2)sc1C] |
| 50A | 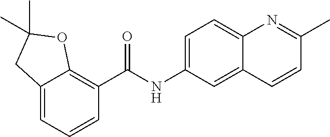 |
| 51A | 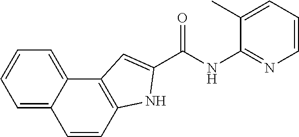 [SMILES: Cc1cccnc1NC(=O)c1cc2c(ccc3ccccc32)[nH]1] |
| 52A | 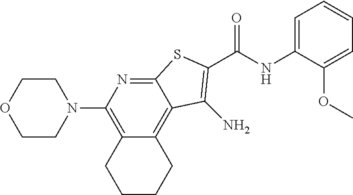 [SMILES: COc1ccccc1NC(=O)c1sc2nc(N3CCOCC3)c3c(c2c1N)CCCC3] |

In some embodiments, the compound represented by Formula (Ia) is a compound or salt thereof of Table 1B below.

In some embodiments, the compound is a compound or a salt thereof of Table 1 below:

TABLE 1B
AHR antagonists

| Compd. | |
|---|---|
| No. | Structure |
| 1B | 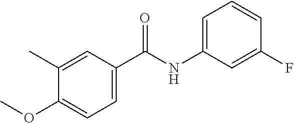 [SMILES: COc1ccc(C(=O)Nc2cccc(F)c2)cc1C] |
| 2B | 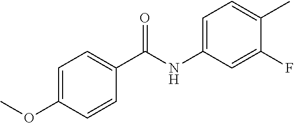 [SMILES: COc1ccc(C(=O)Nc2ccc(C)c(F)c2)cc1] |
| 3B | 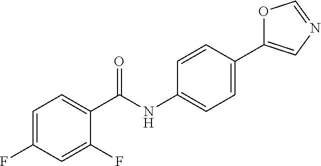 [SMILES: O=C(Nc1ccc(-c2cnco2)cc1)c1ccc(F)cc1F] |
| 4B | 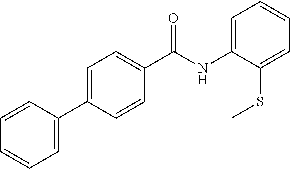 [SMILES: CSc1ccccc1NC(=O)c1ccc(-c2ccccc2)cc1] |
| 5B | 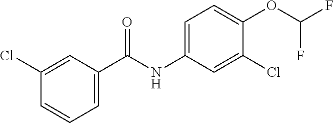 [SMILES: O=C(Nc1ccc(OC(F)F)c(Cl)c1)c1cccc(Cl)c1] |
| 6B | 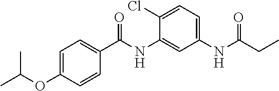 [SMILES: CCC(=O)Nc1ccc(Cl)c(NC(=O)c2ccc(OC(C)C)cc2)c1] |
| 7B | 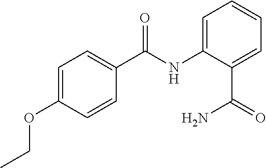 [SMILES: CCOc1ccc(C(=O)Nc2ccccc2C(N)=O)cc1] |
| 8B | 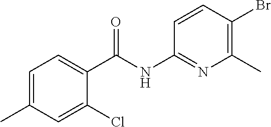 [SMILES: Cc1ccc(C(=O)Nc2ccc(Br)c(C)n2)c(Cl)c1] |
| 9B | 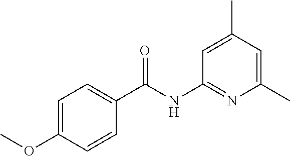 |
| 10B | 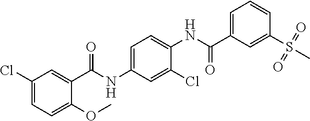 [SMILES: COc1ccc(Cl)cc1C(=O)Nc1ccc(NC(=O)c2cccc(S(C)(=O)=O)c2)c(Cl)c1] |
| 11B | 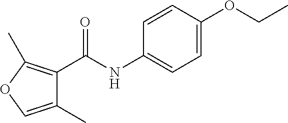 [SMILES: CCOc1ccc(NC(=O)c2c(C)coc2C)cc1] |
| 12B | 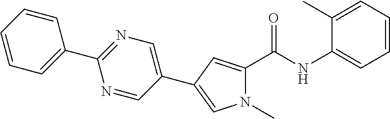 [SMILES: Cc1ccccc1NC(=O)c1cc(-c2cnc(-c3ccccc3)nc2)cn1C] |
| 13B | 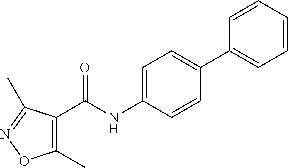 [SMILES: Cc1noc(C)c1C(=O)Nc1ccc(-c2ccccc2)cc1] |
| 14B | 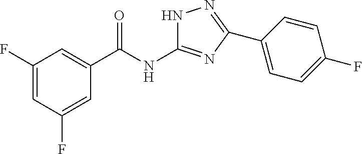 [SMILES: O=C(Nc1nc(-c2ccc(F)cc2)n[nH]1)c1cc(F)cc(F)c1] |
| 15B | 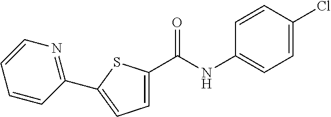 [SMILES: O=C(Nc1ccc(Cl)cc1)c1ccc(-c2ccccn2)s1] |
| 16B | 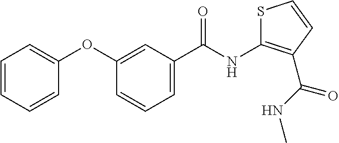 [SMILES: CNC(=O)c1ccsc1NC(=O)c1cccc(Oc2ccccc2)c1] |
| 17B | 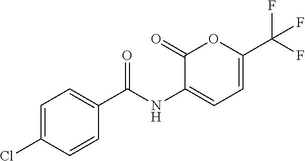 [SMILES: O=C(Nc1ccc(C(F)(F)F)oc1=O)c1ccc(Cl)cc1] |
| 18B | 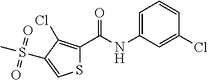 [SMILES: CS(=O)(=O)c1csc(C(=O)Nc2cccc(Cl)c2)c1Cl] |
| 19B | 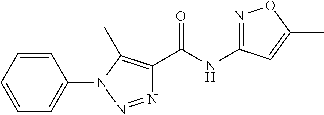 [SMILES: Cc1cc(NC(=O)c2nnn(-c3ccccc3)c2C)no1] |
| 20B | 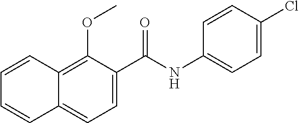 [SMILES: COc1c(C(=O)Nc2ccc(Cl)cc2)ccc2ccccc12] |
| 21B | 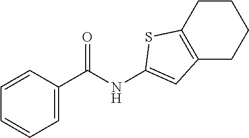 [SMILES: O=C(Nc1cc2c(s1)CCCC2)c1ccccc1] |
| 22B | 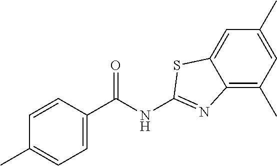 |
| 23B | 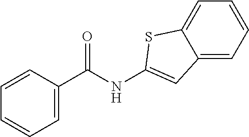 [SMILES: O=C(Nc1cc2ccccc2s1)c1ccccc1] |
| 24B | 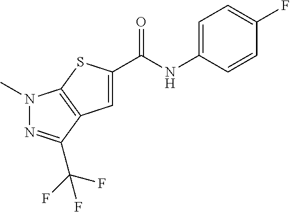 [SMILES: Cn1nc(C(F)(F)F)c2cc(C(=O)Nc3ccc(F)cc3)sc21] |
| 25B | 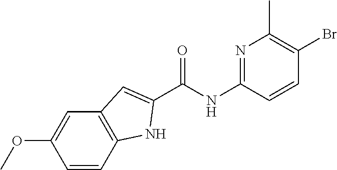 [SMILES: COc1ccc2[nH]c(C(=O)Nc3ccc(Br)c(C)n3)cc2c1] |
| 26B | 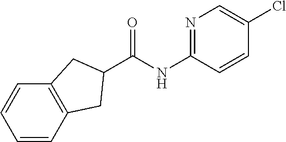 [SMILES: O=C(Nc1ccc(Cl)cn1)C1Cc2ccccc2C1] |
| 27B | 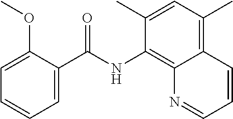 [SMILES: COc1ccccc1C(=O)Nc1c(C)cc(C)c2cccnc12] |
| 28B | 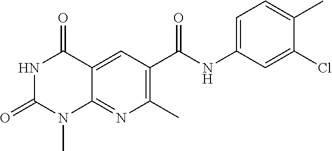 [SMILES: Cc1ccc(NC(=O)c2cc3c(=O)[nH]c(=O)n(C)c3nc2C)cc1Cl] |
| 29B | 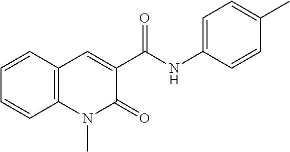 [SMILES: Cc1ccc(NC(=O)c2cc3ccccc3n(C)c2=O)cc1] |
| 30B | 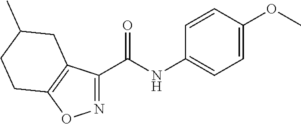 [SMILES: COc1ccc(NC(=O)c2noc3c2CC(C)CC3)cc1] |
| 31B | 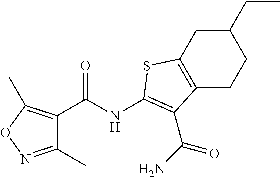 [SMILES: CCC1CCc2c(sc(NC(=O)c3c(C)noc3C)c2C(N)=O)C1] |
| 32B | 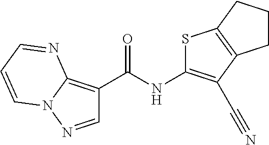 [SMILES: N#Cc1c(NC(=O)c2cnn3cccnc23)sc2c1CCC2] |

In some embodiments, the disclosure features a compound wherein A is

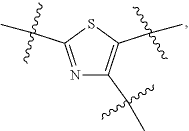 [SMILES: *c1nc(*)c(*)s1]

in which each [symbol] independently denotes the linkage between A and hydrogen, -Lb-B,-Lc-C, or a substituent.

In some embodiments, the disclosure features a compound represented by Formula (Ib) or a salt thereof

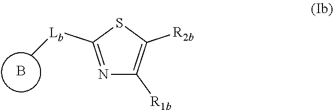

wherein

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O) —**, *—SO₂—**, *=N—**, *—N=**, *=N— *—C**(O)—N=**, *—O—R_{ba}—**, *—Ra—O—**, *—C(O)NR_{bb}—**, *—NR_{bb}C(O)—**, *—NR_{bb}—R_{ba}—(O)—**, *—O—R_{ba}—NR_{ba}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{bb}, —NR_{bb}—R_{ba}—**, in which * denotes the linkage between L_{b} and a thiazole carbon and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R_{1b} is hydrogen or -L_{c}-C;

R_{2b} is hydrogen, an optionally substituted pyrazole ring, or CONR_{3b}R_{4b}, wherein each R_{3b} and R_{4b} is independently hydrogen or C₁-C₆ alkyl;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L and a thiazole carbon and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl; and

R_{1b} and R_{2b} are not both hydrogen.

In some embodiments, the compound is represented by Formula (Ib) and R_{1b} is hydrogen.

In some embodiments, the compound is represented by Formula (Ib) and B is an optionally substituted monocyclic ring selected from the group consisting of benzene, pyridine, 2,3-dihydropyrrole, 1,2,3-triazole, pyrrolidine, thiophene, piperazine, imidazole, tetrazole, pyrrolidin-2-one, and 1,2-dihydro-3H-pyrrol-3-one.

In some embodiments, the compound is represented by Formula (Ib) and B is an optionally substituted monocyclic ring selected from the group consisting of benzene, pyridine, 2,3-dihydropyrrole, 1,2,3-triazole, pyrrolidine, and thiophene.

In some embodiments, the compound is represented by Formula (Ib) and B is an optionally substituted monocyclic ring selected from the group consisting of.

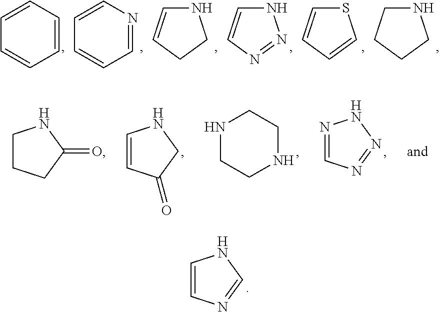

In some embodiments, the compound is represented by Formula (Ib) and B is an optionally substituted monocyclic ring selected from the group consisting of:

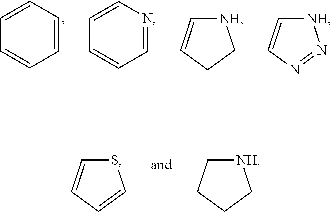

In some embodiments, the compound is represented by Formula (Ib) and B is an optionally substituted bicyclic ring selected from the group consisting of benzo[d]isooxazole, 2,3-dihydrobenzofuran, and imidazo[1,2-a]pyridine.

In some embodiments, the compound is represented by Formula (Ib) and B is an optionally substituted monocyclic ring selected from the group consisting of

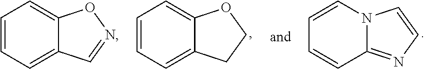

In some embodiments, the compound is represented by Formula (Ib) and L_{b} is selected from the group consisting of a covalent bond, *—NH—**, and *—NR_{bb}C(O)—**.

In some embodiments, the compound is represented by Formula (Ib) and L_{b} is a covalent bond.

In some embodiments, the compound is represented by Formula (Ib), R_{1b} is -L_{c}-C and L_{c} is a covalent bond.

In some embodiments, the compound is represented by Formula (Ib), R_{1b} is -L_{c}-C and C is an optionally substituted monocyclic ring selected from the group consisting of benzene, pyridine, pyrrole, pyrazole, 1,3,4-oxadiazole, 4H-1,2,4-triazole, thiophene, 1H-1,2,4-triazole, 1,2,3,4-tetrahydropyrimidine, and pyrimidine-2,4(1H,3H)-dione.

In some embodiments, the compound is represented by Formula (Ib), R_{1b} is -L_{c}-C and C is an optionally substituted monocyclic ring selected from the group consisting of benzene, pyridine, pyrrole, pyrazole, and 1,3,4-oxadiazole.

In some embodiments, the compound is represented by Formula (Ib), R_{1b} is -L_{c}-C and C is an optionally substituted monocyclic ring selected from the group consisting of:

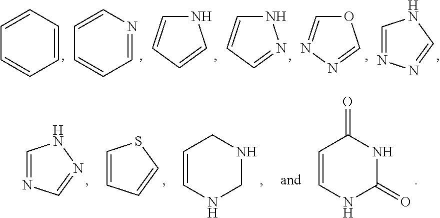

In some embodiments, the compound is represented by Formula (Ib), R_{1b} is -L-C and C is an optionally substituted monocyclic ring selected from the group consisting of

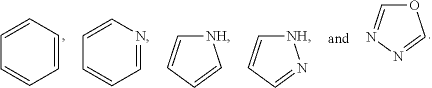

In some embodiments, the compound is represented by Formula (Ib), R_{1b} is -L_{c}-C and C is an optionally substituted bicyclic ring selected from the group consisting of imidazo[1,2-a]pyridine, benzo[d]imidazole, indoline, 1,2,3,4-tetrahydroquinoline, octahydro-1H-benzo[d]imidazole, and octahydro-2h-benzo[d]imidazole-2-one.

In some embodiments, the compound is represented by Formula (Ib), R_{1b} is -L_{c}-C and C is an optionally substituted bicyclic ring selected from the group consisting of imidazo[1,2-a]pyridine and benzo[d]imidazole.

In some embodiments, the compound is represented by Formula (Ib), R_{1b} is -L-C and C is an optionally substituted bicyclic ring selected from the group consisting of:

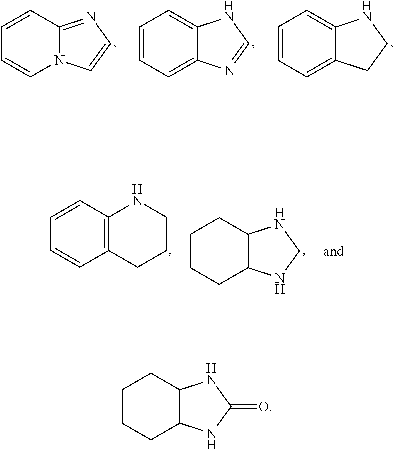

In some embodiments, the compound is represented by Formula (Ib), R_{1b} is -L_{c}-C and C is an optionally substituted bicyclic ring selected from the group consisting of:

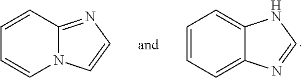 [SMILES: c1ccn2ccnc2c1 ; c1ccc2[nH]cnc2c1]

In some embodiments, the compound is represented by Formula (Ib), R_{1b} is -L_{c}-C and both B and C are an optionally substituted monocyclic ring selected from benzene and pyridine.

In some embodiments, the compound is represented by Formula (Ib), R_{1b} is -L_{c}-C and both B and C are an optionally substituted monocyclic ring selected from:

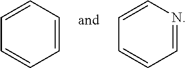 [SMILES: c1ccccc1 ; c1ccncc1]

In some embodiments, the compound represented by Formula (Ib) is a compound or salt thereof of Table 2 below.

In some embodiments, the compound is a compound or a salt thereof of Table 2 below:

TABLE 2
AHR antagonists

| Compd. | |
|---|---|
| No. | Structure |
| 50 | 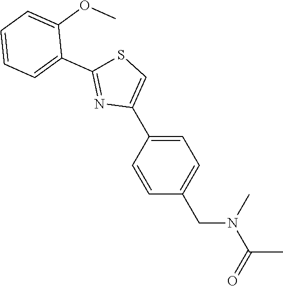 [SMILES: COc1ccccc1-c1nc(-c2ccc(CN(C)C(C)=O)cc2)cs1] |
| 51 | 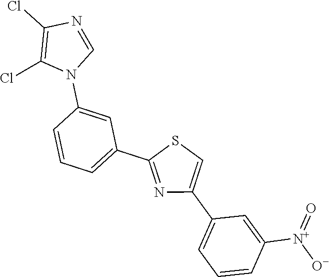 [SMILES: O=[N+]([O-])c1cccc(-c2csc(-c3cccc(-n4cnc(Cl)c4Cl)c3)n2)c1] |
| 52 | 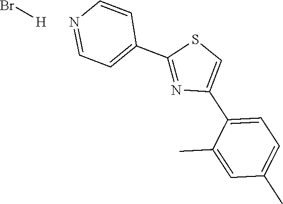 |
| 53 | 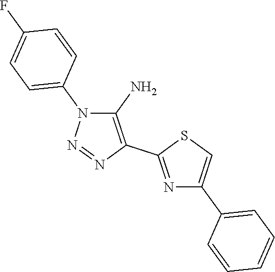 [SMILES: Nc1c(-c2nc(-c3ccccc3)cs2)nnn1-c1ccc(F)cc1] |
| 54 | 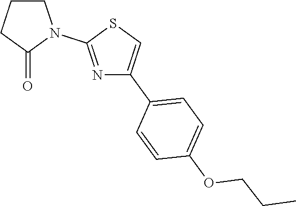 [SMILES: CCCOc1ccc(-c2csc(N3CCCC3=O)n2)cc1] |
| 55 | 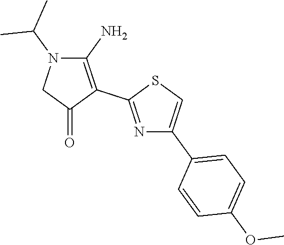 [SMILES: COc1ccc(-c2csc(C3=C(N)N(C(C)C)CC3=O)n2)cc1] |
| 56 | 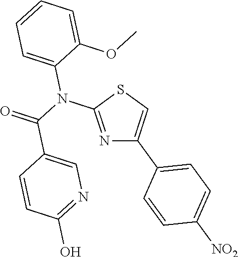 [SMILES: COc1ccccc1N(C(=O)c1ccc(O)nc1)c1nc(-c2ccc([N+](=O)[O-])cc2)cs1] |
| 57 | 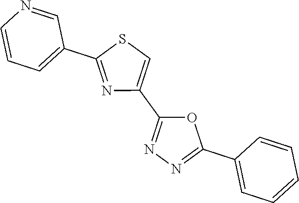 [SMILES: c1ccc(-c2nnc(-c3csc(-c4cccnc4)n3)o2)cc1] |
| 58 | 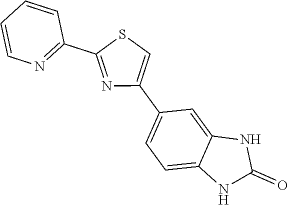 [SMILES: O=c1[nH]c2ccc(-c3csc(-c4ccccn4)n3)cc2[nH]1] |
| 59 | 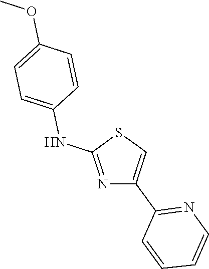 [SMILES: COc1ccc(Nc2nc(-c3ccccn3)cs2)cc1] |
| 60 | 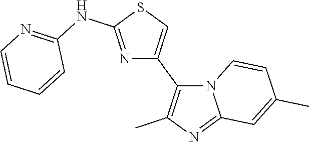 [SMILES: Cc1ccn2c(-c3csc(Nc4ccccn4)n3)c(C)nc2c1] |
| 61 | 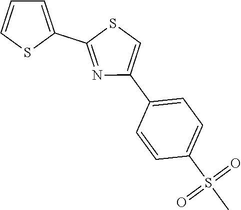 [SMILES: CS(=O)(=O)c1ccc(-c2csc(-c3cccs3)n2)cc1] |
| 62 | 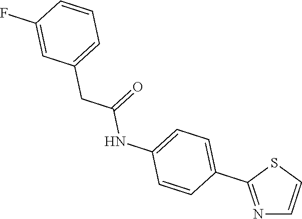 [SMILES: O=C(Cc1cccc(F)c1)Nc1ccc(-c2nccs2)cc1] |
| 63 | 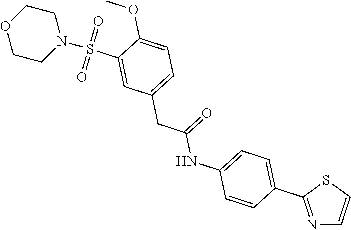 [SMILES: COc1ccc(CC(=O)Nc2ccc(-c3nccs3)cc2)cc1S(=O)(=O)N1CCOCC1] |
| 64 | 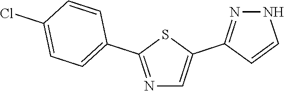 [SMILES: Clc1ccc(-c2ncc(-c3cc[nH]n3)s2)cc1] |
| 65 | 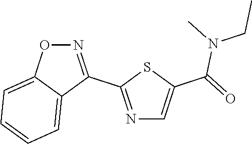 [SMILES: CCN(C)C(=O)c1cnc(-c2noc3ccccc23)s1] |
| 126 | 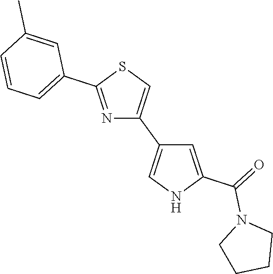 [SMILES: Cc1cccc(-c2nc(-c3c[nH]c(C(=O)N4CCCC4)c3)cs2)c1] |

In some embodiments, the compound represented by Formula (Ib) is a compound or salt thereof of Table 2 below.

In some embodiments, the compound is a compound or a salt thereof of Table 2A below:

TABLE 2A
AHR antagonists

| Compd. | |
|---|---|
| No. | Structure |
| 53A | 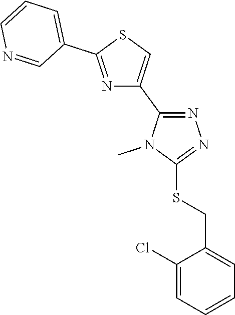 [SMILES: Cn1c(SCc2ccccc2Cl)nnc1-c1csc(-c2cccnc2)n1] |
| 54A | 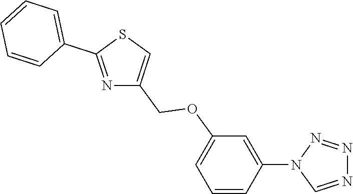 [SMILES: c1ccc(-c2nc(COc3cccc(-n4cnnn4)c3)cs2)cc1] |
| 55A | 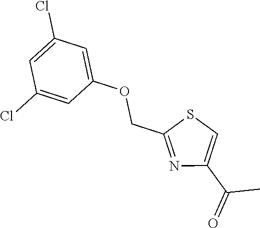 [SMILES: CC(=O)c1csc(COc2cc(Cl)cc(Cl)c2)n1] |
| 56A | 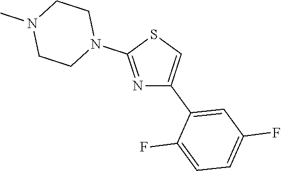 [SMILES: CN1CCN(c2nc(-c3cc(F)ccc3F)cs2)CC1] |
| 57A | 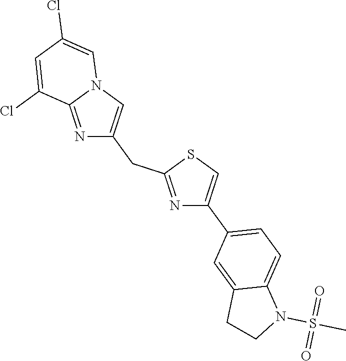 [SMILES: CS(=O)(=O)N1CCc2cc(-c3csc(Cc4cn5cc(Cl)cc(Cl)c5n4)n3)ccc21] |
| 58A | 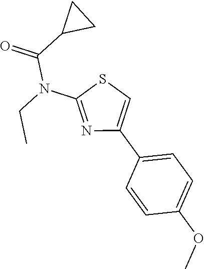 [SMILES: CCN(C(=O)C1CC1)c1nc(-c2ccc(OC)cc2)cs1] |
| 59A | 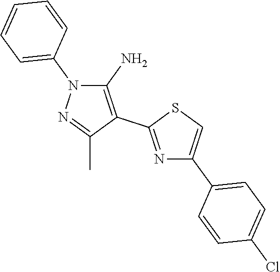 [SMILES: Cc1nn(-c2ccccc2)c(N)c1-c1nc(-c2ccc(Cl)cc2)cs1] |
| 60A | 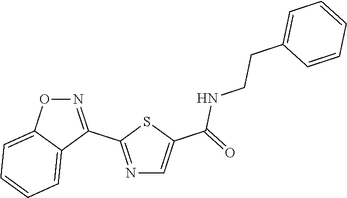 [SMILES: O=C(NCCc1ccccc1)c1cnc(-c2noc3ccccc23)s1] |
| 61A | 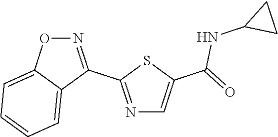 [SMILES: O=C(NC1CC1)c1cnc(-c2noc3ccccc23)s1] |
| 62A | 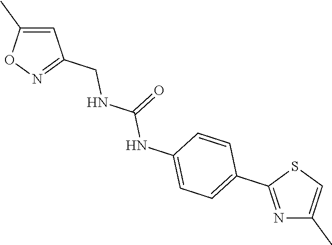 |
| 63A | 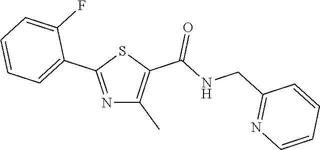 [SMILES: Cc1nc(-c2ccccc2F)sc1C(=O)NCc1ccccn1] |
| 64A | 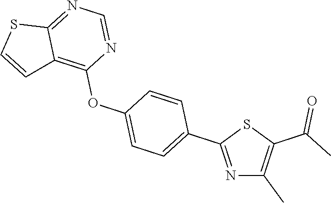 [SMILES: CC(=O)c1sc(-c2ccc(Oc3ncnc4sccc34)cc2)nc1C] |
| 65A | 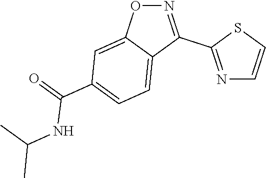 [SMILES: CC(C)NC(=O)c1ccc2c(-c3nccs3)noc2c1] |
| 66A | 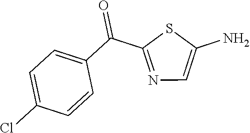 [SMILES: Nc1cnc(C(=O)c2ccc(Cl)cc2)s1] |

In some embodiments, the compound represented by Formula (Ib) is a compound or salt thereof of Table 2 below.

In some embodiments, the compound is a compound or a salt thereof of Table 2B below:

TABLE 2B
AHR antagonists

| Compd. | |
|---|---|
| No. | Structure |
| 33B | 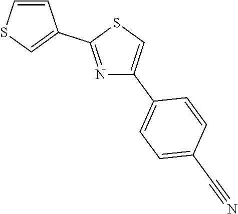 [SMILES: N#Cc1ccc(-c2csc(-c3ccsc3)n2)cc1] |
| 34B | 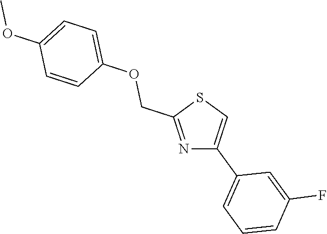 [SMILES: COc1ccc(OCc2nc(-c3cccc(F)c3)cs2)cc1] |
| 35B | 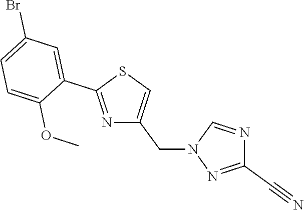 [SMILES: COc1ccc(Br)cc1-c1nc(Cn2cnc(C#N)n2)cs1] |
| 36B | 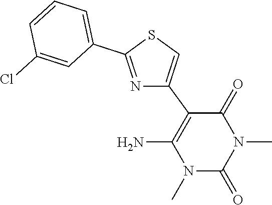 [SMILES: Cn1c(N)c(-c2csc(-c3cccc(Cl)c3)n2)c(=O)n(C)c1=O] |
| 37B | 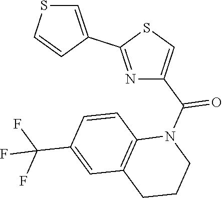 [SMILES: O=C(c1csc(-c2ccsc2)n1)N1CCCc2cc(C(F)(F)F)ccc21] |
| 38B | 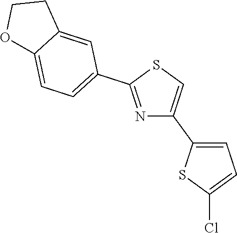 [SMILES: Clc1ccc(-c2csc(-c3ccc4c(c3)CCO4)n2)s1] |
| 39B | 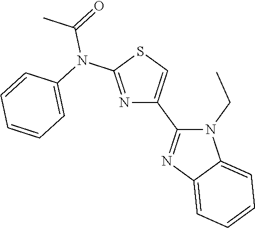 [SMILES: CCn1c(-c2csc(N(C(C)=O)c3ccccc3)n2)nc2ccccc21] |
| 40B | 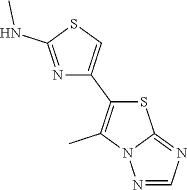 [SMILES: CNc1nc(-c2sc3ncnn3c2C)cs1] |
| 41B | 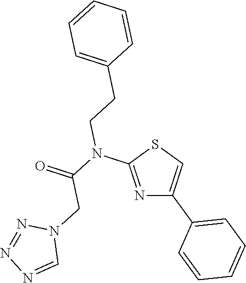 [SMILES: O=C(Cn1cnnn1)N(CCc1ccccc1)c1nc(-c2ccccc2)cs1] |

In some embodiments, the disclosure features a compound wherein A is

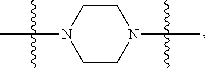 [SMILES: *N1CCN(*)CC1]

in which each [symbol] independently denotes the linkage between A and hydrogen, -Lb-B, -Lc-C, or a substituent.

In some embodiments, the disclosure features a compound represented by Formula (Ic) or a salt thereof

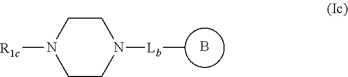

wherein

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a piperazine nitrogen and ** denotes the linkage between L_{b} and B;

each R_{ba} independently is H or C₁-C₃ alkyl optionally substituted with one or more halogen, —CF₃, —CN, —OR_{baa}, —NR_{ba}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R_{1c} is -L_{c}-C, C(O)R₂ₐ, or C(O)OR₂ₐ, wherein each R₂ₐ is C₁-C₆ alkyl;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a piperazine nitrogen and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (Ic) and R_{1c} is selected from the group consisting of C(O)CH₃ and C(O)OCH₂CH₃.

In some embodiments, the compound is represented by Formula (Ic) and B is an optionally substituted monocyclic ring selected from the group consisting of benzene, pyrimidine, pyridine, thiophene, 1,3,5-triazine, 1,3,4-thiadiazole, 4,5-dihydrothiazole, and thiazol-4(5H)-one.

In some embodiments, the compound is represented by Formula (Ic) and B is an optionally substituted monocyclic ring selected from the group consisting of benzene, pyrimidine, pyridine, and thiophene.

In some embodiments, the compound is represented by Formula (Ic) and B is an optionally substituted monocyclic ring selected from the group consisting of:

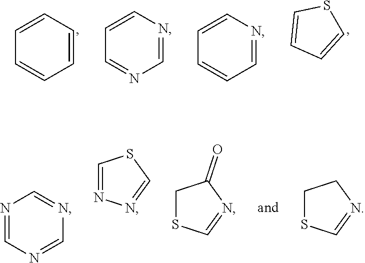

In some embodiments, the compound is represented by Formula (Ic) and B is an optionally substituted monocyclic ring selected from the group consisting of:

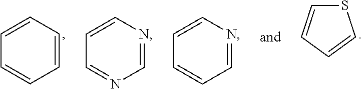

In some embodiments, the compound is represented by Formula (Ic) and B is an optionally substituted bicyclic ring selected from the group consisting of benzo[d]isothiazaole, thieno[2,3-d]pyrimidine, pteridine, [1,2,4]triazolo[4,3-b]pyridazine, 5,6,7,8-tetrahydroquinazoline, 7,8-dihydroquinazolin-5(6H)-one, and 4a,6,7,7a.-tetrahydro-5H-cyclopenta[b]pyridine.

In some embodiments, the compound is represented by Formula (Ic) and B is an optionally substituted bicyclic ring selected from the group consisting of benzo[d]isothiazaole and thieno[2,3-d]pyrimidine.

In some embodiments, the compound is represented by Formula (Ic) and B is an optionally substituted bicyclic ring selected from the group consisting of:

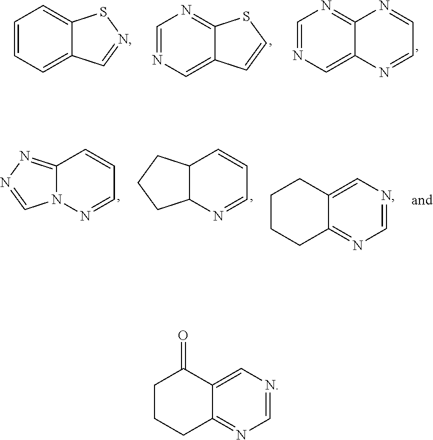

In some embodiments, the compound is represented by Formula (Ic) and B is an optionally substituted bicyclic ring selected from the group consisting of:

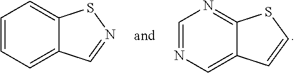 [SMILES: c1ccc2sncc2c1 ; c1ncc2ccsc2n1]

In some embodiments, the compound is represented by Formula (Ic) and L_{b} is selected from the group consisting of a covalent bond and *—SO₂—**.

In some embodiments, the compound is represented by Formula (Ic) and L_{b} is a covalent bond.

In some embodiments, the compound is represented by Formula (Ic), R_{1c} is -L_{c}-C and L_{c} is selected from the group consisting of a covalent bond, *-€(O)—**, *—N═CH₂—**, *—C(O)NH—**.

In some embodiments, the compound is represented by Formula (Ic), R₁ₐ is -L₄-C and C is an optionally substituted monocyclic ring selected from the group consisting of benzene, pyrimidine, thiazole, pyridine, pyridazine, 4,5-dihydrothiazole, 2,3,4,5-tetrahydro-1,2,4-triazine, 1,2,4-triazine-3,5(2H,4H)-dione and 2,4-dimethyl-1,2,4-triazine-3,5(2H, 4H)-dione.

In some embodiments, the compound is represented by Formula (Ic), R₁ₐ is -L_{c}-C and C is an optionally substituted monocyclic ring selected from the group consisting of benzene, pyrimidine, and thiazole.

In some embodiments, the compound is represented by Formula (Ic), R₁ₐ is -L_{c}-C and C is an optionally substituted monocyclic ring selected from the group consisting of:

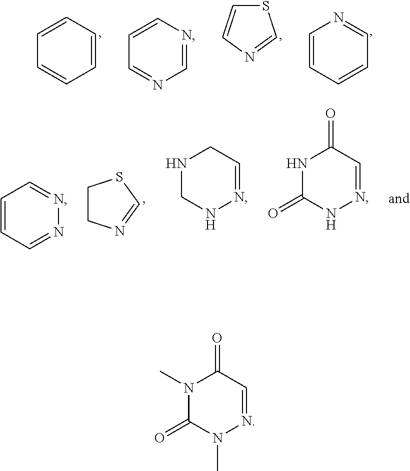

In some embodiments, the compound is represented by Formula (Ic), R₁ₐ is -L-C and C is an optionally substituted monocyclic ring selected from the group consisting of:

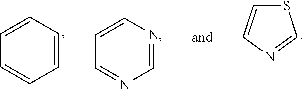

In some embodiments, the compound is represented by Formula (Ic), R₁ₐ is -L_{c}-C and C is an optionally substituted bicyclic ring selected from the group consisting of quinazoline and indole.

In some embodiments, the compound is represented by Formula (Ic), R₁ₐ is -L_{c}-C and C is an optionally substituted bicyclic ring selected from the group consisting of:

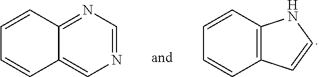 [SMILES: c1ccc2ncncc2c1 ; c1ccc2[nH]ccc2c1]

In some embodiments, the compound represented by Formula (Ic) is a compound or salt thereof of Table 3 below.

In some embodiments, the compound is a compound or a salt thereof of Table 3 below:

TABLE 3
AHR antagonists

| Compd. | |
|---|---|
| No. | Structure |
| 74 | 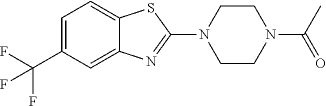 [SMILES: CC(=O)N1CCN(c2nc3cc(C(F)(F)F)ccc3s2)CC1] |
| 75 | 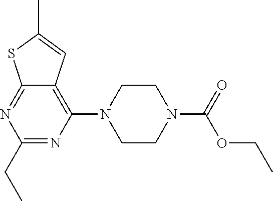 [SMILES: CCOC(=O)N1CCN(c2nc(CC)nc3sc(C)cc23)CC1] |
| 76 | 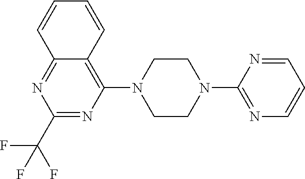 [SMILES: FC(F)(F)c1nc(N2CCN(c3ncccn3)CC2)c2ccccc2n1] |
| 77 | 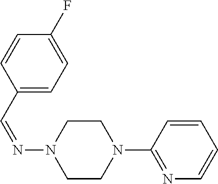 [SMILES: Fc1ccc(/C=N\N2CCN(c3ccccn3)CC2)cc1] |
| 78 | 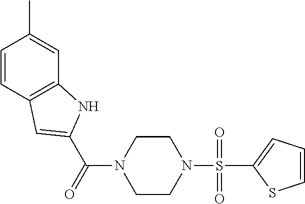 [SMILES: Cc1ccc2cc(C(=O)N3CCN(S(=O)(=O)c4cccs4)CC3)[nH]c2c1] |
| 79 | 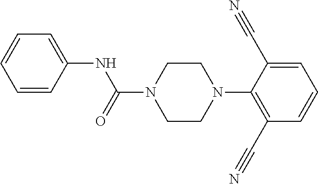 [SMILES: N#Cc1cccc(C#N)c1N1CCN(C(=O)Nc2ccccc2)CC1] |
| 80 | 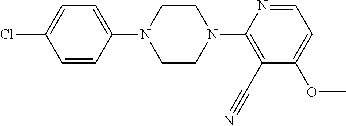 [SMILES: COc1ccnc(N2CCN(c3ccc(Cl)cc3)CC2)c1C#N] |
| 81 | 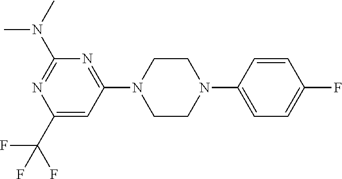 [SMILES: CN(C)c1nc(N2CCN(c3ccc(F)cc3)CC2)cc(C(F)(F)F)n1] |
| 111 | 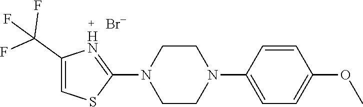 |

In some embodiments, the compound represented by Formula (Ic) is a compound or salt thereof of Table 3A below.

In some embodiments the compound is a compound or a salt thereof of Table 3A below:

TABLE 3A
AHR antagonists

| Compd. | |
|---|---|
| No. | Structure |
| 67A | 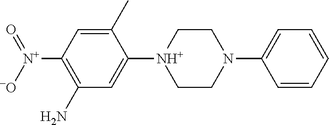 |
| 68A | 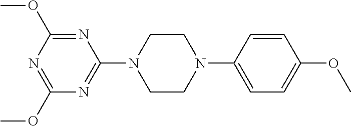 |
| 69A | 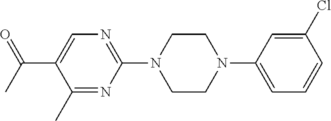 [SMILES: CC(=O)c1cnc(N2CCN(c3cccc(Cl)c3)CC2)nc1C] |
| 70A | 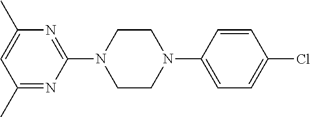 [SMILES: Cc1cc(C)nc(N2CCN(c3ccc(Cl)cc3)CC2)n1] |
| 71A | 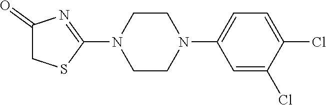 [SMILES: O=C1CSC(N2CCN(c3ccc(Cl)c(Cl)c3)CC2)=N1] |
| 72A | 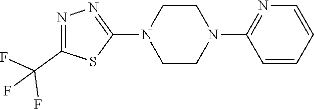 [SMILES: FC(F)(F)c1nnc(N2CCN(c3ccccn3)CC2)s1] |
| 73A | 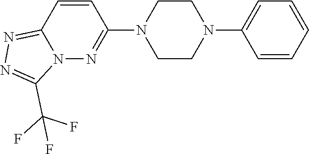 [SMILES: FC(F)(F)c1nnc2ccc(N3CCN(c4ccccc4)CC3)nn12] |
| 74A | 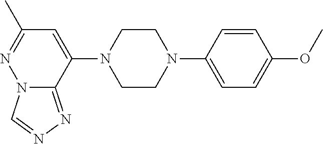 [SMILES: COc1ccc(N2CCN(c3cc(C)nn4cnnc34)CC2)cc1] |
| 75A | 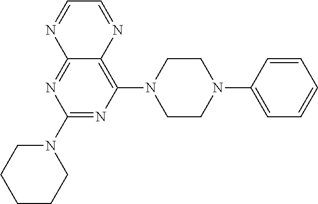 [SMILES: c1ccc(N2CCN(c3nc(N4CCCCC4)nc4nccnc34)CC2)cc1] |
| 76A | 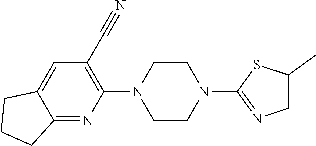 [SMILES: CC1CN=C(N2CCN(c3nc4c(cc3C#N)CCC4)CC2)S1] |
| 77A | 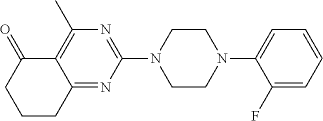 [SMILES: Cc1nc(N2CCN(c3ccccc3F)CC2)nc2c1C(=O)CCC2] |
| 78A | 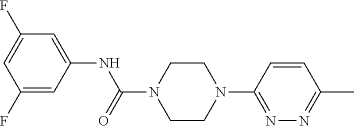 [SMILES: Cc1ccc(N2CCN(C(=O)Nc3cc(F)cc(F)c3)CC2)nn1] |
| 79A | 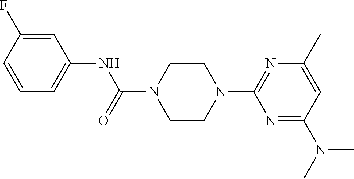 [SMILES: Cc1cc(N(C)C)nc(N2CCN(C(=O)Nc3cccc(F)c3)CC2)n1] |
| 80A | 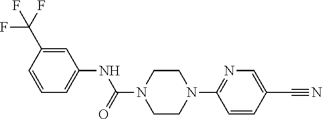 [SMILES: N#Cc1ccc(N2CCN(C(=O)Nc3cccc(C(F)(F)F)c3)CC2)nc1] |
| 81A | 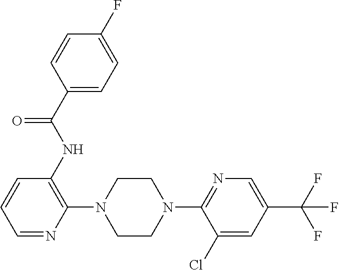 [SMILES: O=C(Nc1cccnc1N1CCN(c2ncc(C(F)(F)F)cc2Cl)CC1)c1ccc(F)cc1] |
| 82A | 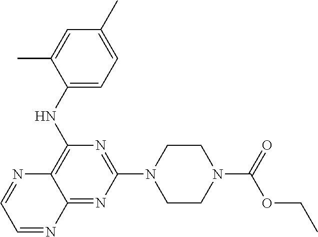 [SMILES: CCOC(=O)N1CCN(c2nc(Nc3ccc(C)cc3C)c3nccnc3n2)CC1] |

In some embodiments, the compound represented by Formula (Ic) is a compound or salt thereof of Table 3B below.

In some embodiments, the compound is a compound or a salt thereof of Table 3B below:

TABLE 3B
AHR antagonists

| Compd. | |
|---|---|
| No. | Structure |
| 42B | 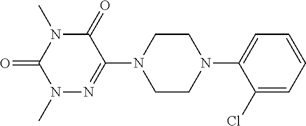 [SMILES: Cn1nc(N2CCN(c3ccccc3Cl)CC2)c(=O)n(C)c1=O] |

In some embodiments, the disclosure features a compound represented by Formula (Id1) or Formula (Id2)

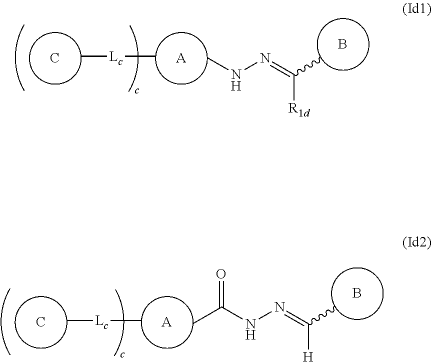

wherein

A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

c is 0 or 1;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NRe—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;

each R_{cb} independently is H, —C(O)Rcba, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl; and

R_{1d} is hydrogen or C₁-C₃ alkyl.

In some embodiments, the compound is represented by Formula (Id1) or Formula (Id2), c is 1, L_{c} is a covalent bond and C is an optionally substituted monocyclic ring selected from the group consisting of benzene and pyridine.

In some embodiments, the compound is represented by Formula (Id1) or Formula (Id2), c is 1, L_{c} is a covalent bond and C is an optionally substituted monocyclic ring selected from the group consisting of:

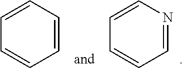 [SMILES: c1ccccc1 ; c1ccncc1]

In some embodiments, the compound is represented by Formula (Id1) or Formula (Id2) and B is an optionally substituted monocyclic ring selected from the group consisting of benzene and furan.

In some embodiments, the compound is represented by Formula (Id1) or Formula (Id2) and B is an optionally substituted benzene.

In some embodiments, the compound is represented by Formula (Id1) or Formula (Id2) and B is an optionally substituted monocyclic ring selected from the group consisting of:

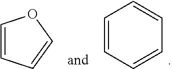 [SMILES: c1ccoc1 ; c1ccccc1]

In some embodiments, the compound is represented by Formula (Id1) or Formula (Id2) and B is an optionally substituted

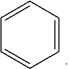

In some embodiments, the compound is represented by Formula (Id1) or Formula (Id2) and B is an optionally substituted benzofuran.

In some embodiments, the compound is represented by Formula (Id1) or Formula (Id2) and B is an optionally substituted

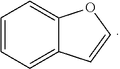

In some embodiments, the compound is represented by Formula (Id) or Formula (Id2) and A is an optionally substituted monocyclic ring selected from the group consisting of pyrimidine, benzene, thiazole, pyridine and furan.

In some embodiments, the compound is represented by Formula (Id1) or Formula (Id2) and A is an optionally substituted monocyclic ring selected from the group consisting of pyrimidine, benzene, and thiazole.

In some embodiments, the compound is represented by Formula (Id) or Formula (Id2) and A is an optionally substituted monocyclic ring selected from the group consisting of:

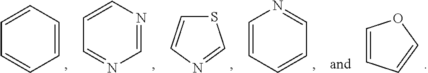

In some embodiments, the compound is represented by Formula (Id) or Formula (Id2) and A is an optionally substituted monocyclic ring selected from the group consisting of:

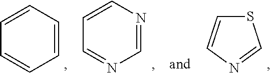

In some embodiments, the compound is represented by Formula (Id1) or Formula (Id2) and A is an optionally substituted 1H-benzo[d]imidazole.

In some embodiments, the compound is represented by Formula (Id1) or Formula (Id2) and A is an optionally substituted

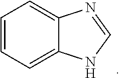

In some embodiments, the compound is represented by Formula (Id1) or Formula (Id2) and A is an optionally substituted 4,5-dihydro-H-benzo[g]indazole.

In some embodiments, the compound is represented by Formula (Id1) or Formula (Id2) and A is an optionally substituted

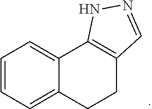 [SMILES: c1ccc2c(c1)CCc1cn[nH]c1-2]

In some embodiments, the compound represented by Formula (Id1) or Formula (Id2) is a compound or salt thereof of Table 4 below.

In some embodiments, the compound is a compound or a salt thereof of Table 4 below:

TABLE 4
AHR antagonists

| Compd. | |
|---|---|
| No. | Structure |
| 98 | 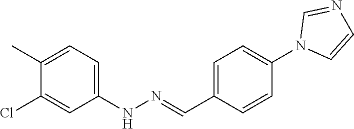 [SMILES: Cc1ccc(N/N=C/c2ccc(-n3ccnc3)cc2)cc1Cl] |
| 99 | 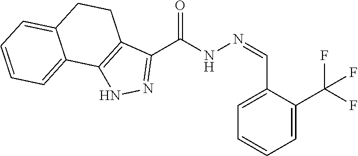 [SMILES: O=C(N/N=C\c1ccccc1C(F)(F)F)c1n[nH]c2c1CCc1ccccc1-2] |
| 112 | 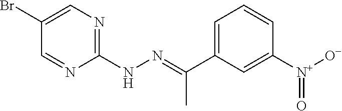 [SMILES: C/C(=N\Nc1ncc(Br)cn1)c1cccc([N+](=O)[O-])c1] |
| 113 | 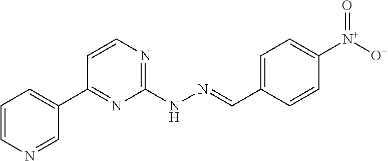 [SMILES: O=[N+]([O-])c1ccc(/C=N/Nc2nccc(-c3cccnc3)n2)cc1] |
| 114 | 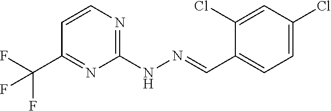 [SMILES: FC(F)(F)c1ccnc(N/N=C/c2ccc(Cl)cc2Cl)n1] |
| 115 | 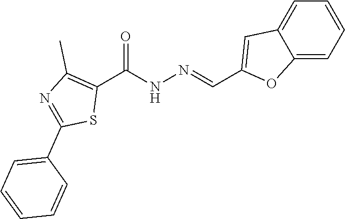 [SMILES: Cc1nc(-c2ccccc2)sc1C(=O)N/N=C/c1cc2ccccc2o1] |

In some embodiments, the compound represented by Formula (Id1) or Formula (Id2) is a compound or salt thereof of Table 4A below.

In some embodiments, the compound is a compound or a salt thereof of Table 4A below:

TABLE 4A
AHR antagonists

| Compd. | |
|---|---|
| No. | Structure |
| 83A | 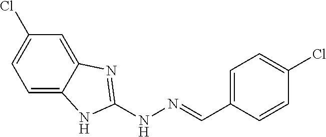 [SMILES: Clc1ccc(/C=N/Nc2nc3cc(Cl)ccc3[nH]2)cc1] |

In some embodiments, the compound represented by Formula (Id1) or Formula (Id2) is a compound or salt thereof of Table 4B below.

In some embodiments, the compound is a compound or a salt thereof of Table 4B below:

TABLE 4B
AHR antagonists

| Compd. | |
|---|---|
| No. | Structure |
| 43B | 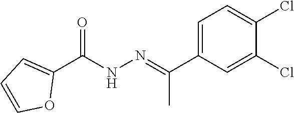 [SMILES: C/C(=N\NC(=O)c1ccco1)c1ccc(Cl)c(Cl)c1] |
| 44B | 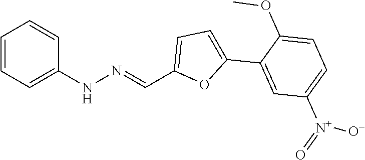 [SMILES: COc1ccc([N+](=O)[O-])cc1-c1ccc(/C=N/Nc2ccccc2)o1] |
| 45B | 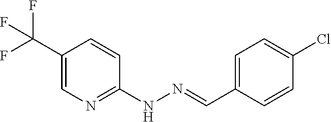 [SMILES: FC(F)(F)c1ccc(N/N=C/c2ccc(Cl)cc2)nc1] |

In some embodiments, the disclosure features a compound wherein A is

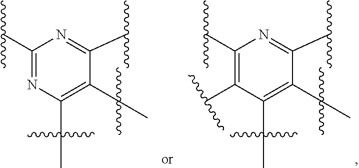 [SMILES: *c1nc(*)c(*)c(*)n1 ; *c1nc(*)c(*)c(*)c1*]

in which each [symbol] independently denotes the linkage between A and hydrogen, -Lb-B, -Lc-C, or a substituent.

In some embodiments, the disclosure features a compound wherein A is

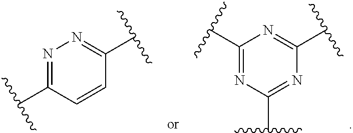 [SMILES: *c1ccc(*)nn1 ; *c1nc(*)nc(*)n1]

in which each [symbol] independently denotes the linkage between A and hydrogen, -Lb-B, -Lc-C, or a substituent.

In some embodiments, the disclosure features a compound represented by Formula (Ie1) or Formula (Ie2)

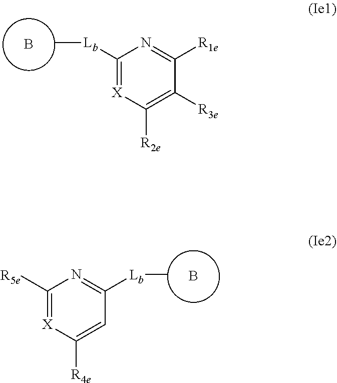

wherein

X is N or CR₆ₑ in which R₆ₑ is hydrogen, halogen, or —CN;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O) *—SO₂—**, *=N—**, *—N=**, *=N—C(O)—**, *—C(O)—N=**, *—O—R_{ba}**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—*, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—Ra—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a pyridine or pyrimidine carbon and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R₁ₑ is hydrogen, —CF₃, or -L_{c}-C;

R₂ₑ is hydrogen, —CF₃, L_{c}-C, or 6-membered aryl optionally substituted with one or more halogen, —CF₃, or —CN;

R₃ₑ is hydrogen or when R₁ₑ is hydrogen and R₂ₑ is hydrogen R₃ₑ is L_{c}-C;

R_{4c} is hydrogen or L_{c}-C;

R₅ₑ is hydrogen or L-C;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, ****—R_{ca}—S—**, *—O—R_{ca}—**, *—R_{ca}—O—**, *—C(O)NR_{cb}NR_{cb}C(O)—**, in which * denotes the linkage between L_{c} and a pyridine or pyrimidine carbon and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (Ie1) wherein X is N.

In some embodiments, the compound is represented by Formula (Ie1) or Formula (Ie2) and B is an optionally substituted monocyclic ring selected from the group consisting of pyrazole, benzene, and pyridine.

In some embodiments, the compound is represented by Formula (Ie1) or Formula (Ie2) and B is an optionally substituted monocyclic ring selected from the group consisting of:

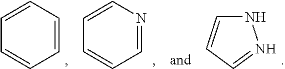

In some embodiments, the compound is represented by Formula (Ie1) or Formula (Ie2) and B is an optionally substituted indole.

In some embodiments, the compound is represented by Formula (Ie1) or Formula (Ie2) and B is an optionally substituted

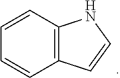

In some embodiments, the compound is represented by Formula (Ie1) or Formula (Ie2) and C is an optionally substituted monocyclic ring selected from the group consisting of benzene and pyridine.

In some embodiments, the compound is represented by Formula (Ie1) or Formula (Ie2) and C is an optionally substituted monocyclic ring selected from the group consisting of: and ON

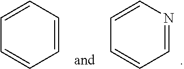 [SMILES: c1ccccc1 ; c1ccncc1]

In some embodiments, the compound is represented by Formula (Ie1) or Formula (Ie2) and L_{b} is selected from the group consisting of a covalent bond, *—NH—**, and *—NHCH2CH(OH)—**.

In some embodiments, the compound is represented by Formula (Ie1) or Formula (Ie2) and L_{b} is a covalent bond.

In some embodiments, the compound is represented by Formula (Ie1) or Formula (Ie2) wherein at least one of R₁ₑ, R₂ₑ, R₁ₑ, R₄ₑ and R₅ₑ is L-C and L_{c} is selected from the group consisting of a covalent bond, *—NH—**, and *—SCH₂—**.

In some embodiments, the compound is represented by Formula (Ie1) or Formula (Ie2) wherein at least one of R₁ₑ, R₂ₑ, R₁ₑ, R₄ₑ and R₅ₑ is L_{c}-C and L_{c} is a covalent bond.

In some embodiments, the compound represented by Formula (Ie1) or Formula (Ie2) is a compound or salt thereof of Table 5 below.

In some embodiments, the compound is a compound or a salt thereof of Table 5 below:

TABLE 5
AHR antagonists

| Compd. | |
|---|---|
| No. | Structure |
| 82 | 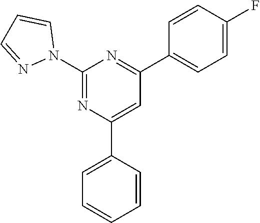 [SMILES: Fc1ccc(-c2cc(-c3ccccc3)nc(-n3cccn3)n2)cc1] |
| 83 | 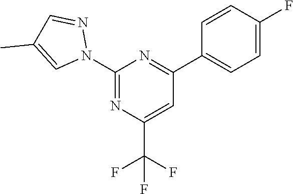 [SMILES: Cc1cnn(-c2nc(-c3ccc(F)cc3)cc(C(F)(F)F)n2)c1] |
| 84 | 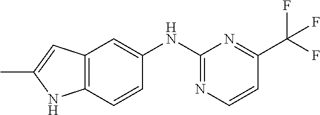 [SMILES: Cc1cc2cc(Nc3nccc(C(F)(F)F)n3)ccc2[nH]1] |
| 85 | 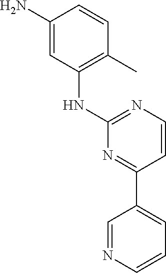 [SMILES: Cc1ccc(N)cc1Nc1nccc(-c2cccnc2)n1] |
| 86 | 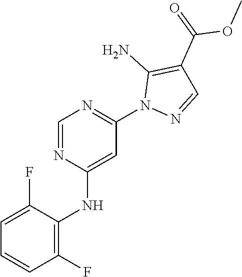 [SMILES: COC(=O)c1cnn(-c2cc(Nc3c(F)cccc3F)ncn2)c1N] |
| 87 | 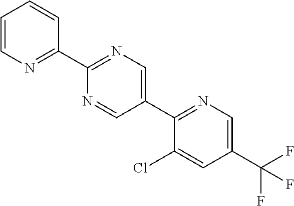 [SMILES: FC(F)(F)c1cnc(-c2cnc(-c3ccccn3)nc2)c(Cl)c1] |
| 88 | 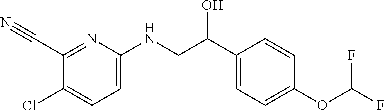 [SMILES: N#Cc1nc(NCC(O)c2ccc(OC(F)F)cc2)ccc1Cl] |
| 89 | 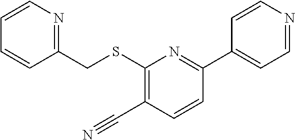 [SMILES: N#Cc1ccc(-c2ccncc2)nc1SCc1ccccn1] |

In some embodiments, the compound represented by Formula (Ie1) or Formula (Ie2) is a compound or salt thereof of Table 5A below.

In some embodiments, the compound is a compound or a salt thereof of Table 5A below:

TABLE 5A
AHR antagonists

| Compd. | |
|---|---|
| No. | Structure |
| 84A | 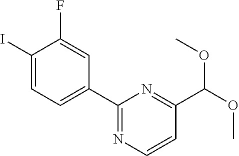 [SMILES: COC(OC)c1ccnc(-c2ccc(I)c(F)c2)n1] |
| 85A | 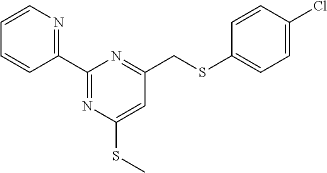 [SMILES: CSc1cc(CSc2ccc(Cl)cc2)nc(-c2ccccn2)n1] |
| 86A | 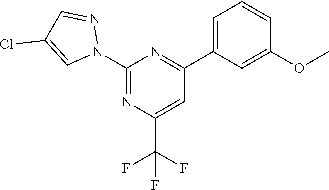 [SMILES: COc1cccc(-c2cc(C(F)(F)F)nc(-n3cc(Cl)cn3)n2)c1] |
| 87A | 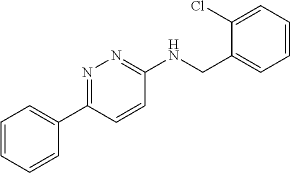 [SMILES: Clc1ccccc1CNc1ccc(-c2ccccc2)nn1] |
| 88A | 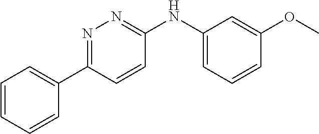 [SMILES: COc1cccc(Nc2ccc(-c3ccccc3)nn2)c1] |
| 89A | 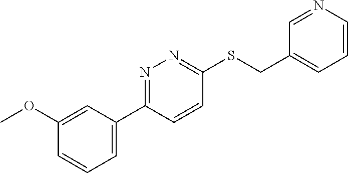 [SMILES: COc1cccc(-c2ccc(SCc3cccnc3)nn2)c1] |
| 90A | 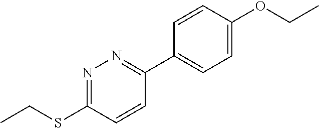 [SMILES: CCOc1ccc(-c2ccc(SCC)nn2)cc1] |
| 91A | 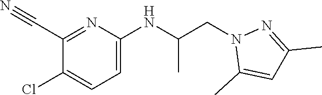 [SMILES: Cc1cc(C)n(CC(C)Nc2ccc(Cl)c(C#N)n2)n1] |
| 92A | 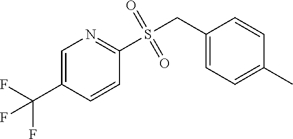 [SMILES: Cc1ccc(CS(=O)(=O)c2ccc(C(F)(F)F)cn2)cc1] |
| 93A | 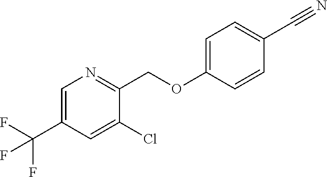 [SMILES: N#Cc1ccc(OCc2ncc(C(F)(F)F)cc2Cl)cc1] |
| 94A | 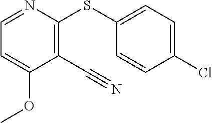 [SMILES: COc1ccnc(Sc2ccc(Cl)cc2)c1C#N] |
| 95A | 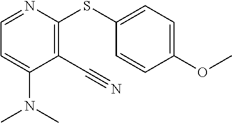 [SMILES: COc1ccc(Sc2nccc(N(C)C)c2C#N)cc1] |

In some embodiments, the compound represented by Formula (Tel) or Formula (Ie2) is a compound or salt thereof of Table 5B below.

In some embodiments, the compound is a compound or a salt thereof of Table 5B below:

TABLE 5B
AHR antagonists

| Compd | |
|---|---|
| No. | Structure |
| 46B | 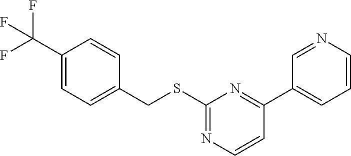 [SMILES: FC(F)(F)c1ccc(CSc2nccc(-c3cccnc3)n2)cc1] |
| 47B | 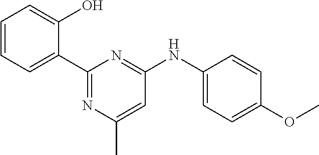 [SMILES: COc1ccc(Nc2cc(C)nc(-c3ccccc3O)n2)cc1] |
| 48B | 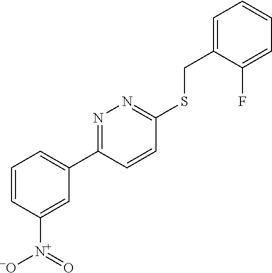 |
| 49B | 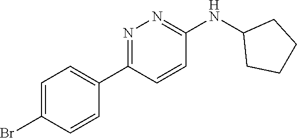 [SMILES: Brc1ccc(-c2ccc(NC3CCCC3)nn2)cc1] |
| 50B | 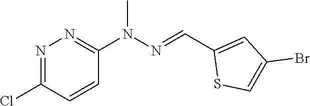 [SMILES: CN(/N=C/c1cc(Br)cs1)c1ccc(Cl)nn1] |
| 51B | 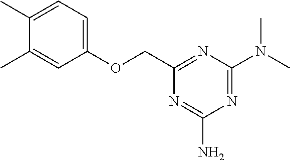 |

In some embodiments, the disclosure features a compound represented by Formula (If)

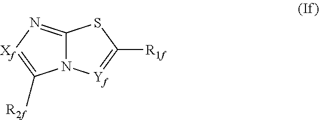

wherein

X_{f} is N or CR_{3f} in which R_{3f} is hydrogen. C₁-C₆ alkyl, or -L_{b}-B;

Y_{f} is N or CR_{4f} in which R_{4f} is hydrogen or C₁-C₆ alkyl;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—**, *—S—R_{ba}—**, *—R_{ba}—S—**, *—SO₂—R_{ba}—**, *—R_{ba}—SO₂—**, *—NR_{bb}—N═CR_{ba}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a imidazo[2,1-b]thiazole or imidazo[2,1-b][1,3,4]thiadiazole carbon and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} is independently H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R_{1f} is CF₃, C₁-C₆ alkyl, -L_{b}-B, or C(O)NHR_{5f} in which R_{5f} is C₁-C₃ alkyl;

R_{2f} is hydrogen or -L_{b}-B when X_{f} is CR_{3f};

R_{2f} is hydrogen or -L_{c}-C when X_{f} is N;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{bb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a [1,2,4]triazolo[3,4-b][1,3,4]thiadiazole carbon and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (If) and B is an optionally substituted monocyclic ring selected from the group consisting of benzene, pyridine, thiazole and pyrazole.

In some embodiments, the compound is represented by Formula (If) and B is an optionally substituted monocyclic ring selected from the group consisting of benzene, pyridine, and pyrazole.

In some embodiments, the compound is represented by Formula (If) and B is an optionally substituted monocyclic ring selected from the group consisting of:

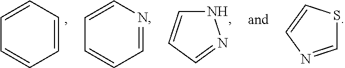

In some embodiments, the compound is represented by Formula (If) and B is an optionally substituted monocyclic ring selected from the group consisting of:

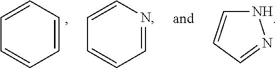

In some embodiments, the compound is represented by Formula (If) and C is an optionally substituted monocyclic ring selected from the group consisting of pyrazole and thiophene.

In some embodiments, the compound is represented by Formula (If) and C is an optionally substituted monocyclic ring selected from the group consisting of:

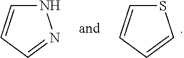 [SMILES: c1cn[nH]c1 ; c1ccsc1]

In some embodiments, the compound is represented by Formula (If) and B is an optionally substituted bicyclic ring selected from the group consisting of 4,5,6,7-tetrahydrobenz[b]thiophene and 2-azabicyclo[2.2.1]heptane.

In some embodiments, the compound is represented by Formula (If) and B is an optionally substituted bicyclic ring selected from the group consisting of:

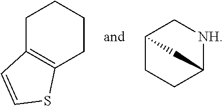 [SMILES: c1cc2c(s1)CCCC2 ; C1C[C@@H]2C[C@@H]1CN2]

In some embodiments, the compound is represented by Formula (If) wherein Y_{f} is N and X_{f} is CR_{3f}.

In some embodiments, the compound is represented by Formula (If) wherein Y_{f} is N, X_{f} is —CCH₃ and R_{1f} is -L_{b}-B.

In some embodiments, the compound is represented by Formula (If) wherein L_{b} is a covalent bond.

In some embodiments, the compound is represented by Formula (If) wherein Y_{f} is N, X_{f} is —CCH₃ and R_{1f} is -L_{b}-B in which L_{b} is *—NHCH₂CH₂O—**.

In some embodiments, the compound is represented by Formula (If) wherein X_{f} is N and Y_{f} is N.

In some embodiments, the compound is represented by Formula (If) and L_{c} is a covalent bond.

In some embodiments, the compound is represented by Formula (If) wherein X_{f} is N, Y_{f} is N, and L_{c} is a covalent bond.

In some embodiments, the compound represented by Formula (If) is a compound or salt thereof of Table 6 below.

In some embodiments, the compound is a compound or a salt thereof of Table 6 below:

TABLE 6
AHR antagonists

| Compd. | |
|---|---|
| No. | Structure |
| 66 | 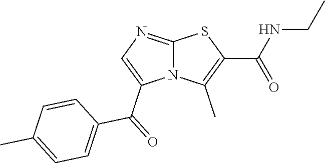 [SMILES: CCNC(=O)c1sc2ncc(C(=O)c3ccc(C)cc3)n2c1C] |
| 67 | 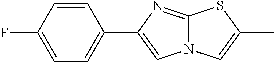 [SMILES: Cc1cn2cc(-c3ccc(F)cc3)nc2s1] |
| 68 | 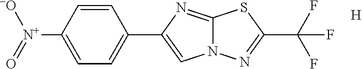 |
| 69 | 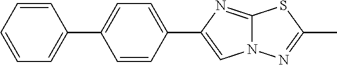 [SMILES: Cc1nn2cc(-c3ccc(-c4ccccc4)cc3)nc2s1] |
| 70 | 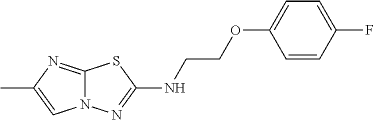 [SMILES: Cc1cn2nc(NCCOc3ccc(F)cc3)sc2n1] |
| 71 | 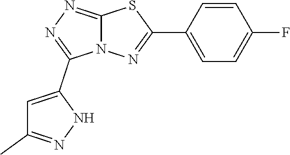 [SMILES: Cc1cc(-c2nnc3sc(-c4ccc(F)cc4)nn23)[nH]n1] |
| 72 | 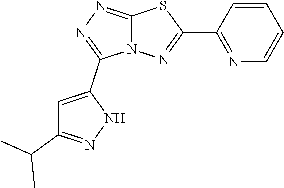 [SMILES: CC(C)c1cc(-c2nnc3sc(-c4ccccn4)nn23)[nH]n1] |
| 73 | 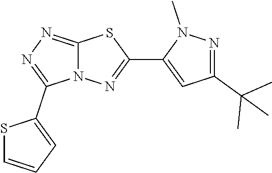 [SMILES: Cn1nc(C(C)(C)C)cc1-c1nn2c(-c3cccs3)nnc2s1] |

In some embodiments, the compound represented by Formula (If) is a compound or salt thereof of Table 6A below.

In some embodiments, the compound is a compound or a salt thereof of Table 6A below:

TABLE 6A
AHR antagonists

| Compd. | |
|---|---|
| No. | Structure |
| 96A | 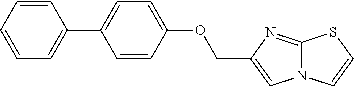 [SMILES: c1ccc(-c2ccc(OCc3cn4ccsc4n3)cc2)cc1] |
| 97A | 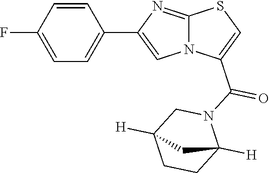 [SMILES: O=C(c1csc2nc(-c3ccc(F)cc3)cn12)N1C[C@@H]2CC[C@@H]1C2] |
| 97A1 | 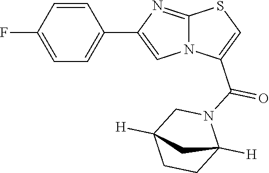 [SMILES: O=C(c1csc2nc(-c3ccc(F)cc3)cn12)N1C[C@H]2CC[C@@H]1C2] |
| 97A2 | 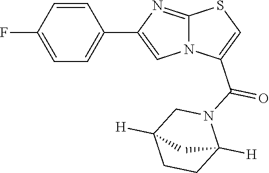 [SMILES: O=C(c1csc2nc(-c3ccc(F)cc3)cn12)N1C[C@@H]2CC[C@H]1C2] |
| 98A | 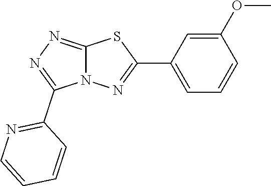 [SMILES: COc1cccc(-c2nn3c(-c4ccccn4)nnc3s2)c1] |
| 99A | 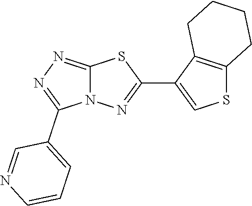 [SMILES: c1cncc(-c2nnc3sc(-c4csc5c4CCCC5)nn23)c1] |

In some embodiments, the compound represented by Formula (If) is a compound or salt thereof of Table 6A below.

In some embodiments, the compound is a compound or a salt thereof of Table 6A below:

TABLE 6B
AHR antagonists

| Compd. | |
|---|---|
| No. | Structure |
| 40B | 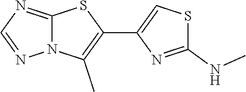 [SMILES: CNc1nc(-c2sc3ncnn3c2C)cs1] |

In some embodiments, the compound is represented by at least one formula selected from the group consisting of Ia, Ib, Ic, Id1, Id2, Ie1, Ie2, and If.

In some embodiments, A is an optionally substituted monocyclic 5-membered heterocycle comprising 2-4 nitrogen heteroatoms.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole.

In some embodiments, A is an optionally substituted monocyclic 5-membered heterocycle comprising 2-4 nitrogen heteroatoms selected from the group consisting of:

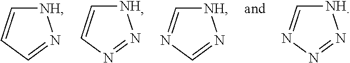

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted benzene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted bicyclic ring selected from the group consisting of benzo[d]isothiazole and naphthalene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted bicyclic ring selected from the group consisting of:

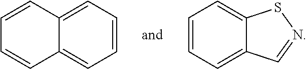 [SMILES: c1ccc2ccccc2c1 ; c1ccc2sncc2c1]

In some embodiments, is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted monocyclic ring selected from the group consisting of benzene, thiophene, and furan.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted monocyclic ring selected from the group consisting of:

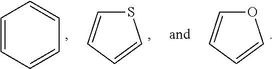

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted 1,2,3,4-tetrahydronaphthalene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and L_{b} is selected from the group consisting of a covalent bond, *—SCH₂—**, and *—R_{ba}—NR_{bb}—**.

In some embodiments, A is an optionally substituted monocyclic 5-membered heterocycle comprising 4 nitrogen heteroatoms selected from the group consisting of pyrazole, 1,2,3-triazole, 1,2,4-triazole, and tetrazole, L_{b} is a covalent bond and Bis an optionally substituted benzene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole, L_{c} is a covalent bond, *—C(O)—**, or *C(O)NHNHC(O)—**.

In some embodiments, the compound is selected from the group consisting of

| Compd. | |
|---|---|
| No. | Structure |
| 90 | 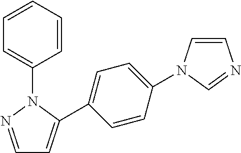 [SMILES: c1ccc(-n2nccc2-c2ccc(-n3ccnc3)cc2)cc1] |
| 91 | 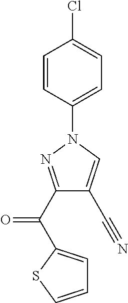 [SMILES: N#Cc1cn(-c2ccc(Cl)cc2)nc1C(=O)c1cccs1] |
| 92 | 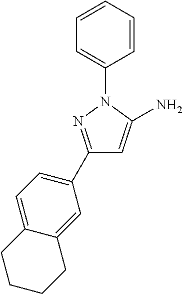 [SMILES: Nc1cc(-c2ccc3c(c2)CCCC3)nn1-c1ccccc1] |
| 93 | 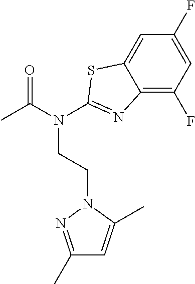 [SMILES: CC(=O)N(CCn1nc(C)cc1C)c1nc2c(F)cc(F)cc2s1] |
| 94 | 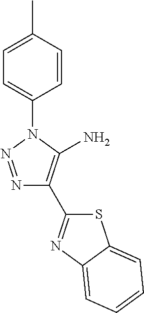 [SMILES: Cc1ccc(-n2nnc(-c3nc4ccccc4s3)c2N)cc1] |
| 95 | 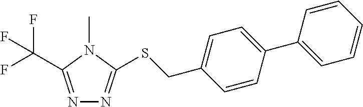 [SMILES: Cn1c(SCc2ccc(-c3ccccc3)cc2)nnc1C(F)(F)F] |
| 96 | 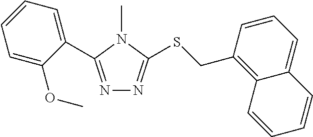 [SMILES: COc1ccccc1-c1nnc(SCc2cccc3ccccc23)n1C] |
| 97 | 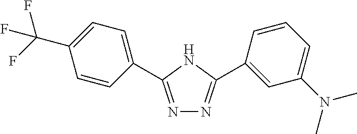 [SMILES: CN(C)c1cccc(-c2nnc(-c3ccc(C(F)(F)F)cc3)[nH]2)c1] |
| 139 | 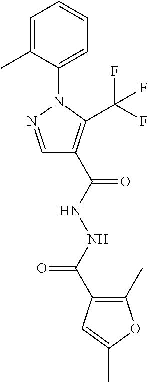 [SMILES: Cc1cc(C(=O)NNC(=O)c2cnn(-c3ccccc3C)c2C(F)(F)F)c(C)o1] |
| | and |
| 154 | 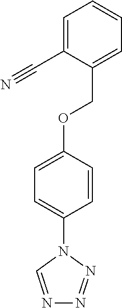 [SMILES: N#Cc1ccccc1COc1ccc(-n2cnnn2)cc1] |

In some embodiments, the compound is selected from the group consisting of

| Compd. | |
|---|---|
| No. | Structure |
| 100A | 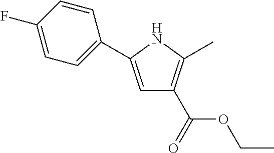 [SMILES: CCOC(=O)c1cc(-c2ccc(F)cc2)[nH]c1C] |
| 101A | 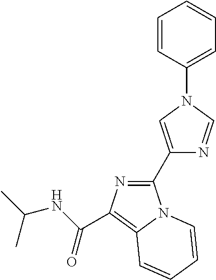 [SMILES: CC(C)NC(=O)c1nc(-c2cn(-c3ccccc3)cn2)n2ccccc12] |
| 102A | 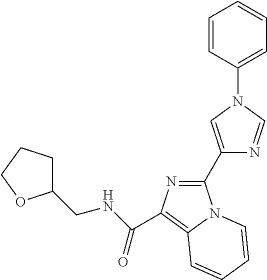 [SMILES: O=C(NCC1CCCO1)c1nc(-c2cn(-c3ccccc3)cn2)n2ccccc12] |
| 103A | 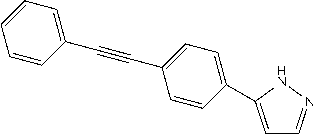 [SMILES: C(#Cc1ccc(-c2ccn[nH]2)cc1)c1ccccc1] |
| 104A | 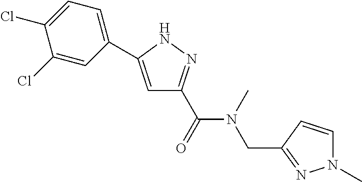 [SMILES: CN(Cc1ccn(C)n1)C(=O)c1cc(-c2ccc(Cl)c(Cl)c2)[nH]n1] |
| 105A | 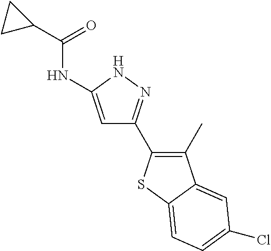 [SMILES: Cc1c(-c2cc(NC(=O)C3CC3)[nH]n2)sc2ccc(Cl)cc12] |
| 106A | 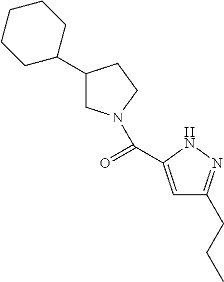 [SMILES: CCCc1cc(C(=O)N2CCC(C3CCCCC3)C2)[nH]n1] |
| 107A | 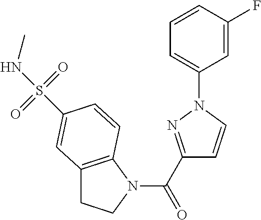 [SMILES: CNS(=O)(=O)c1ccc2c(c1)CCN2C(=O)c1ccn(-c2cccc(F)c2)n1] |
| 108A | 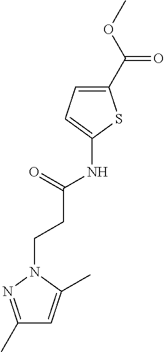 [SMILES: COC(=O)c1ccc(NC(=O)CCn2nc(C)cc2C)s1] |
| 109A | 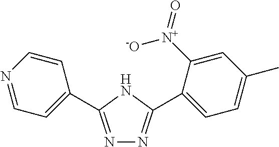 |
| | and |
| 110A | 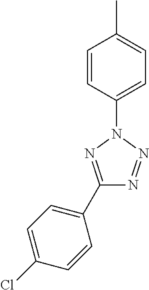 [SMILES: Cc1ccc(-n2nnc(-c3ccc(Cl)cc3)n2)cc1] |

In some embodiments, the compound is selected from the group consisting of

| Compd. | |
|---|---|
| No. | Structure |
| 52B | 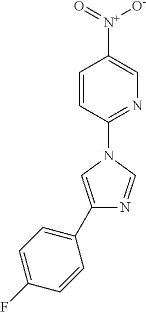 [SMILES: O=[N+]([O-])c1ccc(-n2cnc(-c3ccc(F)cc3)c2)nc1] |
| 53B | 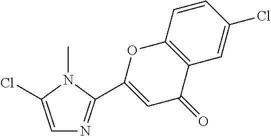 [SMILES: Cn1c(Cl)cnc1-c1cc(=O)c2cc(Cl)ccc2o1] |
| 54B | 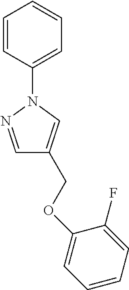 [SMILES: Fc1ccccc1OCc1cnn(-c2ccccc2)c1] |
| 55B | 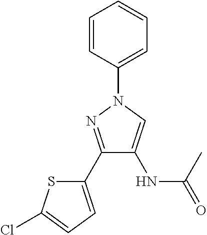 [SMILES: CC(=O)Nc1cn(-c2ccccc2)nc1-c1ccc(Cl)s1] |
| 56B | 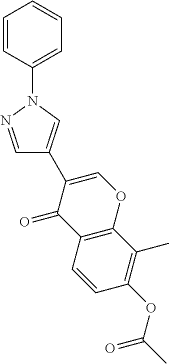 [SMILES: CC(=O)Oc1ccc2c(=O)c(-c3cnn(-c4ccccc4)c3)coc2c1C] |
| 57B | 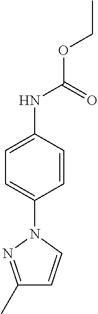 [SMILES: CCOC(=O)Nc1ccc(-n2ccc(C)n2)cc1] |
| 58B | 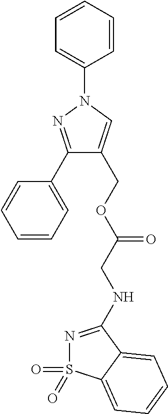 [SMILES: O=C(CNC1=NS(=O)(=O)c2ccccc21)OCc1cn(-c2ccccc2)nc1-c1ccccc1] |
| 59B | 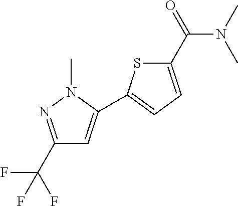 [SMILES: CN(C)C(=O)c1ccc(-c2cc(C(F)(F)F)nn2C)s1] |
| 60B | 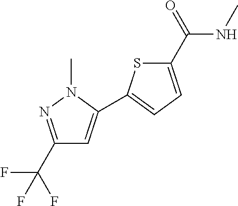 [SMILES: CNC(=O)c1ccc(-c2cc(C(F)(F)F)nn2C)s1] |
| | and |
| 61B | 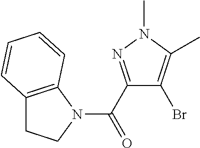 [SMILES: Cc1c(Br)c(C(=O)N2CCc3ccccc32)nn1C] |

In some embodiments, A is an optionally substituted bicyclic 9-membered heterocycle comprising 2-5 nitrogen heteroatoms.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, and tetrazolo[1,5-b]pyridazine.

In some embodiments, A is an optionally substituted bicyclic 9-membered heterocycle comprising 2-5 nitrogen heteroatoms selected from the group consisting of:

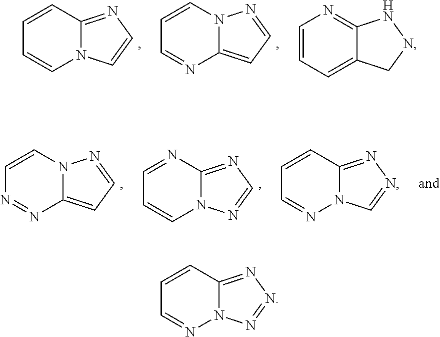

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted monocyclic ring selected from thiophene, pyrrole, benzene, pyridine, imidazole, and 1,2,3,4-tetrahydropyridine.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted monocyclic ring selected from:

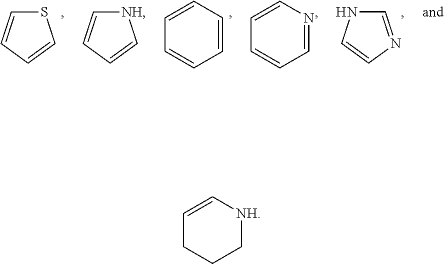

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and C is an optionally substituted benzene.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and L_{b} is selected from the group consisting of a covalent bond, *—NH—**, and *—SCH₂—**.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and L is a covalent bond.

In some embodiments the compound is selected from the group consisting of

| Compd. | |
|---|---|
| No. | Structure |
| 100 | 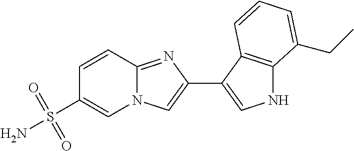 [SMILES: CCc1cccc2c(-c3cn4cc(S(N)(=O)=O)ccc4n3)c[nH]c12] |
| 101 | 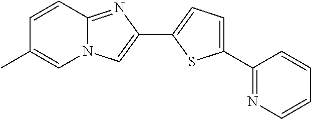 [SMILES: Cc1ccc2nc(-c3ccc(-c4ccccn4)s3)cn2c1] |
| 102 | 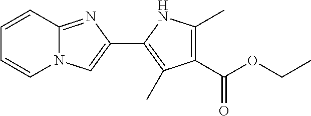 [SMILES: CCOC(=O)c1c(C)[nH]c(-c2cn3ccccc3n2)c1C] |
| 103 | 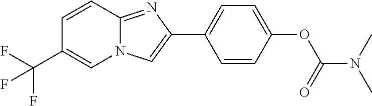 [SMILES: CN(C)C(=O)Oc1ccc(-c2cn3cc(C(F)(F)F)ccc3n2)cc1] |
| 104 | 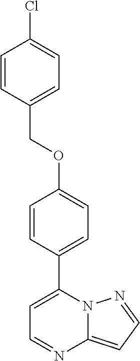 [SMILES: Clc1ccc(COc2ccc(-c3ccnc4ccnn34)cc2)cc1] |
| 105 | 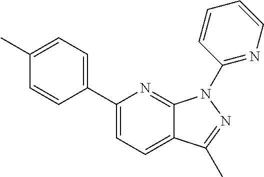 [SMILES: Cc1ccc(-c2ccc3c(C)nn(-c4ccccn4)c3n2)cc1] |
| 106 | 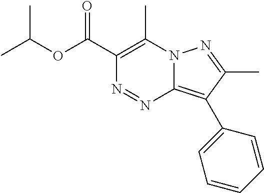 [SMILES: Cc1nn2c(C)c(C(=O)OC(C)C)nnc2c1-c1ccccc1] |
| 107 | 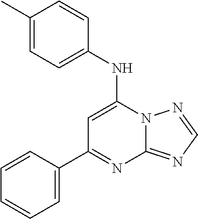 [SMILES: Cc1ccc(Nc2cc(-c3ccccc3)nc3ncnn23)cc1] |
| 108 | 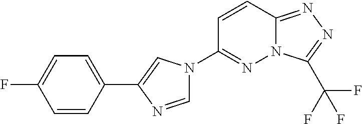 [SMILES: Fc1ccc(-c2cn(-c3ccc4nnc(C(F)(F)F)n4n3)cn2)cc1] |
| 110 | 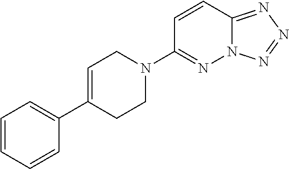 [SMILES: C1=C(c2ccccc2)CCN(c2ccc3nnnn3n2)C1] |
| | and |
| 125 | 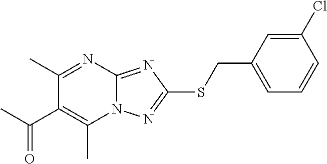 [SMILES: CC(=O)c1c(C)nc2nc(SCc3cccc(Cl)c3)nn2c1C] |

In some embodiments, the compound is selected from the group consisting of

| Compd. | |
|---|---|
| No. | Structure |
| 111A | 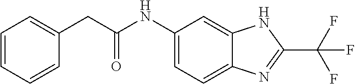 [SMILES: O=C(Cc1ccccc1)Nc1ccc2nc(C(F)(F)F)[nH]c2c1] |
| 112A | 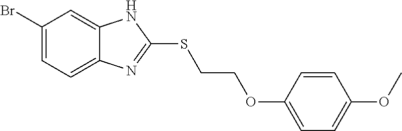 [SMILES: COc1ccc(OCCSc2nc3ccc(Br)cc3[nH]2)cc1] |
| 113A | 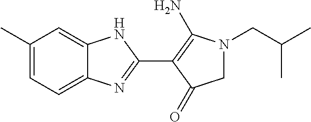 [SMILES: Cc1ccc2nc(C3=C(N)N(CC(C)C)CC3=O)[nH]c2c1] |
| 114A | 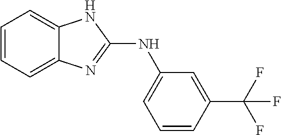 [SMILES: FC(F)(F)c1cccc(Nc2nc3ccccc3[nH]2)c1] |
| 115A | 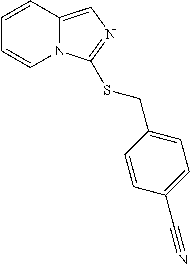 [SMILES: N#Cc1ccc(CSc2ncc3ccccn23)cc1] |
| 116A | 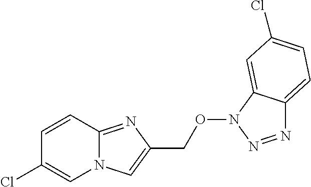 [SMILES: Clc1ccc2nnn(OCc3cn4cc(Cl)ccc4n3)c2c1] |
| 117A | 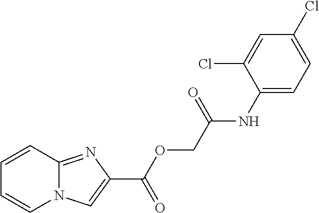 [SMILES: O=C(COC(=O)c1cn2ccccc2n1)Nc1ccc(Cl)cc1Cl] |
| 118A | 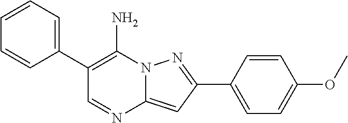 [SMILES: COc1ccc(-c2cc3ncc(-c4ccccc4)c(N)n3n2)cc1] |
| 119A | 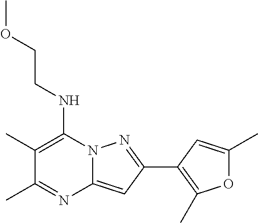 |
| 120A | 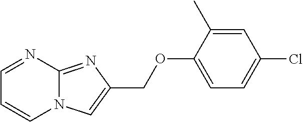 [SMILES: Cc1cc(Cl)ccc1OCc1cn2cccnc2n1] |
| 121A | 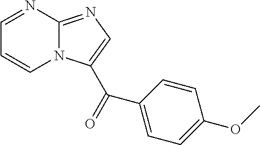 [SMILES: COc1ccc(C(=O)c2cnc3ncccn23)cc1] |
| 122A | 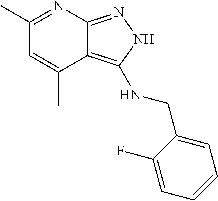 [SMILES: Cc1cc(C)c2c(NCc3ccccc3F)[nH]nc2n1] |
| 123A | 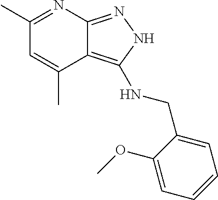 [SMILES: COc1ccccc1CNc1[nH]nc2nc(C)cc(C)c12] |
| 124A | 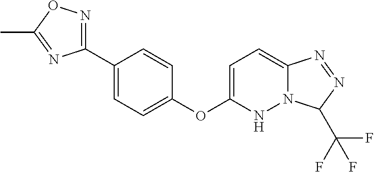 [SMILES: Cc1nc(-c2ccc(OC3=CC=C4N=NC(C(F)(F)F)N4N3)cc2)no1] |
| 125A | 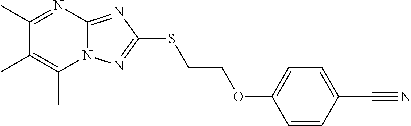 [SMILES: Cc1nc2nc(SCCOc3ccc(C#N)cc3)nn2c(C)c1C] |
| | and |
| 126A | 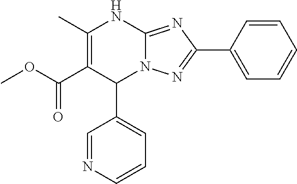 [SMILES: COC(=O)C1=C(C)Nc2nc(-c3ccccc3)nn2C1c1cccnc1] |

In some embodiments, the compound is selected from the group consisting of

| Compd. | |
|---|---|
| No. | Structure |
| 62B | 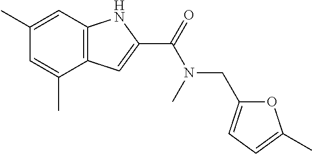 |
| 63B | 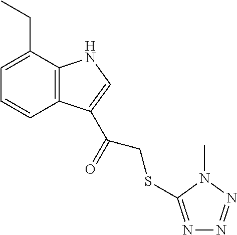 [SMILES: CCc1cccc2c(C(=O)CSc3nnnn3C)c[nH]c12] |
| 64B | 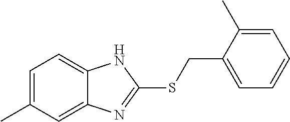 [SMILES: Cc1ccc2[nH]c(SCc3ccccc3C)nc2c1] |
| 65B | 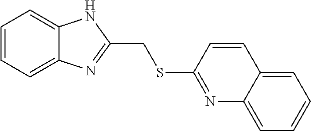 [SMILES: c1ccc2nc(SCc3nc4ccccc4[nH]3)ccc2c1] |
| 66B | 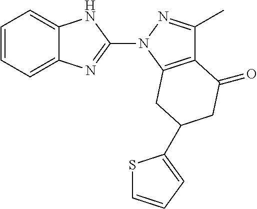 [SMILES: Cc1nn(-c2nc3ccccc3[nH]2)c2c1C(=O)CC(c1cccs1)C2] |
| 67B | 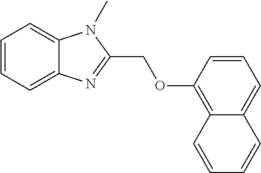 [SMILES: Cn1c(COc2cccc3ccccc23)nc2ccccc21] |
| 68B | 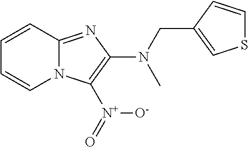 [SMILES: CN(Cc1ccsc1)c1nc2ccccn2c1[N+](=O)[O-]] |
| 69B | 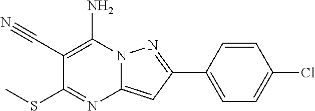 [SMILES: CSc1nc2cc(-c3ccc(Cl)cc3)nn2c(N)c1C#N] |
| 70B | 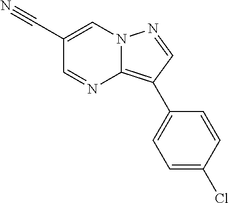 [SMILES: N#Cc1cnc2c(-c3ccc(Cl)cc3)cnn2c1] |
| 71B | 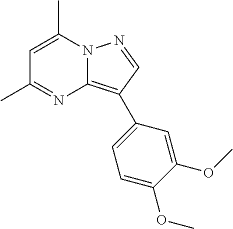 |
| 72B | 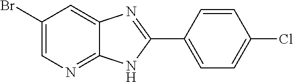 [SMILES: Clc1ccc(-c2nc3cc(Br)cnc3[nH]2)cc1] |
| 73B | 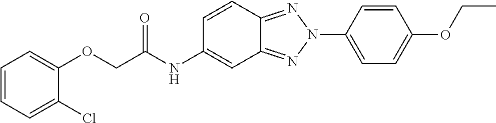 [SMILES: CCOc1ccc(-n2nc3ccc(NC(=O)COc4ccccc4Cl)cc3n2)cc1] |
| 74B | 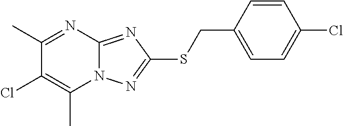 [SMILES: Cc1nc2nc(SCc3ccc(Cl)cc3)nn2c(C)c1Cl] |
| | and |
| 75B | 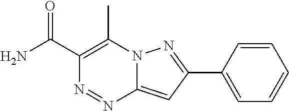 [SMILES: Cc1c(C(N)=O)nnc2cc(-c3ccccc3)nn12] |

In some embodiments, A is an optionally substituted monocyclic 5-membered heterocycle comprising 1 oxygen heteroatom and 1-2 nitrogen heteroatoms.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole.

In some embodiments, A is an optionally substituted monocyclic 5-membered heterocycle comprising 1 oxygen heteroatom and 1-2 nitrogen heteroatoms selected from the group consisting of:

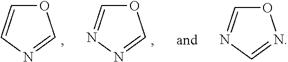

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and B is an optionally substituted monocyclic ring selected from isoxazole, pyridine, pyrazine, thiophene, and benzene.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and B is an optionally substituted monocyclic ring selected from:

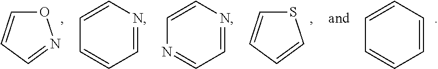

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and C is an optionally substituted monocyclic ring selected from pyrazole and benzene.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and L_{b} is selected from the group consisting of a covalent bond and *—CH₂NH—**.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and L is a covalent bond.

In some embodiments, the compound is selected from the group consisting of

| Compd. | |
|---|---|
| No. | Structure |
| 122 | 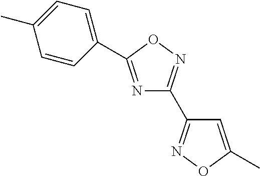 |
| 123 | 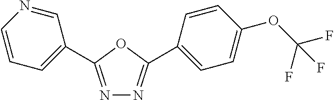 [SMILES: FC(F)(F)Oc1ccc(-c2nnc(-c3cccnc3)o2)cc1] |
| 140 | 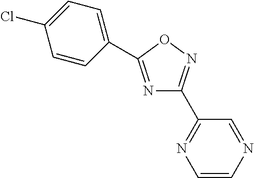 [SMILES: Clc1ccc(-c2nc(-c3cnccn3)no2)cc1] |
| 141 | 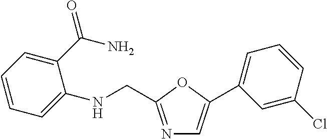 [SMILES: NC(=O)c1ccccc1NCc1ncc(-c2cccc(Cl)c2)o1] |
| 144 | 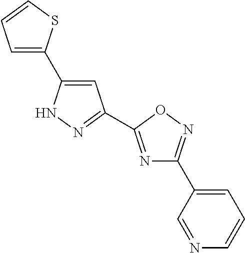 [SMILES: c1cncc(-c2noc(-c3cc(-c4cccs4)[nH]n3)n2)c1] |
| 149 | 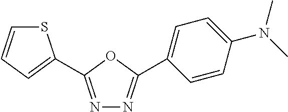 [SMILES: CN(C)c1ccc(-c2nnc(-c3cccs3)o2)cc1] |
| | and |
| 157 | 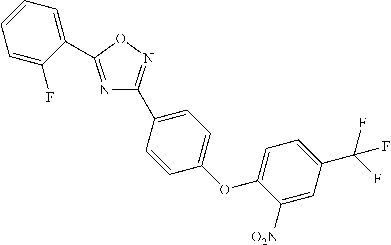 [SMILES: O=[N+]([O-])c1cc(C(F)(F)F)ccc1Oc1ccc(-c2noc(-c3ccccc3F)n2)cc1] |

In some embodiments, the compound is selected from the group consisting of

| Compd. | |
|---|---|
| No. | Structure |
| 127A | 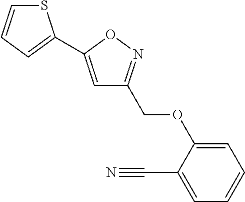 [SMILES: N#Cc1ccccc1OCc1cc(-c2cccs2)on1] |
| 128A | 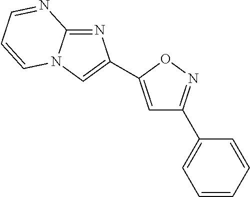 [SMILES: c1ccc(-c2cc(-c3cn4cccnc4n3)on2)cc1] |
| 129A | 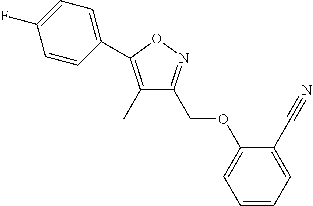 [SMILES: Cc1c(COc2ccccc2C#N)noc1-c1ccc(F)cc1] |
| 130A | 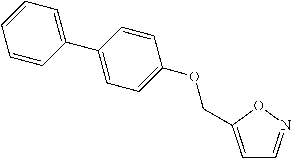 [SMILES: c1ccc(-c2ccc(OCc3ccno3)cc2)cc1] |
| 131A | 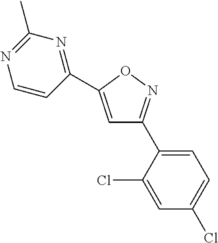 [SMILES: Cc1nccc(-c2cc(-c3ccc(Cl)cc3Cl)no2)n1] |
| 132A | 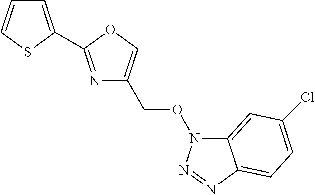 [SMILES: Clc1ccc2nnn(OCc3coc(-c4cccs4)n3)c2c1] |
| 133A | 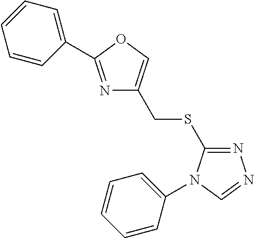 [SMILES: c1ccc(-c2nc(CSc3nncn3-c3ccccc3)co2)cc1] |
| 134A | 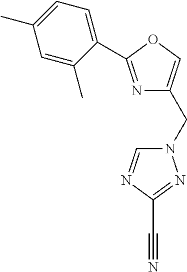 [SMILES: Cc1ccc(-c2nc(Cn3cnc(C#N)n3)co2)c(C)c1] |
| 135A | 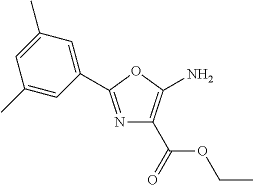 [SMILES: CCOC(=O)c1nc(-c2cc(C)cc(C)c2)oc1N] |
| 136A | 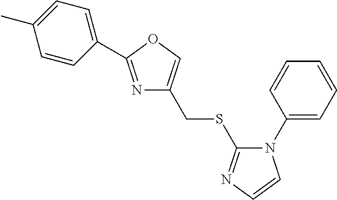 [SMILES: Cc1ccc(-c2nc(CSc3nccn3-c3ccccc3)co2)cc1] |
| 137A | 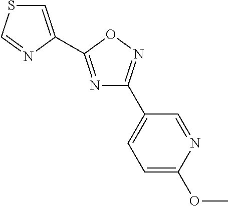 [SMILES: COc1ccc(-c2noc(-c3cscn3)n2)cn1] |
| 138A | 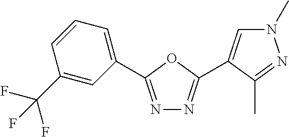 [SMILES: Cc1nn(C)cc1-c1nnc(-c2cccc(C(F)(F)F)c2)o1] |
| 139A | 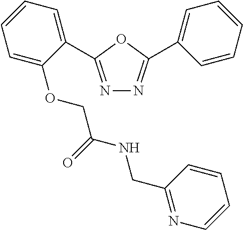 [SMILES: O=C(COc1ccccc1-c1nnc(-c2ccccc2)o1)NCc1ccccn1] |
| | and |
| 140A | 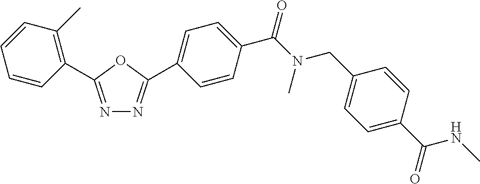 [SMILES: CNC(=O)c1ccc(CN(C)C(=O)c2ccc(-c3nnc(-c4ccccc4C)o3)cc2)cc1] |

In some embodiments, the compound is selected from the group consisting of

| Compd. | |
|---|---|
| No. | Structure |
| 76B | 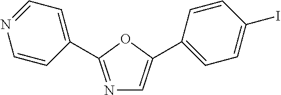 [SMILES: Ic1ccc(-c2cnc(-c3ccncc3)o2)cc1] |
| 77B | 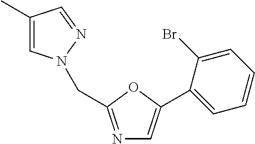 [SMILES: Cc1cnn(Cc2ncc(-c3ccccc3Br)o2)c1] |
| 78B | 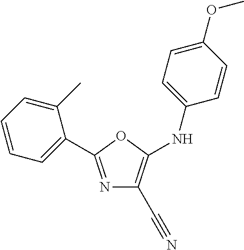 [SMILES: COc1ccc(Nc2oc(-c3ccccc3C)nc2C#N)cc1] |
| 79B | 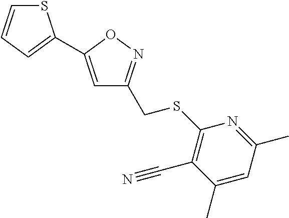 [SMILES: Cc1cc(C)c(C#N)c(SCc2cc(-c3cccs3)on2)n1] |
| 80B | 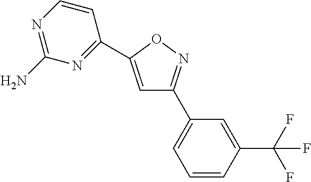 [SMILES: Nc1nccc(-c2cc(-c3cccc(C(F)(F)F)c3)no2)n1] |
| 81B | 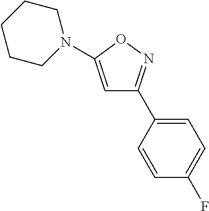 [SMILES: Fc1ccc(-c2cc(N3CCCCC3)on2)cc1] |
| 82B | 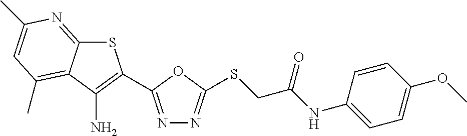 |
| 83B | 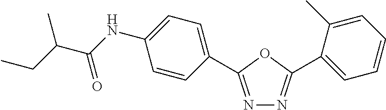 [SMILES: CCC(C)C(=O)Nc1ccc(-c2nnc(-c3ccccc3C)o2)cc1] |
| 84B | 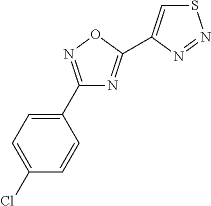 [SMILES: Clc1ccc(-c2noc(-c3csnn3)n2)cc1] |
| 85B | 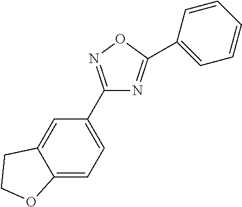 [SMILES: c1ccc(-c2nc(-c3ccc4c(c3)CCO4)no2)cc1] |
| 86B | 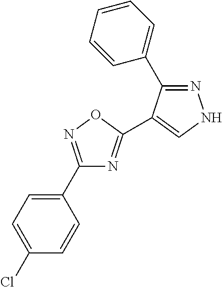 [SMILES: Clc1ccc(-c2noc(-c3c[nH]nc3-c3ccccc3)n2)cc1] |
| | and |
| 87B | 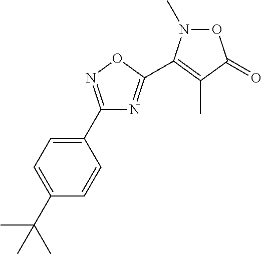 [SMILES: Cc1c(-c2nc(-c3ccc(C(C)(C)C)cc3)no2)n(C)oc1=O] |

In some embodiments, A is an optionally substituted benzene.

In some embodiments, A is an optionally substituted benzene and B is an optionally substituted monocyclic ring selected from the group consisting of benzene, thiophene, 2,3-dihydrothiazole, and 1,2,3,6-tetrahydropyridine.

In some embodiments, A is an optionally substituted benzene and B is an optionally substituted monocyclic ring selected from the group consisting of:

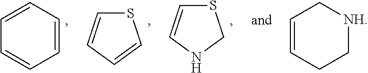

In some embodiments, A is an optionally substituted benzene and C is an optionally substituted monocyclic ring selected from the group consisting of benzene and isoxazole.

In some embodiments, A is an optionally substituted benzene and L_{b} is selected from the group consisting of a covalent bond, *—C(O)—N=**, *—OCH₂C(O)NH—**, and *—NHC(O)CH₂NH—**.

In some embodiments, A is an optionally substituted benzene and L is *—OCH₂—**.

In some embodiments the compound is selected from the group consisting of

| Compd. | |
|---|---|
| No. | Structure |
| 118 | 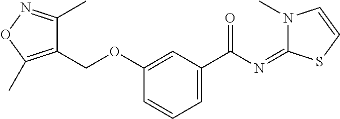 [SMILES: Cc1noc(C)c1COc1cccc(C(=O)/N=c2/sccn2C)c1] |
| 119 | 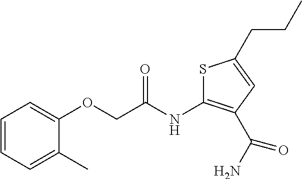 [SMILES: CCCc1cc(C(N)=O)c(NC(=O)COc2ccccc2C)s1] |
| 127 | 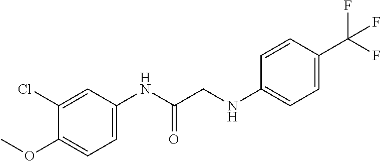 [SMILES: COc1ccc(NC(=O)CNc2ccc(C(F)(F)F)cc2)cc1Cl] |
| | and |
| 143 | 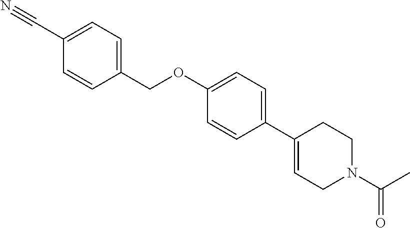 [SMILES: CC(=O)N1CC=C(c2ccc(OCc3ccc(C#N)cc3)cc2)CC1] |

In some embodiments, the compound is selected from the group consisting of

| Compd. | |
|---|---|
| No. | Structure |
| 141A | 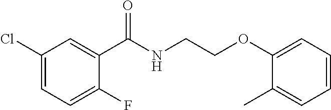 [SMILES: Cc1ccccc1OCCNC(=O)c1cc(Cl)ccc1F] |
| 142A | 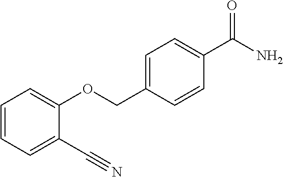 [SMILES: N#Cc1ccccc1OCc1ccc(C(N)=O)cc1] |
| | and |
| 143A | 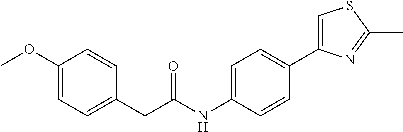 |

In some embodiments, the compound is selected from the group consisting of

| Compd. | |
|---|---|
| No. | Structure |
| 88B | 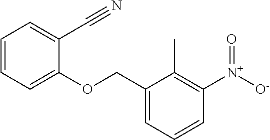 [SMILES: Cc1c(COc2ccccc2C#N)cccc1[N+](=O)[O-]] |
| 89B | 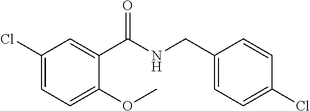 [SMILES: COc1ccc(Cl)cc1C(=O)NCc1ccc(Cl)cc1] |
| 90B | 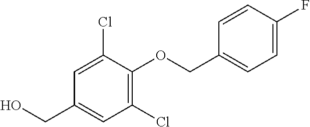 [SMILES: OCc1cc(Cl)c(OCc2ccc(F)cc2)c(Cl)c1] |
| 91B | 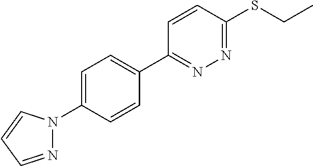 [SMILES: CCSc1ccc(-c2ccc(-n3cccn3)cc2)nn1] |
| 92B | 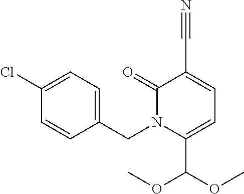 [SMILES: COC(OC)c1ccc(C#N)c(=O)n1Cc1ccc(Cl)cc1] |
| 93B | 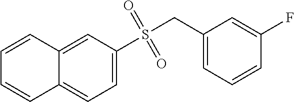 [SMILES: O=S(=O)(Cc1cccc(F)c1)c1ccc2ccccc2c1] |
| 94B | 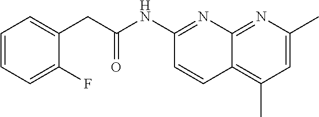 [SMILES: Cc1cc(C)c2ccc(NC(=O)Cc3ccccc3F)nc2n1] |
| 95B | 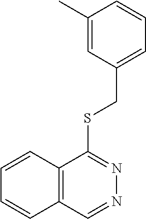 [SMILES: Cc1cccc(CSc2nncc3ccccc23)c1] |
| 96B | 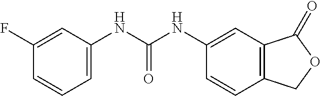 [SMILES: O=C(Nc1cccc(F)c1)Nc1ccc2c(c1)C(=O)OC2] |
| | and |
| 97B | 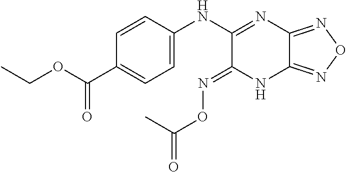 [SMILES: CCOC(=O)c1ccc(Nc2nc3nonc3[nH]/c2=N\OC(C)=O)cc1] |

In some embodiments, A is an optionally substituted monocyclic 5-membered heterocycle comprising 1-3 heteroatoms selected from nitrogen, oxygen, and sulfur.

In some embodiments, the compound is selected from the group consisting of

| Compd. | |
|---|---|
| No. | Structure |
| 144A | 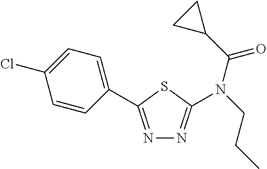 [SMILES: CCCN(C(=O)C1CC1)c1nnc(-c2ccc(Cl)cc2)s1] |
| 145A | 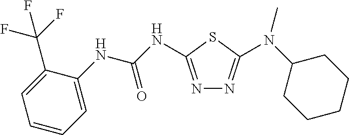 [SMILES: CN(c1nnc(NC(=O)Nc2ccccc2C(F)(F)F)s1)C1CCCCC1] |
| 146A | 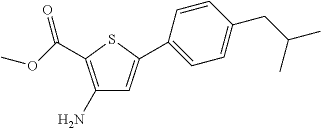 [SMILES: COC(=O)c1sc(-c2ccc(CC(C)C)cc2)cc1N] |
| 147A | 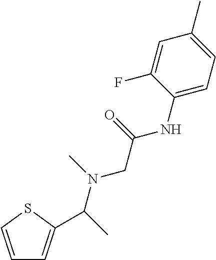 [SMILES: Cc1ccc(NC(=O)CN(C)C(C)c2cccs2)c(F)c1] |
| 148A | 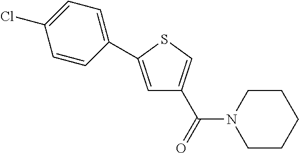 [SMILES: O=C(c1csc(-c2ccc(Cl)cc2)c1)N1CCCCC1] |
| 149A | 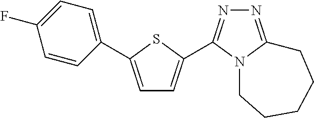 [SMILES: Fc1ccc(-c2ccc(-c3nnc4n3CCCCC4)s2)cc1] |
| 150A | 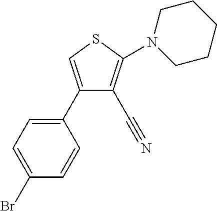 [SMILES: N#Cc1c(-c2ccc(Br)cc2)csc1N1CCCCC1] |
| 151A | 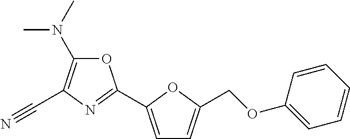 [SMILES: CN(C)c1oc(-c2ccc(COc3ccccc3)o2)nc1C#N] |
| 152A | 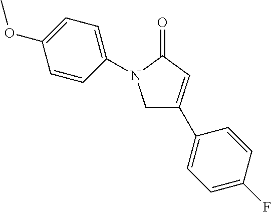 [SMILES: COc1ccc(N2CC(c3ccc(F)cc3)=CC2=O)cc1] |
| | and |
| 153A | 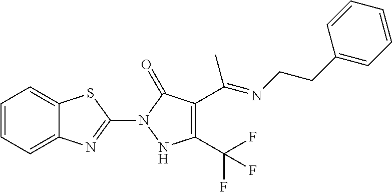 [SMILES: C/C(=N\CCc1ccccc1)c1c(C(F)(F)F)[nH]n(-c2nc3ccccc3s2)c1=O] |

In some embodiments, the compound is selected from the group consisting of

| Compd. | |
|---|---|
| No. | Structure |
| 98B | 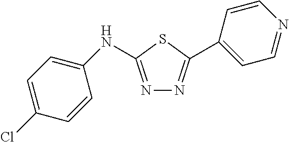 [SMILES: Clc1ccc(Nc2nnc(-c3ccncc3)s2)cc1] |
| 99B | 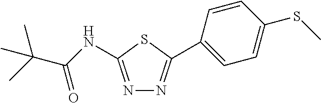 [SMILES: CSc1ccc(-c2nnc(NC(=O)C(C)(C)C)s2)cc1] |
| 100B | 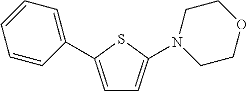 [SMILES: c1ccc(-c2ccc(N3CCOCC3)s2)cc1] |
| 101B | 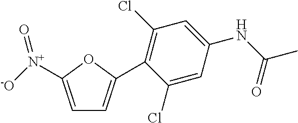 |
| 102B | 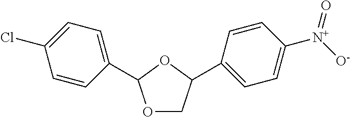 [SMILES: O=[N+]([O-])c1ccc(C2COC(c3ccc(Cl)cc3)O2)cc1] |
| 103B | 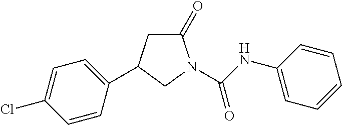 [SMILES: O=C1CC(c2ccc(Cl)cc2)CN1C(=O)Nc1ccccc1] |

In some embodiments, A is an optionally substituted bicyclic 10-membered heterocycle comprising 1-2 nitrogen heteroatoms.

In some embodiments, A is an optionally substituted bicyclic 10-membered heterocycle comprising 1-2 nitrogen heteroatoms selected from the group consisting of quinolone, quinoxaline, and pthalazine.

In some embodiments, A is an optionally substituted bicyclic 10-membered heterocycle comprising 1-2 nitrogen heteroatoms selected from the group consisting of:

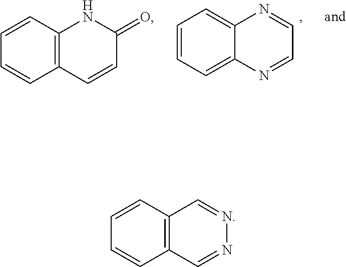

In some embodiments, A is an optionally substituted bicyclic 10-membered heterocycle comprising 1-2 nitrogen heteroatoms selected from the group consisting of quinolone, quinoxaline, and pthalazine and B is an optionally substituted monocyclic ring selected from benzene and pyrimidine.

In some embodiments, A is an optionally substituted bicyclic 10-membered heterocycle comprising 1-2 nitrogen heteroatoms selected from the group consisting of quinolone, quinoxaline, and pthalazine and C is an optionally substituted benzene.

In some embodiments, A is an optionally substituted bicyclic 10-membered heterocycle comprising 1-2 nitrogen heteroatoms selected from the group consisting of quinolone, quinoxaline, and pthalazine and L_{b} is selected from the group consisting of a covalent bond and *—NH—**.

In some embodiments, A is an optionally substituted bicyclic 10-membered heterocycle comprising 1-2 nitrogen heteroatoms selected from the group consisting of quinolone, quinoxaline, and pthalazine and L_{c} is a covalent bond.

In some embodiments, the compound is selected from the group consisting of

| Compd. | |
|---|---|
| No. | Structure |
| 116 | 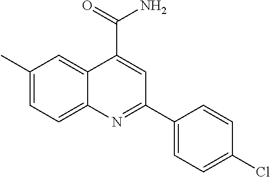 [SMILES: Cc1ccc2nc(-c3ccc(Cl)cc3)cc(C(N)=O)c2c1] |
| 124 | 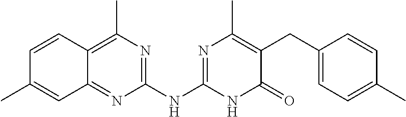 |
| 130 | 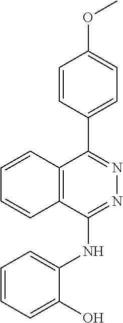 [SMILES: COc1ccc(-c2nnc(Nc3ccccc3O)c3ccccc23)cc1] |
| 128 | 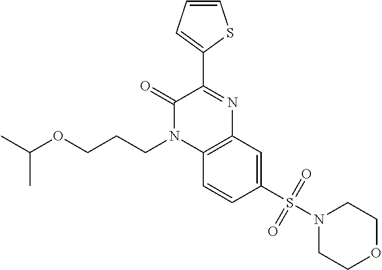 [SMILES: CC(C)OCCCn1c(=O)c(-c2cccs2)nc2cc(S(=O)(=O)N3CCOCC3)ccc21] |
| 129 | 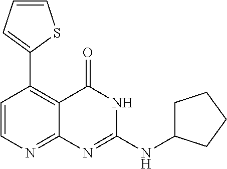 [SMILES: O=c1[nH]c(NC2CCCC2)nc2nccc(-c3cccs3)c12] |
| 131 | 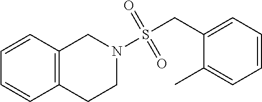 [SMILES: Cc1ccccc1CS(=O)(=O)N1CCc2ccccc2C1] |
| 132 | 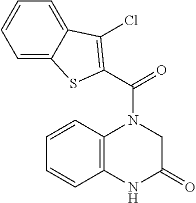 [SMILES: O=C1CN(C(=O)c2sc3ccccc3c2Cl)c2ccccc2N1] |
| 134 | 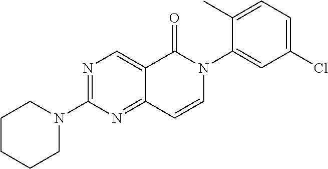 [SMILES: Cc1ccc(Cl)cc1-n1ccc2nc(N3CCCCC3)ncc2c1=O] |
| 151 | 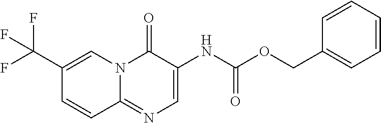 [SMILES: O=C(Nc1cnc2ccc(C(F)(F)F)cn2c1=O)OCc1ccccc1] |
| | and |
| 156 | 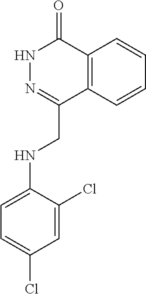 [SMILES: O=c1[nH]nc(CNc2ccc(Cl)cc2Cl)c2ccccc12] |

In some embodiments, the compound is selected from the group consisting of

| Compd. | |
|---|---|
| No. | Structure |
| 154A | 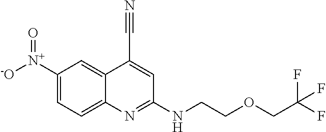 |
| 155A | 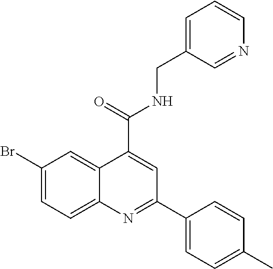 [SMILES: Cc1ccc(-c2cc(C(=O)NCc3cccnc3)c3cc(Br)ccc3n2)cc1] |
| 156A | 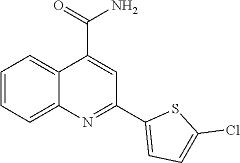 [SMILES: NC(=O)c1cc(-c2ccc(Cl)s2)nc2ccccc12] |
| 157A | 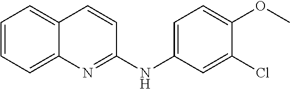 [SMILES: COc1ccc(Nc2ccc3ccccc3n2)cc1Cl] |
| 158A | 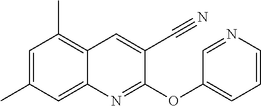 [SMILES: Cc1cc(C)c2cc(C#N)c(Oc3cccnc3)nc2c1] |
| 159A | 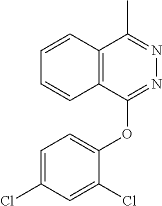 [SMILES: Cc1nnc(Oc2ccc(Cl)cc2Cl)c2ccccc12] |
| 160A | 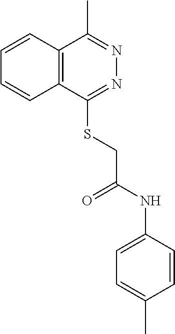 [SMILES: Cc1ccc(NC(=O)CSc2nnc(C)c3ccccc23)cc1] |
| 161A | 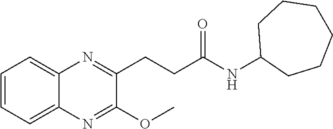 [SMILES: COc1nc2ccccc2nc1CCC(=O)NC1CCCCCC1] |
| 162A | 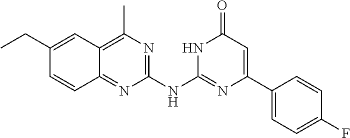 [SMILES: CCc1ccc2nc(Nc3nc(-c4ccc(F)cc4)cc(=O)[nH]3)nc(C)c2c1] |
| 163A | 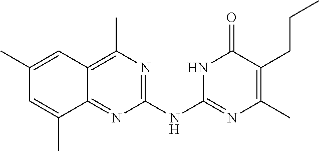 [SMILES: CCCc1c(C)nc(Nc2nc(C)c3cc(C)cc(C)c3n2)[nH]c1=O] |
| 164A | 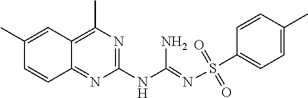 |
| 165A | 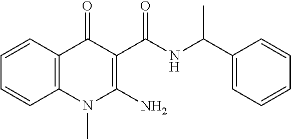 [SMILES: CC(NC(=O)c1c(N)n(C)c2ccccc2c1=O)c1ccccc1] |
| 166A | 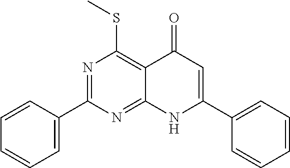 [SMILES: CSc1nc(-c2ccccc2)nc2[nH]c(-c3ccccc3)cc(=O)c12] |
| 167A | 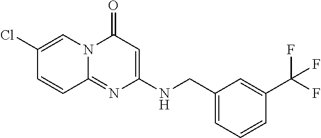 [SMILES: O=c1cc(NCc2cccc(C(F)(F)F)c2)nc2ccc(Cl)cn12] |
| | and |
| 168A | 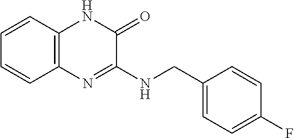 [SMILES: O=c1[nH]c2ccccc2nc1NCc1ccc(F)cc1] |

In some embodiments, the compound is selected from the group consisting of

| Compd. | |
|---|---|
| No. | Structure |
| 104B | 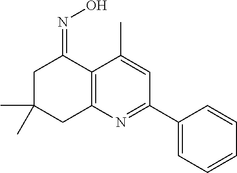 [SMILES: Cc1cc(-c2ccccc2)nc2c1/C(=N\O)CC(C)(C)C2] |
| | and |
| 105B | 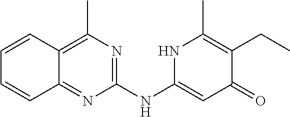 [SMILES: CCc1c(C)[nH]c(Nc2nc(C)c3ccccc3n2)cc1=O] |

In some embodiments, A is an optionally substituted bicyclic 10-membered heterocycle comprising 1-3 nitrogen heteroatoms.

In some embodiments, A is an optionally substituted bicyclic 10-membered heterocycle comprising 1-3 nitrogen heteroatoms and B is an optionally substituted monocyclic ring selected from the group consisting of benzene and thiophene.

In some embodiments, A is an optionally substituted bicyclic 10-membered heterocycle comprising 1-3 nitrogen heteroatoms and B is an optionally substituted benzo[ ]thiophene.

In some embodiments, A is an optionally substituted bicyclic 10-membered heterocycle comprising 1-3 nitrogen heteroatoms and C is an optionally substituted monocyclic ring selected from the group consisting of piperidine and morpholine.

In some embodiments, A is an optionally substituted bicyclic 10-membered heterocycle comprising 1-3 nitrogen heteroatoms and L_{b} is selected from the group consisting of a covalent bond, *—NHC(O)OCH₂—**, *—CH₂NH—**, *—SO₂CH₂—**, and *—C(O)—**.

In some embodiments, A is an optionally substituted bicyclic 10-membered heterocycle comprising 1-3 nitrogen heteroatoms and L_{c} is selected from the group consisting of a covalent bond and *—SO₂—**.

In some embodiments, A is an optionally substituted bicyclic 9-membered heterocycle comprising 1-2 nitrogen heteroatoms and 1 sulfur heteroatom.

In some embodiments, A is an optionally substituted bicyclic 9-membered heterocycle comprising 1-2 nitrogen heteroatoms and 1 sulfur heteroatom and B is an optionally substituted benzene.

In some embodiments, A is an optionally substituted bicyclic 9-membered heterocycle comprising 1-2 nitrogen heteroatoms and 1 sulfur heteroatom and c is 0.

In some embodiments, A is an optionally substituted bicyclic 9-membered heterocycle comprising 1-2 nitrogen heteroatoms and 1 sulfur heteroatom and L_{b} is selected from the group consisting of a covalent bond, *—O—**, and *—NHC(O)NH—**.

In some embodiments, the compound is selected from the group consisting of

| Compd. | |
|---|---|
| No. | Structure |
| 121 | 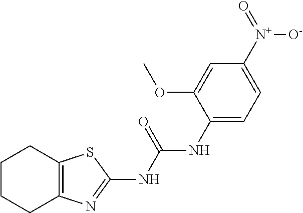 [SMILES: COc1cc([N+](=O)[O-])ccc1NC(=O)Nc1nc2c(s1)CCCC2] |
| 136 | 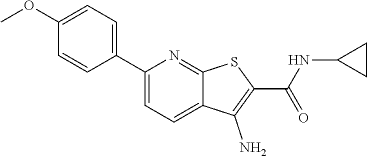 [SMILES: COc1ccc(-c2ccc3c(N)c(C(=O)NC4CC4)sc3n2)cc1] |
| 138 | 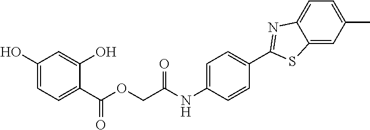 [SMILES: Cc1ccc2nc(-c3ccc(NC(=O)COC(=O)c4ccc(O)cc4O)cc3)sc2c1] |
| 147 | 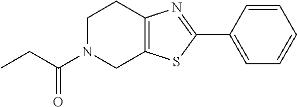 [SMILES: CCC(=O)N1CCc2nc(-c3ccccc3)sc2C1] |
| | and |
| 150 | 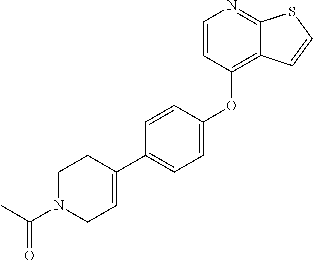 [SMILES: CC(=O)N1CC=C(c2ccc(Oc3ccnc4sccc34)cc2)CC1] |

In some embodiments, the compound is selected from the group consisting of

| Compd. | |
|---|---|
| No. | Structure |
| 169A | 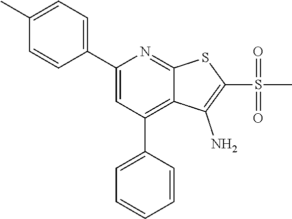 |
| 170A | 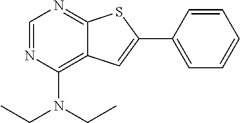 [SMILES: CCN(CC)c1ncnc2sc(-c3ccccc3)cc12] |
| | and |
| 171A | 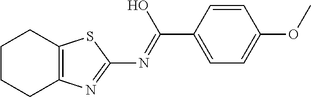 [SMILES: COc1ccc(/C(O)=N/c2nc3c(s2)CCCC3)cc1] |

In some embodiments, the compound is selected from the group consisting of

| Compd. | |
|---|---|
| No. | Structure |
| 106B | 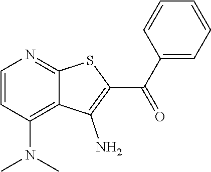 [SMILES: CN(C)c1ccnc2sc(C(=O)c3ccccc3)c(N)c12] |
| 107B | 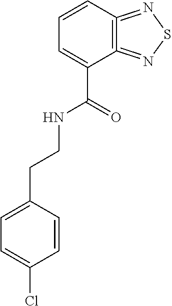 |
| | and |
| 108B | 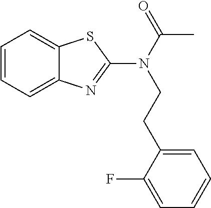 [SMILES: CC(=O)N(CCc1ccccc1F)c1nc2ccccc2s1] |

In some embodiments, A is an optionally substituted bicyclic 8- to 10-membered heterocycle comprising 1-4 heteroatoms selected from N, O, and S.

In some embodiments, A is an optionally substituted bicyclic 9-membered heterocycle comprising 1-4 nitrogen heteroatoms.

In some embodiments, A is an optionally substituted bicyclic 9-membered heterocycle comprising 1-4 nitrogen heteroatoms and B is an optionally substituted benzene.

In some embodiments, A is an optionally substituted bicyclic 9-membered heterocycle comprising 1-4 nitrogen heteroatoms and C is an optionally substituted benzene.

In some embodiments, A is an optionally substituted bicyclic 9-membered heterocycle comprising 1-4 nitrogen heteroatoms and L_{b} is covalent bond.

In some embodiments, A is an optionally substituted bicyclic 9-membered heterocycle comprising 1-4 nitrogen heteroatoms and L_{c} is covalent bond.

In some embodiments the compound is selected from the group consisting of

| Compd. | |
|---|---|
| No. | Structure |
| 109 | 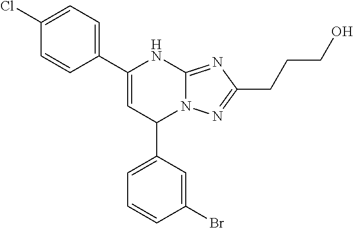 [SMILES: OCCCc1nc2n(n1)C(c1cccc(Br)c1)C=C(c1ccc(Cl)cc1)N2] |
| 117 | 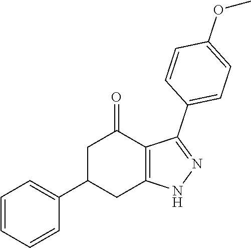 [SMILES: COc1ccc(-c2n[nH]c3c2C(=O)CC(c2ccccc2)C3)cc1] |
| 135 | 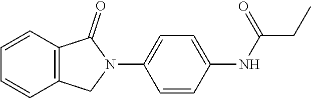 [SMILES: CCC(=O)Nc1ccc(N2Cc3ccccc3C2=O)cc1] |
| | and |
| 137 | 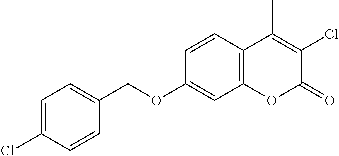 [SMILES: Cc1c(Cl)c(=O)oc2cc(OCc3ccc(Cl)cc3)ccc12] |

In some embodiments, the compound is selected from the group consisting of

| Compd. | |
|---|---|
| No. | Structure |
| 172A | 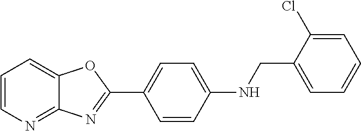 [SMILES: Clc1ccccc1CNc1ccc(-c2nc3ncccc3o2)cc1] |
| 173A | 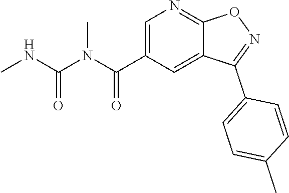 [SMILES: CNC(=O)N(C)C(=O)c1cnc2onc(-c3ccc(C)cc3)c2c1] |
| 174A | 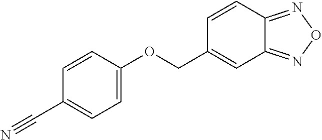 [SMILES: N#Cc1ccc(OCc2ccc3nonc3c2)cc1] |
| 175A | 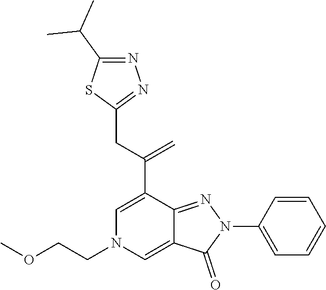 [SMILES: C=C(Cc1nnc(C(C)C)s1)c1cn(CCOC)cc2c(=O)n(-c3ccccc3)nc1-2] |
| 176A | 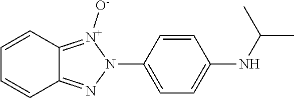 [SMILES: CC(C)Nc1ccc(-n2nc3ccccc3[n+]2[O-])cc1] |
| 177A | 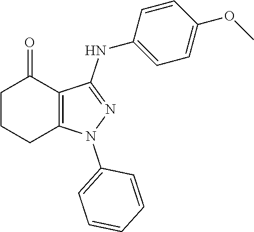 [SMILES: COc1ccc(Nc2nn(-c3ccccc3)c3c2C(=O)CCC3)cc1] |
| 178A | 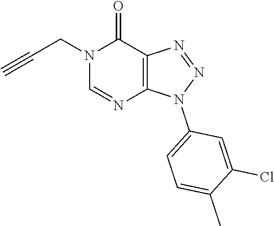 [SMILES: C#CCn1cnc2c(nnn2-c2ccc(C)c(Cl)c2)c1=O] |
| 179A | 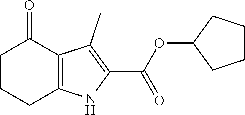 [SMILES: Cc1c(C(=O)OC2CCCC2)[nH]c2c1C(=O)CCC2] |
| 180A | 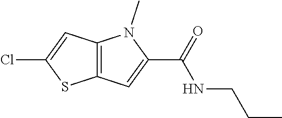 [SMILES: CCCNC(=O)c1cc2sc(Cl)cc2n1C] |
| 181A | 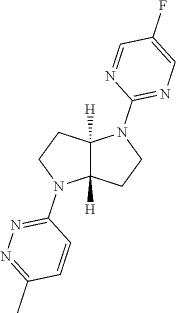 [SMILES: Cc1ccc(N2CC[C@@H]3[C@@H]2CCN3c2ncc(F)cn2)nn1] |
| | and |
| 182A | 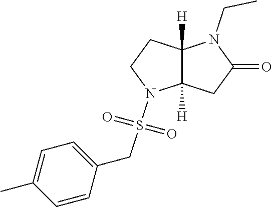 [SMILES: CCN1C(=O)C[C@@H]2[C@@H]1CCN2S(=O)(=O)Cc1ccc(C)cc1] |

In some embodiments, the compound is selected from the group consisting of

| Compd. | |
|---|---|
| No. | Structure |
| 109B | 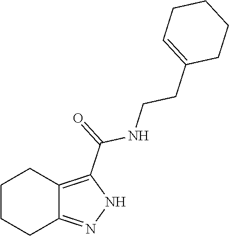 [SMILES: O=C(NCCC1=CCCCC1)c1[nH]nc2c1CCCC2] |
| 110B | 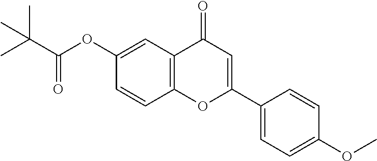 [SMILES: COc1ccc(-c2cc(=O)c3cc(OC(=O)C(C)(C)C)ccc3o2)cc1] |
| 111B | 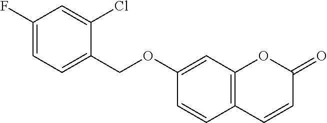 [SMILES: O=c1ccc2ccc(OCc3ccc(F)cc3Cl)cc2o1] |
| 112B | 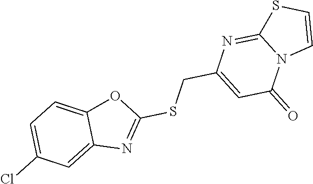 [SMILES: O=c1cc(CSc2nc3cc(Cl)ccc3o2)nc2sccn12] |
| 113B | 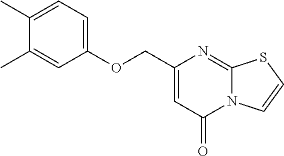 [SMILES: Cc1ccc(OCc2cc(=O)n3ccsc3n2)cc1C] |
| 114B | 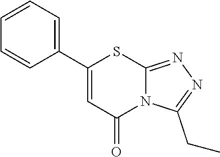 [SMILES: CCc1nnc2sc(-c3ccccc3)cc(=O)n12] |
| 115B | 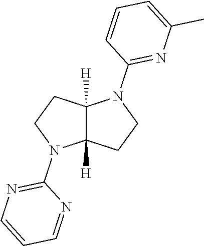 [SMILES: Cc1cccc(N2CC[C@H]3[C@H]2CCN3c2ncccn2)n1] |
| | and |
| 116B | 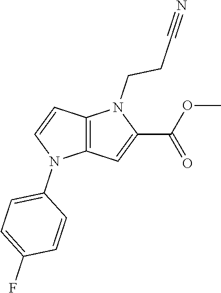 [SMILES: COC(=O)c1cc2c(ccn2-c2ccc(F)cc2)n1CCC#N] |

In some embodiments, A is an optionally substituted tricyclic 11- to 15-membered ring comprising 1-4 heteroatoms selected from the group consisting of nitrogen, oxygen and sulfur.

In some embodiments, A is an optionally substituted tricyclic 13-membered ring comprising 2 heteroatoms selected from the group consisting of nitrogen and sulfur.

In some embodiments, A is an optionally substituted tricyclic 13-membered ring comprising 2 heteroatoms selected from the group consisting of nitrogen and sulfur and B is an optionally substituted monocyclic ring selected from the group consisting of benzene and 1,3,4-oxadiazole.

In some embodiments, A is an optionally substituted tricyclic 13-membered ring comprising 2 heteroatoms selected from the group consisting of nitrogen and sulfur and L_{b} is a covalent bond.

In some embodiments, A is an optionally substituted tricyclic 13-membered ring comprising 2 heteroatoms selected from the group consisting of nitrogen and sulfur and c is 0.

In some embodiments, A is an optionally substituted bicyclic 10-membered heterocycle comprising 1 oxygen heteroatom.

In some embodiments, A is an optionally substituted 2H-chromene and B is an optionally substituted benzene.

In some embodiments, A is an optionally substituted 2H-chromene, B is an optionally substituted benzene and L_{b} is *—OCH₂—**.

In some embodiments, b is 0, c is 0 and A is an optionally substituted tricyclic ring selected from the group consisting of 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-c]quinolone.

In some embodiments, b is 0, c is 0 and A is an optionally substituted tricyclic ring selected from the group consisting of:

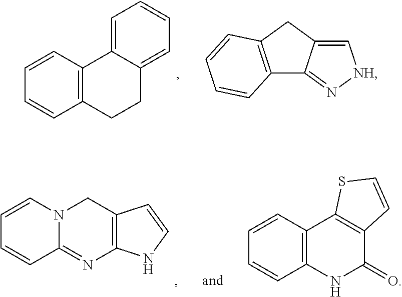

In some embodiments, the compound is selected from the group consisting of

| Compd. | |
|---|---|
| No. | Structure |
| 120 | 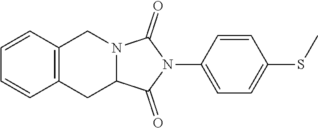 [SMILES: CSc1ccc(N2C(=O)C3Cc4ccccc4CN3C2=O)cc1] |
| 155 | 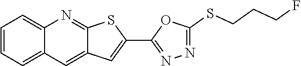 [SMILES: FCCCSc1nnc(-c2cc3cc4ccccc4nc3s2)o1] |
| 152 | 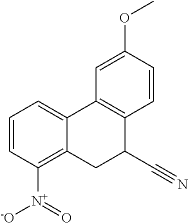 |
| 148 | 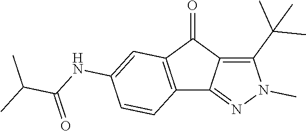 [SMILES: CC(C)C(=O)Nc1ccc2c(c1)C(=O)c1c-2nn(C)c1C(C)(C)C] |
| 146 | 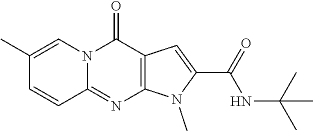 [SMILES: Cc1ccc2nc3c(cc(C(=O)NC(C)(C)C)n3C)c(=O)n2c1] |
| | and |
| 145 | 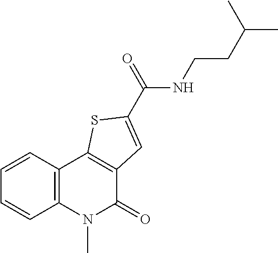 [SMILES: CC(C)CCNC(=O)c1cc2c(=O)n(C)c3ccccc3c2s1] |

In some embodiments, the compound is selected from the group consisting of

| Compd. | |
|---|---|
| No. | Structure |
| 183A | 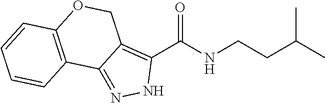 [SMILES: CC(C)CCNC(=O)c1[nH]nc2c1COc1ccccc1-2] |
| 184A | 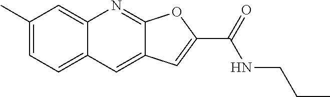 [SMILES: CCCNC(=O)c1cc2cc3ccc(C)cc3nc2o1] |
| 185A | 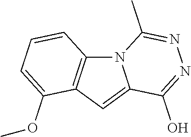 [SMILES: COc1cccc2c1cc1c(O)nnc(C)n12] |
| 186A | 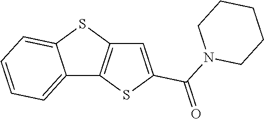 [SMILES: O=C(c1cc2sc3ccccc3c2s1)N1CCCCC1] |
| 187A | 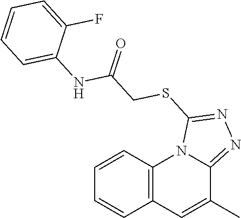 [SMILES: Cc1cc2ccccc2n2c(SCC(=O)Nc3ccccc3F)nnc12] |
| 188A | 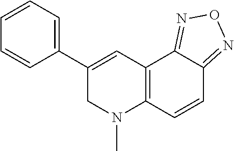 [SMILES: CN1CC(c2ccccc2)=Cc2c1ccc1nonc21] |
| 189A | 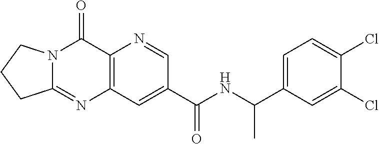 [SMILES: CC(NC(=O)c1cnc2c(=O)n3c(nc2c1)CCC3)c1ccc(Cl)c(Cl)c1] |
| 190A | 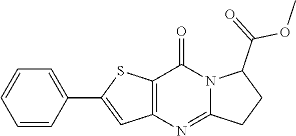 [SMILES: COC(=O)C1CCc2nc3cc(-c4ccccc4)sc3c(=O)n21] |
| 191A | 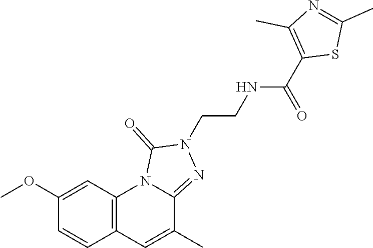 |
| 192A | 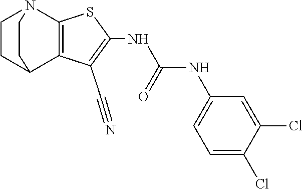 [SMILES: N#Cc1c(NC(=O)Nc2ccc(Cl)c(Cl)c2)sc2c1C1CCN2CC1] |
| | and |
| 193A | 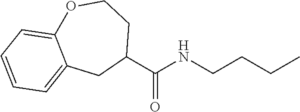 [SMILES: CCCCNC(=O)C1CCOc2ccccc2C1] |

In some embodiments, the compound is selected from the group consisting of

| Compd. | |
|---|---|
| No. | Structure |
| 118B | 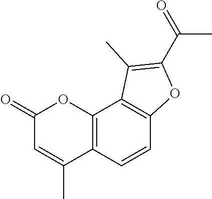 [SMILES: CC(=O)c1oc2ccc3c(C)cc(=O)oc3c2c1C] |
| 119B | 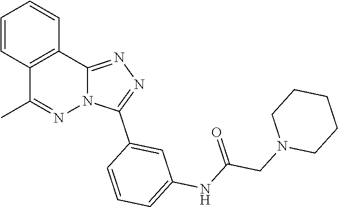 [SMILES: Cc1nn2c(-c3cccc(NC(=O)CN4CCCCC4)c3)nnc2c2ccccc12] |
| 120B | 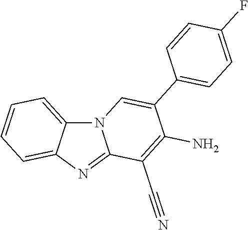 [SMILES: N#Cc1c(N)c(-c2ccc(F)cc2)cn2c1nc1ccccc12] |
| 121B | 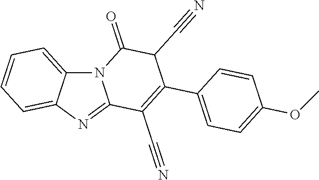 [SMILES: COc1ccc(C2=C(C#N)c3nc4ccccc4n3C(=O)C2C#N)cc1] |
| 122B | 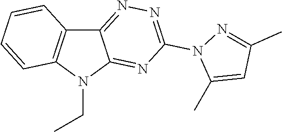 [SMILES: CCn1c2ccccc2c2nnc(-n3nc(C)cc3C)nc21] |
| 123B | 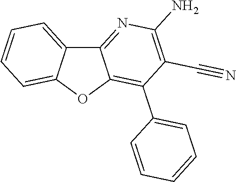 [SMILES: N#Cc1c(N)nc2c(oc3ccccc32)c1-c1ccccc1] |
| 124B | 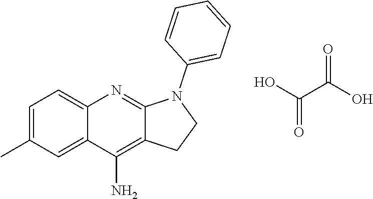 [SMILES: Cc1ccc2nc3c(c(N)c2c1)CCN3c1ccccc1 ; O=C(O)C(=O)O] |
| 125B | 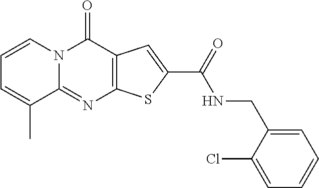 [SMILES: Cc1cccn2c(=O)c3cc(C(=O)NCc4ccccc4Cl)sc3nc12] |
| 126B | 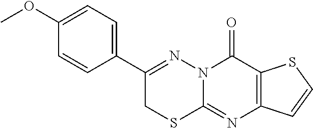 [SMILES: COc1ccc(C2=Nn3c(nc4ccsc4c3=O)SC2)cc1] |
| 127B | 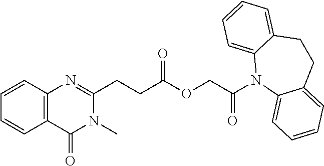 [SMILES: Cn1c(CCC(=O)OCC(=O)N2c3ccccc3CCc3ccccc32)nc2ccccc2c1=O] |
| | and |
| 117B | 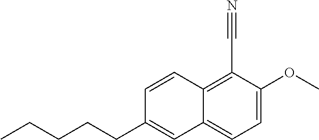 [SMILES: CCCCCc1ccc2c(C#N)c(OC)ccc2c1] |

In some embodiments, the compound is selected from the group consisting of

| Compd. | |
|---|---|
| No. | Structure |
| 133 | 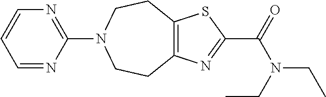 [SMILES: CCN(CC)C(=O)c1nc2c(s1)CCN(c1ncccn1)CC2] |
| 142 | 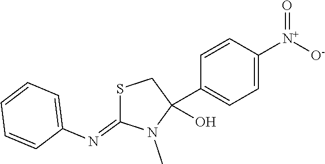 [SMILES: CN1/C(=N/c2ccccc2)SCC1(O)c1ccc([N+](=O)[O-])cc1] |
| | and |
| 153 | 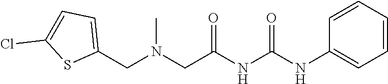 [SMILES: CN(CC(=O)NC(=O)Nc1ccccc1)Cc1ccc(Cl)s1] |

In some embodiments, the compound is selected from the group consisting of

| Compd. | |
|---|---|
| No. | Structure |
| 194A | 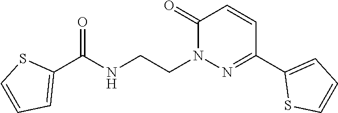 [SMILES: O=C(NCCn1nc(-c2cccs2)ccc1=O)c1cccs1] |
| 195A | 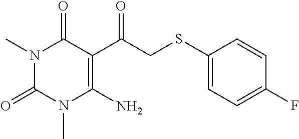 [SMILES: Cn1c(N)c(C(=O)CSc2ccc(F)cc2)c(=O)n(C)c1=O] |
| 196A | 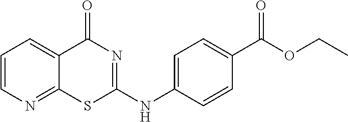 [SMILES: CCOC(=O)c1ccc(Nc2nc(=O)c3cccnc3s2)cc1] |
| | and |
| 197A | 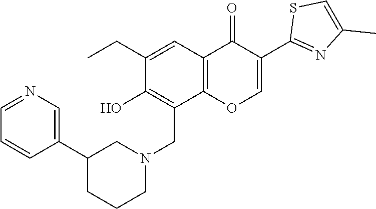 [SMILES: CCc1cc2c(=O)c(-c3nc(C)cs3)coc2c(CN2CCCC(c3cccnc3)C2)c1O] |

In another aspect, the disclosure features a method of producing an expanded population of hematopoietic stem cells ex vivo, the method including contacting a population of hematopoietic stem cells with the compound of any one of the above aspects or embodiments in an amount sufficient to produce an expanded population of hematopoietic stem cells.

In another aspect, the disclosure features a method of enriching a population of cells with hematopoietic stem cells ex vivo, the method including contacting a population of hematopoietic stem cells with the compound of any one of the above aspects or embodiments in an amount sufficient to produce a population of cells enriched with hematopoietic stem cells.

In another aspect, the disclosure features a method of maintaining the hematopoietic stem cell functional potential of a population of hematopoietic stem cells ex vivo for two or more days, the method including contacting a first population of hematopoietic stem cells with the compound of any one of the above aspects or embodiments, wherein the first population of hematopoietic stem cells exhibits a hematopoietic stem ceil functional potential after two or more days that is greater than that of a control population of hematopoietic stem cells cultured under the same conditions and for the same time as the first population of hematopoietic stem cells but not contacted with the compound.

In some embodiments, the first population of hematopoietic stem cells exhibits a hematopoietic stem cell functional potential after three or more days (for example, three days, ten days, thirty days, sixty days, or more) of culture that is greater than that of the control population of hematopoietic stem cells.

In some embodiments, the hematopoietic stem cells are mammalian cells, such as human cells. In some embodiments, the human cells are CD34+ ceils, such as CD34+ cells are CD34+, CD34+CD38−, CD34+CD38−CD90+, CD34+CD38−CD90+CD45RA−, CD34+CD38−CD90+CD45RA−CD49F+, or CD34+CD90+CD45RA− cells.

In some embodiments, the hematopoietic stem cells are CD34+ hematopoietic stem cells. In some embodiments, the hematopoietic stem cells are CD90+ hematopoietic stem ceils. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells. In some embodiments, the hematopoietic stem cells are CD90+CD45RA− hematopoietic stem cells. In some embodiments, the hematopoietic stem cells are CD34+CD90+CD45RA− hematopoietic stem cells.

In some embodiments, the hematopoietic stem cells are obtained from human cord blood, mobilized human peripheral blood, or human bone marrow. The hematopoietic stem cells may, for example, be freshly isolated from the human or may have been previously cryopreserved.

In some embodiments, the hematopoietic stem cells or progeny thereof maintain hematopoietic stem cell functional potential after two or more days upon transplantation of the hematopoietic stem cells into a human subject.

In some embodiments, the hematopoietic stem cells or progeny thereof are capable of localizing to hematopoietic tissue and reestablishing hematopoiesis upon transplantation of the hematopoietic stem cells into a human subject.

In some embodiments, upon transplantation into a human subject, the hematopoietic stem cells give rise to a population of cells selected from the group consisting of megakaryocytes, thrombocytes, platelets, erythrocytes, mast ceils, myoblasts, basophils, neutrophils, eosinophils, microglia, granulocytes, monocytes, osteoclasts, antigen-presenting cells, macrophages, dendritic ceils, natural killer cells, T-lymphocytes, and B-lymphocytes.

In another aspect, the disclosure features a method of treating a patient (e.g., a human patient) suffering from a stem cell disorder, the method including administering to the patient a population of hematopoietic stem cells, wherein the hematopoietic stem cells were produced by contacting the hematopoietic stem cells or progenitors thereof with a compound of any of the above aspects or embodiments.

In another aspect, the disclosure features a method of preparing an expanded population of hematopoietic stem cells for transplantation into a patient (e.g., a human patient) suffering from a stem cell disorder, the method including contacting a first population of hematopoietic stem cells with a compound of any of the above aspects or embodiments for a time sufficient to produce the expanded population of hematopoietic stem cells.

In another aspect, the disclosure features a method of treating a patient (e.g., a human patient) suffering from a stem cell disorder, the method including:
a. preparing an expanded population of hematopoietic stem cells by contacting a first population of hematopoietic stem cells with a compound of any of the above aspects or embodiments, and
b. administering the expanded population of hematopoietic stem cells to the patient.

In yet another aspect, provided herein is a method of treating a stem cell disorder in a patient (e.g., a human patient) in need thereof, comprising administering an expanded population of hematopoietic stem cells to the patient, wherein the expanded population of hematopoietic stem cells is prepared by contacting a first population of hematopoietic stem cells with a compound of any of the above aspects or embodiments for a time sufficient to produce the expanded population of hematopoietic stem cells.

In some embodiments, the stem cell disorder is a hemoglobinopathy.

In some embodiments, the stem cell disorder is selected from the group consisting of sickle cell anemia, thalassemia, Fanconi anemia, and Wiskott-Aldrich syndrome.

In some embodiments, the stem cell disorder is Fanconi anemia.

In some embodiments, the stem cell disorder is a myelodysplastic disorder.

In some embodiments, the stem cell disorder is an immunodeficiency disorder, such as a congenital immunodeficiency or an acquired immunodeficiency. The acquired immunodeficiency may be, for example, human immunodeficiency virus (HIV) or acquired immune deficiency syndrome (AIDS).

In some embodiments, the stem cell disorder is a metabolic disorder, such as a glycogen storage disease, a mucopolysaccharidose, Gaucher's Disease, Hurlers Disease, a sphingolipidose, or metachromatic leukodystrophy.

In some embodiments, the stem cell disorder is cancer, such as a hematological cancer. The cancer may be, for example, leukemia, lymphoma, multiple myeloma, or neuroblastoma. In some embodiments, the cancer is acute myeloid leukemia, acute lymphoid leukemia, chronic myeloid leukemia, chronic lymphoid leukemia, multiple myeloma, diffuse large B-cell lymphoma, or non-Hodgkin's lymphoma.

In some embodiments, the stem cell disorder is a disorder selected from the group consisting of adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

In some embodimetns, the stem cell disorder is an autoimmune disorder. For example, the stem cell disorder may be multiple sclerosis, human systemic lupus, rheumatoid arthritis, inflammatory bowel disease, treating psoriasis, Type 1 diabetes mellitus, acute disseminated encephalomyelitis, Addison's disease, alopecia universalis, ankylosing spondylitisis, antiphospholipid antibody syndrome, aplastic anemia, autoimmune hemolytic anemia, autoimmune hepatitis, autoimmune inner ear disease, autoimmune lymphoproliferative syndrome, autoimmune oophoritis, Balo disease, Behcet's disease, bullous pemphigoid, cardiomyopathy, Chagas' disease, chronic fatigue immune dysfunction syndrome, chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guiliain-Barre syndrome, Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease, myasthenia gravis, neuromyotonia, opsoclonus myoclonus syndrome, optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis, ulcerative colitis, uveitis, vasculitis, vitiligo, vulvodynia, or Wegener's granulomatosis.

In some embodiments, the stem cell disorder is a neurological disorder, such as Parkinson's disease, Alzheimer's disease, multiple sclerosis, Amyotrophic lateral sclerosis, Huntington's disease, mild cognitive impairment, amyloidosis, AIDS-related dementia, encephalitis, stroke, head trauma, epilepsy, mood disorders, or dementia.

In another aspect, provided herein is a method of producing microglia in the central nervous system of a patient (e.g., a human patient) in need thereof, comprising administering an expanded population of hematopoietic stem cells to the patient, wherein the expanded population of hematopoietic stem cells is prepared by contacting a first population of hematopoietic stem cells with a compound of any of the above aspects or embodiments for a time sufficient to produce the expanded population of hematopoietic stem cells, and wherein administration of the expanded population of hematopoietic stem cells results in formation of microglia in the central nervous system of the patient.

In another aspect, provided herein is a method of producing an expanded population comprising genetically modified hematopoietic stem or progenitor ceils ex vivo, the method comprising contacting the population comprising genetically modified hematopoietic stem or progenitor cells with an expanding amount of a compound of any one of the preceding claims.

In some embodiments, the method further comprises disrupting an endogenous gene in a plurality of hematopoietic stem or progenitor cells (e.g., autologous hematopoietic stem or progenitor cells), thereby producing a population comprising genetically modified hematopoietic stem or progenitor cells.

In some embodiments, the method further comprises repairing an endeogenous gene in a plurality of hematopoietic stem or progenitor cells (e.g., autologous hematopoietic stem or progenitor cells), thereby producing a population comprising genetically modified hematopoietic stem or progenitor cells.

In some embodiments, the method further comprises introducing a polynucleotide into a plurality of hematopoietic stem or progenitor cells, thereby producing a population comprising genetically modified hematopoietic stem or progenitor cells that express the polynucleotide.

In another aspect, the disclosure features a composition comprising a population of hematopoietic stem cells, wherein the hematopoietic stem cells or progenitors thereof have been contacted with the compound of any one of the above aspects or embodiments, thereby expanding the hematopoietic stem cells or progenitors thereof.

In another aspect, the disclosure features a kit including the compound of any one of the above aspects or embodiments and a package insert, wherein the package insert instructs a user of the kit to contact a population of hematopoietic stem cells with the compound for a time sufficient to produce an expanded population of hematopoietic stem cells.

In another aspect, the disclosure features a kit including the compound of any one of the above aspects or embodiments and a package insert, wherein the package insert instructs a user of the kit to contact a population of cells including hematopoietic stem cells with the compound for a time sufficient to produce a population of cells enriched with hematopoietic stem cells.

In another aspect, the disclosure features a kit including the compound of any one of the above aspects or embodiments and a package insert, wherein the package insert instructs a user of the kit to contact a population of hematopoietic stem cells with the compound for a time sufficient to maintain the hematopoietic stem cell functional potential of the population of hematopoietic stem cells ex vivo for two or more days.

In some embodiments, the kit further includes a population of cells including hematopoietic stem cells.

In another aspect, the disclosure features a pharmaceutical composition comprising a compound of any one of the above aspects, or a pharmaceutically acceptable salt, hydrate, or solvate thereof, and a pharmaceutically acceptable carrier.

In another aspect, the disclosure features a method of modulating the activity of an aryl hydrocarbon receptor, comprising administering to a subject in need thereof an effective amount of a compound of any one of the above aspects, or a pharmaceutically acceptable salt, hydrate, or solvate thereof.

In another aspect, the disclosure features a method of treating or preventing a disease or disorder, comprising administering to a subject in need thereof an effective amount of a compound of any one of the above aspects, or a pharmaceutically acceptable salt, hydrate, or solvate thereof.

In some embodiments, the disease or disorder is characterized by the production of an and hydrocarbon receptor agonist.

In some embodiments, the disease or disorder is a cancer, a cancerous condition, or a tumor.

In some embodiments, the tumor is an invasive tumor.

In some embodiments, the tumor is a solid tumor.

In some embodiments, the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

In some embodiments, the method further comprises administering one or more additional anti-cancer therapies.

In another aspect, the disclosure features a method of identifying a compound as an aryl hydrocarbon receptor antagonist, the method comprising (i) activating luciferase transcription in a cell line transfected with a dioxin-response element luciferase reporter construct with an aryl hydrocarbon receptor agonist and measuring a first level of luciferase transcription; (ii ) contacting the cell line with the compound, and (iii) measuring a second level of luciferase transcription, wherein when the first level of luciferase transcription is greater than the second level of luciferase transcription the compound is identified as an and hydrocarbon receptor antagonist.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by references. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The compositions and methods described herein provide tools for expanding hematopoietic stem cells, for instance, by culturing hematopoietic stem cells ex vivo in the presence of an aryl hydrocarbon receptor antagonist represented by Formula (I), (Ia), (Ib), (Ic), (Id1), (Id2), (Ie1), (Ie2), and (If) described herein. It has presently been discovered that aryl hydrocarbon receptor antagonists of the Formula (I), (Ia), (Ib), (Ic), (Id1), (Id2), (Ie1), (Ie2), and (If) described herein are capable of inducing the proliferation of hematopoietic stem cells while maintaining the hematopoietic stem cell functional potential of the ensuing cells. As hematopoietic stem cells exhibit the ability to differentiate into a multitude of ceil types within the hematopoietic lineage, the aryl hydrocarbon receptor antagonists described herein can be used to amplify a population of hematopoietic stem cells prior to transplantation of the hematopoietic stem cells to a patient in need thereof. Exemplary patients in need of a hematopoietic stem cell transplant are those suffering from a hemoglobinopathy, immunodeficiency, or metabolic disease, such as one of the various pathologies described herein.

Despite the promise of hematopoietic stem cell transplant therapy, methods of expanding hematopoietic stem cells ex vivo to produce quantities sufficient for transplantation has been challenging due to the propensity of hematopoietic stem cells to differentiate upon proliferation. The aryl hydrocarbon receptor antagonists described herein represent a solution to this long-standing difficulty, as the compounds set forth herein are capable of inducing the expansion hematopoietic stem cells while preserving their capacity for reconstituting various populations of cells in the hematopoietic family. The compositions described herein therefore provide useful tools for the proliferation of hematopoietic stem cells prior to hematopoietic stem cell transplant therapy, and thus constitute methods of treating a variety of hematopoietic conditions, such as sickle cell anemia, thalassemia, Fanconi anemia, Wiskott-Aldrich syndrome, adenosine deaminase deficiency-severe combined immunodeficiency, metachromatic leukodystrophy, Diamond-Blackfan anemia and Schwachman-Diamond syndrome, human immunodeficiency virus infection, and acquired immune deficiency syndrome, among others.

### Definitions

Listed below are definitions of various terms used in this application. These definitions apply to terms as they are used throughout this specification and claims, unless otherwise limited in specific instances, either individually or as part of a larger group.

As used herein, the term “about” refers to a value that is within 10% above or below the value being described. For example, the term “about 5 nM” indicates a range of from 4.5 nM to 5.5 nM.

As used herein, the term “donor” refers to a human or animal from which one or more cells are isolated prior to administration of the cells, or progeny thereof, into a recipient. The one or more cells may be, for example, a population of hematopoietic stem cells.

As used herein, the term “endogenous” describes a substance, such as a molecule, cell, tissue, or organ (for example, a hematopoietic stem cell or a cell of hematopoietic lineage, such as a megakaryocyte, thrombocyte, platelet, erythrocyte, mast cell, myoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is found naturally in a particular organism, such as a human patient,

As used herein, the term “exogenous” describes a substance, such as a molecule, cell, tissue, or organ (for example, a hematopoietic stem cell or a cell of hematopoietic lineage, such as a megakaryocyte, thrombocyte, platelet, erythrocyte, mast cell, myoblast, basophil, neutrophil, eosinophil, microglial cell, granulocyte, monocyte, osteoclast, antigen-presenting cell, macrophage, dendritic cell, natural killer cell, T-lymphocyte, or B-lymphocyte) that is not found naturally in a particular organism, such as a human patient. Exogenous substances include those that are provided from an external source to an organism or to cultured matter extracted therefrom.

As used herein, the term “engraftment potential” is used to refer to the ability of hematopoietic stem and progenitor cells to repopulate a tissue, whether such cells are naturally circulating or are provided by transplantation. The term encompasses all events surrounding or leading up to engraftment, such as tissue homing of cells and colonization of cells within the tissue of interest. The engraftment efficiency or rate of engraftment can be evaluated or quantified using any clinically acceptable parameter as known to those of skill in the art and can include, for example, assessment of competitive repopulating units (CRU); incorporation or expression of a marker in tissue(s) into which stem cells have homed, colonized, or become engrafted; or by evaluation of the progress of a subject through disease progression, survival of hematopoietic stem and progenitor ceils, or survival of a recipient. Engraftment can also be determined by measuring white blood cell counts in peripheral blood during a post-transplant period. Engraftment can also be assessed by measuring recovery of marrow cells by donor cells in a bone marrow aspirate sample.

As used herein, the term “expanding amount” refers to a quantity or concentration of an agent, such as an aryl hydrocarbon receptor antagonist described herein, sufficient to induce the proliferation of a population of CD34+ cells (e.g., a CD34+CD90+ cells), for example, by from about 1.1-fold to about 1,000-fold, about 1.1-fold to about 5,000-fold, or more (e.g., about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3,6-fold, 3,7-fold, 3,8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6,1-fold, 6.2-fold, 6.3-fold, 6.4-fold, 6,5-fold, 6,6-fold, 6.7-fold, 6.8-fold, 6.9-fold, 7-fold, 7.1-fold, 7,2-fold, 7,3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 50-fold, 100-fold, 200-fold, 300-fold, 400-fold, 500-fold, 600-fold, 700-fold, 800-fold, 900-fold, 1,000-fold, or more). In one embodiment, the expanding amount, referring to a quantity or concentration of an agent, such as an aryl hydrocarbon receptor antagonist described herein, sufficient to induce the proliferation of a population of CD34+ cells (e.g., a CD34+CD90+ cells), for example, by from about 60-fold to about 900-fold, from about 80-fold to about 800-fold, from about 100-fold to about 700-fold, from about 150-fold to about 600-fold, from about 200-fold to about 500-fold, from about 250-fold to about 400-fold, from about 275-fold to about 350-fold, or about 325-fold.

As used herein, the term “hematopoietic stem cells” (“HSCs”) refers to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-ceils). Such cells may include CD34⁺ cells. CD34⁺ cells are immature cells that express the CD34 cell surface marker. In humans, CD34+ cells are believed to include a subpopulation of cells with the stem ceil properties defined above, whereas in mice, HSCs are CD34-. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38-, CD45RA−, CD90+, CD49F+, and lin− (negative for mature lineage markers including CD2, CD3, CD4, CD7, CD8, CD10, CD11B, CD19, CD20, CD56, CD235A). In mice, hone marrow LT-HSCs are CD34-, SCA-1+, C-kit+, CD135-, Slamfl/CD150+, CD48−, and lin−(negative for mature lineage markers including Ter119, CD1 lb, Grl, CD3, CD4, CD8, B220, IL7ra), whereas ST-HSCs are CD34+, SCA-1+, C-kit+, CD135-, Slamfl/CD150+, and lin− (negative for mature lineage markers including Terl 19, CDllb, Grl, CD3, CD4, CDS, B220, IL7ra). In addition, ST-HSCs are less quiescent and more proliferative than LT-HSCs under homeostatic conditions. However, LT-HSC have greater self renewal potential (i.e., they survive throughout adulthood, and can be serially transplanted through successive recipients), whereas ST-HSCs have limited self renewal (i.e., they survive for only a limited period of time, and do not possess serial transplantation potential). Any of these HSCs can be used in the methods described herein, ST-HSCs are particularly useful because they are highly proliferative and thus, can more quickly give rise to differentiated progeny.

As used herein, the term “hematopoietic progenitor cells” includes pluripotent cells capable of differentiating into several cell types of the hematopoietic system, including, without limitation, granulocytes, monocytes, erythrocytes, megakaryocytes, B-cells and T-cells, among others. Hematopoietic progenitor cells are committed to the hematopoietic cell lineage and generally do not self-renew. Hematopoietic progenitor cells can be identified, for example, by expression patterns of cell surface antigens, and include cells having the following immunophenotype: CD34+ or CD34+CD90-. Hematopoietic progenitor cells include short-term hematopoietic stem cells, multi-potent progenitor cells, common myeloid progenitor cells, granulocyte-monocyte progenitor cells, and megakaryocyte-erythrocyte progenitor cells. The presence of hematopoietic progenitor cells can be determined functionally, for instance, by detecting colony-forming unit cells, e.g., in complete methylcellulose assays, or phenotypically through the detection of ceil surface markers using flow cytometry⁷ and cell sorting assays described herein and known in the art.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.

As used herein, the term “aryl hydrocarbon receptor (AHR) modulator” refers to an agent that causes or facilitates a qualitative or quantitative change, alteration, or modification in one or more processes, mechanisms, effects, responses, functions, activities or pathways mediated by the AHR receptor. Such changes mediated by an AHR modulator, such as an inhibitor or a non-constitutive agonist of the AHR described herein, can refer to a decrease or an increase in the activity or function of the AHR, such as a decrease in, inhibition of, or diversion of, constitutive activity of the AHR.

An “AHR antagonist” refers to an AHR inhibitor that does not provoke a biological response itself upon specifically binding to the AHR polypeptide or polynucleotide encoding the AHR, but blocks or dampens agonist-mediated or ligand-mediated responses, i.e., an AHR antagonist can bind but does not activate the AHR polypeptide or polynucleotide encoding the AHR, and the binding disrupts the interaction, displaces an AHR agonist, and/or inhibits the function of an AHR agonist. Thus, as used herein, an AHR antagonist does not function as an inducer of AHR activity when bound to the AHR, i.e., they function as pure AHR inhibitors.

The term “cancer” includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposrs sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangioc-arcinoma, hepatoblastoma, angiosarcoma, hepatocellular adenoma, hemangioma, biliar)passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-theeal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous ceil carcinoma, botryoid sarcoma (embryonal rhabdomyosarcoma), fallopian tubes (carcinoma), breast; Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders, Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullar)⁷ thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

“Treat”, “treating” and “treatment” refer to a method of alleviating or abating a disease and/or its attendant symptoms.

As used herein, “preventing” or “prevent” describes reducing or eliminating the onset of the symptoms or complications of the disease, condition or disorder.

As used herein, the term “alkyl” refers to a straight- or branched-chain alkyl group having, for example, from 1 to 20 carbon atoms in the chain, or, in certain embodiments, from 1 to 6 carbon atoms in the chain. Examples of alkyl groups include, but are not limited to, methyl, ethyl, n-propyl, isopropyl, butyl, isobutyl, sec-butyl, tert-butyl, pentyl, isopentyl, tert-pentyl, neopentyl, isopentyl, tert-pentyl, hexyl, isohexyl, and the like.

As used herein, the term “alkylene” refers to a straight- or branched-chain divalent alkyl group. The divalent positions may be on the same or different atoms within the alkyl chain. Examples of alkylene include methylene, ethylene, propylene, isopropylene, and the like.

As used herein, the term “heteroalkyl” refers to a straight or branched-chain alkyl group having, for example, from 1 to 20 carbon atoms in the chain, and further containing one or more heteroatoms (e.g., oxygen, nitrogen, or sulfur, among others) in the chain.

As used herein, the term “heteroalkylene” refers to a straight- or branched-chain divalent heteroalkyl group. The divalent positions may be on the same or different atoms within the heteroalkyl chain. The divalent positions may be one or more heteroatoms.

As used herein, the term “alkenyl” refers to a straight- or branched-chain alkenyl group having, for example, from 2 to 20 carbon atoms in the chain. It denotes a monovalent group derived from a hydrocarbon moiety containing, for example, from two to six carbon atoms having at least one carbon-carbon double bond. The double bond may or may not be the point of attachment to another group. Examples of alkenyl groups include, but are not limited to, vinyl, propenyl, isopropenyl, butenyl, tert-butylenyl, 1-methyl-2-buten-1-yl, hexenyl, and the like.

As used herein, the term “alkenylene” refers to a straight- or branched-chain divalent alkenyl group. The divalent positions may be on the same or different atoms within the alkenyl chain. Examples of alkenylene include ethenylene, propenylene, isopropenylene, butenylene, and the like.

As used herein, the term “heteroalkenyl” refers to a straight- or branched-chain alkenyl group having, for example, from 2 to 20 carbon atoms in the chain, and further containing one or more heteroatoms (e.g., oxygen, nitrogen, or sulfur, among others) in the chain.

As used herein, the term “heteroalkenylene” refers to a straight- or branched-chain divalent heteroalkenyl group. The divalent positions may be on the same or different atoms within the heteroalkenyl chain. The divalent positions may be one or more heteroatoms.

As used herein, the term “alkynyl” refers to a straight- or branched-chain alkynyl group having, for example, from 2 to 20 carbon atoms in the chain and at least one carbon-carbon triple bond. Examples of alkynyl groups include, but are not limited to, propargyl, butynyi, pentynyi, hexynyl, and the like.

As used herein, the term “alkynylene” refers to a straight- or branched-chain divalent alkynyl group. The divalent positions may be on the same or different atoms within the alkynyl chain.

As used herein, the term “heteroalkynyl” refers to a straight- or branched-chain alkynyl group having, for example, from 2 to 20 carbon atoms in the chain, and further containing one or more heteroatoms (e.g., oxygen, nitrogen, or sulfur, among others) in the chain.

As used herein, the term “heteroalkynylene” refers to a straight- or branched-chain divalent heteroalkynyl group. The divalent positions may be on the same or different atoms within the heteroalkynyl chain. The divalent positions may be one or more heteroatoms.

As used herein, the term “cycloalkyl” refers to a monocyclic, or fused, bridged, or spiro polycyclic ring structure that is saturated and has, for example, from 3 to 12 carbon ring atoms. Examples of cycloalkyl groups include cyclopropyl, cyclobutyl, cyclopentyl, cyclohexyl, cycloheptyl, cyclooctyl, bicyclo[3.1.0]hexane, and the like. Also contemplated is a monovalent group derived from a monocyclic or polycyclic carbocyclic ring compound having at least one carbon-carbon double bond by the removal of at least one or two hydrogen atoms. Examples of such groups include, but are not limited to, cyclopropenyl, cyclobutenyl, cyclopentenyl, and the like.

As used herein, the term “cycloalkylene” refers to a divalent cycloalkyl group. The divalent positions may be on the same or different atoms within the ring structure. Examples of cycloalkylene include cyclopropylene, cyclobutylene, cyclopentylene, cyclohexylene, and the like.

As used herein, the term “heterocyloalkyl” or “heterocyclyl” refers to a monocyclic, or fused, bridged, or spiro polycyclic ring structure that is saturated and has, for example, from 3 to 12 ring atoms per ring structure selected from carbon atoms and heteroatoms selected from, e.g., nitrogen, oxygen, and sulfur, among others. The ring structure may contain, for example, one or more oxo groups on carbon, nitrogen, or sulfur ring members. Exemplary heterocycloalkyl groups include, but are not limited to, [1,3] dioxolane, pyrroiidinyl, pyrazolinyl, pyrazolidinyl, imidazolinyl, imidazolidinyl, piperazinyl, piperidinyl, oxazolidinyl, isooxazolidinyl, morpholinyl, thiazololidinyl, isothiazolidinyl, and tetrahydrofuryl.

As used herein, the term “heterocycloalkylene” refers to a divalent heterocycloialkyl group. The divalent positions may be on the same or different atoms within the ring structure.

As used herein, the term “aryl” refers to a monocyclic or multi cyclic aromatic ring system containing, for example, from 6 to 19 carbon atoms. Aryl groups include, but are not limited to, phenyl, fluorenyl, naphthyl, tetrahydronaphthyl, indanyl, indenyl, and the like. The divalent positions may be one or more heteroatoms.

As used herein, the term “arylene” refers to a divalent aryl group. The divalent positions may be on the same or different atoms.

As used herein, the term “heteroaryl” refers to a monocyclic heteroaromatic, or a bicyclic or a tricyclic fused-ring heteroaromatic group. In certain embodiments, the heteroaryl group contains five to ten ring atoms of which one ring atom is selected from S, O, and N; zero, one, or two ring atoms are additional heteroatoms independently selected from S, O, and N; and the remaining ring atoms are carbon. Heteroaryl groups include, but are not limited to, pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heteroaryl ene” refers to a divalent heteroaryl group. The divalent positions may be on the same or different atoms. The divalent positions may be one or more heteroatoms.

Unless otherwise constrained by the definition of the individual substituent, the foregoing chemical moieties, such as “alkyl”, “alkylene”, “heteroalkyl”, “heteroalkylene”, “alkenyl”, “alkenylene”, “heteroalkenyl”, “heteroalkenylene”, “alkynyl”, “alkynylene”, “heteroalkynyl”, “heteroalkynylene”, “cycloalkyl”, “cycloalkylene”, “heterocyclolalkyl”, heterocycloalkylene”, “aryl,” “arylene”, “heteroaryl”, and “heteroarylene” groups can optionally be substituted. As used herein, the term “optionally substituted” refers to a compound or moiety containing one or more (for example, 1, 2, 3, 4, 5, 6, 7, 8, 9, 10, or more) substituents, as permitted by the valence of the compound or moiety or a site thereof, such as a substituent selected from the group consisting of alkyl, alkenyl, alkynyl, cycloalkyl, heterocycloalkyl, alkyl aryl, alkyl heteroaryl, alkyl cycloalkyl, alkyl heterocycloalkyl, amino, ammonium, acyl, acyloxy, acylamino, aminocarbonyl, alkoxycarbonyl, ureido, carbamate, aryl, heteroaryl, sulfinyl, sulfonyl, alkoxy, sulfanyl, halogen, carboxy, trihalomethyl, cyano, hydroxy, mercapto, nitro, and the like. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

As used herein, the term “optionally substituted” refers to a chemical moiety that may have one or more chemical substituents, as valency permits, such as C₁-4 alkyl, C₁-4 alkenyl, C₁-4 alkynyl, C₂-10 cycloalkyl, C₂-10 heterocyclolalkyl, C₂-10 aryl, C₂-10 alkylaryl, C₂-10 heteroaryl, C₂-10 alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

In accordance with the application, any of the aryls, substituted aryls, heteroaryls and substituted heteroaryls described herein, can be any aromatic group.

The terms “hal,” “halo,” and “halogen,” as used herein, refer to an atom selected from fluorine, chlorine, bromine and iodine.

As described herein, compounds of the application and moieties present in the compounds may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to: —F, —Cl, Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH -heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O— heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH— C₂-C₁₂-alkenyl, —OCONH— C₂-C₁₂-alkenyl, —OCONH—C₃-C₁₂-cycloalkyl, —OCONH-aryl, —OCONH-heteroaryl, —OCONH— heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO—C₁-C₁₂-alkyl, —NHC(O)₂—C₂-C₁₂alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂— heterocycloalkyl, NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH-heterocycloalkyl, —NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH— heteroaryl, —NHC(S)NH-heterocycloalkyl, —NHC(NH)NH₂, —NHC(NH)NH— C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NHheterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)— heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH—heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S— heterocycloalkyl, or methylthiomethyl.

### Compounds of Formula (I)

In a first aspect the disclosure features an aryl hydrocarbon receptor (AHR) modulator compound represented by Formula (I) or a salt thereof

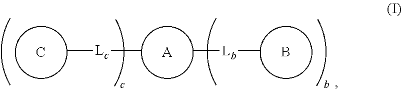

wherein:

A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;

b is 0 or 1;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂**, *═N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and A and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

c is 0 or 1;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cd}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl.

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl;

when c is 1, b is 1; and

when b is 0 and c is 0, A is an optionally substituted tricyclic ring selected from 14-membered aryl and 12- to 14-membered saturated or unsaturated heterocycle comprising 1-3 heteroatoms selected from N, O and S.

In some embodiments, b is 1 and c is 0.

In some embodiments, A is an optionally substituted monocyclic ring selected from the group consisting of benzene, pyridine, thiazole, piperazine, pyrimidine. 1,2,3-triazole, pyrazole, furan, isoxazole, 4H-pyridazine, thiophene, oxazole, and 2H-pyridine.

In some embodiments, A is an optionally substituted monocyclic ring selected from the group consisting of:

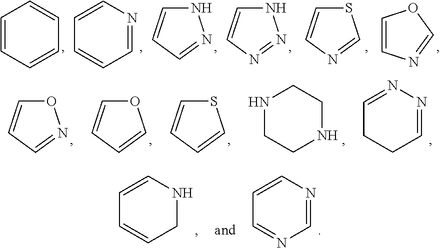

In some embodiments, A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, and [1,2,4]triazolo[1,5-a]pyrimidine.

In some embodiments, A is an optionally substituted bicyclic ring selected from the group consisting of:

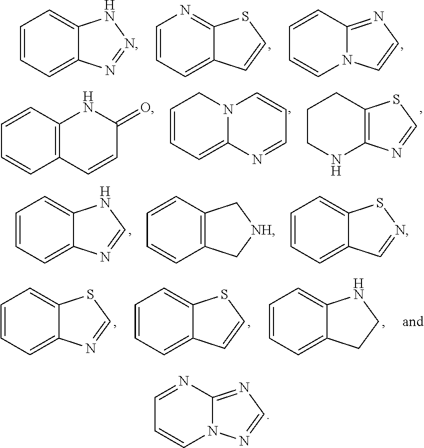

In some embodiments, A is an optionally substituted tricyclic ring selected from the group consisting of 4H-pyrido[1,2-a]thieno[2,3-d]pyrimidine, 2,4-dihydrothiochromeno[4,3-c]pyrazole, 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-c]quinolone.

In some embodiments, A is an optionally substituted tricyclic ring selected from the group consisting of

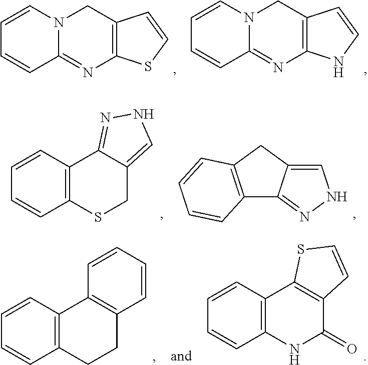

In some embodiments, A is an optionally substituted tricyclic 13-membered ring comprising 2 heteroatoms selected from the group consisting of nitrogen and sulfur.

In some embodiments, B is an optionally substituted monocyclic ring selected from the group consisting of benzene, pyridine, pyrazole, thiophene, 1,2,3-triazole, pyrimidine, pyrrole, imidazole, pyrazine, pyrrolidine, 2,3-dihydropyrrole, 2,3-dihydrothiazole, 1,2,3,4-tetrahydropyridine, 1,2,3,6-tetrahydropyridine, isoxazole, and 1,3,4-oxadiazole.

In some embodiments, B is an optionally substituted monocyclic ring selected from the group consisting of:

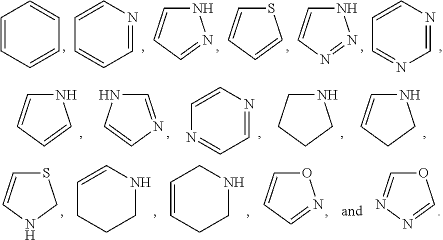

In some embodiments, B is an optionally substituted bicyclic ring selected from the group consisting of quinolone, benzo[d]imidazole, benzo[d]oxazole, indoline, thieno[2,3-d]pyrimidine, benzo[d]isothiazole, indole, naphthalene, and benzofuran.

In some embodiments, B is an optionally substituted monocyclic ring selected from the group consisting of:

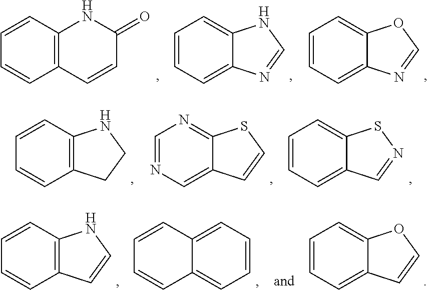

In some embodiments, B is an optionally substituted tricyclic dibenzo[b,d]furan.

In some embodiments, B is an optionally substituted

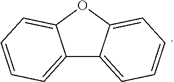 [SMILES: c1ccc2c(c1)oc1ccccc12]

In some embodiments, C is an optionally substituted monocyclic ring selected from the group consisting of benzene, isoxazole, pyridazine, thiazole, 1,3,4-oxadiazole, pyridine, pyrazole, pyrrole, thiophene, pyrimidine, morpholine, furan, and piperidine.

In some embodiments, C is an optionally substituted monocyclic ring selected from the group consisting of:

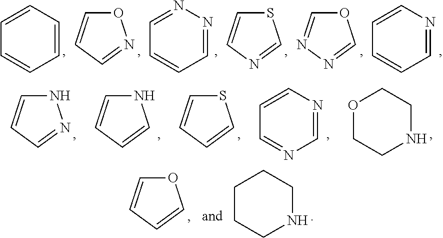

In some embodiments, C is an optionally substituted benzene.

In some embodiments, C is an optionally substituted

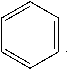

In some embodiments, C is an optionally substituted bicyclic ring selected from the group consisting of benzo[d]oxazole, imidazo[1,2-a]pyridine, quinazoline, indole, 1,2,3,4-tetrahydronaphthalene, benzo[d] imidazole and benzo[d] thiazole.

In some embodiments, C is an optionally substituted bicyclic ring selected from the group consisting of:

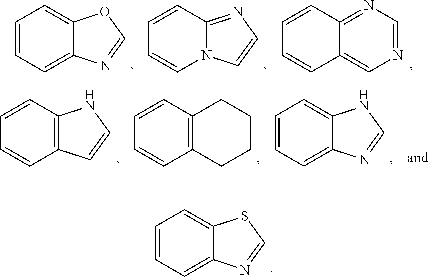

In some embodiments, L_{b} is a covalent bond, *—O—**, *—NH—**, *—NHC(O)NH—**, *—C(O)—**, *—SO₂—**, *=N—**, *—C(O)—N=**, *—OCH₂—**, *—C(O)NH—**, *—NR_{bb}C(O)—**, *—NH(CH₂)₂O—**, *—NH—R_{ba}—**, *—R_{ba}—NR_{bb}—**, *—SCH₂—**, *—SO₂CH₂—**, *—NH—N═CR_{bb}—**, *—C(O)NH—N═CH—**, *—CH₂C(O)NH—**, *—NHC(O)CH₂NH—**, *—NHC(O)OCH₂—**, or *—CH₂N(CH₃)CH₂C(O)NHC(O)NH—**.

In some embodiments, L_{b} is a covalent bond or *—C(O)NH—**.

In some embodiments, L_{b} is a covalent bond.

In some embodiments, L_{b} is *—C(O)NH—**.

In some embodiments, L_{c} is a covalent bond, *—NH—**, C₁-C₃ alkyl, *—C(O)—**, *—N═CH₂—**, *—C(O)NH—**, *—SO₂—**, *—SCH₂—**, or *—OCH₂—**.

In some embodiments, L is a covalent bond.

In some embodiments, A is optionally substituted with one or more of: —CF₃, —OCF₃, —CN, —NO₂, —N(R)₂, —OR, —SR—C(O)N(R)₂, —S(O)₂N(R)₂, —NRS(O)₂R, halo, oxo, ═NOR—NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, B is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)N(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, C is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)F, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is represented by at least one formula selected from the group consisting of Ia, Ib, Ic, Id1, Id2, Ie1, Ie2, and If.

In some embodiments, the disclosure features a compound wherein A is

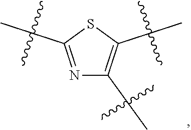 [SMILES: *c1nc(*)c(*)s1]

in which each [symbol] independently denotes the linkage between A and hydrogen, -L_{b}-B, -L_{c}-C, or a substituent.

In some embodiments, the disclosure features a compound wherein A is

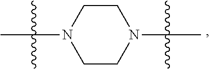 [SMILES: *N1CCN(*)CC1]

in which each [symbol] independently denotes the linkage between A and hydrogen, -L_{b}-B, -L_{c}-C, or a substituent.

In some embodiments, the disclosure features a compound wherein A is

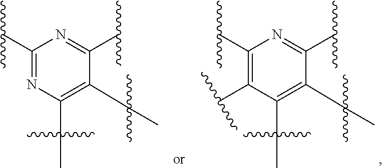 [SMILES: *c1nc(*)c(*)c(*)n1 ; *c1nc(*)c(*)c(*)c1*]

in which each [symbol] independently denotes the linkage between A and hydrogen, -L_{b}-B, -L_{c}-C, or a substituent.

In some embodiments, the disclosure features a compound wherein A is

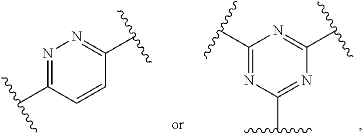 [SMILES: *c1ccc(*)nn1 ; *c1nc(*)nc(*)n1]

in which each [symbol] independently denotes the linkage between A and hydrogen, -L_{b}-B, -L_{c}-C, or a substituent.

In some embodiments, A is an optionally substituted monocyclic 5-membered heterocycle comprising 2-4 nitrogen heteroatoms.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole.

In some embodiments, A is an optionally substituted monocyclic 5-membered heterocycle comprising 2-4 nitrogen heteroatoms selected from the group consisting of:

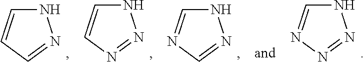

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted benzene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted bicyclic ring selected from the group consisting of benzo[d]isothiazole and naphthalene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted bicyclic ring selected from the group consisting of:

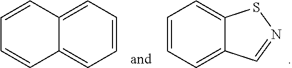 [SMILES: c1ccc2ccccc2c1 ; c1ccc2sncc2c1]

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted monocyclic ring selected from the group consisting of benzene, thiophene, and furan.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted monocyclic ring selected from the group consisting of:

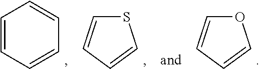

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted 1,2,3,4-tetrahydronaphthalene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole and L_{b} is selected from the group consisting of a covalent bond, *—SCH₂—**, and *—R_{ba}—NR_{bb}—**.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole, L_{b} is a covalent bond and B is an optionally substituted benzene.

In some embodiments, A is an optionally substituted monocyclic 5-membered heterocycle comprising 2-4 nitrogen heteroatoms selected from the group consisting of pyrazole, 1,2,3-triazole, 1,2,4-triazole, and tetrazole, L_{C} is a covalent bond, *—C(O)—**, or *—C(O)NHNHC(O)—**.

In some embodiments the compound is selected from the group consisting of

| Compd. | |
|---|---|
| No. | Structure |
| 90 | 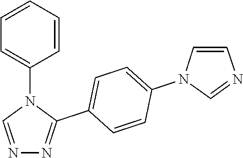 [SMILES: c1ccc(-n2cnnc2-c2ccc(-n3ccnc3)cc2)cc1] |
| 91 | 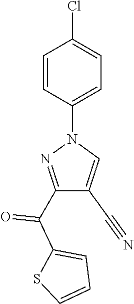 [SMILES: N#Cc1cn(-c2ccc(Cl)cc2)nc1C(=O)c1cccs1] |
| 92 | 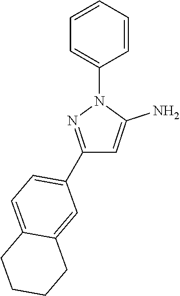 [SMILES: Nc1cc(-c2ccc3c(c2)CCCC3)nn1-c1ccccc1] |
| 93 | 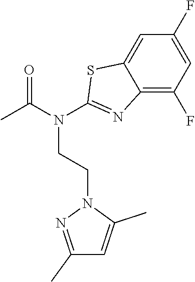 [SMILES: CC(=O)N(CCn1nc(C)cc1C)c1nc2c(F)cc(F)cc2s1] |
| 94 | 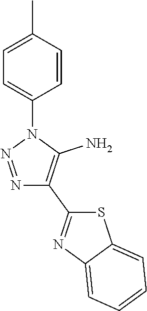 [SMILES: Cc1ccc(-n2nnc(-c3nc4ccccc4s3)c2N)cc1] |
| 95 | 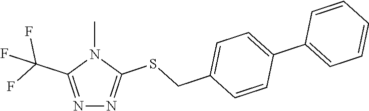 [SMILES: Cn1c(SCc2ccc(-c3ccccc3)cc2)nnc1C(F)(F)F] |
| 96 | 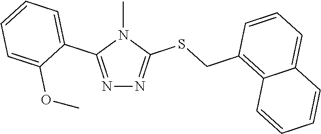 [SMILES: COc1ccccc1-c1nnc(SCc2cccc3ccccc23)n1C] |
| 97 | 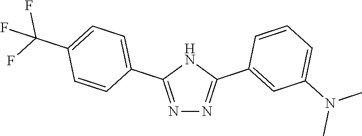 [SMILES: CN(C)c1cccc(-c2nnc(-c3ccc(C(F)(F)F)cc3)[nH]2)c1] |
| 139 | 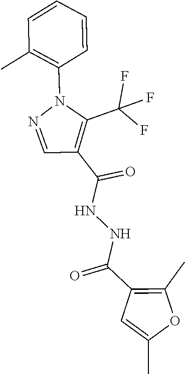 |
| | and |
| 154 | 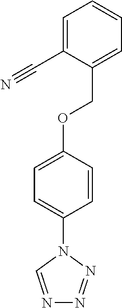 [SMILES: N#Cc1ccccc1COc1ccc(-n2cnnn2)cc1] |

In some embodiments the compound is selected from the group consisting of

| Compd. | |
|---|---|
| No. | Structure |
| 100A | 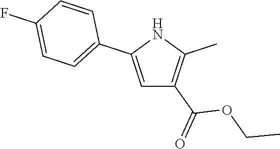 [SMILES: CCOC(=O)c1cc(-c2ccc(F)cc2)[nH]c1C] |
| 101A | 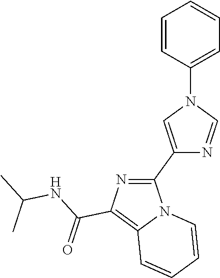 [SMILES: CC(C)NC(=O)c1nc(-c2cn(-c3ccccc3)cn2)n2ccccc12] |
| 102A | 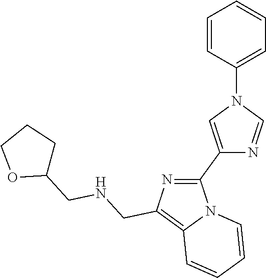 [SMILES: c1ccc(-n2cnc(-c3nc(CNCC4CCCO4)c4ccccn34)c2)cc1] |
| 103A | 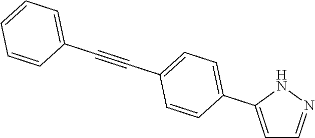 [SMILES: C(#Cc1ccc(-c2ccn[nH]2)cc1)c1ccccc1] |
| 104A | 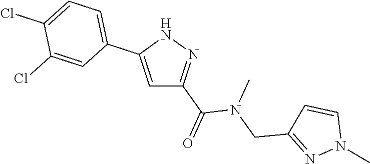 [SMILES: CN(Cc1ccn(C)n1)C(=O)c1cc(-c2ccc(Cl)c(Cl)c2)[nH]n1] |
| 105A | 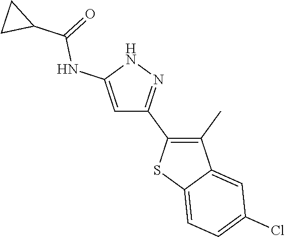 [SMILES: Cc1c(-c2cc(NC(=O)C3CC3)[nH]n2)sc2ccc(Cl)cc12] |
| 106A | 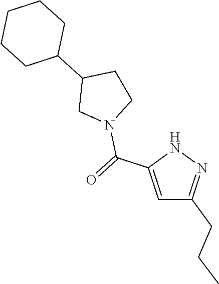 [SMILES: CCCc1cc(C(=O)N2CCC(C3CCCCC3)C2)[nH]n1] |
| 107A | 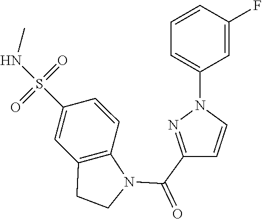 [SMILES: CNS(=O)(=O)c1ccc2c(c1)CCN2C(=O)c1ccn(-c2cccc(F)c2)n1] |
| 108A | 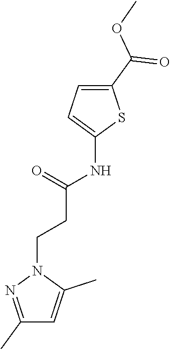 [SMILES: COC(=O)c1ccc(NC(=O)CCn2nc(C)cc2C)s1] |
| 109A | 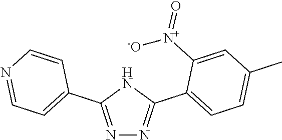 |
| | and |
| 110A | 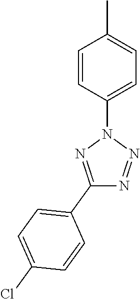 [SMILES: Cc1ccc(-n2nnc(-c3ccc(Cl)cc3)n2)cc1] |

In some embodiments the compound is selected from the group consisting of

| Compd. | |
|---|---|
| No. | Structure |
| 52B | 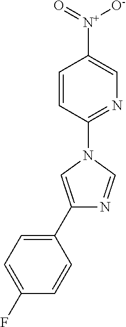 [SMILES: O=[N+]([O-])c1ccc(-n2cnc(-c3ccc(F)cc3)c2)nc1] |
| 53B | 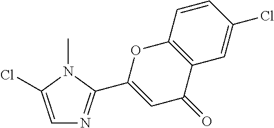 [SMILES: Cn1c(Cl)cnc1-c1cc(=O)c2cc(Cl)ccc2o1] |
| 54B | 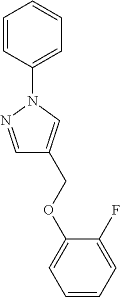 [SMILES: Fc1ccccc1OCc1cnn(-c2ccccc2)c1] |
| 55B | 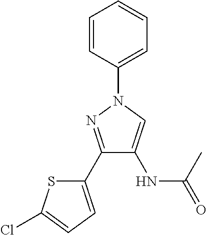 [SMILES: CC(=O)Nc1cn(-c2ccccc2)nc1-c1ccc(Cl)s1] |
| 56B | 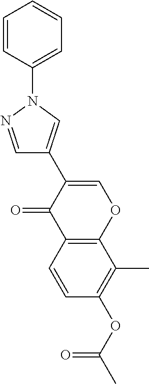 [SMILES: CC(=O)Oc1ccc2c(=O)c(-c3cnn(-c4ccccc4)c3)coc2c1C] |
| 57B | 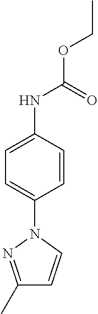 [SMILES: CCOC(=O)Nc1ccc(-n2ccc(C)n2)cc1] |
| 58B | 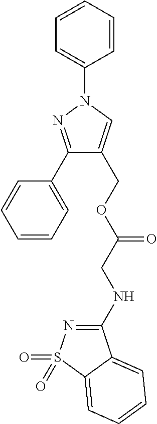 [SMILES: O=C(CNC1=NS(=O)(=O)c2ccccc21)OCc1cn(-c2ccccc2)nc1-c1ccccc1] |
| 59B | 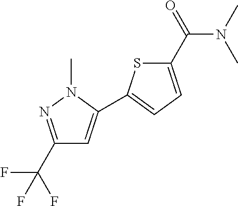 [SMILES: CN(C)C(=O)c1ccc(-c2cc(C(F)(F)F)nn2C)s1] |
| 60B | 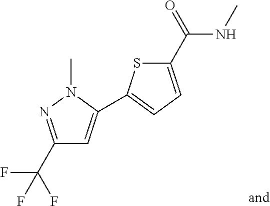 [SMILES: CNC(=O)c1ccc(-c2cc(C(F)(F)F)nn2C)s1] |
| 61B | 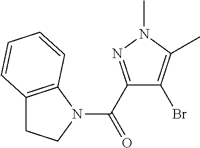 [SMILES: Cc1c(Br)c(C(=O)N2CCc3ccccc32)nn1C] |

In some embodiments, A is an optionally substituted bicyclic 9-membered heterocycle comprising 2-5 nitrogen heteroatoms.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, and tetrazolo[1,5-b]pyridazine.

In some embodiments, A is an optionally substituted bicyclic 9-membered heterocycle comprising 2-5 nitrogen heteroatoms selected from the group consisting of:

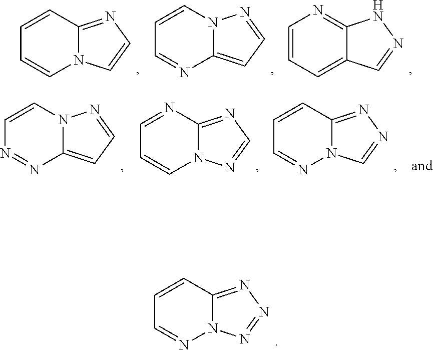

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted monocyclic ring selected from thiophene, pyrrole, benzene, pyridine, imidazole, and 1,2,3,4-tetrahydropyridine.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted monocyclic ring selected from:

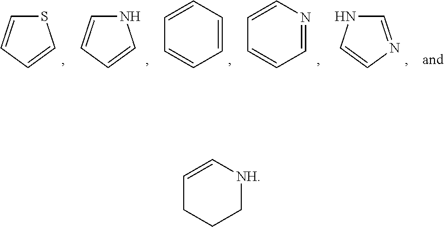

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted indole.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and C is an optionally substituted benzene.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and L_{b} is selected from the group consisting of a covalent bond, *—NH—**, and *—SCH₂—**.

In some embodiments, A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and L is a covalent bond.

In some embodiments, the compound is selected from the group consisting of

| Compd. | |
|---|---|
| No. | Structure |
| 100 | 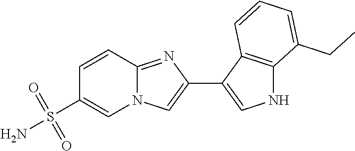 [SMILES: CCc1cccc2c(-c3cn4cc(S(N)(=O)=O)ccc4n3)c[nH]c12] |
| 101 | 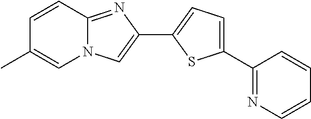 [SMILES: Cc1ccc2nc(-c3ccc(-c4ccccn4)s3)cn2c1] |
| 102 | 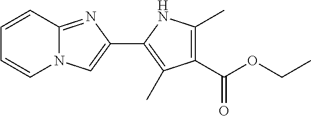 [SMILES: CCOC(=O)c1c(C)[nH]c(-c2cn3ccccc3n2)c1C] |
| 103 | 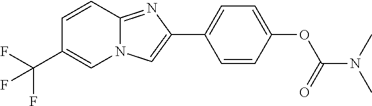 [SMILES: CN(C)C(=O)Oc1ccc(-c2cn3cc(C(F)(F)F)ccc3n2)cc1] |
| 104 | 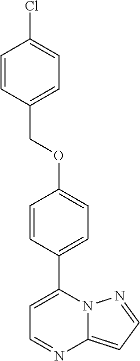 [SMILES: Clc1ccc(COc2ccc(-c3ccnc4ccnn34)cc2)cc1] |
| 105 | 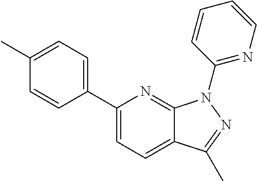 [SMILES: Cc1ccc(-c2ccc3c(C)nn(-c4ccccn4)c3n2)cc1] |
| 106 | 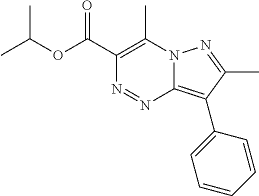 [SMILES: Cc1nn2c(C)c(C(=O)OC(C)C)nnc2c1-c1ccccc1] |
| 107 | 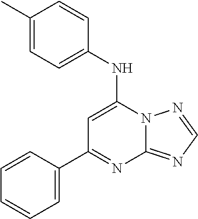 [SMILES: Cc1ccc(Nc2cc(-c3ccccc3)nc3ncnn23)cc1] |
| 108 | 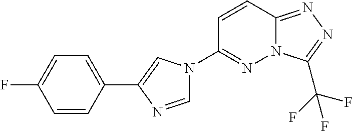 [SMILES: Fc1ccc(-c2cn(-c3ccc4nnc(C(F)(F)F)n4n3)cn2)cc1] |
| 110 | 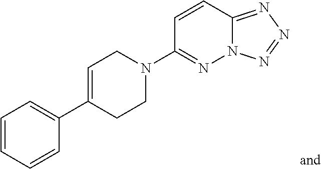 [SMILES: C1=C(c2ccccc2)CCN(c2ccc3nnnn3n2)C1] |
| 125 | 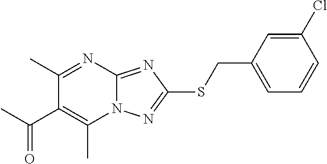 [SMILES: CC(=O)c1c(C)nc2nc(SCc3cccc(Cl)c3)nn2c1C] |

In some embodiments, the compound is selected from the group consisting of

| Compd. | |
|---|---|
| No. | Structure |
| 111A | 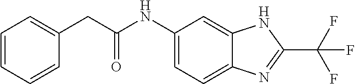 [SMILES: O=C(Cc1ccccc1)Nc1ccc2nc(C(F)(F)F)[nH]c2c1] |
| 112A | 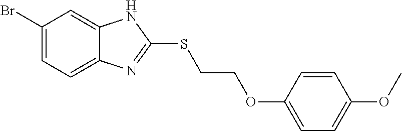 [SMILES: COc1ccc(OCCSc2nc3ccc(Br)cc3[nH]2)cc1] |
| 113A | 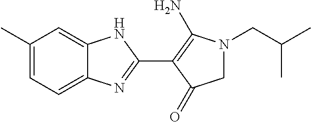 [SMILES: Cc1ccc2nc(C3=C(N)N(CC(C)C)CC3=O)[nH]c2c1] |
| 114A | 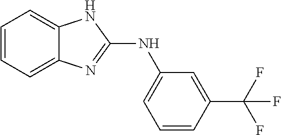 [SMILES: FC(F)(F)c1cccc(Nc2nc3ccccc3[nH]2)c1] |
| 115A | 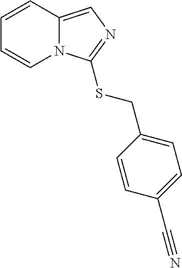 [SMILES: N#Cc1ccc(CSc2ncc3ccccn23)cc1] |
| 116A | 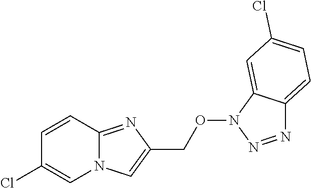 [SMILES: Clc1ccc2nnn(OCc3cn4cc(Cl)ccc4n3)c2c1] |
| 117A | 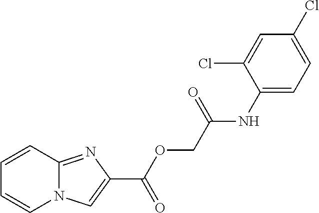 [SMILES: O=C(COC(=O)c1cn2ccccc2n1)Nc1ccc(Cl)cc1Cl] |
| 118A | 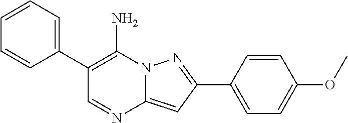 [SMILES: COc1ccc(-c2cc3ncc(-c4ccccc4)c(N)n3n2)cc1] |
| 119A | 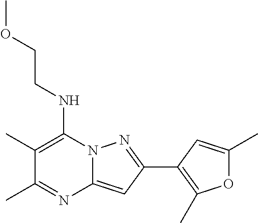 |
| 120A | 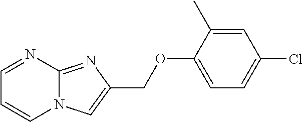 [SMILES: Cc1cc(Cl)ccc1OCc1cn2cccnc2n1] |
| 121A | 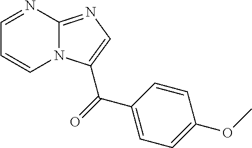 [SMILES: COc1ccc(C(=O)c2cnc3ncccn23)cc1] |
| 122A | 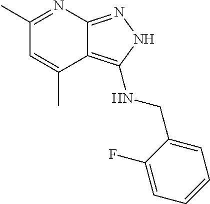 [SMILES: Cc1cc(C)c2c(NCc3ccccc3F)[nH]nc2n1] |
| 123A | 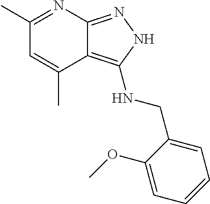 [SMILES: COc1ccccc1CNc1[nH]nc2nc(C)cc(C)c12] |
| 124A | 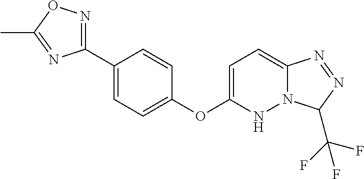 [SMILES: Cc1nc(-c2ccc(OC3=CC=C4N=NC(C(F)(F)F)N4N3)cc2)no1] |
| 125A | 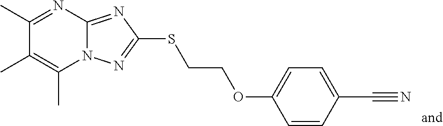 [SMILES: Cc1nc2nc(SCCOc3ccc(C#N)cc3)nn2c(C)c1C] |
| 126A | 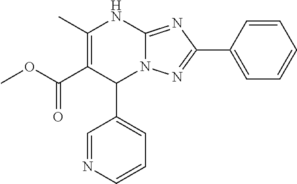 [SMILES: COC(=O)C1=C(C)Nc2nc(-c3ccccc3)nn2C1c1cccnc1] |

In some embodiments, the compound is selected from the group consisting of

| Compd. | |
|---|---|
| No. | Structure |
| 62B | 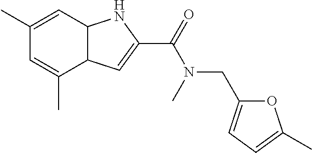 |
| 63B | 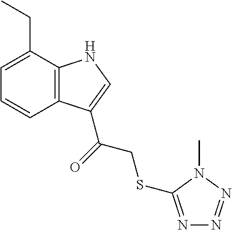 [SMILES: CCc1cccc2c(C(=O)CSc3nnnn3C)c[nH]c12] |
| 64B | 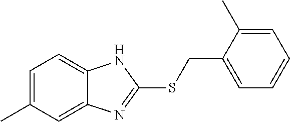 [SMILES: Cc1ccc2[nH]c(SCc3ccccc3C)nc2c1] |
| 65B | 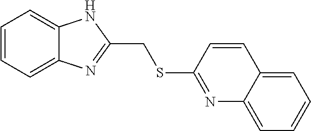 [SMILES: c1ccc2nc(SCc3nc4ccccc4[nH]3)ccc2c1] |
| 66B | 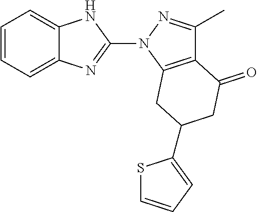 [SMILES: Cc1nn(-c2nc3ccccc3[nH]2)c2c1C(=O)CC(c1cccs1)C2] |
| 67B | 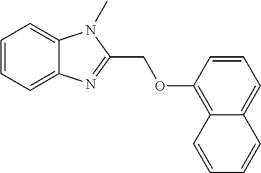 [SMILES: Cn1c(COc2cccc3ccccc23)nc2ccccc21] |
| 68B | 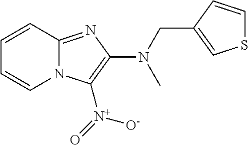 [SMILES: CN(Cc1ccsc1)c1nc2ccccn2c1[N+](=O)[O-]] |
| 69B | 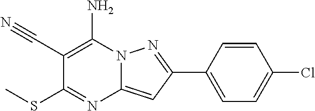 [SMILES: CSc1nc2cc(-c3ccc(Cl)cc3)nn2c(N)c1C#N] |
| 70B | 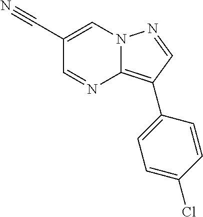 [SMILES: N#Cc1cnc2c(-c3ccc(Cl)cc3)cnn2c1] |
| 71B | 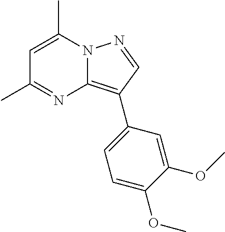 |
| 72B | 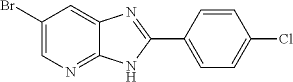 [SMILES: Clc1ccc(-c2nc3cc(Br)cnc3[nH]2)cc1] |
| 73B | 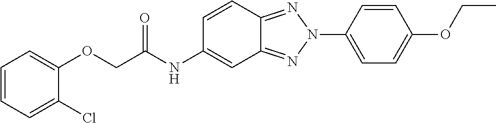 [SMILES: CCOc1ccc(-n2nc3ccc(NC(=O)COc4ccccc4Cl)cc3n2)cc1] |
| 74B | 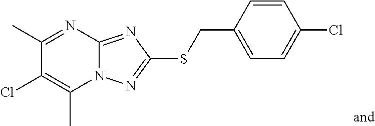 [SMILES: Cc1nc2nc(SCc3ccc(Cl)cc3)nn2c(C)c1Cl] |
| 75B | 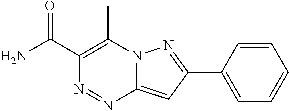 [SMILES: Cc1c(C(N)=O)nnc2cc(-c3ccccc3)nn12] |

In some embodiments, A is an optionally substituted monocyclic 5-membered heterocycle comprising 1 oxygen heteroatom and 1-2 nitrogen heteroatoms.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole.

In some embodiments, A is an optionally substituted monocyclic 5-membered heterocycle comprising 1 oxygen heteroatom and 1-2 nitrogen heteroatoms selected from the group consisting of:

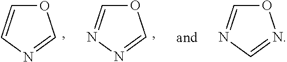

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and B is an optionally substituted monocyclic ring selected from isoxazole, pyridine, pyrazine, thiophene, and benzene.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and B is an optionally substituted monocyclic ring selected from:

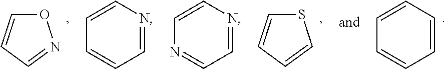

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and C is an optionally substituted monocyclic ring selected from pyrazole and benzene.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and L_{c} is selected from the group consisting of a covalent bond and *—CH₂NH—**.

In some embodiments, A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and L_{c} is a covalent bond.

In some embodiments, the compound is selected from the group consisting of

| Compd. | |
|---|---|
| No. | Structure |
| 122 | 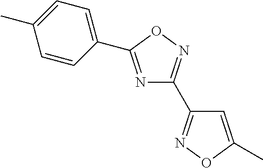 |
| 123 | 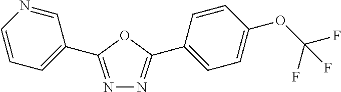 [SMILES: FC(F)(F)Oc1ccc(-c2nnc(-c3cccnc3)o2)cc1] |
| 140 | 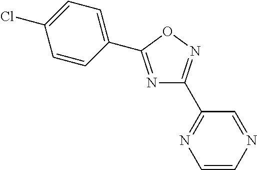 [SMILES: Clc1ccc(-c2nc(-c3cnccn3)no2)cc1] |
| 141 | 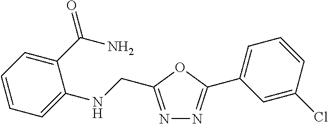 [SMILES: NC(=O)c1ccccc1NCc1nnc(-c2cccc(Cl)c2)o1] |
| 144 | 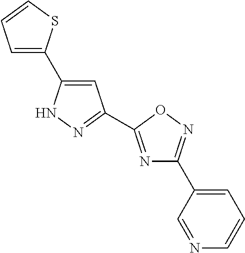 [SMILES: c1cncc(-c2noc(-c3cc(-c4cccs4)[nH]n3)n2)c1] |
| 149 | 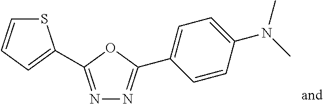 [SMILES: CN(C)c1ccc(-c2nnc(-c3cccs3)o2)cc1] |
| 157 | 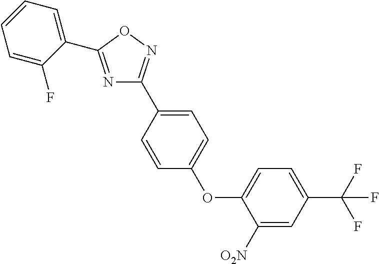 [SMILES: O=[N+]([O-])c1cc(C(F)(F)F)ccc1Oc1ccc(-c2noc(-c3ccccc3F)n2)cc1] |

In some embodiments, the compound is selected from the group consisting of

| Compd. | |
|---|---|
| No. | Structure |
| 127A | 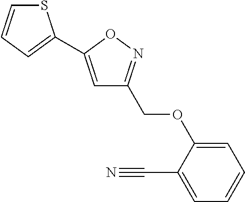 [SMILES: N#Cc1ccccc1OCc1cc(-c2cccs2)on1] |
| 128A | 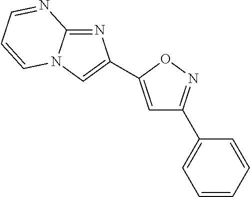 [SMILES: c1ccc(-c2cc(-c3cn4cccnc4n3)on2)cc1] |
| I29A | 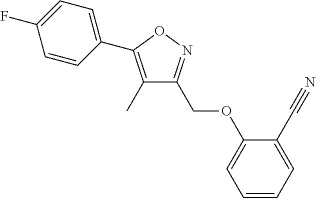 [SMILES: Cc1c(COc2ccccc2C#N)noc1-c1ccc(F)cc1] |
| 130A | 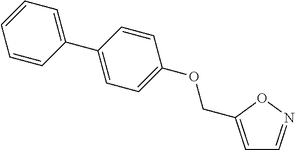 [SMILES: c1ccc(-c2ccc(OCc3ccno3)cc2)cc1] |
| 131A | 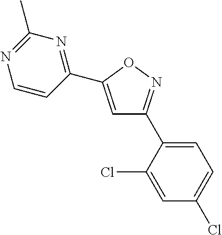 [SMILES: Cc1nccc(-c2cc(-c3ccc(Cl)cc3Cl)no2)n1] |
| 132A | 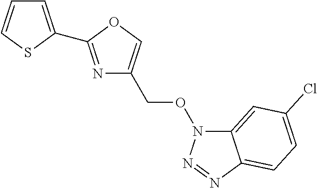 [SMILES: Clc1ccc2nnn(OCc3coc(-c4cccs4)n3)c2c1] |
| 133A | 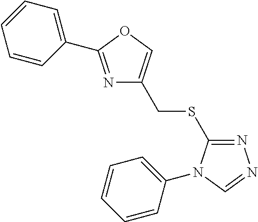 [SMILES: c1ccc(-c2nc(CSc3nncn3-c3ccccc3)co2)cc1] |
| 134A | 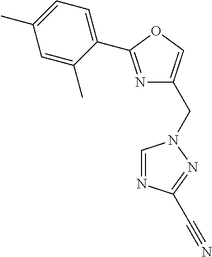 [SMILES: Cc1ccc(-c2nc(Cn3cnc(C#N)n3)co2)c(C)c1] |
| 135A | 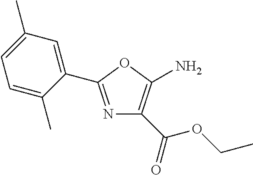 [SMILES: CCOC(=O)c1nc(-c2cc(C)ccc2C)oc1N] |
| 136A | 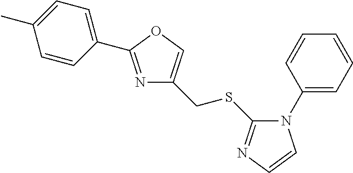 [SMILES: Cc1ccc(-c2nc(CSc3nccn3-c3ccccc3)co2)cc1] |
| 137A | 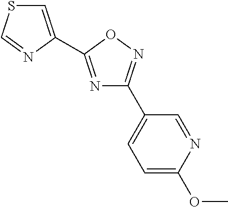 [SMILES: COc1ccc(-c2noc(-c3cscn3)n2)cn1] |
| 138A | 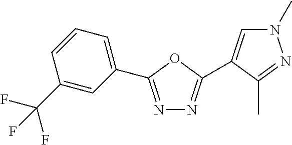 [SMILES: Cc1nn(C)cc1-c1nnc(-c2cccc(C(F)(F)F)c2)o1] |
| 139A | 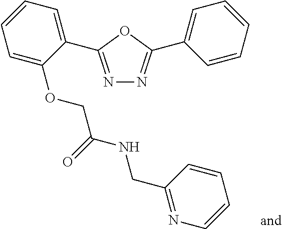 [SMILES: O=C(COc1ccccc1-c1nnc(-c2ccccc2)o1)NCc1ccccn1] |
| 140A | 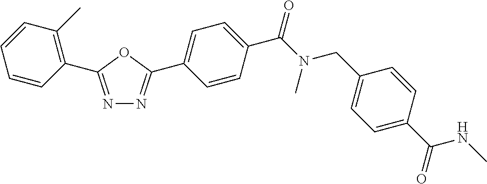 [SMILES: CNC(=O)c1ccc(CN(C)C(=O)c2ccc(-c3nnc(-c4ccccc4C)o3)cc2)cc1] |

In some embodiments, the compound is selected from the group consisting of

| Compd. | |
|---|---|
| No. | Structure |
| 76B | 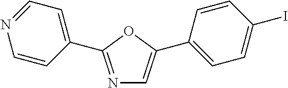 [SMILES: Ic1ccc(-c2cnc(-c3ccncc3)o2)cc1] |
| 77B | 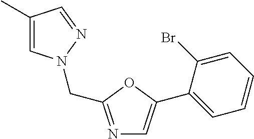 [SMILES: Cc1cnn(Cc2ncc(-c3ccccc3Br)o2)c1] |
| 78B | 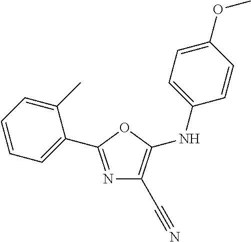 [SMILES: COc1ccc(Nc2oc(-c3ccccc3C)nc2C#N)cc1] |
| 79B | 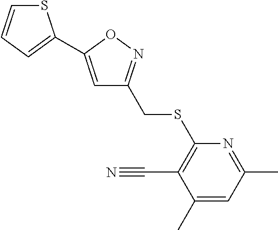 [SMILES: Cc1cc(C)c(C#N)c(SCc2cc(-c3cccs3)on2)n1] |
| 80B | 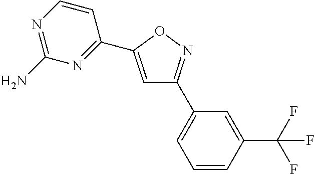 [SMILES: Nc1nccc(-c2cc(-c3cccc(C(F)(F)F)c3)no2)n1] |
| 81B | 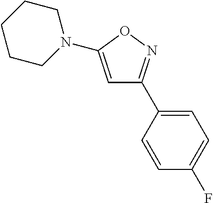 [SMILES: Fc1ccc(-c2cc(N3CCCCC3)on2)cc1] |
| 82B | 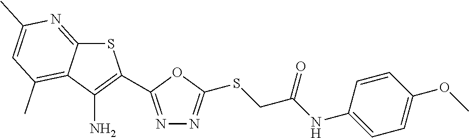 |
| 83B | 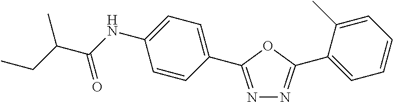 [SMILES: CCC(C)C(=O)Nc1ccc(-c2nnc(-c3ccccc3C)o2)cc1] |
| 84B | 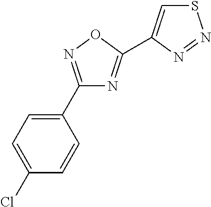 [SMILES: Clc1ccc(-c2noc(-c3csnn3)n2)cc1] |
| 85B | 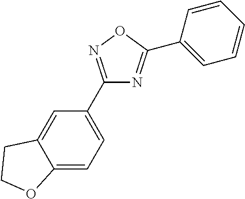 [SMILES: c1ccc(-c2nc(-c3ccc4c(c3)CCO4)no2)cc1] |
| 86B | 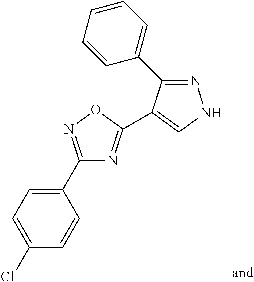 [SMILES: Clc1ccc(-c2noc(-c3c[nH]nc3-c3ccccc3)n2)cc1] |
| 87B | 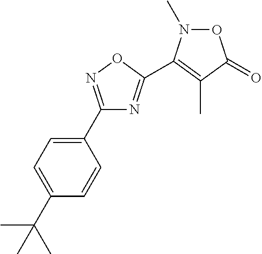 [SMILES: Cc1c(-c2nc(-c3ccc(C(C)(C)C)cc3)no2)n(C)oc1=O] |

In some embodiments, A is an optionally substituted benzene.

In some embodiments, A is an optionally substituted benzene and Bis an optionally substituted monocyclic ring selected from the group consisting of benzene, thiophene, 2,3-dihydrothiazole, and 1,2,3,6-tetrahydropyridine.

In some embodiments, A is an optionally substituted benzene and B is an optionally substituted monocyclic ring selected from the group consisting of:

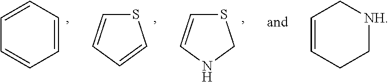

In some embodiments, A is an optionally substituted benzene and C is an optionally substituted monocyclic ring selected from the group consisting of benzene and isoxazole.

In some embodiments, A is an optionally substituted benzene and L_{b} is selected from the group consisting of a covalent bond, *—C(O)—N=**, *—OCH₂C(O)NH—**, and *—NHC(O)CH₂NH—**.

In some embodiments, A is an optionally substituted benzene and L_{c} is *—OCH₂—**.

In some embodiments, the compound is selected from the group consisting of

| Compd. | |
|---|---|
| No. | Structure |
| 118 | 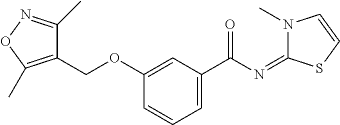 [SMILES: Cc1noc(C)c1COc1cccc(C(=O)/N=c2/sccn2C)c1] |
| 119 | 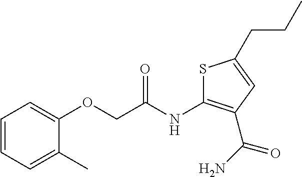 [SMILES: CCCc1cc(C(N)=O)c(NC(=O)COc2ccccc2C)s1] |
| 127 | 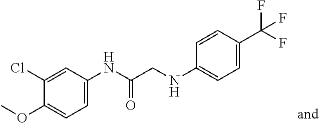 [SMILES: COc1ccc(NC(=O)CNc2ccc(C(F)(F)F)cc2)cc1Cl] |
| 143 | 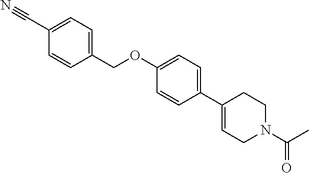 [SMILES: CC(=O)N1CC=C(c2ccc(OCc3ccc(C#N)cc3)cc2)CC1] |

In some embodiments, the compound is selected from the group consisting of

| Compd. | |
|---|---|
| No. | Structure |
| 141A | 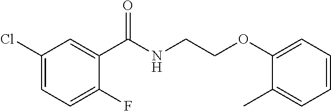 [SMILES: Cc1ccccc1OCCNC(=O)c1cc(Cl)ccc1F] |
| 142A | 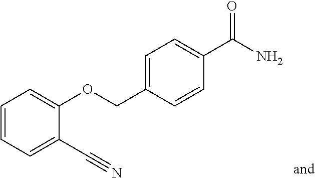 [SMILES: N#Cc1ccccc1OCc1ccc(C(N)=O)cc1] |
| 143A | 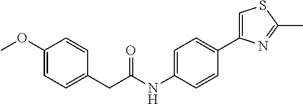 |

In some embodiments, the compound is selected from the group consisting of

| Compd | |
|---|---|
| No. | Structure |
| 88B | 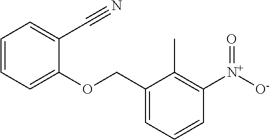 [SMILES: Cc1c(COc2ccccc2C#N)cccc1[N+](=O)[O-]] |
| 89B | 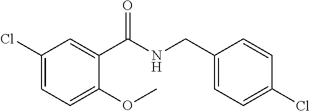 [SMILES: COc1ccc(Cl)cc1C(=O)NCc1ccc(Cl)cc1] |
| 90B | 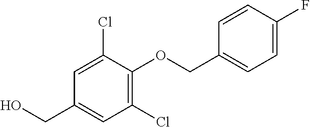 [SMILES: OCc1cc(Cl)c(OCc2ccc(F)cc2)c(Cl)c1] |
| 91B | 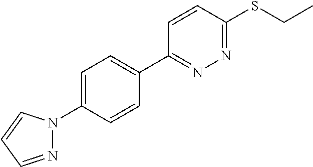 [SMILES: CCSc1ccc(-c2ccc(-n3cccn3)cc2)nn1] |
| 92B | 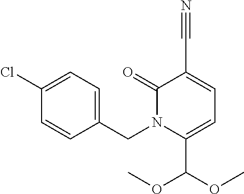 [SMILES: COC(OC)c1ccc(C#N)c(=O)n1Cc1ccc(Cl)cc1] |
| 93B | 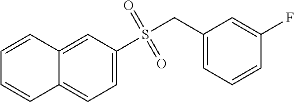 [SMILES: O=S(=O)(Cc1cccc(F)c1)c1ccc2ccccc2c1] |
| 94B | 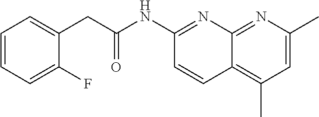 [SMILES: Cc1cc(C)c2ccc(NC(=O)Cc3ccccc3F)nc2n1] |
| 95B | 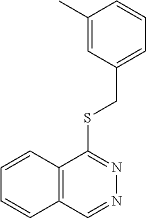 [SMILES: Cc1cccc(CSc2nncc3ccccc23)c1] |
| 96B | 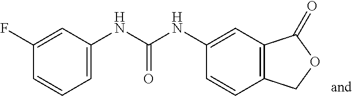 [SMILES: O=C(Nc1cccc(F)c1)Nc1ccc2c(c1)C(=O)OC2] |
| 97B | 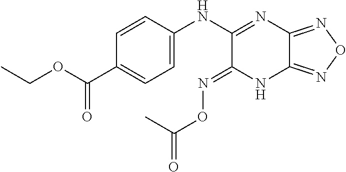 [SMILES: CCOC(=O)c1ccc(Nc2nc3nonc3[nH]/c2=N\OC(C)=O)cc1] |

In some embodiments, A is an optionally substituted monocyclic 5-membered heterocycle comprising 1-3 heteroatoms selected from nitrogen, oxygen, and sulfur.

In some embodiments, the compound is selected from the group consisting of

| Compd. | |
|---|---|
| No. | Structure |
| 144A | 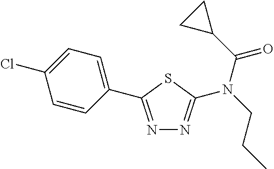 [SMILES: CCCN(C(=O)C1CC1)c1nnc(-c2ccc(Cl)cc2)s1] |
| 145A | 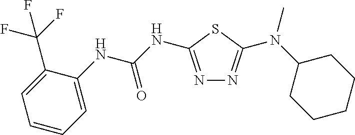 [SMILES: CN(c1nnc(NC(=O)Nc2ccccc2C(F)(F)F)s1)C1CCCCC1] |
| 146A | 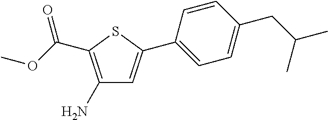 [SMILES: COC(=O)c1sc(-c2ccc(CC(C)C)cc2)cc1N] |
| 147A | 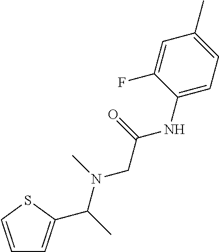 [SMILES: Cc1ccc(NC(=O)CN(C)C(C)c2cccs2)c(F)c1] |
| 148A | 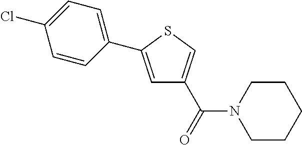 [SMILES: O=C(c1csc(-c2ccc(Cl)cc2)c1)N1CCCCC1] |
| 149A | 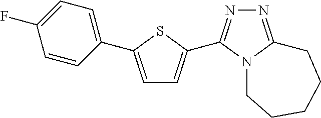 [SMILES: Fc1ccc(-c2ccc(-c3nnc4n3CCCCC4)s2)cc1] |
| 150A | 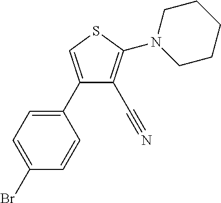 [SMILES: N#Cc1c(-c2ccc(Br)cc2)csc1N1CCCCC1] |
| 151A | 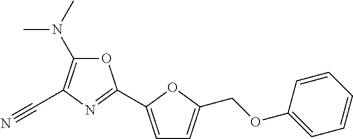 [SMILES: CN(C)c1oc(-c2ccc(COc3ccccc3)o2)nc1C#N] |
| 152A | 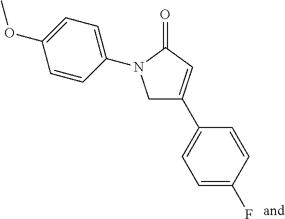 [SMILES: COc1ccc(N2CC(c3ccc(F)cc3)=CC2=O)cc1] |
| 153A | 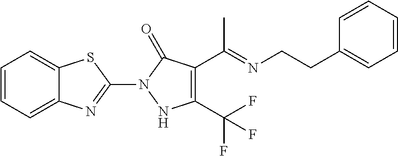 [SMILES: C/C(=N\CCc1ccccc1)c1c(C(F)(F)F)[nH]n(-c2nc3ccccc3s2)c1=O] |

In some embodiments, the compound is selected from the group consisting of

| Compd. | |
|---|---|
| No. | Structure |
| 98B | 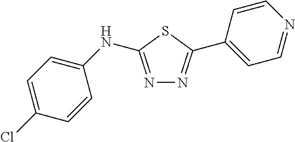 [SMILES: Clc1ccc(Nc2nnc(-c3ccncc3)s2)cc1] |
| 99B | 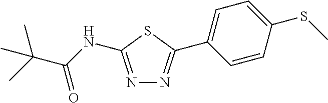 [SMILES: CSc1ccc(-c2nnc(NC(=O)C(C)(C)C)s2)cc1] |
| 100B | 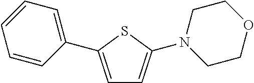 [SMILES: c1ccc(-c2ccc(N3CCOCC3)s2)cc1] |
| 101B | 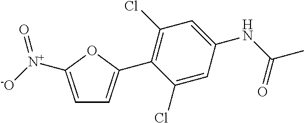 |
| 102B | 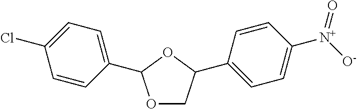 [SMILES: O=[N+]([O-])c1ccc(C2COC(c3ccc(Cl)cc3)O2)cc1] |
| 103B | 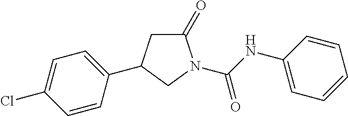 [SMILES: O=C1CC(c2ccc(Cl)cc2)CN1C(=O)Nc1ccccc1] |

In some embodiments, A is an optionally substituted bicyclic 10-membered heterocycle comprising 1-2 nitrogen heteroatoms.

In some embodiments, A is an optionally substituted bicyclic 10-membered heterocycle comprising 1-2 nitrogen heteroatoms selected from the group consisting of quinolone, quinoxaline, and pthalazine.

In some embodiments, A is an optionally substituted bicyclic 10-membered heterocycle comprising 1-2 nitrogen heteroatoms selected from the group consisting of:

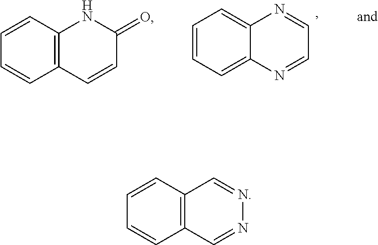 [SMILES: O=c1ccc2ccccc2[nH]1 ; c1ccc2nccnc2c1 ; "," ; c1ccc2cnncc2c1]

In some embodiments, A is an optionally substituted bicyclic 10-membered heterocycle comprising 1-2 nitrogen heteroatoms selected from the group consisting of quinolone, quinoxaline, and pthalazine and B is an optionally substituted monocyclic ring selected from benzene and pyrimidine.

In some embodiments, A is an optionally substituted bicyclic 10-membered heterocycle comprising 1-2 nitrogen heteroatoms selected from the group consisting of quinolone, quinoxaline, and pthalazine and C is an optionally substituted benzene.

In some embodiments, A is an optionally substituted bicyclic 10-membered heterocycle comprising 1-2 nitrogen heteroatoms selected from the group consisting of quinolone, quinoxaline, and pthalazine and L_{b} is selected from the group consisting of a covalent bond and *—NH—**.

In some embodiments, A is an optionally substituted bicyclic 10-membered heterocycle comprising 1-2 nitrogen heteroatoms selected from the group consisting of quinolone, quinoxaline, and pthalazine and L_{c} is a covalent bond.

In some embodiments, the compound is selected from the group consisting of

| Compd. | |
|---|---|
| No. | Structure |
| 116 | 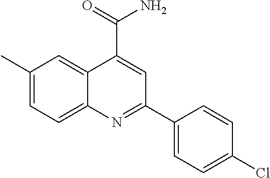 [SMILES: Cc1ccc2nc(-c3ccc(Cl)cc3)cc(C(N)=O)c2c1] |
| 124 | 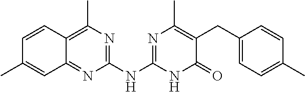 |
| 130 | 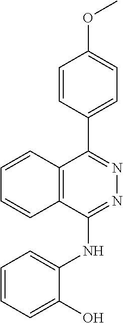 [SMILES: COc1ccc(-c2nnc(Nc3ccccc3O)c3ccccc23)cc1] |
| 128 | 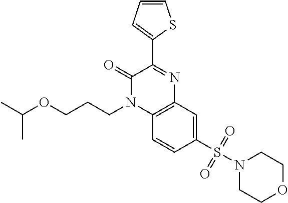 [SMILES: CC(C)OCCCn1c(=O)c(-c2cccs2)nc2cc(S(=O)(=O)N3CCOCC3)ccc21] |
| 129 | 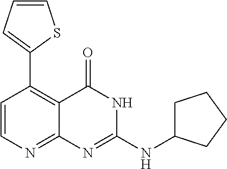 [SMILES: O=c1[nH]c(NC2CCCC2)nc2nccc(-c3cccs3)c12] |
| 131 | 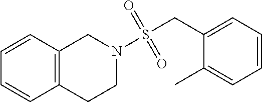 [SMILES: Cc1ccccc1CS(=O)(=O)N1CCc2ccccc2C1] |
| 132 | 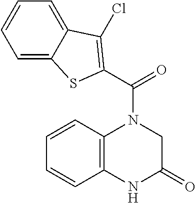 [SMILES: O=C1CN(C(=O)c2sc3ccccc3c2Cl)c2ccccc2N1] |
| 134 | 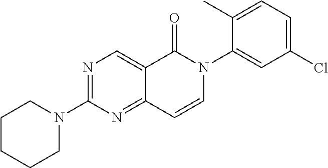 [SMILES: Cc1ccc(Cl)cc1-n1ccc2nc(N3CCCCC3)ncc2c1=O] |
| 151 | 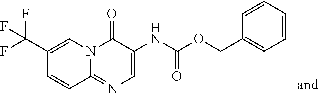 [SMILES: O=C(Nc1cnc2ccc(C(F)(F)F)cn2c1=O)OCc1ccccc1] |
| 156 | 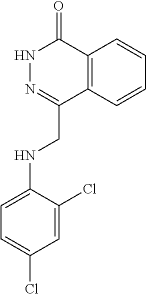 [SMILES: O=c1[nH]nc(CNc2ccc(Cl)cc2Cl)c2ccccc12] |

In some embodiments, the compound is selected from the group consisting of

| Compd. | |
|---|---|
| No. | Structure |
| 154A | 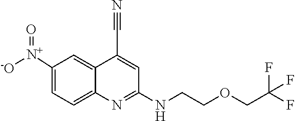 |
| 155A | 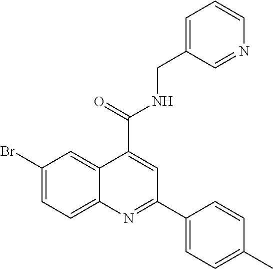 [SMILES: Cc1ccc(-c2cc(C(=O)NCc3cccnc3)c3cc(Br)ccc3n2)cc1] |
| 156A | 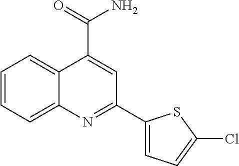 [SMILES: NC(=O)c1cc(-c2ccc(Cl)s2)nc2ccccc12] |
| 157A | 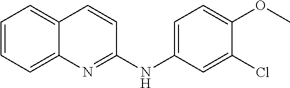 [SMILES: COc1ccc(Nc2ccc3ccccc3n2)cc1Cl] |
| 158A | 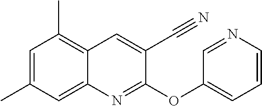 [SMILES: Cc1cc(C)c2cc(C#N)c(Oc3cccnc3)nc2c1] |
| 159A | 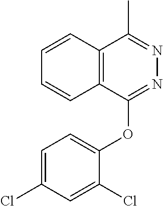 [SMILES: Cc1nnc(Oc2ccc(Cl)cc2Cl)c2ccccc12] |
| 160A | 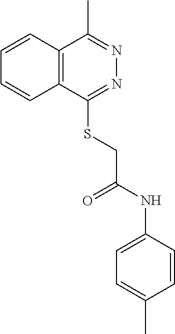 [SMILES: Cc1ccc(NC(=O)CSc2nnc(C)c3ccccc23)cc1] |
| 161A | 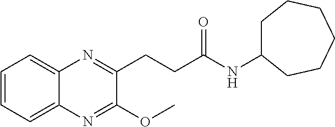 [SMILES: COc1nc2ccccc2nc1CCC(=O)NC1CCCCCC1] |
| 162A | 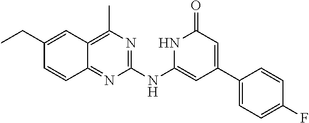 [SMILES: CCc1ccc2nc(Nc3cc(-c4ccc(F)cc4)cc(=O)[nH]3)nc(C)c2c1] |
| 163A | 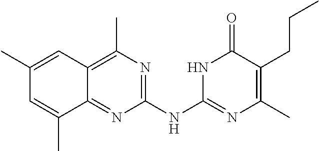 [SMILES: CCCc1c(C)nc(Nc2nc(C)c3cc(C)cc(C)c3n2)[nH]c1=O] |
| 164A | 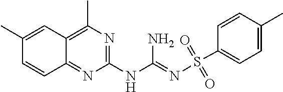 |
| 165A | 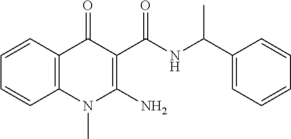 [SMILES: CC(NC(=O)c1c(N)n(C)c2ccccc2c1=O)c1ccccc1] |
| 166A | 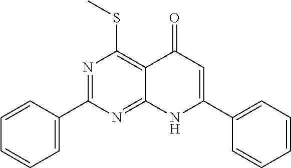 [SMILES: CSc1nc(-c2ccccc2)nc2[nH]c(-c3ccccc3)cc(=O)c12] |
| 167A | 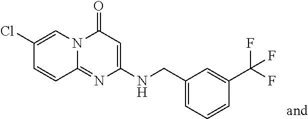 [SMILES: O=c1cc(NCc2cccc(C(F)(F)F)c2)nc2ccc(Cl)cn12] |
| 168A | 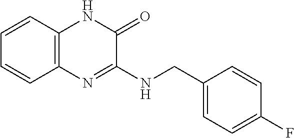 [SMILES: O=c1[nH]c2ccccc2nc1NCc1ccc(F)cc1] |

In some embodiments, the compound is selected from the group consisting of

| Compd. | |
|---|---|
| No. | Structure |
| 104B | 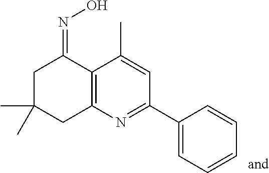 [SMILES: Cc1cc(-c2ccccc2)nc2c1/C(=N\O)CC(C)(C)C2] |
| 105B | 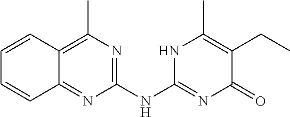 [SMILES: CCc1c(C)[nH]c(Nc2nc(C)c3ccccc3n2)nc1=O] |

In some embodiments, A is an optionally substituted bicyclic 10-membered heterocycle comprising 1-3 nitrogen heteroatoms.

In some embodiments, A is an optionally substituted bicyclic 10-membered heterocycle comprising 1-3 nitrogen heteroatoms and B is an optionally substituted monocyclic ring selected from the group consisting of benzene and thiophene.

In some embodiments, A is an optionally substituted bicyclic 10-membered heterocycle comprising 1-3 nitrogen heteroatoms and B is an optionally substituted benzo[b]thiophene.

In some embodiments, A is an optionally substituted bicyclic 10-membered heterocycle comprising 1-3 nitrogen heteroatoms and C is an optionally substituted monocyclic ring selected from the group consisting of piperidine and morpholine.

In some embodiments, A is an optionally substituted bicyclic 10-membered heterocycle comprising 1-3 nitrogen heteroatoms and L_{b} is selected from the group consisting of a covalent bond, *—NHC(O)OCH₂—**, *—CH₂NH—**, *—SO₂CH₂—**, and *—C(O)—**.

In some embodiments, A is an optionally substituted bicyclic 10-membered heterocycle comprising 1-3 nitrogen heteroatoms and L_{c} is selected from the group consisting of a covalent bond and *—SO₂—**.

In some embodiments, A is an optionally substituted bicyclic 9-membered heterocycle comprising 1-2 nitrogen heteroatoms and 1 sulfur heteroatom.

In some embodiments, A is an optionally substituted bicyclic 9-membered heterocycle comprising 1-2 nitrogen heteroatoms and 1 sulfur heteroatom and B is an optionally substituted benzene.

In some embodiments, A is an optionally substituted bicyclic 9-membered heterocycle comprising 1-2 nitrogen heteroatoms and 1 sulfur heteroatom and c is 0.

In some embodiments, A is an optionally substituted bicyclic 9-membered heterocycle comprising 1-2 nitrogen heteroatoms and 1 sulfur heteroatom and L_{b} is selected from the group consisting of a covalent bond, *—O—**, and *—NHC(O)NH—**.

In some embodiments the compound is selected from the group consisting of

| Compd. | |
|---|---|
| No. | Structure |
| 121 | 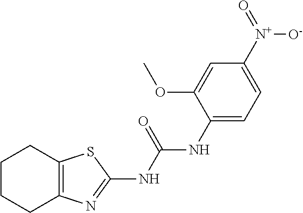 [SMILES: COc1cc([N+](=O)[O-])ccc1NC(=O)Nc1nc2c(s1)CCCC2] |
| 136 | 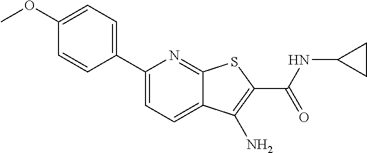 [SMILES: COc1ccc(-c2ccc3c(N)c(C(=O)NC4CC4)sc3n2)cc1] |
| 138 | 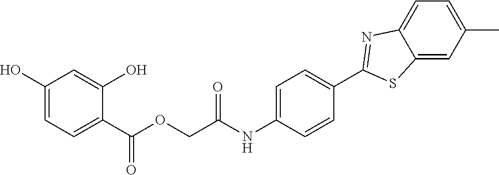 [SMILES: Cc1ccc2nc(-c3ccc(NC(=O)COC(=O)c4ccc(O)cc4O)cc3)sc2c1] |
| 147 | 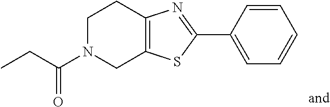 [SMILES: CCC(=O)N1CCc2nc(-c3ccccc3)sc2C1] |
| 150 | 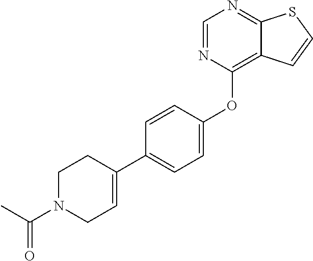 [SMILES: CC(=O)N1CC=C(c2ccc(Oc3ncnc4sccc34)cc2)CC1] |

In some embodiments, the compound is selected from the group consisting of

| Compd. | |
|---|---|
| No. | Structure |
| 169A | 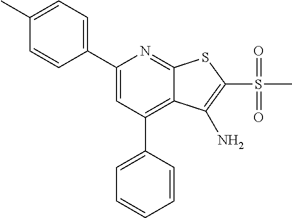 |
| 170A | 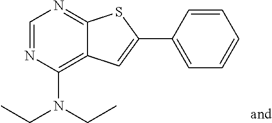 [SMILES: CCN(CC)c1ncnc2sc(-c3ccccc3)cc12] |
| 171A | 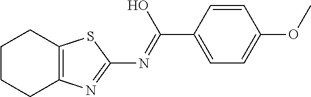 [SMILES: COc1ccc(/C(O)=N/c2nc3c(s2)CCCC3)cc1] |

In some embodiments, the compound is selected from the group consisting of

| Compd. | |
|---|---|
| No. | Structure |
| 106B | 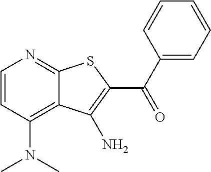 [SMILES: CN(C)c1ccnc2sc(C(=O)c3ccccc3)c(N)c12] |
| 107B | 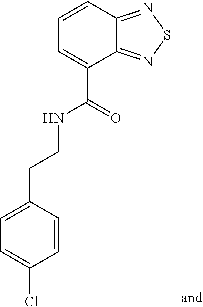 [SMILES: O=C(NCCc1ccc(Cl)cc1)c1cccc2nsnc12] |
| 108B | 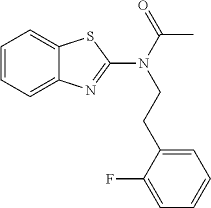 [SMILES: CC(=O)N(CCc1ccccc1F)c1nc2ccccc2s1] |

In some embodiments, A is an optionally substituted bicyclic 8- to 10-membered heterocycle comprising 14 heteroatoms selected from N, O, and S.

In some embodiments, A is an optionally substituted bicyclic 9-membered heterocycle comprising 1-4 nitrogen heteroatoms.

In some embodiments. A is an optionally substituted bicyclic 9-membered heterocycle comprising 1-4 nitrogen heteroatoms and B is an optionally substituted benzene.

In some embodiments, A is an optionally substituted bicyclic 9-membered heterocycle comprising 1-4 nitrogen heteroatoms and C is an optionally substituted benzene.

In some embodiments, A is an optionally substituted bicyclic 9-membered heterocycle comprising 1-4 nitrogen heteroatoms and L_{b} is covalent bond.

In some embodiments, A is an optionally substituted bicyclic 9-membered heterocycle comprising 1-4 nitrogen heteroatoms and L_{c} is covalent bond.

In some embodiments, the compound is selected from the group consisting of

| Compd. | |
|---|---|
| No. | Structure |
| 109 | 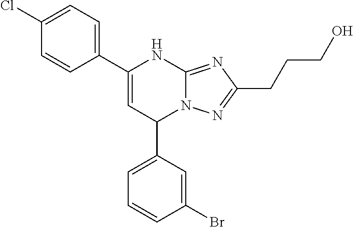 [SMILES: OCCCc1nc2n(n1)C(c1cccc(Br)c1)C=C(c1ccc(Cl)cc1)N2] |
| 117 | 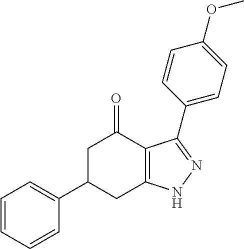 [SMILES: COc1ccc(-c2n[nH]c3c2C(=O)CC(c2ccccc2)C3)cc1] |
| 135 | 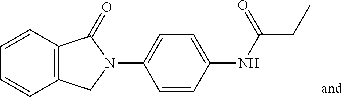 [SMILES: CCC(=O)Nc1ccc(N2Cc3ccccc3C2=O)cc1] |
| 137 | 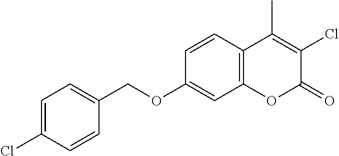 [SMILES: Cc1c(Cl)c(=O)oc2cc(OCc3ccc(Cl)cc3)ccc12] |

In some embodiments, the compound is selected from the group consisting of

| Compd. | |
|---|---|
| No. | Structure |
| 172A | 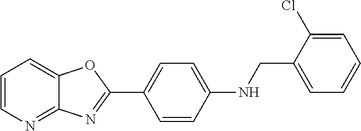 [SMILES: Clc1ccccc1CNc1ccc(-c2nc3ncccc3o2)cc1] |
| 173A | 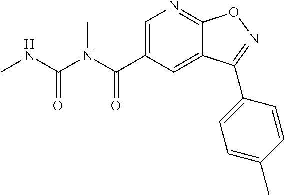 [SMILES: CNC(=O)N(C)C(=O)c1cnc2onc(-c3ccc(C)cc3)c2c1] |
| 174A | 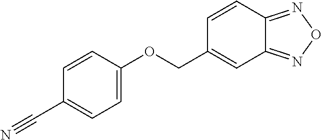 [SMILES: N#Cc1ccc(OCc2ccc3nonc3c2)cc1] |
| 175A | 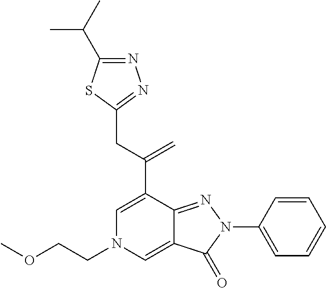 [SMILES: C=C(Cc1nnc(C(C)C)s1)c1cn(CCOC)cc2c(=O)n(-c3ccccc3)nc1-2] |
| 176A | 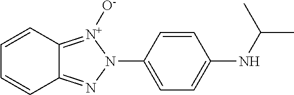 [SMILES: CC(C)Nc1ccc(-n2nc3ccccc3[n+]2[O-])cc1] |
| 177A | 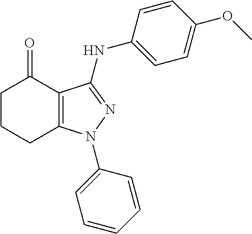 [SMILES: COc1ccc(Nc2nn(-c3ccccc3)c3c2C(=O)CCC3)cc1] |
| 178A | 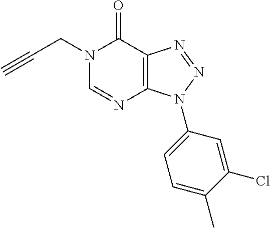 [SMILES: C#CCn1cnc2c(nnn2-c2ccc(C)c(Cl)c2)c1=O] |
| 179A | 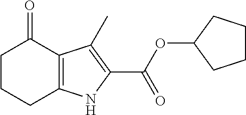 [SMILES: Cc1c(C(=O)OC2CCCC2)[nH]c2c1C(=O)CCC2] |
| 180A | 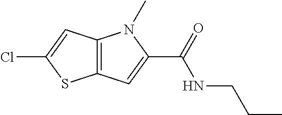 [SMILES: CCCNC(=O)c1cc2sc(Cl)cc2n1C] |
| 181A | 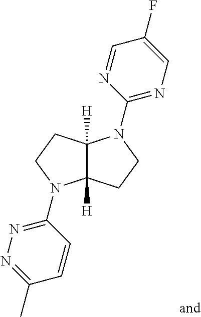 [SMILES: Cc1ccc(N2CC[C@@H]3[C@@H]2CCN3c2ncc(F)cn2)nn1] |
| 182A | 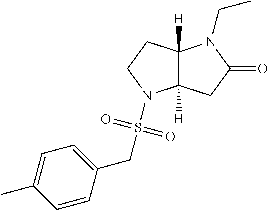 [SMILES: CCN1C(=O)C[C@@H]2[C@@H]1CCN2S(=O)(=O)Cc1ccc(C)cc1] |

In some embodiments, the compound is selected from the group consisting of

| Compd. | |
|---|---|
| No. | Structure |
| 109B | 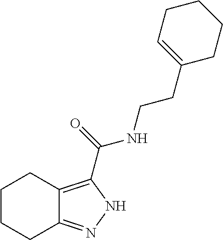 [SMILES: O=C(NCCC1=CCCCC1)c1[nH]nc2c1CCCC2] |
| 110B | 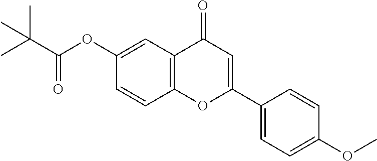 [SMILES: COc1ccc(-c2cc(=O)c3cc(OC(=O)C(C)(C)C)ccc3o2)cc1] |
| 111B | 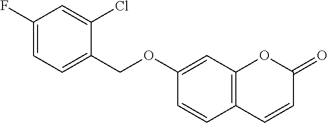 [SMILES: O=c1ccc2ccc(OCc3ccc(F)cc3Cl)cc2o1] |
| 112B | 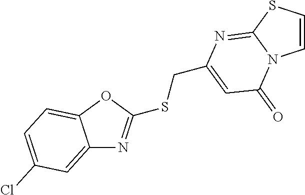 [SMILES: O=c1cc(CSc2nc3cc(Cl)ccc3o2)nc2sccn12] |
| 113B | 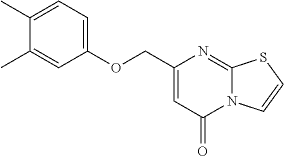 [SMILES: Cc1ccc(OCc2cc(=O)n3ccsc3n2)cc1C] |
| 114B | 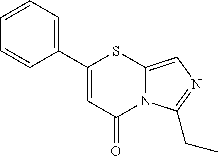 [SMILES: CCc1ncc2sc(-c3ccccc3)cc(=O)n12] |
| 115B | 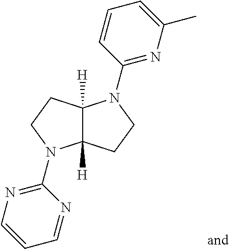 [SMILES: Cc1cccc(N2CC[C@H]3[C@H]2CCN3c2ncccn2)n1] |
| 116B | 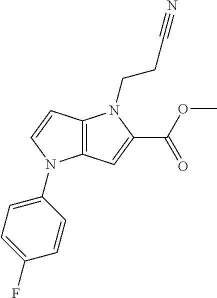 [SMILES: COC(=O)c1cc2c(ccn2-c2ccc(F)cc2)n1CCC#N] |

In some embodiments, A is an optionally substituted tricyclic 11- to 15-membered ring comprising 1-4 heteroatoms selected from the group consisting of nitrogen, oxygen and sulfur.

In some embodiments, A is an optionally substituted tricyclic 13-membered ring comprising 2 heteroatoms selected from the group consisting of nitrogen and sulfur.

In some embodiments, A is an optionally substituted tricyclic 13-membered ring comprising 2 heteroatoms selected from the group consisting of nitrogen and sulfur and B is an optionally substituted monocyclic ring selected from the group consisting of benzene and 1,3,4-oxadiazole.

In some embodiments, A is an optionally substituted tricyclic 13-membered ring comprising 2 heteroatoms selected from the group consisting of nitrogen and sulfur and L_{b} is a covalent bond.

In some embodiments, A is an optionally substituted tricyclic 13-membered ring comprising 2 heteroatoms selected from the group consisting of nitrogen and sulfur and c is 0.

In some embodiments, A is an optionally substituted bicyclic 10-membered heterocycle comprising 1 oxygen heteroatom.

In some embodiments, A is an optionally substituted 2H-chromene and B is an optionally substituted benzene.

In some embodiments, A is an optionally substituted 2H-chromene, B is an optionally substituted benzene and L_{b} is *—OCH₂—**.

In some embodiments, b is 0, c is 0 and A is an optionally substituted tricyclic ring selected from the group consisting of 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-c]quinolone.

In some embodiments, b is 0, c is 0 and A is an optionally substituted tricyclic ring selected from the group consisting of:

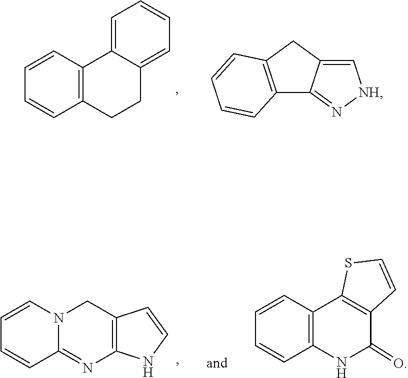

In some embodiments, the compound is selected from the group consisting of

| Compd. | |
|---|---|
| No. | Structure |
| 120 | 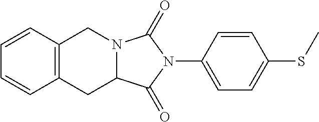 [SMILES: CSc1ccc(N2C(=O)C3Cc4ccccc4CN3C2=O)cc1] |
| 155 | 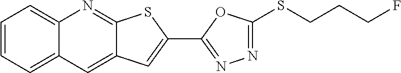 [SMILES: FCCCSc1nnc(-c2cc3cc4ccccc4nc3s2)o1] |
| 152 | 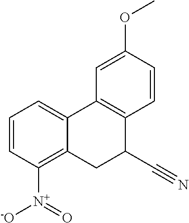 |
| 148 | 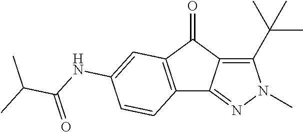 [SMILES: CC(C)C(=O)Nc1ccc2c(c1)C(=O)c1c-2nn(C)c1C(C)(C)C] |
| 146 | 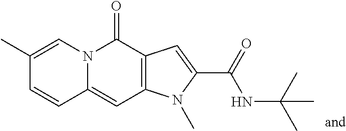 [SMILES: Cc1ccc2cc3c(cc(C(=O)NC(C)(C)C)n3C)c(=O)n2c1] |
| 145 | 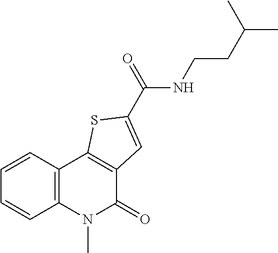 [SMILES: CC(C)CCNC(=O)c1cc2c(=O)n(C)c3ccccc3c2s1] |

In some embodiments, the compound is selected from the group consisting of

| Compd. | |
|---|---|
| No. | Structure |
| 183A | 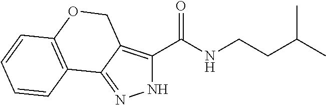 [SMILES: CC(C)CCNC(=O)c1[nH]nc2c1COc1ccccc1-2] |
| 184A | 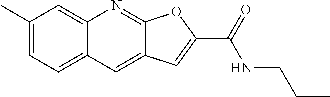 [SMILES: CCCNC(=O)c1cc2cc3ccc(C)cc3nc2o1] |
| 185A | 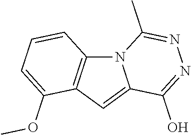 [SMILES: COc1cccc2c1cc1c(O)nnc(C)n12] |
| 186A | 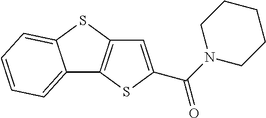 [SMILES: O=C(c1cc2sc3ccccc3c2s1)N1CCCCC1] |
| 187A | 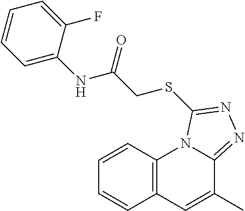 [SMILES: Cc1cc2ccccc2n2c(SCC(=O)Nc3ccccc3F)nnc12] |
| 188A | 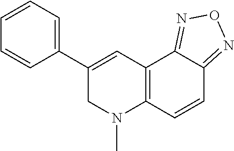 [SMILES: CN1CC(c2ccccc2)=Cc2c1ccc1nonc21] |
| 189A | 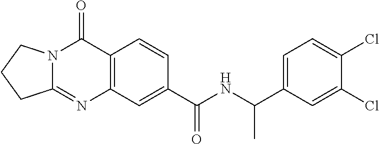 [SMILES: CC(NC(=O)c1ccc2c(=O)n3c(nc2c1)CCC3)c1ccc(Cl)c(Cl)c1] |
| 190A | 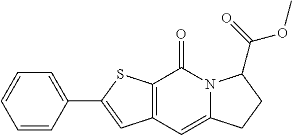 [SMILES: COC(=O)C1CCc2cc3cc(-c4ccccc4)sc3c(=O)n21] |
| 191A | 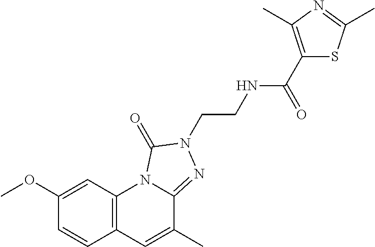 |
| 192A | 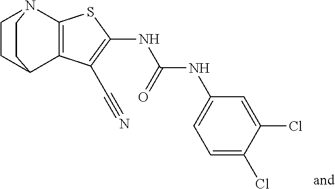 [SMILES: N#Cc1c(NC(=O)Nc2ccc(Cl)c(Cl)c2)sc2c1C1CCN2CC1] |
| 193A | 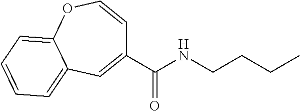 [SMILES: CCCCNC(=O)C1=Cc2ccccc2OC=C1] |

In some embodiments, the compound is selected from the group consisting of

| Compd. | |
|---|---|
| No. | Structure |
| 118B | 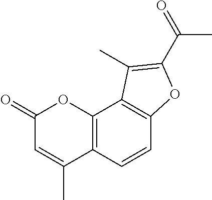 [SMILES: CC(=O)c1oc2ccc3c(C)cc(=O)oc3c2c1C] |
| 119B | 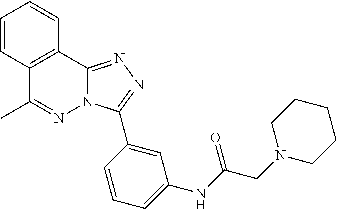 [SMILES: Cc1nn2c(-c3cccc(NC(=O)CN4CCCCC4)c3)nnc2c2ccccc12] |
| 120B | 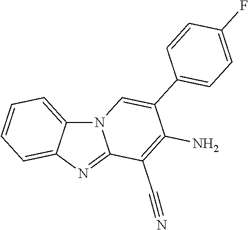 [SMILES: N#Cc1c(N)c(-c2ccc(F)cc2)cn2c1nc1ccccc12] |
| 121B | 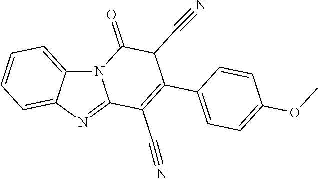 [SMILES: COc1ccc(C2=C(C#N)c3nc4ccccc4n3C(=O)C2C#N)cc1] |
| 122B | 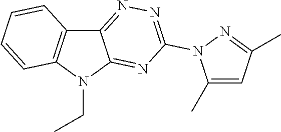 [SMILES: CCn1c2ccccc2c2nnc(-n3nc(C)cc3C)nc21] |
| 123B | 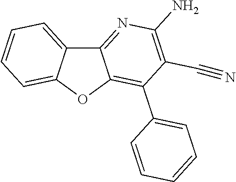 [SMILES: N#Cc1c(N)nc2c(oc3ccccc32)c1-c1ccccc1] |
| 124B | 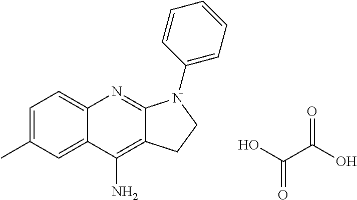 [SMILES: Cc1ccc2nc3c(c(N)c2c1)CCN3c1ccccc1 ; O=C(O)C(=O)O] |
| 125B | 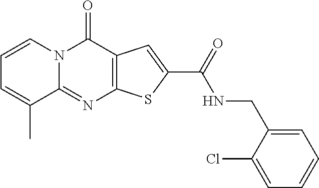 [SMILES: Cc1cccn2c(=O)c3cc(C(=O)NCc4ccccc4Cl)sc3nc12] |
| 126B | 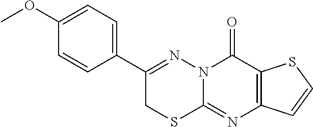 [SMILES: COc1ccc(C2=Nn3c(nc4ccsc4c3=O)SC2)cc1] |
| 127B | 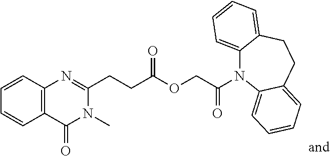 [SMILES: Cn1c(CCC(=O)OCC(=O)N2c3ccccc3CCc3ccccc32)nc2ccccc2c1=O] |
| 117B | 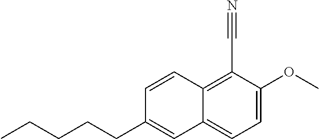 [SMILES: CCCCCc1ccc2c(C#N)c(OC)ccc2c1] |

In some embodiments, the compound is selected from the group consisting of

| Compd. | |
|---|---|
| No. | Structure |
| 133 | 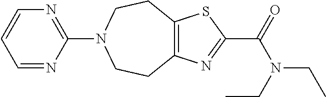 [SMILES: CCN(CC)C(=O)c1nc2c(s1)CCN(c1ncccn1)CC2] |
| 142 | 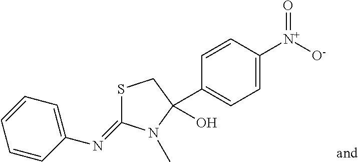 [SMILES: CN1/C(=N/c2ccccc2)SCC1(O)c1ccc([N+](=O)[O-])cc1] |
| 153 | 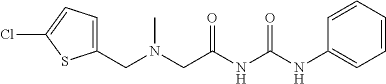 [SMILES: CN(CC(=O)NC(=O)Nc1ccccc1)Cc1ccc(Cl)s1] |

In some embodiments, the compound is selected from the group consisting of

| Compd. | |
|---|---|
| No. | Structure |
| 194A | 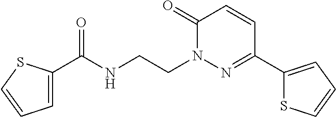 [SMILES: O=C(NCCn1nc(-c2cccs2)ccc1=O)c1cccs1] |
| 195A | 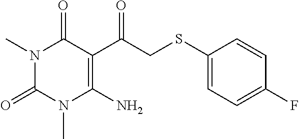 [SMILES: Cn1c(N)c(C(=O)CSc2ccc(F)cc2)c(=O)n(C)c1=O] |
| 196A | 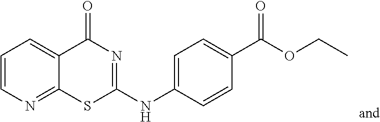 [SMILES: CCOC(=O)c1ccc(Nc2nc(=O)c3cccnc3s2)cc1] |
| 197A | 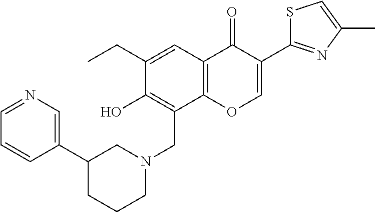 [SMILES: CCc1cc2c(=O)c(-c3nc(C)cs3)coc2c(CN2CCCC(c3cccnc3)C2)c1O] |

### Compounds of Formula (Ia)

In some embodiments, the disclousre features a compound represented by Formula (Ia) or a salt thereof

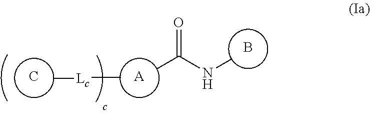

A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

c is 0 or 1,

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{ba}, or —NR_{cba}R_{cba}, in which each R_{cba} is independently H or C₁-C₆ alkyl.

In some embodiments, the compound is represented by Formula (Ia) and A is an optionally substituted monocyclic ring selected from the group consisting of benzene, pyridine, thiazole, 1,2,3-triazole, pyrazole, furan, isoxazole, 4H-pyridazine, thiophene, oxazole, 2H-pyridine, thizaole, pyrole, and pyridinone.

In some embodiments, the compound is represented by Formula (Ia) and A is an optionally substituted monocyclic ring selected from the group consisting of benzene, pyridine, thiazole, 1,2,3-triazole, pyrazole, furan, isoxazole, 4H-pyridazine, thiophene, oxazole, and 2H-pyridine.

In some embodiments, the compound is represented by Formula (Ia) and A is an optionally substituted monocyclic ring selected from the group consisting of:

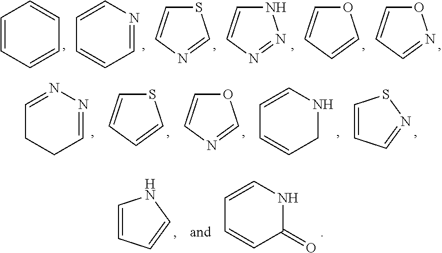

In some embodiments, the compound is represented by Formula (Ia) and A is an optionally substituted monocyclic ring selected from the group consisting of:

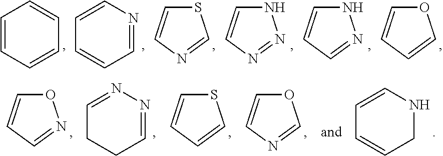

In some embodiments, the compound is represented by Formula (Ia) and A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, [1,2,4]triazolo[1,5-a]pyrimidine, naphthalene, thieno[3,2-d]imidazole, imidazo[1,5-a]pyridine, thieneo[3,2-d]pyrazole, indole, 2,3-dihydro-1H-indene, 5,6-dihydro-4H-cyclopenta[b]thiophene, and 2,3-dihydrobenzofuran.

In some embodiments, the compound is represented by Formula (Ia) and A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, and [1,2,4]triazolo[1,5-a]pyrimidine.

In some embodiments, the compound is represented by Formula (Ia) and A is an optionally substituted bicyclic ring selected from the group consisting of:

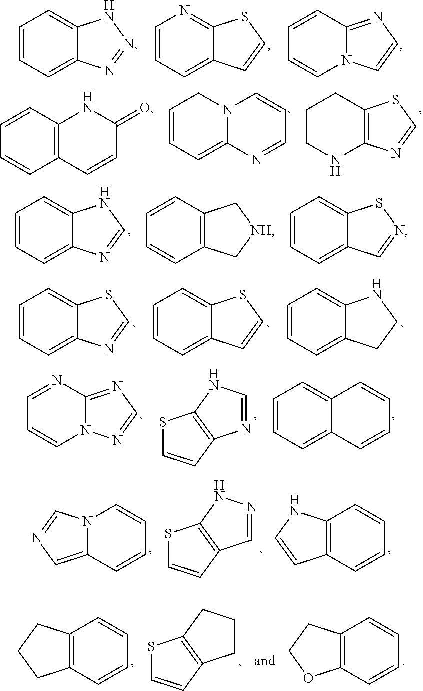

In some embodiments, the compound is represented by Formula (Ia) and A is an optionally substituted bicyclic ring selected from the group consisting of:

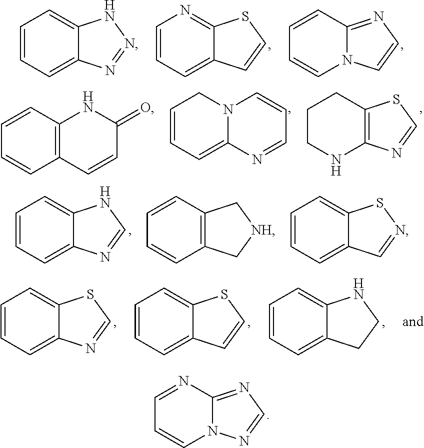

In some embodiments, the compound is represented by Formula (Ia) and A is an optionally substituted tricyclic ring selected from the group consisting of 4H-pyrido[1,2-a]thieno[2,3-d]pyrimidine, 4H-pyrido[1,2-a]pyrrolo[2,3-d]pyrimidine, 2,4-dihydrothiochromeno[4,3-c]pyrazole, 3H-benz[e]indole, and 6,7,8,9=tetrahydrothieno[2,3-c]isoquinoline.

In some embodiments, the compound is represented by Formula (Ia) and A is an optionally substituted tricyclic ring selected from the group consisting of 4H-pyrido[1,2-a]thieno[2,3-d]pyrimidine, 4H-pyrido[1,2-a]pyrrolo[2,3-d]pyrimidine, and 2,4-dihydrothiochromeno[4,3-c]pyrazole.

In some embodiments, the compound is represented by Formula (Ia) and A is an optionally substituted tricyclic ring selected from the group consisting of:

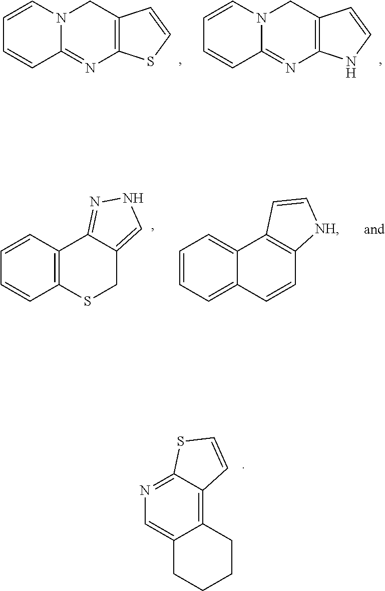

In some embodiments, the compound is represented by Formula (Ia) and A is an optionally substituted tricyclic ring selected from the group consisting of:

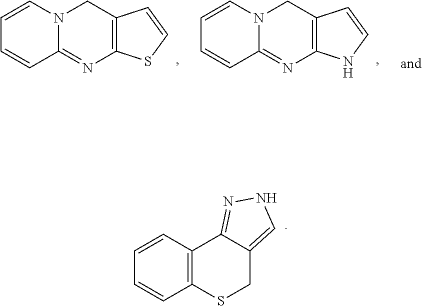

In some embodiments, the compound is represented by Formula (Ia) and B is an optionally substituted monocyclic ring selected from the group consisting of benzene, pyridine, pyrazole, thiophene, pyrimidine, thiazole, isoxazole, imidazole, 1,2,4-triazole, 1,3,4-triazole, pyridine-2-one, and pyran-2-one.

In some embodiments, the compound is represented by Formula (Ia) and B is an optionally substituted monocyclic ring selected from the group consisting of benzene, pyridine, pyrazole, and thiophene.

In some embodiments, the compound is represented by Formula (Ia) and B is an optionally substituted monocyclic ring selected from the group consisting of:

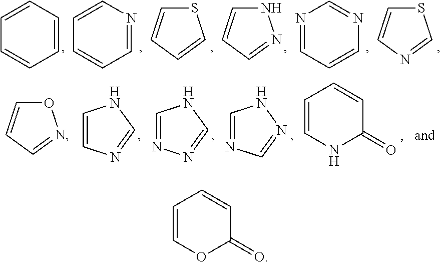

In some embodiments, the compound is represented by Formula (Ia) and B is an optionally substituted monocyclic ring selected from the group consisting of:

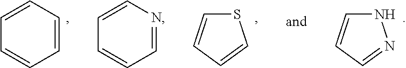

In some embodiments, the compound is represented by Formula (Ia) and B is an optionally substituted bicyclic ring selected from the group consisting of indoline, quinolone, benzo[d]imidazole, benzo[d]oxazole, benzo[b]thiophene, benzo[d]thiazole, naphthalene, quinolone, 4H-chromen-4-one, 5,6-dihydro-4H-cyclopenta[b]thiophene, 4,5,6,7-tetrahydrobenzo[b]thiophene, and 7,8-2H-1-quinoline-2,5(6H)-dione.

In some embodiments, the compound is represented by Formula (Ia) and B is an optionally substituted bicyclic ring selected from the group consisting of indoline, quinolone, benzo[d]imidazole, and benzo[d]oxazole.

In some embodiments, the compound is represented by Formula (Ia) and B is an optionally substituted bicyclic ring selected from the group consisting of:

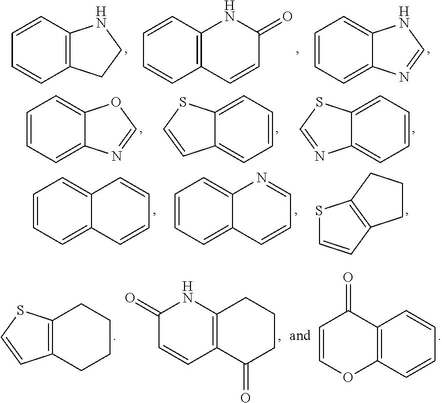

In some embodiments, the compound is represented by Formula (Ia) and B is an optionally substituted bicyclic ring selected from the group consisting of:

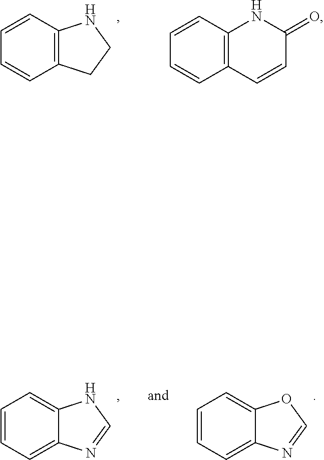 [SMILES: c1ccc2c(c1)CCN2 ; O=c1ccc2ccccc2[nH]1 ; c1ccc2[nH]cnc2c1 ; c1ccc2ocnc2c1]

In some embodiments, the compound is represented by Formula (Ia) and B is an optionally substituted dibenzo[b,d]furan.

In some embodiments, the compound is represented by Formula (Ia) and B is an optionally substituted

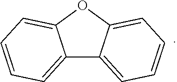 [SMILES: c1ccc2c(c1)oc1ccccc12]

In some embodiments, the compound is represented by Formula (Ia) and L is selected from the group consisting of a covalent bond, *—NH—**, and C₁-C₃ alkyl.

In some embodiments, the compound is represented by Formula (Ia) and C is an optionally substituted monocyclic ring selected from the group consisting of benzene, isoxazole, pyridazine, thiazole, pyrazole, imidazole, pyrimidine, pyridine, morpholine, and imidazolidine-2,4-dione.

In some embodiments, the compound is represented by Formula (Ia) and C is an optionally substituted monocyclic ring selected from the group consisting of benzene, isoxazole, pyridazine, and thiazole.

In some embodiments, the compound is represented by Formula (Ia) and C is an optionally substituted monocyclic ring selected from the group consisting of:

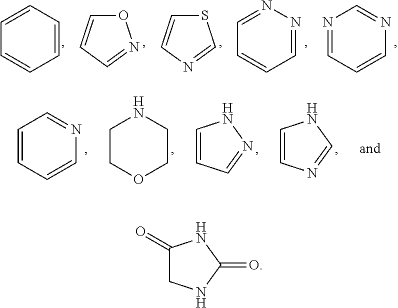

In some embodiments, the compound is represented by Formula (Ia) and C is an optionally substituted monocyclic ring selected from the group consisting of:

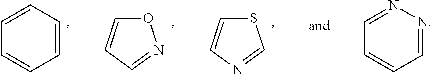

In some embodiments, the compound is represented by Formula (Ia) and C is an optionally substituted benzo[d]oxazole.

In some embodiments, the compound is represented by Formula (Ia) and C is an optionally substituted

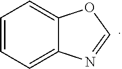

In some embodiments, the compound is represented by Formula (Ia) and A, B, or both A and B is an optionally substituted benzene.

In some embodiments, the compound is represented by Formula (Ia) and A, B, or both A and B is an optionally substituted

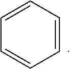

In some embodiments, the compound is represented by Formula (Ia) and A or B is an optionally substituted thiophene.

In some embodiments, the compound is represented by Formula (Ia) and A or B is an optionally substituted

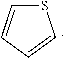

In some embodiments, the compound is represented by Formula (Ia) and c is 0.

In some embodiments, the compound is of Formula (Ia) and A is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ia) and A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ia) and B is optionally substituted with one or more of: —CF₃, —CF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ia) and B is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is of Formula (Ia) and C is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ia) and C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound represented by Formula (Ia) is a compound or salt thereof of Table 1 below.

In some embodiments, the compound is a compound or a salt thereof of Table 1 below:

TABLE 1
AHR antagonists

| Compd. | |
|---|---|
| No. | Structure |
| 1 | 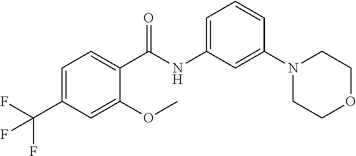 [SMILES: COc1cc(C(F)(F)F)ccc1C(=O)Nc1cccc(N2CCOCC2)c1] |
| 2 | 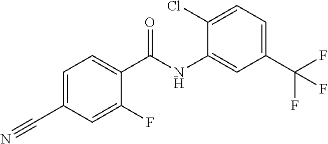 [SMILES: N#Cc1ccc(C(=O)Nc2cc(C(F)(F)F)ccc2Cl)c(F)c1] |
| 3 | 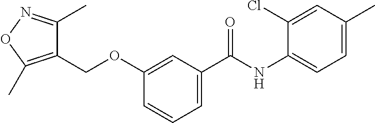 [SMILES: Cc1ccc(NC(=O)c2cccc(OCc3c(C)noc3C)c2)c(Cl)c1] |
| 4 | 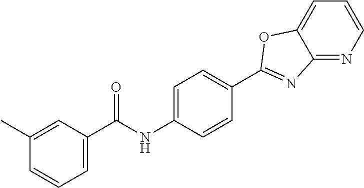 [SMILES: Cc1cccc(C(=O)Nc2ccc(-c3nc4ncccc4o3)cc2)c1] |
| 5 | 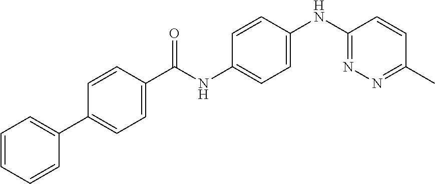 [SMILES: Cc1ccc(Nc2ccc(NC(=O)c3ccc(-c4ccccc4)cc3)cc2)nn1] |
| 6 | 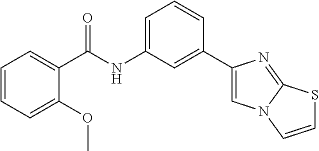 [SMILES: COc1ccccc1C(=O)Nc1cccc(-c2cn3ccsc3n2)c1] |
| 7 | 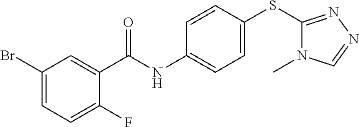 [SMILES: Cn1cnnc1Sc1ccc(NC(=O)c2cc(Br)ccc2F)cc1] |
| 8 | 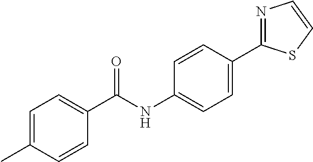 [SMILES: Cc1ccc(C(=O)Nc2ccc(-c3nccs3)cc2)cc1] |
| 9 | 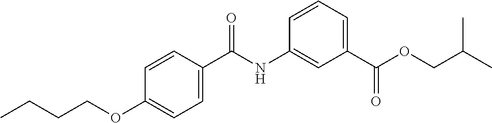 [SMILES: CCCCOc1ccc(C(=O)Nc2cccc(C(=O)OCC(C)C)c2)cc1] |
| 10 | 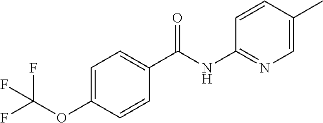 [SMILES: Cc1ccc(NC(=O)c2ccc(OC(F)(F)F)cc2)nc1] |
| 11 | 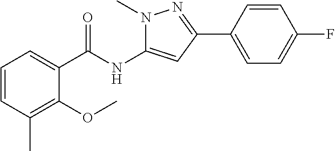 [SMILES: COc1c(C)cccc1C(=O)Nc1cc(-c2ccc(F)cc2)nn1C] |
| 12 | 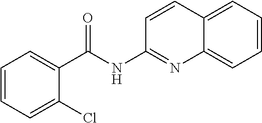 [SMILES: O=C(Nc1ccc2ccccc2n1)c1ccccc1Cl] |
| 13 | 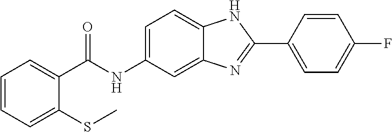 [SMILES: CSc1ccccc1C(=O)Nc1ccc2[nH]c(-c3ccc(F)cc3)nc2c1] |
| 14 | 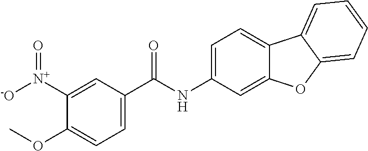 |
| 15 | 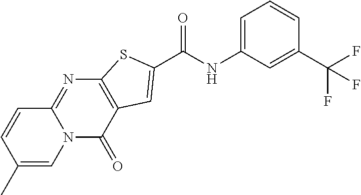 [SMILES: Cc1ccc2nc3sc(C(=O)Nc4cccc(C(F)(F)F)c4)cc3c(=O)n2c1] |
| 16 | 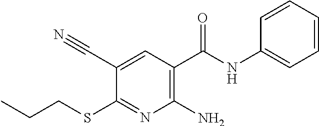 [SMILES: CCCSc1nc(N)c(C(=O)Nc2ccccc2)cc1C#N] |
| 17 | 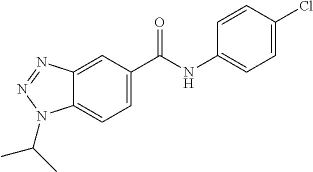 [SMILES: CC(C)n1nnc2cc(C(=O)Nc3ccc(Cl)cc3)ccc21] |
| 18 | 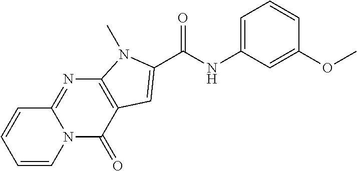 [SMILES: COc1cccc(NC(=O)c2cc3c(=O)n4ccccc4nc3n2C)c1] |
| 19 | 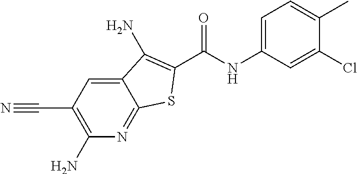 [SMILES: Cc1ccc(NC(=O)c2sc3nc(N)c(C#N)cc3c2N)cc1Cl] |
| 20 | 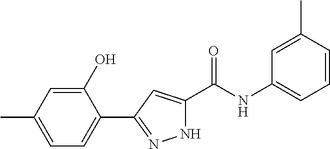 [SMILES: Cc1cccc(NC(=O)c2cc(-c3ccc(C)cc3O)n[nH]2)c1] |
| 21 | 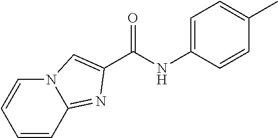 [SMILES: Cc1ccc(NC(=O)c2cn3ccccc3n2)cc1] |
| 22 | 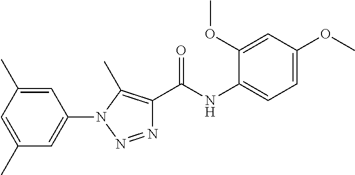 [SMILES: COc1ccc(NC(=O)c2nnn(-c3cc(C)cc(C)c3)c2C)c(OC)c1] |
| 23 | 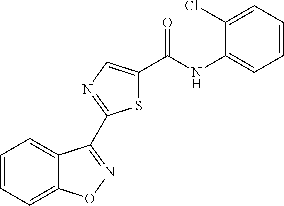 [SMILES: O=C(Nc1ccccc1Cl)c1cnc(-c2noc3ccccc23)s1] |
| 24 | 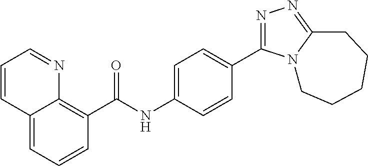 [SMILES: O=C(Nc1ccc(-c2nnc3n2CCCCC3)cc1)c1cccc2cccnc12] |
| 25 | 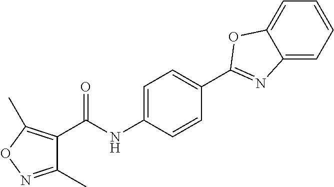 [SMILES: Cc1noc(C)c1C(=O)Nc1ccc(-c2nc3ccccc3o2)cc1] |
| 26 | 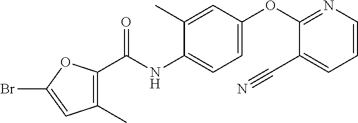 [SMILES: Cc1cc(Oc2ncccc2C#N)ccc1NC(=O)c1oc(Br)cc1C] |
| 27 | 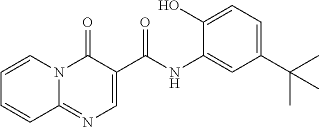 [SMILES: CC(C)(C)c1ccc(O)c(NC(=O)c2cnc3ccccn3c2=O)c1] |
| 28 | 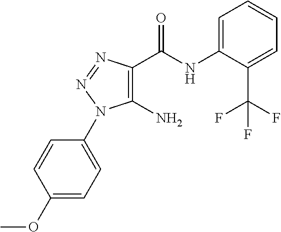 [SMILES: COc1ccc(-n2nnc(C(=O)Nc3ccccc3C(F)(F)F)c2N)cc1] |
| 29 | 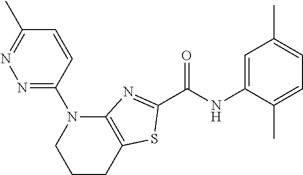 [SMILES: Cc1ccc(C)c(NC(=O)c2nc3c(s2)CCCN3c2ccc(C)nn2)c1] |
| 30 | 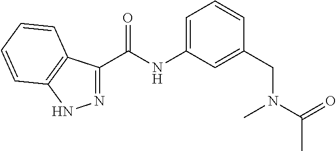 [SMILES: CC(=O)N(C)Cc1cccc(NC(=O)c2n[nH]c3ccccc23)c1] |
| 31 | 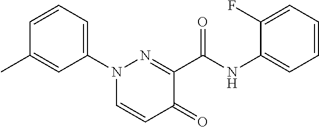 [SMILES: Cc1cccc(-n2ccc(=O)c(C(=O)Nc3ccccc3F)n2)c1] |
| 32 | 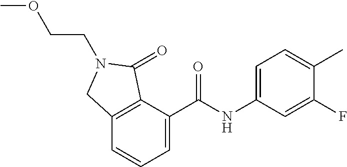 |
| 33 | 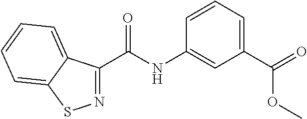 [SMILES: COC(=O)c1cccc(NC(=O)c2nsc3ccccc23)c1] |
| 34 | 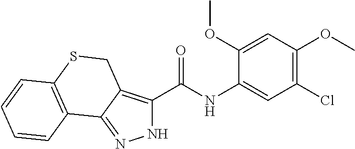 [SMILES: COc1cc(OC)c(NC(=O)c2[nH]nc3c2CSc2ccccc2-3)cc1Cl] |
| 35 | 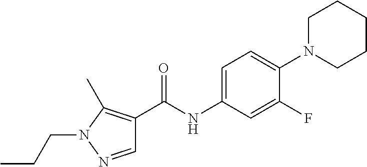 [SMILES: CCCn1ncc(C(=O)Nc2ccc(N3CCCCC3)c(F)c2)c1C] |
| 36 | 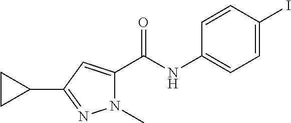 [SMILES: Cn1nc(C2CC2)cc1C(=O)Nc1ccc(I)cc1] |
| 37 | 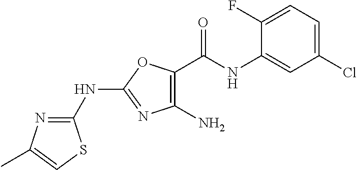 [SMILES: Cc1csc(Nc2nc(N)c(C(=O)Nc3cc(Cl)ccc3F)o2)n1] |
| 38 | 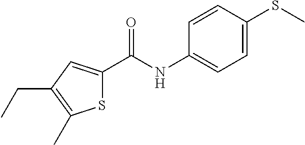 [SMILES: CCc1cc(C(=O)Nc2ccc(SC)cc2)sc1C] |
| 39 | 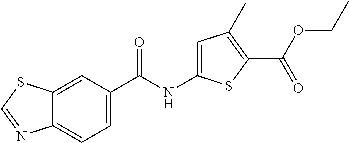 [SMILES: CCOC(=O)c1sc(NC(=O)c2ccc3ncsc3c2)cc1C] |
| 40 | 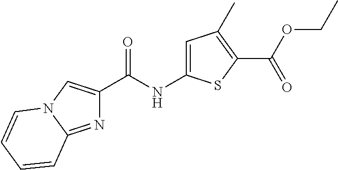 [SMILES: CCOC(=O)c1sc(NC(=O)c2cn3ccccc3n2)cc1C] |
| 41 | 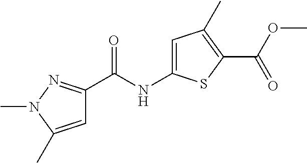 |
| 42 | 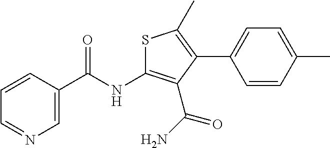 [SMILES: Cc1ccc(-c2c(C)sc(NC(=O)c3cccnc3)c2C(N)=O)cc1] |
| 43 | 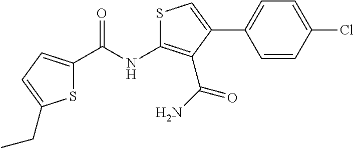 [SMILES: CCc1ccc(C(=O)Nc2scc(-c3ccc(Cl)cc3)c2C(N)=O)s1] |
| 44 | 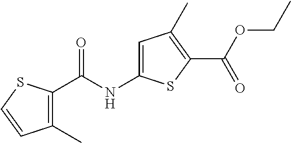 [SMILES: CCOC(=O)c1sc(NC(=O)c2sccc2C)cc1C] |
| 45 | 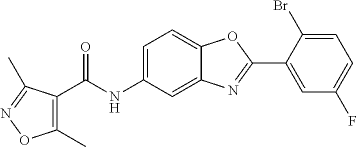 [SMILES: Cc1noc(C)c1C(=O)Nc1ccc2oc(-c3cc(F)ccc3Br)nc2c1] |
| 46 | 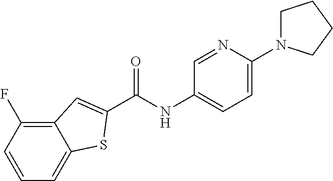 [SMILES: O=C(Nc1ccc(N2CCCC2)nc1)c1cc2c(F)cccc2s1] |
| 47 | 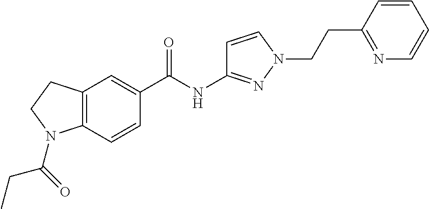 [SMILES: CCC(=O)N1CCc2cc(C(=O)Nc3ccn(CCc4ccccn4)n3)ccc21] |
| 48 | 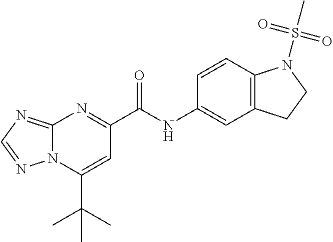 [SMILES: CC(C)(C)c1cc(C(=O)Nc2ccc3c(c2)CCN3S(C)(=O)=O)nc2ncnn12] |
| 49 | 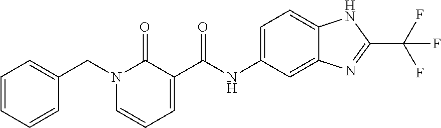 [SMILES: O=C(Nc1ccc2[nH]c(C(F)(F)F)nc2c1)c1cccn(Cc2ccccc2)c1=O] |

In some embodiments, the compound represented by Formula (Ia) is a compound or salt thereof of Table 1A below.

In some embodiments, the compound is a compound or a salt thereof of Table 1A below:

TABLE 1A
AHR antagonists

| Compd. | |
|---|---|
| No. | Structure |
| 1A | 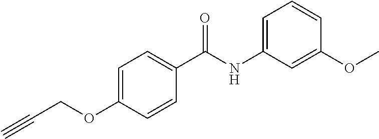 [SMILES: C#CCOc1ccc(C(=O)Nc2cccc(OC)c2)cc1] |
| 2A | 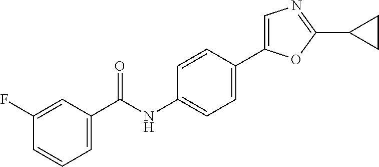 [SMILES: O=C(Nc1ccc(-c2cnc(C3CC3)o2)cc1)c1cccc(F)c1] |
| 3A | 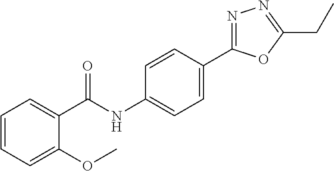 [SMILES: CCc1nnc(-c2ccc(NC(=O)c3ccccc3OC)cc2)o1] |
| 4A | 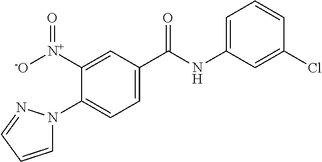 |
| 5A | 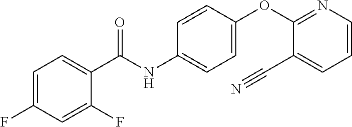 [SMILES: N#Cc1cccnc1Oc1ccc(NC(=O)c2ccc(F)cc2F)cc1] |
| 6A | 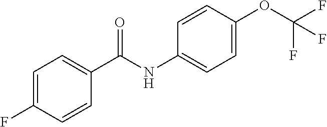 [SMILES: O=C(Nc1ccc(OC(F)(F)F)cc1)c1ccc(F)cc1] |
| 7A | 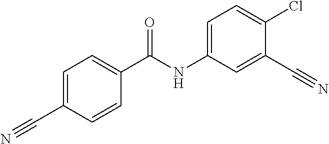 [SMILES: N#Cc1ccc(C(=O)Nc2ccc(Cl)c(C#N)c2)cc1] |
| 8A | 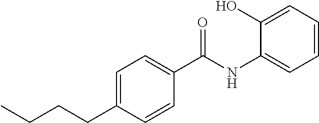 [SMILES: CCCCc1ccc(C(=O)Nc2ccccc2O)cc1] |
| 9A | 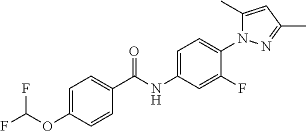 [SMILES: Cc1cc(C)n(-c2ccc(NC(=O)c3ccc(OC(F)F)cc3)cc2F)n1] |
| 10A | 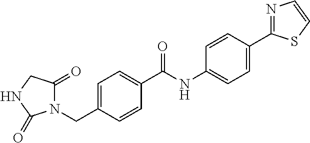 [SMILES: O=C(Nc1ccc(-c2nccs2)cc1)c1ccc(CN2C(=O)CNC2=O)cc1] |
| 11A | 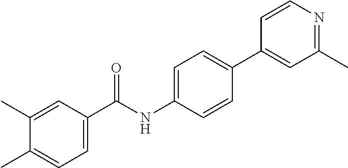 |
| 12A | 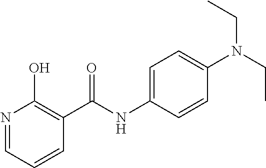 [SMILES: CCN(CC)c1ccc(NC(=O)c2cccnc2O)cc1] |
| 13A | 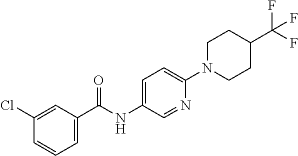 [SMILES: O=C(Nc1ccc(N2CCC(C(F)(F)F)CC2)nc1)c1cccc(Cl)c1] |
| 14A | 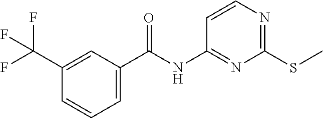 [SMILES: CSc1nccc(NC(=O)c2cccc(C(F)(F)F)c2)n1] |
| 15A | 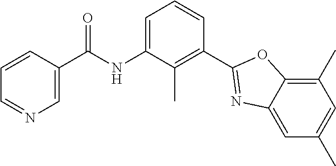 [SMILES: Cc1cc(C)c2oc(-c3cccc(NC(=O)c4cccnc4)c3C)nc2c1] |
| 16A | 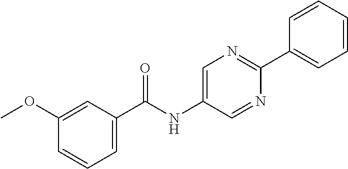 [SMILES: COc1cccc(C(=O)Nc2cnc(-c3ccccc3)nc2)c1] |
| 17A | 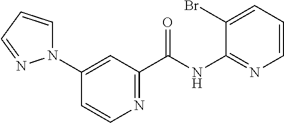 [SMILES: O=C(Nc1ncccc1Br)c1cc(-n2cccn2)ccn1] |
| 18A | 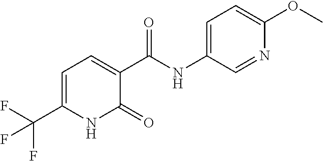 [SMILES: COc1ccc(NC(=O)c2ccc(C(F)(F)F)[nH]c2=O)cn1] |
| 19A | 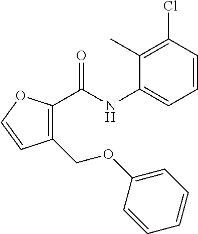 [SMILES: Cc1c(Cl)cccc1NC(=O)c1occc1COc1ccccc1] |
| 20A | 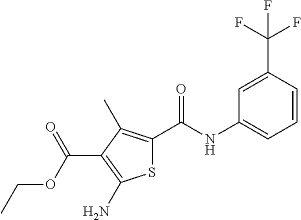 [SMILES: CCOC(=O)c1c(N)sc(C(=O)Nc2cccc(C(F)(F)F)c2)c1C] |
| 21A | 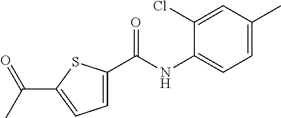 [SMILES: CC(=O)c1ccc(C(=O)Nc2ccc(C)cc2Cl)s1] |
| 22A | 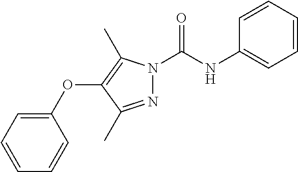 [SMILES: Cc1nn(C(=O)Nc2ccccc2)c(C)c1Oc1ccccc1] |
| 23A | 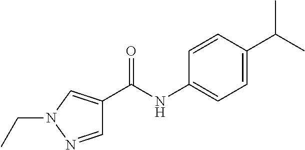 [SMILES: CCn1cc(C(=O)Nc2ccc(C(C)C)cc2)cn1] |
| 24A | 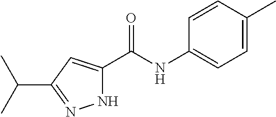 [SMILES: Cc1ccc(NC(=O)c2cc(C(C)C)n[nH]2)cc1] |
| 25A | 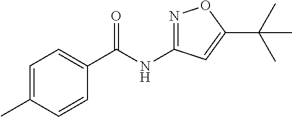 [SMILES: Cc1ccc(C(=O)Nc2cc(C(C)(C)C)on2)cc1] |
| 26A | 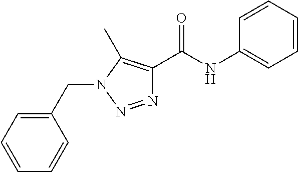 [SMILES: Cc1c(C(=O)Nc2ccccc2)nnn1Cc1ccccc1] |
| 27A | 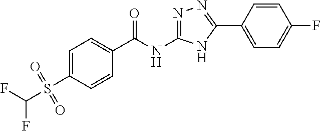 [SMILES: O=C(Nc1nnc(-c2ccc(F)cc2)[nH]1)c1ccc(S(=O)(=O)C(F)F)cc1] |
| 28A | 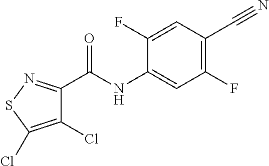 [SMILES: N#Cc1cc(F)c(NC(=O)c2nsc(Cl)c2Cl)cc1F] |
| 29A | 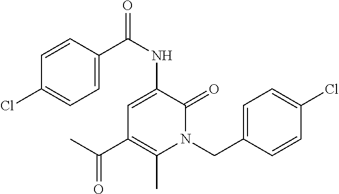 [SMILES: CC(=O)c1cc(NC(=O)c2ccc(Cl)cc2)c(=O)n(Cc2ccc(Cl)cc2)c1C] |
| 30A | 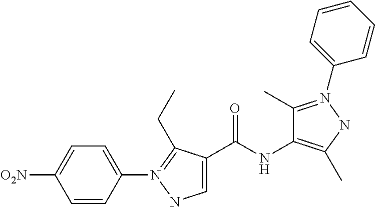 |
| 31A | 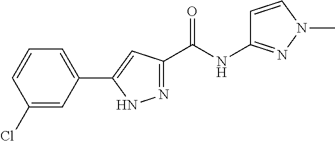 [SMILES: Cn1ccc(NC(=O)c2cc(-c3cccc(Cl)c3)[nH]n2)n1] |
| 32A | 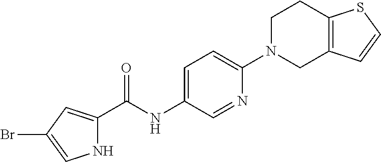 [SMILES: O=C(Nc1ccc(N2CCc3sccc3C2)nc1)c1cc(Br)c[nH]1] |
| 33A | 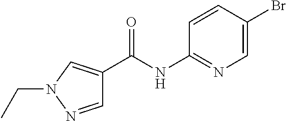 [SMILES: CCn1cc(C(=O)Nc2ccc(Br)cn2)cn1] |
| 34A | 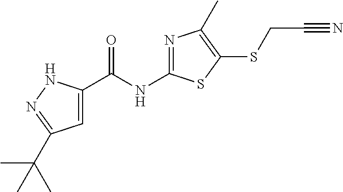 [SMILES: Cc1nc(NC(=O)c2cc(C(C)(C)C)n[nH]2)sc1SCC#N] |
| 35A | 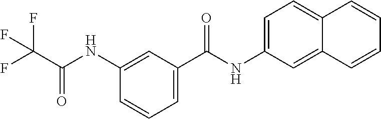 [SMILES: O=C(Nc1ccc2ccccc2c1)c1cccc(NC(=O)C(F)(F)F)c1] |
| 36A | 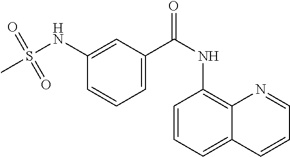 [SMILES: CS(=O)(=O)Nc1cccc(C(=O)Nc2cccc3cccnc23)c1] |
| 37A | 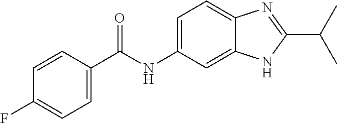 [SMILES: CC(C)c1nc2ccc(NC(=O)c3ccc(F)cc3)cc2[nH]1] |
| 38A | 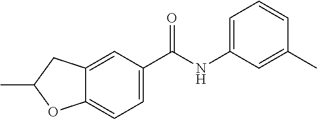 |
| 39A | 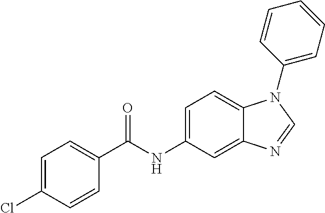 [SMILES: O=C(Nc1ccc2c(c1)ncn2-c1ccccc1)c1ccc(Cl)cc1] |
| 40A | 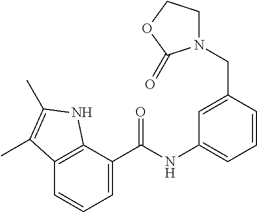 [SMILES: Cc1[nH]c2c(C(=O)Nc3cccc(CN4CCOC4=O)c3)cccc2c1C] |
| 41A | 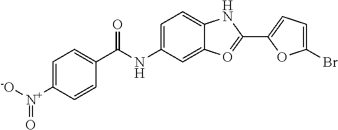 |
| 42A | 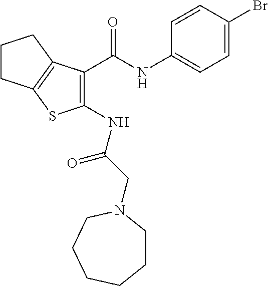 [SMILES: O=C(CN1CCCCCC1)Nc1sc2c(c1C(=O)Nc1ccc(Br)cc1)CCC2] |
| 43A | 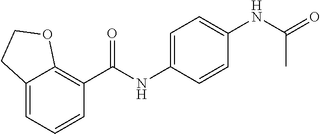 [SMILES: CC(=O)Nc1ccc(NC(=O)c2cccc3c2OCC3)cc1] |
| 44A | 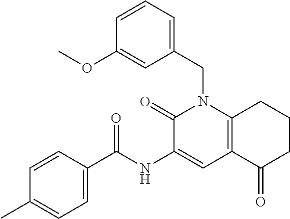 [SMILES: COc1cccc(Cn2c3c(cc(NC(=O)c4ccc(C)cc4)c2=O)C(=O)CCC3)c1] |
| 45A | 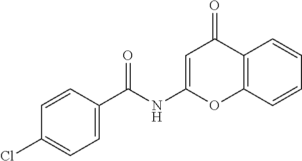 [SMILES: O=C(Nc1cc(=O)c2ccccc2o1)c1ccc(Cl)cc1] |
| 46A | 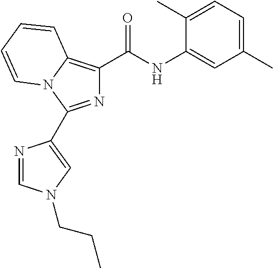 [SMILES: CCCn1cnc(-c2nc(C(=O)Nc3cc(C)ccc3C)c3ccccn23)c1] |
| 47A | 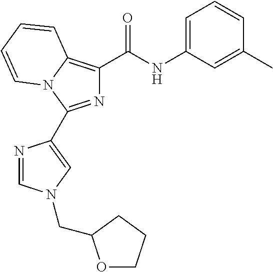 [SMILES: Cc1cccc(NC(=O)c2nc(-c3cn(CC4CCCO4)cn3)n3ccccc23)c1] |
| 48A | 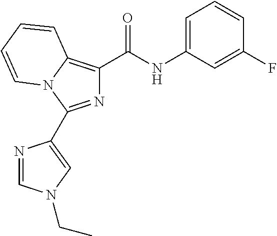 [SMILES: CCn1cnc(-c2nc(C(=O)Nc3cccc(F)c3)c3ccccn23)c1] |
| 49A | 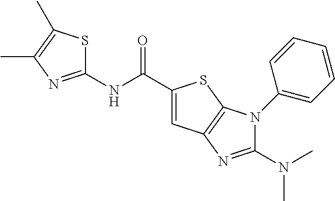 [SMILES: Cc1nc(NC(=O)c2cc3nc(N(C)C)n(-c4ccccc4)c3s2)sc1C] |
| 50A | 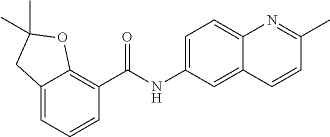 |
| 51A | 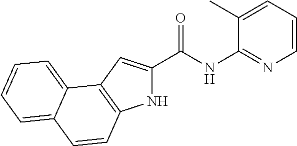 [SMILES: Cc1cccnc1NC(=O)c1cc2c(ccc3ccccc32)[nH]1] |
| 52A | 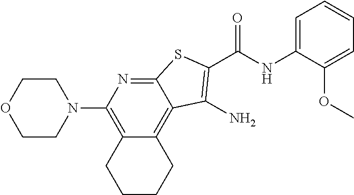 [SMILES: COc1ccccc1NC(=O)c1sc2nc(N3CCOCC3)c3c(c2c1N)CCCC3] |

In some embodiments, the compound represented by Formula (Ia) is a compound or salt thereof of Table 1B below.

In some embodiments, the compound is a compound or a salt thereof of Table 11B below:

TABLE 1B
AHR antagonists

| Compd. | |
|---|---|
| No. | Structure |
| 1B | 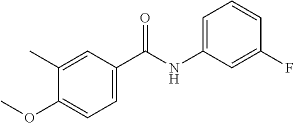 [SMILES: COc1ccc(C(=O)Nc2cccc(F)c2)cc1C] |
| 2B | 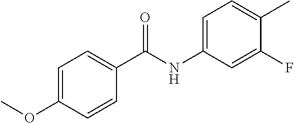 |
| 3B | 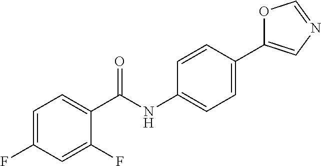 [SMILES: O=C(Nc1ccc(-c2cnco2)cc1)c1ccc(F)cc1F] |
| 4B | 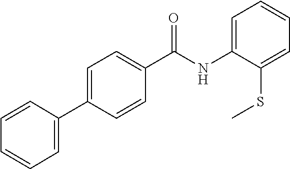 [SMILES: CSc1ccccc1NC(=O)c1ccc(-c2ccccc2)cc1] |
| 5B | 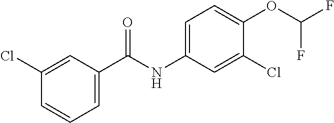 [SMILES: O=C(Nc1ccc(OC(F)F)c(Cl)c1)c1cccc(Cl)c1] |
| 6B | 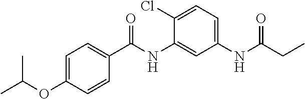 [SMILES: CCC(=O)Nc1ccc(Cl)c(NC(=O)c2ccc(OC(C)C)cc2)c1] |
| 7B | 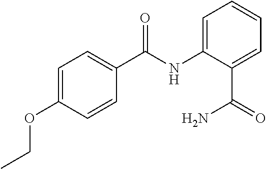 [SMILES: CCOc1ccc(C(=O)Nc2ccccc2C(N)=O)cc1] |
| 8B | 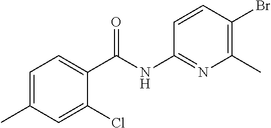 [SMILES: Cc1ccc(C(=O)Nc2ccc(Br)c(C)n2)c(Cl)c1] |
| 9B | 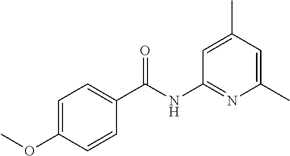 |
| 10B | 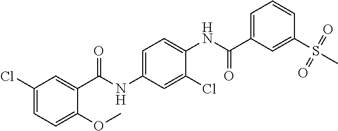 [SMILES: COc1ccc(Cl)cc1C(=O)Nc1ccc(NC(=O)c2cccc(S(C)(=O)=O)c2)c(Cl)c1] |
| 11B | 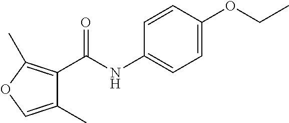 [SMILES: CCOc1ccc(NC(=O)c2c(C)coc2C)cc1] |
| 12B | 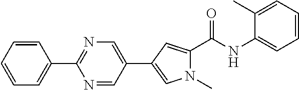 [SMILES: Cc1ccccc1NC(=O)c1cc(-c2cnc(-c3ccccc3)nc2)cn1C] |
| 13B | 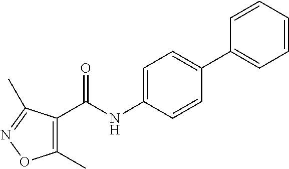 [SMILES: Cc1noc(C)c1C(=O)Nc1ccc(-c2ccccc2)cc1] |
| 14B | 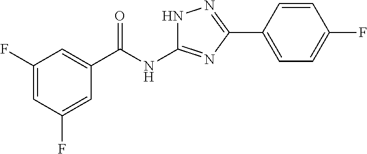 [SMILES: O=C(Nc1nc(-c2ccc(F)cc2)n[nH]1)c1cc(F)cc(F)c1] |
| 15B | 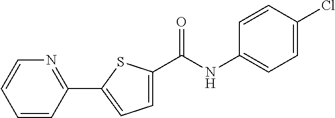 [SMILES: O=C(Nc1ccc(Cl)cc1)c1ccc(-c2ccccn2)s1] |
| 16B | 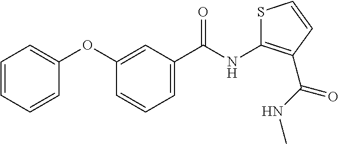 [SMILES: CNC(=O)c1ccsc1NC(=O)c1cccc(Oc2ccccc2)c1] |
| 17B | 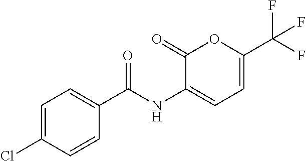 [SMILES: O=C(Nc1ccc(C(F)(F)F)oc1=O)c1ccc(Cl)cc1] |
| 18B | 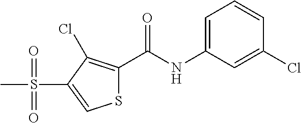 [SMILES: CS(=O)(=O)c1csc(C(=O)Nc2cccc(Cl)c2)c1Cl] |
| 19B | 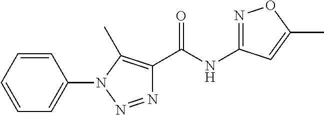 [SMILES: Cc1cc(NC(=O)c2nnn(-c3ccccc3)c2C)no1] |
| 20B | 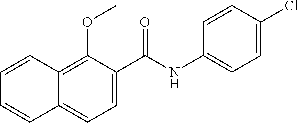 [SMILES: COc1c(C(=O)Nc2ccc(Cl)cc2)ccc2ccccc12] |
| 21B | 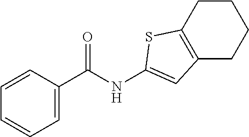 [SMILES: O=C(Nc1cc2c(s1)CCCC2)c1ccccc1] |
| 22B | 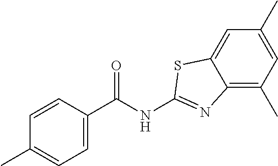 |
| 23B | 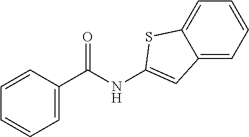 [SMILES: O=C(Nc1cc2ccccc2s1)c1ccccc1] |
| 24B | 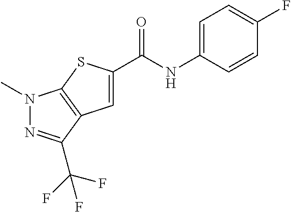 [SMILES: Cn1nc(C(F)(F)F)c2cc(C(=O)Nc3ccc(F)cc3)sc21] |
| 25B | 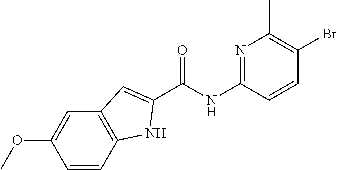 [SMILES: COc1ccc2[nH]c(C(=O)Nc3ccc(Br)c(C)n3)cc2c1] |
| 26B | 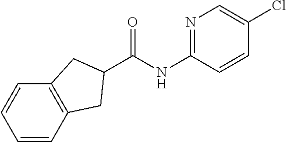 [SMILES: O=C(Nc1ccc(Cl)cn1)C1Cc2ccccc2C1] |
| 27B | 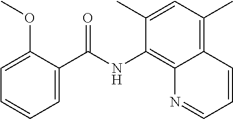 [SMILES: COc1ccccc1C(=O)Nc1c(C)cc(C)c2cccnc12] |
| 28B | 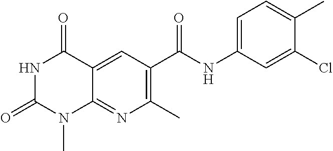 [SMILES: Cc1ccc(NC(=O)c2cc3c(=O)[nH]c(=O)n(C)c3nc2C)cc1Cl] |
| 29B | 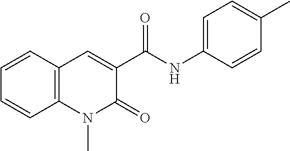 [SMILES: Cc1ccc(NC(=O)c2cc3ccccc3n(C)c2=O)cc1] |
| 30B | 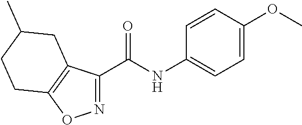 [SMILES: COc1ccc(NC(=O)c2noc3c2CC(C)CC3)cc1] |
| 31B | 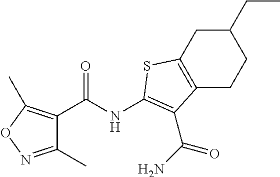 [SMILES: CCC1CCc2c(sc(NC(=O)c3c(C)noc3C)c2C(N)=O)C1] |
| 32B | 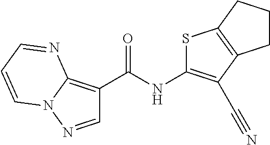 [SMILES: N#Cc1c(NC(=O)c2cnn3cccnc23)sc2c1CCC2] |

### Compounds of Formula (Ib)

In some embodiments, the disclosure features a compound represented by Formula (Ib) or a salt thereof

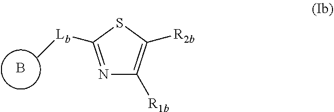

wherein

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—*, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{ba}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a thiazole carbon and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R_{1b} is hydrogen or -L_{c}-C;

R_{2b} is hydrogen, an optionally substituted pyrazole ring, or CONR_{3b}R_{4b}, wherein each R_{3b} and R_{4b} is independently hydrogen or C₁-C₆ alkyl;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a thiazole carbon and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl; and

R_{1b} and R_{2b} are not both hydrogen.

In some embodiments, the compound is represented by Formula (Ib) and R_{1b} is hydrogen.

In some embodiments, the compound is represented by Formula (Ib) and B is an optionally substituted monocyclic ring selected from the group consisting of benzene, pyridine, 2,3-dihydropyrrole, 1,2,3-triazole, pyrrolidine, thiophene, piperazine, imidazole, tetrazole, pyrrolidin-2-one, and 1,2-dihydro-3H-pyrrol-3-one.

In some embodiments, the compound is represented by Formula (Ib) and B is an optionally substituted monocyclic ring selected from the group consisting of benzene, pyridine, 2,3-dihydropyrrole, 1,2,3-triazole, pyrrolidine, and thiophene.

In some embodiments, the compound is represented by Formula (Ib) and B is an optionally substituted monocyclic ring selected from the group consisting of:

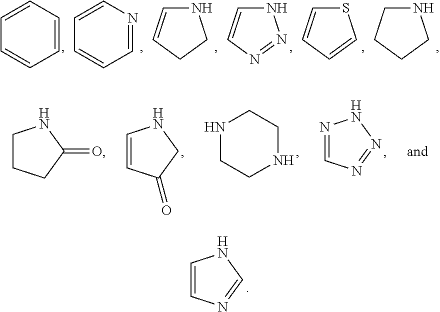

In some embodiments, the compound is represented by Formula (Ib) and B is an optionally substituted monocyclic ring selected from the group consisting of:

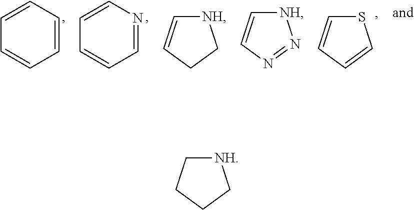

In some embodiments, the compound is represented by Formula (Ib) and B is an optionally substituted bicyclic ring selected from the group consisting of benzo[d]isooxazole, 2,3-dihydrobenzofuran, and imidazo[1,2-a]pyridine.

In some embodiments, the compound is represented by Formula (Ib) and B is an optionally substituted monocyclic ring selected from the group consisting of

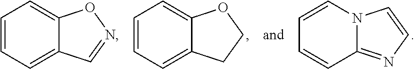

In some embodiments, the compound is represented by Formula (Ib) and L_{b} is selected from the group consisting of a covalent bond, *—NH—**, and *—NR_{bb}C(O)—**.

In some embodiments, the compound is represented by Formula (Ib) and L_{b} is a covalent bond.

In some embodiments, the compound is represented by Formula (Ib), R_{1b} is -L_{c}-C and L_{c} is a covalent bond.

In some embodiments, the compound is represented by Formula (Ib), R_{1b} is -L_{c}-C and C is an optionally substituted monocyclic ring selected from the group consisting of benzene, pyridine, pyrrole, pyrazole, 1,3,4-oxadiazole, 4H-1,2,4-triazole, thiophene, 1H-1,2,4-triazole, 1,2,3,4-tetrahydropyrimidine, and pyrimidine-2,4(1H,3H)-dione.

In some embodiments, the compound is represented by Formula (Ib), R_{1b} is -L_{c}-C and C is an optionally substituted monocyclic ring selected from the group consisting of benzene, pyridine, pyrrole, pyrazole, and 1,3,4-oxadiazole.

In some embodiments, the compound is represented by Formula (Ib), R_{1b} is -L_{c}-C and C is an optionally substituted monocyclic ring selected from the group consisting of:

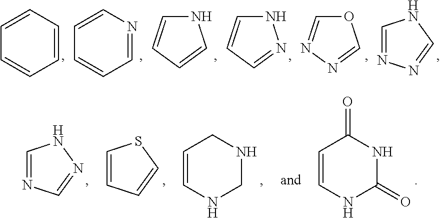

In some embodiments, the compound is represented by Formula (Ib), R_{1b} is -L_{c}-C and C is an optionally substituted monocyclic ring selected from the group consisting of:

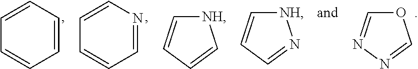

In some embodiments, the compound is represented by Formula (Ib), R_{1b} is -L_{c}-C and C is an optionally substituted bicyclic ring selected from the group consisting of imidazo[1,2-a]pyridine, benzo[d]imidazole, indoline, 1,2,3,4-tetrahydroquinoline, octahydro-1H-benzo[d]imidazole, and octahydro-2h-benzo[d]imidazole-2-one.

In some embodiments, the compound is represented by Formula (Ib), R_{1b} is -L_{c}-C and C is an optionally substituted bicyclic ring selected from the group consisting of imidazo[1,2-a]pyridine and benzo[d]imidazole.

In some embodiments, the compound is represented by Formula (Ib), R_{1b} is -L_{c}-C and C is an optionally substituted bicyclic ring selected from the group consisting of:

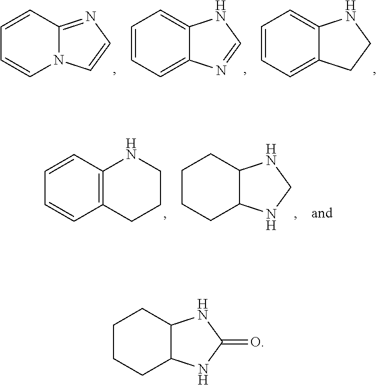

In some embodiments, the compound is represented by Formula (Ib), R_{1b} is -L_{c}-C and C is an optionally substituted bicyclic ring selected from the group consisting of:

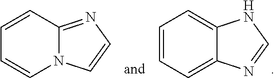 [SMILES: c1ccn2ccnc2c1 ; c1ccc2[nH]cnc2c1]

In some embodiments, the compound is represented by Formula (Ib), R_{1b} is -L_{c}-C and both B and C are an optionally substituted monocyclic ring selected from benzene and pyridine.

In some embodiments, the compound is represented by Formula (Ib), R_{1b} is -L_{c}-C and both B and C are an optionally substituted monocyclic ring selected from:

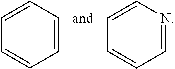 [SMILES: c1ccccc1 ; c1ccncc1]

In some embodiments, the compound is of Formula (Ib) and A is optionally substituted with one or more of:—CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ib) and A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ib) and B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR—N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ib) and B is optionally substituted with one or more of:—CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is of Formula (Ib) and C is optionally substituted with one or more of:—CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.In some embodiments, the compound is of Formula (Ib) and C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)F, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound represented by Formula (Ib) is a compound or salt thereof of Table 2 below.

In some embodiments, the compound is a compound or a salt thereof of Table 2 below:

TABLE 2
AHR antagonists

| Compd. | |
|---|---|
| No. | Structure |
| 50 | 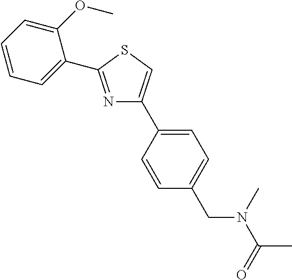 [SMILES: COc1ccccc1-c1nc(-c2ccc(CN(C)C(C)=O)cc2)cs1] |
| 51 | 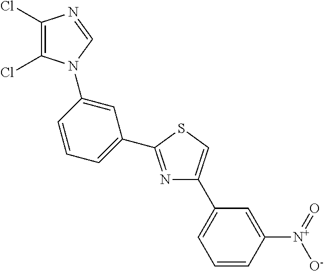 [SMILES: O=[N+]([O-])c1cccc(-c2csc(-c3cccc(-n4cnc(Cl)c4Cl)c3)n2)c1] |
| 52 | 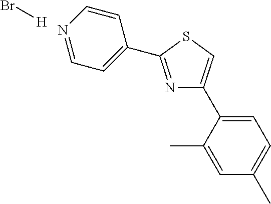 |
| 53 | 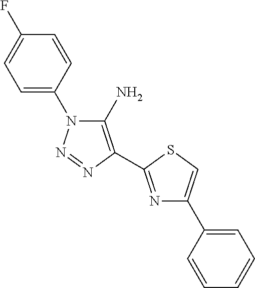 [SMILES: Nc1c(-c2nc(-c3ccccc3)cs2)nnn1-c1ccc(F)cc1] |
| 54 | 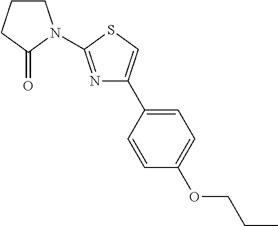 [SMILES: CCCOc1ccc(-c2csc(N3CCCC3=O)n2)cc1] |
| 55 | 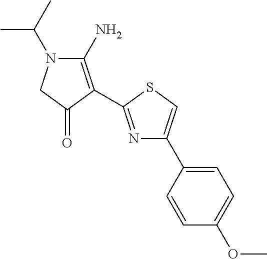 [SMILES: COc1ccc(-c2csc(C3=C(N)N(C(C)C)CC3=O)n2)cc1] |
| 56 | 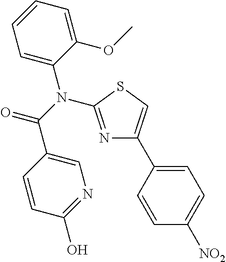 [SMILES: COc1ccccc1N(C(=O)c1ccc(O)nc1)c1nc(-c2ccc([N+](=O)[O-])cc2)cs1] |
| 57 | 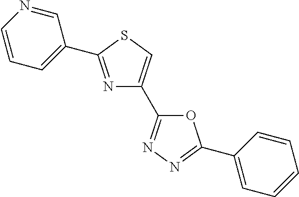 [SMILES: c1ccc(-c2nnc(-c3csc(-c4cccnc4)n3)o2)cc1] |
| 58 | 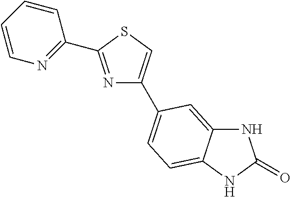 [SMILES: O=c1[nH]c2ccc(-c3csc(-c4ccccn4)n3)cc2[nH]1] |
| 59 | 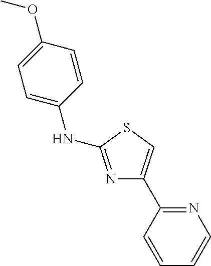 [SMILES: COc1ccc(Nc2nc(-c3ccccn3)cs2)cc1] |
| 60 | 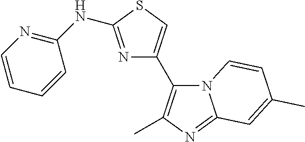 [SMILES: Cc1ccn2c(-c3csc(Nc4ccccn4)n3)c(C)nc2c1] |
| 61 | 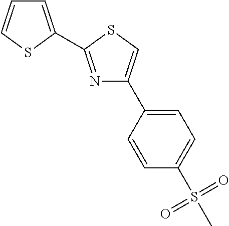 [SMILES: CS(=O)(=O)c1ccc(-c2csc(-c3cccs3)n2)cc1] |
| 62 | 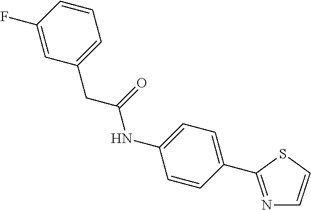 [SMILES: O=C(Cc1cccc(F)c1)Nc1ccc(-c2nccs2)cc1] |
| 63 | 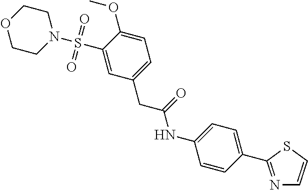 [SMILES: COc1ccc(CC(=O)Nc2ccc(-c3nccs3)cc2)cc1S(=O)(=O)N1CCOCC1] |
| 64 | 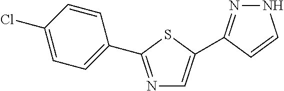 [SMILES: Clc1ccc(-c2ncc(-c3cc[nH]n3)s2)cc1] |
| 126 | 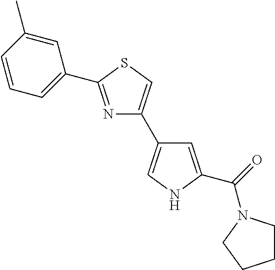 [SMILES: Cc1cccc(-c2nc(-c3c[nH]c(C(=O)N4CCCC4)c3)cs2)c1] |
| 65 | 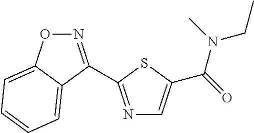 [SMILES: CCN(C)C(=O)c1cnc(-c2noc3ccccc23)s1] |

In some embodiments, the compound represented by Formula (Ib) is a compound or salt thereof of Table 2 below.

In some embodiments, the compound is a compound or a salt thereof of Table 2A below:

TABLE 2A
AHR antagonists

| Compd. | |
|---|---|
| No. | Structure |
| 53A | 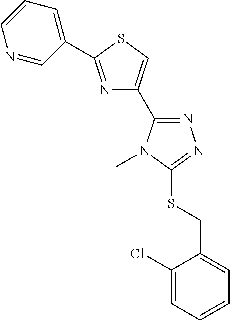 [SMILES: Cn1c(SCc2ccccc2Cl)nnc1-c1csc(-c2cccnc2)n1] |
| 54A | 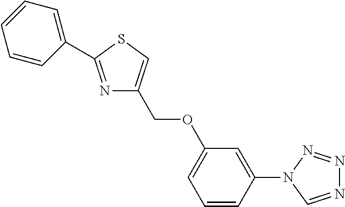 [SMILES: c1ccc(-c2nc(COc3cccc(-n4cnnn4)c3)cs2)cc1] |
| 55A | 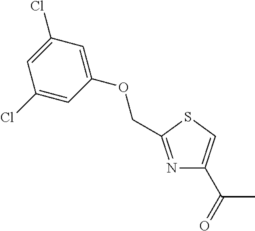 [SMILES: CC(=O)c1csc(COc2cc(Cl)cc(Cl)c2)n1] |
| 56A | 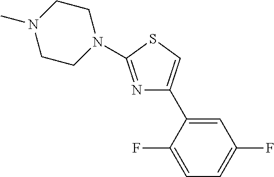 [SMILES: CN1CCN(c2nc(-c3cc(F)ccc3F)cs2)CC1] |
| 57A | 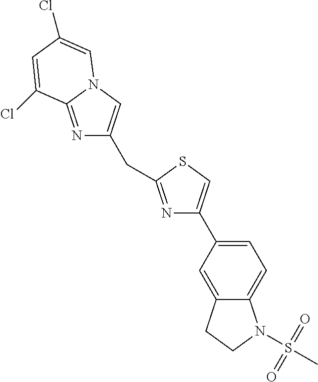 [SMILES: CS(=O)(=O)N1CCc2cc(-c3csc(Cc4cn5cc(Cl)cc(Cl)c5n4)n3)ccc21] |
| 58A | 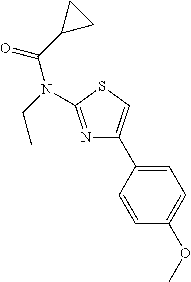 [SMILES: CCN(C(=O)C1CC1)c1nc(-c2ccc(OC)cc2)cs1] |
| 59A | 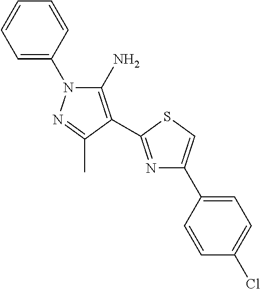 [SMILES: Cc1nn(-c2ccccc2)c(N)c1-c1nc(-c2ccc(Cl)cc2)cs1] |
| 60A | 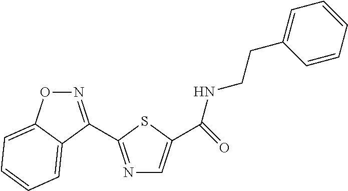 [SMILES: O=C(NCCc1ccccc1)c1cnc(-c2noc3ccccc23)s1] |
| 61A | 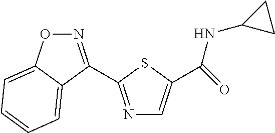 [SMILES: O=C(NC1CC1)c1cnc(-c2noc3ccccc23)s1] |
| 62A | 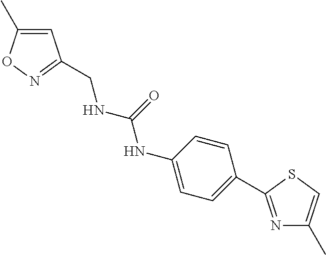 |
| 63A | 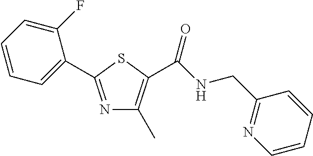 [SMILES: Cc1nc(-c2ccccc2F)sc1C(=O)NCc1ccccn1] |
| 64A | 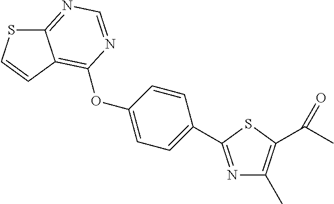 [SMILES: CC(=O)c1sc(-c2ccc(Oc3ncnc4sccc34)cc2)nc1C] |
| 65A | 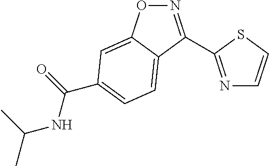 [SMILES: CC(C)NC(=O)c1ccc2c(-c3nccs3)noc2c1] |
| 66A | 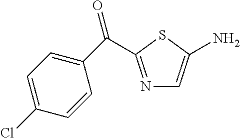 [SMILES: Nc1cnc(C(=O)c2ccc(Cl)cc2)s1] |

In some embodiments, the compound represented by Formula (Ib) is a compound or salt thereof of Table 2 below.

In some embodiments the compound is a compound or a salt thereof of Table 2B below:

TABLE 2B
AHR antagonists

| Compd. | |
|---|---|
| No. | Structure |
| 33B | 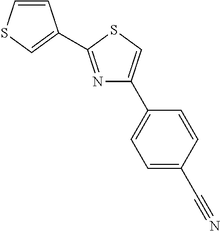 [SMILES: N#Cc1ccc(-c2csc(-c3ccsc3)n2)cc1] |
| 34B | 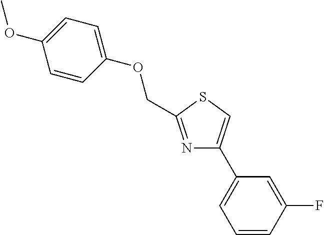 [SMILES: COc1ccc(OCc2nc(-c3cccc(F)c3)cs2)cc1] |
| 35B | 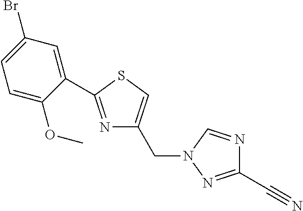 [SMILES: COc1ccc(Br)cc1-c1nc(Cn2cnc(C#N)n2)cs1] |
| 36B | 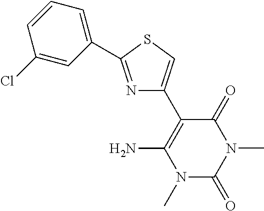 [SMILES: Cn1c(N)c(-c2csc(-c3cccc(Cl)c3)n2)c(=O)n(C)c1=O] |
| 37B | 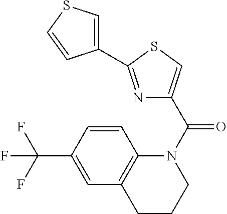 [SMILES: O=C(c1csc(-c2ccsc2)n1)N1CCCc2cc(C(F)(F)F)ccc21] |
| 38B | 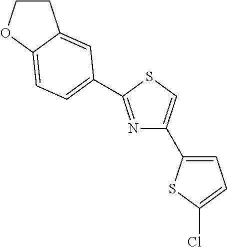 [SMILES: Clc1ccc(-c2csc(-c3ccc4c(c3)CCO4)n2)s1] |
| 39B | 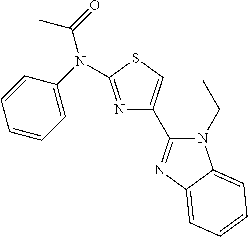 [SMILES: CCn1c(-c2csc(N(C(C)=O)c3ccccc3)n2)nc2ccccc21] |
| 40B | 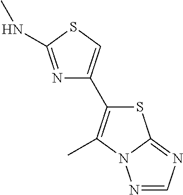 [SMILES: CNc1nc(-c2sc3ncnn3c2C)cs1] |
| 41B | 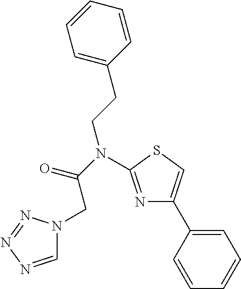 [SMILES: O=C(Cn1cnnn1)N(CCc1ccccc1)c1nc(-c2ccccc2)cs1] |

### Compounds of Formula (Ic)

In some embodiments, the disclosure features a compound represented by Formula (Ic) or a salt thereof

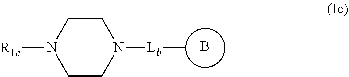

wherein

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a piperazine nitrogen and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R_{1c} is -L_{c}-C, C(O)R₂ₐ, or C(O)OR₂ₐ, wherein each R₂ₐ is C₁-C₆ alkyl;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a piperazine nitrogen and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (Ic) and R_{1c} is selected from the group consisting of C(O)CH₃ and C(O)OCH₂CH₃.

In some embodiments, the compound is represented by Formula (Ic) and B is an optionally substituted monocyclic ring selected from the group consisting of benzene, pyrimidine, pyridine, thiophene, 1,3,5-triazine, 1,3,4-thiadiazole, 4,5-dihydrothiazole, and thiazol-4(5H)-one.

In some embodiments, the compound is represented by Formula (Ic) and B is an optionally substituted monocyclic ring selected from the group consisting of benzene, pyrimidine, pyridine, and thiophene.

In some embodiments, the compound is represented by Formula (Ic) and B is an optionally substituted monocyclic ring selected from the group consisting of:

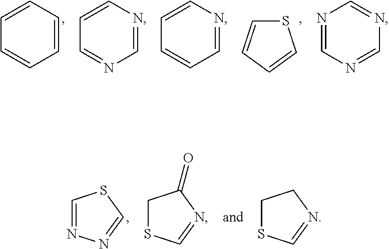

In some embodiments, the compound is represented by Formula (Ic) and B is an optionally substituted monocyclic ring selected from the group consisting of:

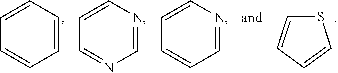

In some embodiments, the compound is represented by Formula (Ic) and B is an optionally substituted bicyclic ring selected from the group consisting of benzo[d]isothiazaole, thieno[2,3-d]pyrimidine, pteridine, [1,2,4]triazolo[4,3-b]pyridazine, 5,6,7,8-tetrahydroquinazoline, 7,8-dihydroquinazolin-5(6H)-one, and 4a,6,7,7a.-tetrahydro-5H-cyclopenta[b]pyridine.

In some embodiments, the compound is represented by Formula (Ic) and B is an optionally substituted bicyclic ring selected from the group consisting of benzo[d]isothiazaole and thieno[2,3-d]pyrimidine.

In some embodiments, the compound is represented by Formula (Ic) and B is an optionally substituted bicyclic ring selected from the group consisting of:

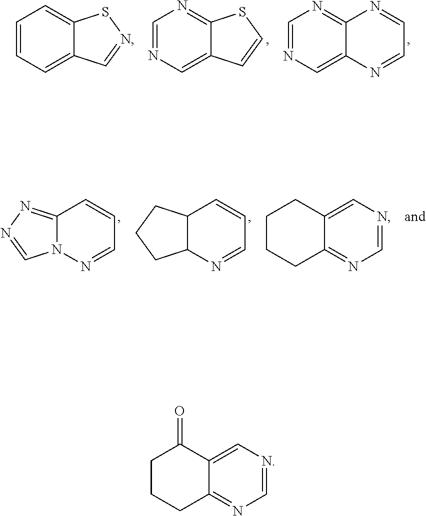

In some embodiments, the compound is represented by Formula (Ic) and B is an optionally substituted bicyclic ring selected from the group consisting of:

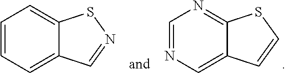 [SMILES: c1ccc2sncc2c1 ; c1ncc2ccsc2n1]

In some embodiments, the compound is represented by Formula (Ic) and L_{b} is selected from the group consisting of a covalent bond and *—SO₂—**.

In some embodiments, the compound is represented by Formula (Ic) and L_{b} is a covalent bond.

In some embodiments, the compound is represented by Formula (Ic), R_{1c} is -L_{c}-C and L is selected from the group consisting of a covalent bond, *—C(O)—**, *—N═CH₂—**, *—C(O)NH—**.

In some embodiments, the compound is represented by Formula (Ic), R₁ₐ is -L_{c}-C and C is an optionally substituted monocyclic ring selected from the group consisting of benzene, pyrimidine, thiazole, pyridine, pyridazine, 4,5-dihydrothiazole, 2,3,4,5-tetrahydro-1,2,4-triazine, 1,2,4-triazine-3,5(2H,4H)-dione and 2,4-dimethyl-1,2,4-triazine-3,5(2H, 4H)-dione.

In some embodiments, the compound is represented by Formula (Ic), R₁ₐ is -L_{c}-C and C is an optionally substituted monocyclic ring selected from the group consisting of benzene, pyrimidine, and thiazole.

In some embodiments, the compound is represented by Formula (Ic), R₁ₐ is -L_{c}-C and C is an optionally substituted monocyclic ring selected from the group consisting of:

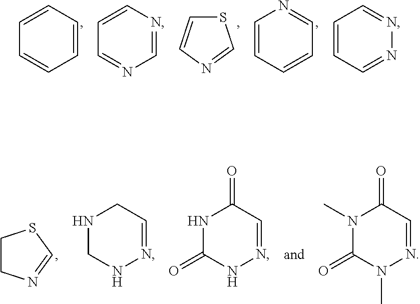

In some embodiments, the compound is represented by Formula (Ic), R₁ₐ is -L-C and C is an optionally substituted monocyclic ring selected from the group consisting of:

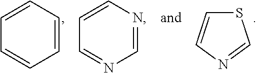

In some embodiments, the compound is represented by Formula (Ic), R₁ₐ is -L_{c}-C and C is an optionally substituted bicyclic ring selected from the group consisting of quinazoline and indole.

In some embodiments, the compound is represented by Formula (Ic), R₁ₐ is -L_{c}-C and C is an optionally substituted bicyclic ring selected from the group consisting of:

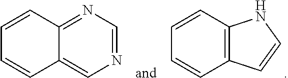 [SMILES: c1ccc2ncncc2c1 ; c1ccc2[nH]ccc2c1]

In some embodiments, the compound is of Formula (Ic) and A is optionally substituted with one or more of:—CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ic) and A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ic) and B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ic) and B is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is of Formula (Ic) and C is optionally substituted with one or more of:—CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ic) and C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound represented by Formula (Ic) is a compound or salt thereof of Table 3 below.

In some embodiments, the compound is a compound or a salt thereof of Table 3 below:

TABLE 3
AHR antagonists

| Compd. | |
|---|---|
| No. | Structure |
| 74 | 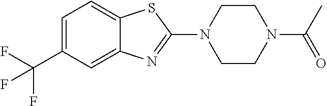 [SMILES: CC(=O)N1CCN(c2nc3cc(C(F)(F)F)ccc3s2)CC1] |
| 75 | 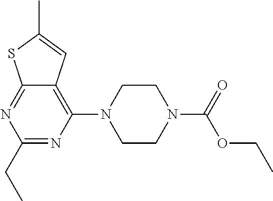 [SMILES: CCOC(=O)N1CCN(c2nc(CC)nc3sc(C)cc23)CC1] |
| 76 | 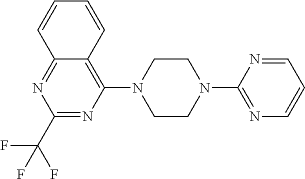 [SMILES: FC(F)(F)c1nc(N2CCN(c3ncccn3)CC2)c2ccccc2n1] |
| 77 | 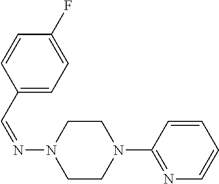 [SMILES: Fc1ccc(/C=N\N2CCN(c3ccccn3)CC2)cc1] |
| 78 | 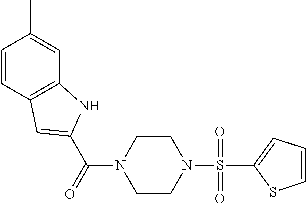 [SMILES: Cc1ccc2cc(C(=O)N3CCN(S(=O)(=O)c4cccs4)CC3)[nH]c2c1] |
| 79 | 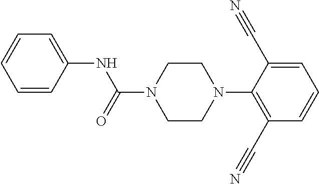 [SMILES: N#Cc1cccc(C#N)c1N1CCN(C(=O)Nc2ccccc2)CC1] |
| 80 | 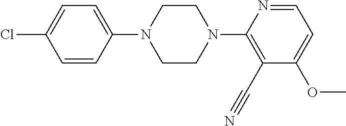 [SMILES: COc1ccnc(N2CCN(c3ccc(Cl)cc3)CC2)c1C#N] |
| 81 | 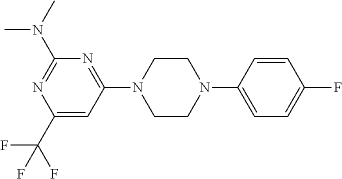 [SMILES: CN(C)c1nc(N2CCN(c3ccc(F)cc3)CC2)cc(C(F)(F)F)n1] |
| 111 | 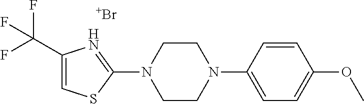 |

In some embodiments, the compound represented by Formula (Ic) is a compound or salt thereof of Table 3A below.

In some embodiments, the compound is a compound or a salt thereof of Table 3A below:

TABLE 3A
AHR antagonists

| Compd. | |
|---|---|
| No. | Structure |
| 67A | 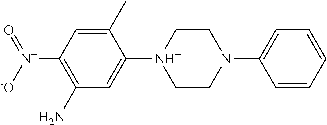 |
| 68A | 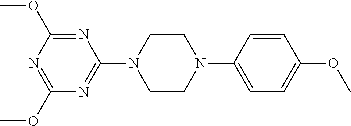 |
| 69A | 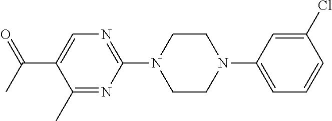 [SMILES: CC(=O)c1cnc(N2CCN(c3cccc(Cl)c3)CC2)nc1C] |
| 70A | 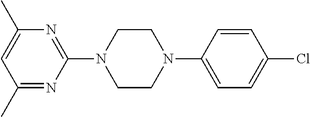 [SMILES: Cc1cc(C)nc(N2CCN(c3ccc(Cl)cc3)CC2)n1] |
| 71A | 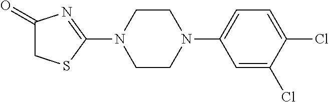 [SMILES: O=C1CSC(N2CCN(c3ccc(Cl)c(Cl)c3)CC2)=N1] |
| 72A | 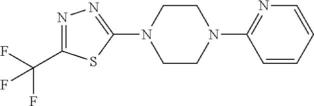 [SMILES: FC(F)(F)c1nnc(N2CCN(c3ccccn3)CC2)s1] |
| 73A | 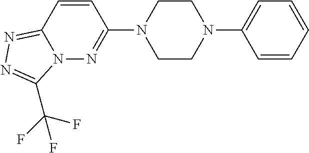 [SMILES: FC(F)(F)c1nnc2ccc(N3CCN(c4ccccc4)CC3)nn12] |
| 74A | 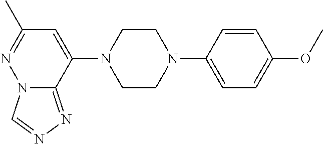 [SMILES: COc1ccc(N2CCN(c3cc(C)nn4cnnc34)CC2)cc1] |
| 75A | 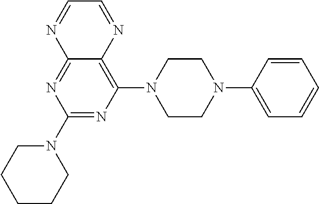 [SMILES: c1ccc(N2CCN(c3nc(N4CCCCC4)nc4nccnc34)CC2)cc1] |
| 76A | 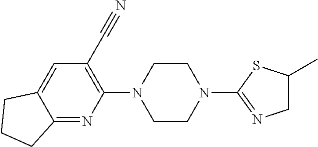 [SMILES: CC1CN=C(N2CCN(c3nc4c(cc3C#N)CCC4)CC2)S1] |
| 77A | 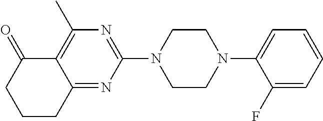 [SMILES: Cc1nc(N2CCN(c3ccccc3F)CC2)nc2c1C(=O)CCC2] |
| 78A | 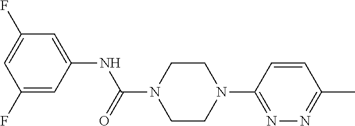 [SMILES: Cc1ccc(N2CCN(C(=O)Nc3cc(F)cc(F)c3)CC2)nn1] |
| 79A | 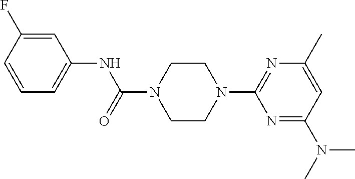 [SMILES: Cc1cc(N(C)C)nc(N2CCN(C(=O)Nc3cccc(F)c3)CC2)n1] |
| 80A | 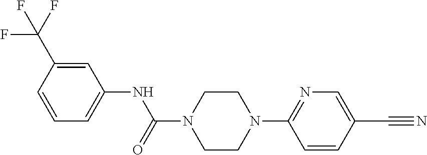 [SMILES: N#Cc1ccc(N2CCN(C(=O)Nc3cccc(C(F)(F)F)c3)CC2)nc1] |
| 81A | 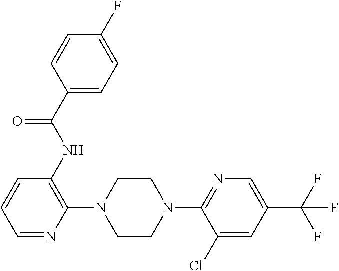 [SMILES: O=C(Nc1cccnc1N1CCN(c2ncc(C(F)(F)F)cc2Cl)CC1)c1ccc(F)cc1] |
| 82A | 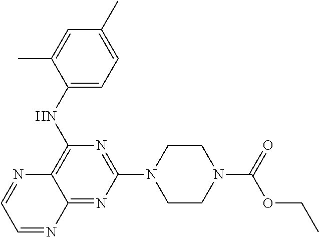 [SMILES: CCOC(=O)N1CCN(c2nc(Nc3ccc(C)cc3C)c3nccnc3n2)CC1] |

In some embodiments, the compound represented by Formula (Ic) is a compound or salt thereof of Table 3B below.

In some embodiments, the compound is a compound or a salt thereof of Table 3B below:

TABLE 3B
AHR antagonists

| Compd. | |
|---|---|
| No. | Structure |
| 42B | 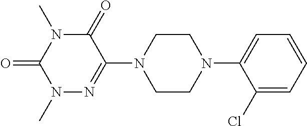 [SMILES: Cn1nc(N2CCN(c3ccccc3Cl)CC2)c(=O)n(C)c1=O] |

### Compounds of Formula (Id1) or Formula (Id2)

In some embodiments, the disclosure features a compound represented by Formula (Id1) or Formula (Id2)

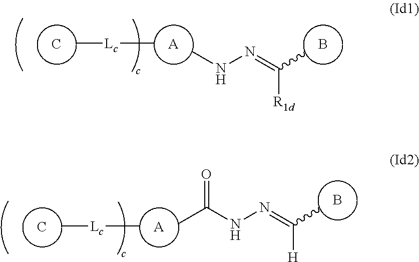

wherein

A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

c is 0 or 1;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NRe—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl; and

R_{1d} is hydrogen or C₁-C₃ alkyl.

In some embodiments, the compound is represented by Formula (Id1) or Formula (Id2), c is 1, L_{c} is a covalent bond and C is an optionally substituted monocyclic ring selected from the group consisting of benzene and pyridine.

In some embodiments, the compound is represented by Formula (Id1) or Formula (Id2), c is 1, L_{c} is a covalent bond and C is an optionally substituted monocyclic ring selected from the group consisting of:

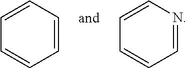 [SMILES: c1ccccc1 ; "and" ; c1ccncc1]

In some embodiments, the compound is represented by Formula (Id1) or Formula (Id2) and B is an optionally substituted monocyclic ring selected from the group consisting of benzene and furan.

In some embodiments, the compound is represented by Formula (Id1) or Formula (Id2) and B is an optionally substituted benzene.

In some embodiments, the compound is represented by Formula (Id1) or Formula (Id2) and B is an optionally substituted monocyclic ring selected from the group consisting of:

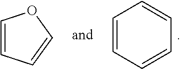 [SMILES: c1ccoc1 ; c1ccccc1]

In some embodiments, the compound is represented by Formula (Id1) or Formula (Id2) and B is an optionally substituted

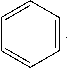

In some embodiments, the compound is represented by Formula (Id1) or Formula (Id2) and B is an optionally substituted benzofuran.

In some embodiments, the compound is represented by Formula (Id1) or Formula (Id2) and B is an optionally substituted

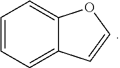

In some embodiments, the compound is represented by Formula (Id1) or Formula (Id2) and A is an optionally substituted monocyclic ring selected from the group consisting of pyrimidine, benzene, thiazole, pyridine and furan.

In some embodiments, the compound is represented by Formula (Id1) or Formula (Id2) and A is an optionally substituted monocyclic ring selected from the group consisting of pyrimidine, benzene, and thiazole.

In some embodiments, the compound is represented by Formula (Id1) or Formula (Id2) and A is an optionally substituted monocyclic ring selected from the group consisting of:

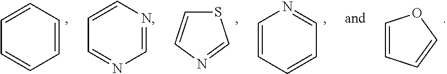

In some embodiments, the compound is represented by Formula (Id1) or Formula (Id2) and A is an optionally substituted monocyclic ring selected from the group consisting of:

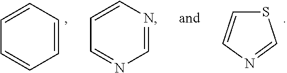

In some embodiments, the compound is represented by Formula (Id1) or Formula (Id2) and A is an optionally substituted 1H-benzo[d]imidazole.

In some embodiments, the compound is represented by Formula (Id1) or Formula (Id2) and A is an optionally substituted

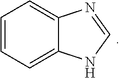

In some embodiments, the compound is represented by Formula (Id1) or Formula (Id2) and A is an optionally substituted 4,5-dihydro-H-benzo[g]indazole.

In some embodiments, the compound is represented by Formula (Id1) or Formula (Id2) and A is an optionally substituted

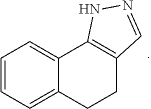 [SMILES: c1ccc2c(c1)CCc1cn[nH]c1-2]

In some embodiments, the compound is of Formula (Id1) or Formula (Id2) and A is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Id1) or Formula (Id2) and A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Id1) or Formula (Id2) and B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Id1) or Formula (Id2) and B is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is of Formula (Id1) or Formula (Id2) and C is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Id1) or Formula (Id2) and C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound represented by Formula (Id1) or Formula (Id2) is a compound or salt thereof of Table 4 below.

In some embodiments, the compound is a compound or a salt thereof of Table 4 below:

TABLE 4
AHR antagonists

| Compd. | |
|---|---|
| No. | Structure |
| 112 | 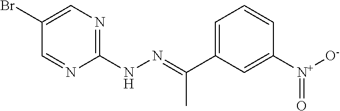 [SMILES: C/C(=N\Nc1ncc(Br)cn1)c1cccc([N+](=O)[O-])c1] |
| 98 | 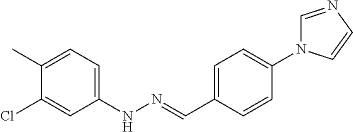 [SMILES: Cc1ccc(N/N=C/c2ccc(-n3ccnc3)cc2)cc1Cl] |
| 113 | 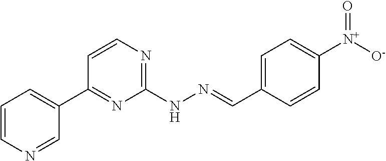 [SMILES: O=[N+]([O-])c1ccc(/C=N/Nc2nccc(-c3cccnc3)n2)cc1] |
| 114 | 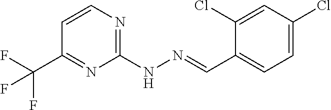 [SMILES: FC(F)(F)c1ccnc(N/N=C/c2ccc(Cl)cc2Cl)n1] |
| 99 | 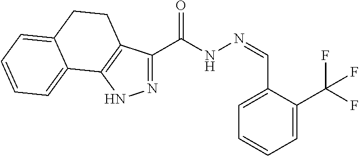 [SMILES: O=C(N/N=C\c1ccccc1C(F)(F)F)c1n[nH]c2c1CCc1ccccc1-2] |
| 115 | 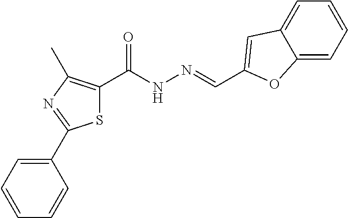 [SMILES: Cc1nc(-c2ccccc2)sc1C(=O)N/N=C/c1cc2ccccc2o1] |

In some embodiments, the compound represented by Formula (Id1) or Formula (Id2) is a compound or salt thereof of Table 4A below.

In some embodiments, the compound is a compound or a salt thereof of Table 4A below:

TABLE 4A
AHR antagonists

| Compd. | |
|---|---|
| No. | Structure |
| 83A | 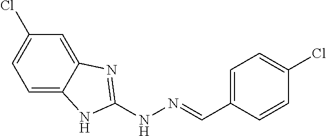 [SMILES: Clc1ccc(/C=N/Nc2nc3cc(Cl)ccc3[nH]2)cc1] |

In some embodiments, the compound represented by Formula (Id1) or Formula (Id2) is a compound or salt thereof of Table 4B below.

In some embodiments, the compound is a compound or a salt thereof of Table 4B below:

TABLE 4B
AHR antagonists

| Compd. | |
|---|---|
| No. | Structure |
| 43B | 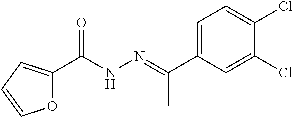 [SMILES: C/C(=N\NC(=O)c1ccco1)c1ccc(Cl)c(Cl)c1] |
| 44B | 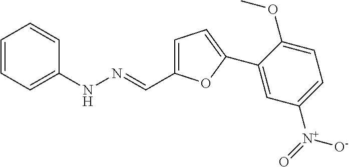 [SMILES: COc1ccc([N+](=O)[O-])cc1-c1ccc(/C=N/Nc2ccccc2)o1] |
| 45B | 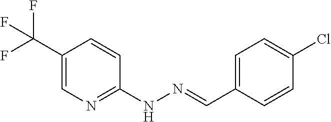 [SMILES: FC(F)(F)c1ccc(N/N=C/c2ccc(Cl)cc2)nc1] |

### Compounds of Formula (Ie1) and Formula (Ie2)

In some embodiments, the disclosure features a compound represented by Formula (Ie1) or Formula (Ie2)

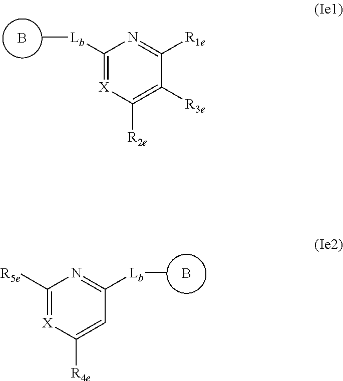

wherein

X is N or CR₆ₑ in which R₆ₑ is hydrogen, halogen, or —CN;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{b}C(O)—R_{ba}—NR_{bb}—R_{ba}—**, in which * denotes the linkage between L_{b} and a pyridine or pyrimidine carbon and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} independently is H, —C(O)R_{bba}, or a 6- to 10-membered aryl optionally substituted with one or more halogen, —CF₃, —CN, —OR_{ba}, or —NR_{bba}R_{bba}, in which each R_{bba} is independently H or C₁-C₆ alkyl;

R₁ₑ is hydrogen, —CF₃, or -L_{c}-C;

R₂ₑ is hydrogen, —CF₃, L_{c}-C, or 6-membered aryl optionally substituted with one or more halogen, —CF₃, or —CN;

R₃ₑ is hydrogen or when R₁ₑ is hydrogen and R₂ₑ is hydrogen R₃ₑ is L_{c}-C;

R_{4c} is hydrogen or L_{c}-C;

R₅ₑ is hydrogen or L_{c}-C;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O **, *—C(O)NR_{cb}NR_{cb}C(O)—**, in which * denotes the linkage between L and a pyridine or pyrimidine carbon and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (Ie1) wherein X is N.

In some embodiments, the compound is represented by Formula (Ie1) or Formula (Ie2) and B is an optionally substituted monocyclic ring selected from the group consisting of pyrazole, benzene, and pyridine.

In some embodiments, the compound is represented by Formula (Ie1) or Formula (Ie2) and B is an optionally substituted monocyclic ring selected from the group consisting of:

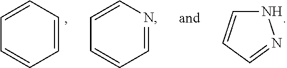

In some embodiments, the compound is represented by Formula (Ie1) or Formula (Ie2) and B is an optionally substituted indole.

In some embodiments, the compound is represented by Formula (Ie1) or Formula (Ie2) and B is an optionally substituted

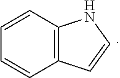

In some embodiments, the compound is represented by Formula (Ie1) or Formula (Ie2) and C is an optionally substituted monocyclic ring selected from the group consisting of benzene and pyridine.

In some embodiments, the compound is represented by Formula (Ie1) or Formula (Ie2) and C is an optionally substituted monocyclic ring selected from the group consisting of:

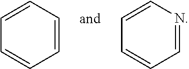 [SMILES: c1ccccc1 ; c1ccncc1]

In some embodiments, the compound is represented by Formula (Ie1) or Formula (Ie2) and L_{b} is selected from the group consisting of a covalent bond, *—NH—**, and *—NHCH₂CH(OH)—**.

In some embodiments, the compound is represented by Formula (Ie1) or Formula (Ie2) and L_{b} is a covalent bond.

In some embodiments, the compound is represented by Formula (Ie1) or Formula (Ie2) wherein at least one of R₁ₑ, R₂ₑ, R₃ₑ, R₄ₑ and R₅ₑ is L_{c}-C and L_{c} is selected from the group consisting of a covalent bond, *—NH—**, and *—SCH₂—**.

In some embodiments, the compound is represented by Formula (Ie1) or Formula (e2) wherein at least one of R₁ₑ, R₂ₑ, R₃ₑ, R₄ₑ and R₅ₑ is L_{c}-C and L_{c} is a covalent bond.

In some embodiments, the compound is of Formula (Ie1) or Formula (Ie2) and A is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ie1) or Formula (Ie2) and A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —C(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ie1) or Formula (Ie2) and B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ie1) or Formula (Ie2) and B is optionally substituted with one or more of: —CF₃, —CF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is of Formula (Ie1) or Formula (Ie2) and C is optionally substituted with one or more of: —CF₃, —CF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (Ie1) or Formula (Ie2) and C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound represented by Formula (Ie1) or Formula (Ie2) is a compound or salt thereof of Table 5 below.

In some embodiments, the compound is a compound or a salt thereof of Table 5 below:

TABLE 5
AHR antagonists

| Compd. | |
|---|---|
| No. | Structure |
| 82 | 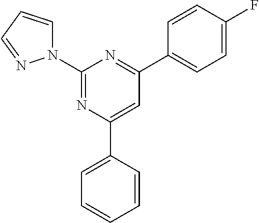 [SMILES: Fc1ccc(-c2cc(-c3ccccc3)nc(-n3cccn3)n2)cc1] |
| 83 | 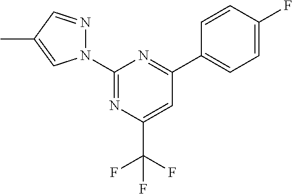 [SMILES: Cc1cnn(-c2nc(-c3ccc(F)cc3)cc(C(F)(F)F)n2)c1] |
| 84 | 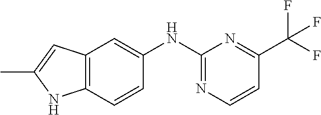 [SMILES: Cc1cc2cc(Nc3nccc(C(F)(F)F)n3)ccc2[nH]1] |
| 85 | 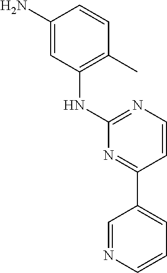 [SMILES: Cc1ccc(N)cc1Nc1nccc(-c2cccnc2)n1] |
| 86 | 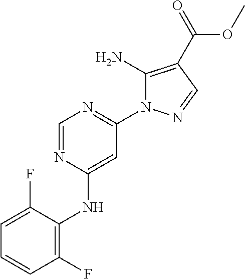 [SMILES: COC(=O)c1cnn(-c2cc(Nc3c(F)cccc3F)ncn2)c1N] |
| 87 | 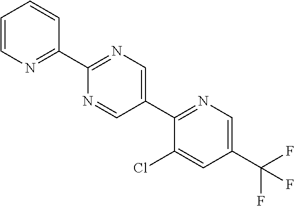 [SMILES: FC(F)(F)c1cnc(-c2cnc(-c3ccccn3)nc2)c(Cl)c1] |
| 88 | 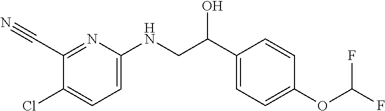 [SMILES: N#Cc1nc(NCC(O)c2ccc(OC(F)F)cc2)ccc1Cl] |
| 89 | 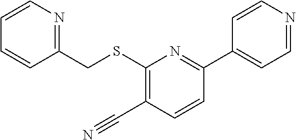 [SMILES: N#Cc1ccc(-c2ccncc2)nc1SCc1ccccn1] |

In some embodiments, the compound represented by Formula (Ie1) or Formula (Ie2) is a compound or salt thereof of Table 5A below.

In some embodiments, the compound is a compound or a salt thereof of Table 5A below:

TABLE 5A
AHR antagonists

| Compd. | |
|---|---|
| No. | Structure |
| 84A | 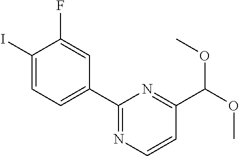 [SMILES: COC(OC)c1ccnc(-c2ccc(I)c(F)c2)n1] |
| 85A | 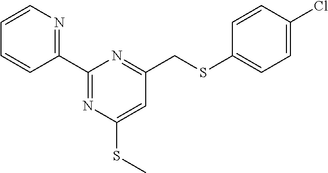 [SMILES: CSc1cc(CSc2ccc(Cl)cc2)nc(-c2ccccn2)n1] |
| 86A | 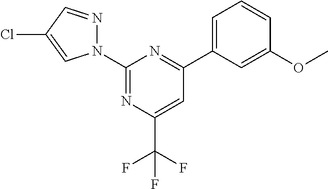 [SMILES: COc1cccc(-c2cc(C(F)(F)F)nc(-n3cc(Cl)cn3)n2)c1] |
| 87A | 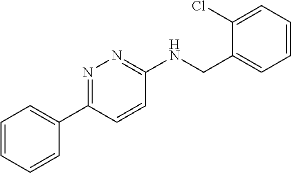 [SMILES: Clc1ccccc1CNc1ccc(-c2ccccc2)nn1] |
| 88A | 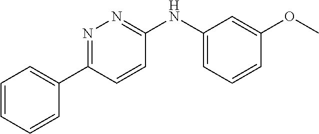 [SMILES: COc1cccc(Nc2ccc(-c3ccccc3)nn2)c1] |
| 89A | 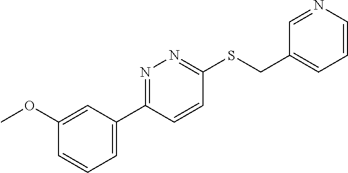 [SMILES: COc1cccc(-c2ccc(SCc3cccnc3)nn2)c1] |
| 90A | 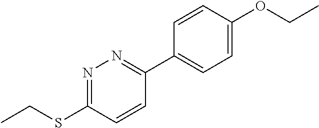 [SMILES: CCOc1ccc(-c2ccc(SCC)nn2)cc1] |
| 91A | 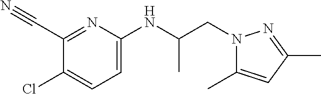 [SMILES: Cc1cc(C)n(CC(C)Nc2ccc(Cl)c(C#N)n2)n1] |
| 92A | 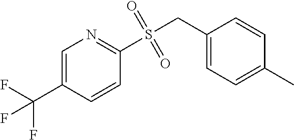 [SMILES: Cc1ccc(CS(=O)(=O)c2ccc(C(F)(F)F)cn2)cc1] |
| 93A | 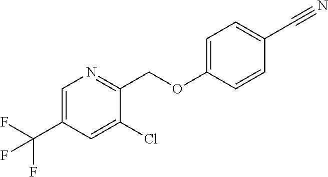 [SMILES: N#Cc1ccc(OCc2ncc(C(F)(F)F)cc2Cl)cc1] |
| 94A | 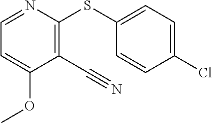 [SMILES: COc1ccnc(Sc2ccc(Cl)cc2)c1C#N] |
| 95A | 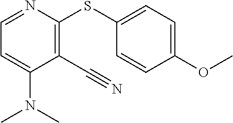 [SMILES: COc1ccc(Sc2nccc(N(C)C)c2C#N)cc1] |

In some embodiments, the compound represented by Formula (Ie1) or Formula (Ie2) is a compound or salt thereof of Table 5B below.

In some embodiments, the compound is a compound or a salt thereof of Table 5B below:

TABLE 5B
AHR antagonists

| Compd | |
|---|---|
| No. | Structure |
| 46B | 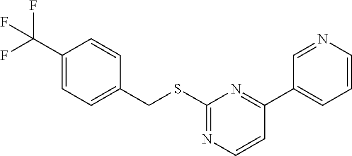 [SMILES: FC(F)(F)c1ccc(CSc2nccc(-c3cccnc3)n2)cc1] |
| 47B | 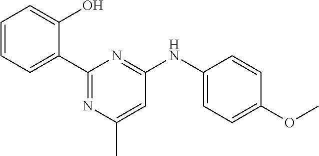 [SMILES: COc1ccc(Nc2cc(C)nc(-c3ccccc3O)n2)cc1] |
| 48B | 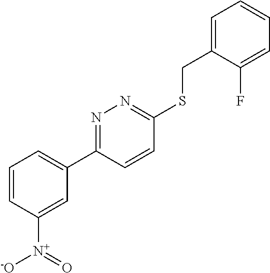 |
| 49B | 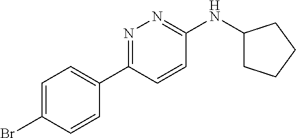 [SMILES: Brc1ccc(-c2ccc(NC3CCCC3)nn2)cc1] |
| 50B | 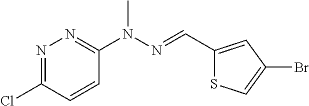 [SMILES: CN(/N=C/c1cc(Br)cs1)c1ccc(Cl)nn1] |
| 51B | 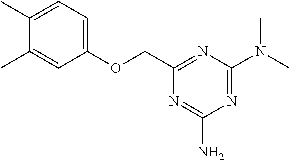 |

### Compounds of Formula (If)

In some embodiments, the disclosure features a compound represented by Formula (If)

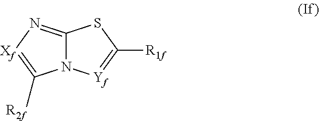

wherein

X_{f} is N or CR_{3f} in which R_{3f} is hydrogen. C₁-C₆ alkyl, or -L_{b}-B;

Y_{f} is N or CR_{4f} in which R_{4f} is hydrogen or C₁-C₆ alkyl;

B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;

L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—*, *—Ra—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a imidazo[2,1-b]thiazole or imidazo[2,1-b][1,3,4]thiadiazole carbon and ** denotes the linkage between L_{b} and B;

each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;

each R_{bb} is independently H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;

R_{1f} is CF₃, C₁-C₆ alkyl, -L_{b}-B, or C(O)NHR_{5f} in which R_{5f} is C₁-C₃ alkyl;

R_{2f} is hydrogen or -L_{b}-B when X_{f} is CR_{3f};

R_{2f} is hydrogen or -L_{c}-C when X_{f} is N;

C is an optionally substituted monocyclic or bicyclic ring selected from 6- to 10-membered aryl and 5- to 10-membered saturated or unsaturated heterocyclyl comprising 1-5 heteroatoms selected from N, O and S;

L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a [1,2,4]triazolo[3,4-b][1,3,4]thiadiazole carbon and ** denotes the linkage between L_{c} and C;

each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and

each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

In some embodiments, the compound is represented by Formula (If) and B is an optionally substituted monocyclic ring selected from the group consisting of benzene, pyridine, thiazole and pyrazole.

In some embodiments, the compound is represented by Formula (If) and B is an optionally substituted monocyclic ring selected from the group consisting of benzene, pyridine, and pyrazole.

In some embodiments, the compound is represented by Formula (If) and B is an optionally substituted monocyclic ring selected from the group consisting of:

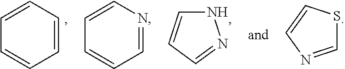

In some embodiments, the compound is represented by Formula (If) and B is an optionally substituted monocyclic ring selected from the group consisting of:

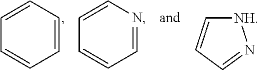

In some embodiments, the compound is represented by Formula (If) and C is an optionally substituted monocyclic ring selected from the group consisting of pyrazole and thiophene.

In some embodiments, the compound is represented by Formula (If) and C is an optionally monocyclic ring selected from the group consisting of:

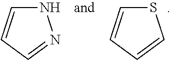 [SMILES: c1cn[nH]c1 ; c1ccsc1]

In some embodiments, the compound is represented by Formula (If) and B is an optionally substituted bicyclic ring selected from the group consisting of 4,5,6,7-tetrahydrobenz[b]thiophene and 2-azabicyclo[2.2.1]heptane.

In some embodiments, the compound is represented by Formula (If) and B is an optionally substituted bicyclic ring selected from the group consisting of:

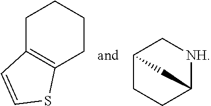 [SMILES: c1cc2c(s1)CCCC2 ; C1C[C@@H]2C[C@@H]1CN2]

In some embodiments, the compound is represented by Formula (If) wherein Y_{f} is N and X_{f} is CR_{3F}.

In some embodiments, the compound is represented by Formula (If) wherein Y_{f} is N, X_{f} is —CCH₃ and R₁ᵣ is -L_{b}-B.

In some embodiments, the compound is represented by Formula (If) wherein L_{b} is a covalent bond.

In some embodiments, the compound is represented by Formula (If) wherein Y_{f} is N, X_{f} is —CCH₃ and Rif is -L_{b}-B in which L_{b} is *—NHCH₂CH₂O—**.

In some embodiments, the compound is represented by Formula (If) wherein X_{f} is N and Y_{f} is N.

In some embodiments, the compound is represented by Formula (If) and L_{c} is a covalent bond.

In some embodiments, the compound is represented by Formula (If) wherein X_{f} is N, Y_{f} is N, and L is a covalent bond.

In some embodiments, the compound is of Formula (If) and A is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (If) and A is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (If) and B is optionally substituted with one or more of: —CF₃, —CF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (If) and B is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound is of Formula (If) and C is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

In some embodiments, the compound is of Formula (If) and C is optionally substituted with one or more of: —CF₃, —OCF₃, —CN, —NO₂, —N(R)₂, —OR, —SR, —C(O)N(R)₂, —S(O)₂N(R)₂, halo, oxo, C₃-C₆ cycloalkyl, —S(CH₂)oF, —S(O)₂R, —C(O)R, —C(O)OR, —N(R)C(O)R, —OC(O)N(R)₂, —(CH₂)ₙN(R)C(O)R, phenyl optionally substituted with halogen, and C₁-C₆ alkyl optionally substituted with halogen or —OR in which each R is independently selected from the group consisting of H, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S, and C₁-C₆ alkyl optionally substituted with halogen; and each n is independently an integer from 1 to 4.

In some embodiments, the compound represented by Formula (If) is a compound or salt thereof of Table 6 below.

In some embodiments, the compound is a compound or a salt thereof of Table 6 below:

TABLE 6
AHR antagonists

| Compd. | |
|---|---|
| No. | Structure |
| 66 | 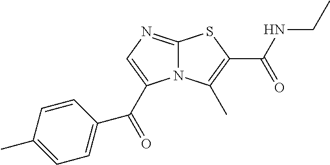 [SMILES: CCNC(=O)c1sc2ncc(C(=O)c3ccc(C)cc3)n2c1C] |
| 67 | 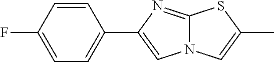 [SMILES: Cc1cn2cc(-c3ccc(F)cc3)nc2s1] |
| 68 | 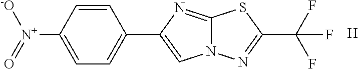 |
| 69 | 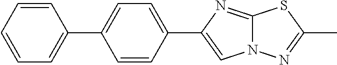 [SMILES: Cc1nn2cc(-c3ccc(-c4ccccc4)cc3)nc2s1] |
| 70 | 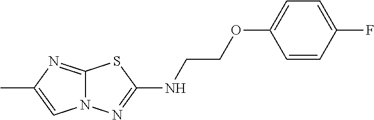 [SMILES: Cc1cn2nc(NCCOc3ccc(F)cc3)sc2n1] |
| 71 | 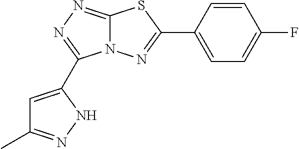 [SMILES: Cc1cc(-c2nnc3sc(-c4ccc(F)cc4)nn23)[nH]n1] |
| 72 | 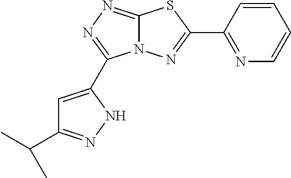 [SMILES: CC(C)c1cc(-c2nnc3sc(-c4ccccn4)nn23)[nH]n1] |
| 73 | 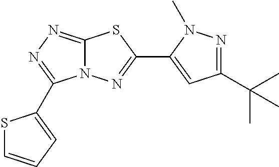 [SMILES: Cn1nc(C(C)(C)C)cc1-c1nn2c(-c3cccs3)nnc2s1] |

In some embodiments, the compound represented by Formula (If) is a compound or salt thereof of Table 6A below.

In some embodiments, the compound is a compound or a salt thereof of Table 6A below:

TABLE 6A
AHR antagonists

| Compd. | |
|---|---|
| No. | Structure |
| 96A | 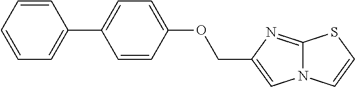 [SMILES: c1ccc(-c2ccc(OCc3cn4ccsc4n3)cc2)cc1] |
| 97A | 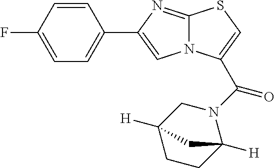 [SMILES: O=C(c1csc2nc(-c3ccc(F)cc3)cn12)N1C[C@@H]2CC[C@@H]1C2] |
| 97A1 | 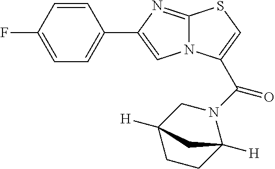 [SMILES: O=C(c1csc2nc(-c3ccc(F)cc3)cn12)N1C[C@H]2CC[C@@H]1C2] |
| 98A | 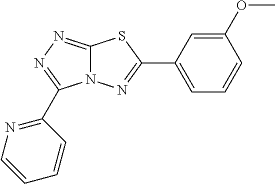 [SMILES: COc1cccc(-c2nn3c(-c4ccccn4)nnc3s2)c1] |
| 99A | 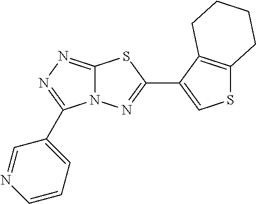 [SMILES: c1cncc(-c2nnc3sc(-c4csc5c4CCCC5)nn23)c1] |
| 97A2 | 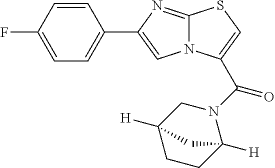 [SMILES: O=C(c1csc2nc(-c3ccc(F)cc3)cn12)N1C[C@@H]2CC[C@H]1C2] |

In some embodiments, the compound represented by Formula (If) is a compound or salt thereof of Table 6A below.

In some embodiments, the compound is a compound or a salt thereof of Table 6A below:

TABLE 6B
AHR antagonists

| Compd. | |
|---|---|
| No. | Structure |
| 40B | 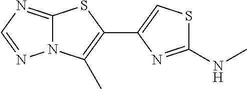 [SMILES: CNc1nc(-c2sc3ncnn3c2C)cs1] |

In some embodiments, the compound is represented by at least one formula selected from the group consisting of Ia, Ib, Ic, Id1, Id2, Ie1, Ie2, and If.

### Stem Cells

In some embodiments, the stem cells of which the population is modified (e.g., expanded) with the compositions and methods described are capable of being expanded upon contacting the aryl hydrocarbon receptor antagonist. In some embodiments, the stem cells are not genetically modified stem cells. In some embodiments, the stem cells are genetically modified stem cells.

In some embodiments, the stem cells are embryonic stem cells or adult stem cells. In some embodiments, the stem cells are totipotent stem cells, pluripotent stem cells, multipotent stem cells, oligopotent stem cells, or unipotent stem cells. In some embodiments, the stem cells are tissue-specific stem cells.

In some embodiments, the stem cells are hematopoietic stem cells, intestinal stem cells, osteoblastic stem cells, mesenchymal stem cells (i.e., lung mesenchymal stem cells, bone marrow-derived mesenchymal stromal cells, or bone marrow stromal cells), neural stem cells (i.e., neuronal dopaminergic stem cells or motor-neuronal stem cells), epithelial stem cells (i.e., lung epithelial stem cells, breast epithelial stem cells, vascular epithelial stem cells, or intestinal epithelial stem cells), cardiac myocyte progenitor stem cells, skin stem cells (i.e., epidermal stem cells or follicular stem cells (hair follicle stem cells)), skeletal muscle stem cells, adipose stem cells, liver stem cells, induced pluripotent stem cells, umbilical cord stem cells, amniotic fluid stem cells, limbal stem cells, dental pulp stem cells, placental stem cells, myoblasts, endothelial progenitor cells, exfoliated teeth derived stem cells, or hair follicle stem cells.

In some embodiments, the stem cells are hematopoietic stem cells.

In some embodiments, the stem cells are primary stem cells. For example, the stem cells are obtained from bone marrow, adipose tissue, or blood. In some embodiments, the the stem cells are cultured stem cells.

In some embodiments, the stem cells are CD34+ cells. In some embodiments, the stem cells are CD90+ cells. In some embodiments, the stem cells are CD45RA− cells. In some embodiments, the stem cells are CD34+CD90+ cells. In some embodiments, the stem cells are CD34+CD45RA− cells. In some embodiments, the stem cells are CD90+CD45RA− cells. In some embodiments, the stem cells are CD34+CD90+CD45RA− cells.

In some embodiments, the hematopoietic stem cells are extracted from the bone marrow, mobilized into the peripheral blood and then collected by apheresis, or isolated from umbilical cord blood units.

In some embodiments, the hematopoietic stem cells are CD34+ hematopoietic stem cells. In some embodiments, the hematopoietic stem cells are CD90+ hematopoietic stem cells. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells. In some embodiments, the hematopoietic stem cells are CD90+CD45RA− hematopoietic stem cells. In some embodiments, the hematopoietic stem cells are CD34+CD90+CD45RA− hematopoietic stem cells.

### Methods for Expanding Hematopoietic Stem Cells

In another aspect, the disclosure features a method of producing an expanded population of hematopoietic stem cells ex vivo, the method including contacting a population of hematopoietic stem cells with the compound of any one of the above aspects or embodiments in an amount sufficient to produce an expanded population of hematopoietic stem cells.

In another aspect, the disclosure features a method of enriching a population of cells with hematopoietic stem cells ex vivo, the method including contacting a population of hematopoietic stem cells with the compound of any one of the above aspects or embodiments in an amount sufficient to produce a population of cells enriched with hematopoietic stem cells.

In another aspect, the disclosure features a method of maintaining the hematopoietic stem cell functional potential of a population of hematopoietic stem cells ex vivo for two or more days, the method including contacting a first population of hematopoietic stem cells with the compound of any one of the above aspects or embodiments, wherein the first population of hematopoietic stem cells exhibits a hematopoietic stem cell functional potential after two or more days that is greater than that of a control population of hematopoietic stem cells cultured under the same conditions and for the same time as the first population of hematopoietic stem cells but not contacted with the compound.

In one embodiment, said method for expanding hematopoietic stem cells, comprises (a) providing a starting cell population comprising hematopoietic stem cells and (b) culturing said starting cell population ex vivo in the presence of an AHR antagonist agent compound of any one of the above aspects or embodiments.

The starting cell population comprising hematopoietic stem cells will be selected by the person skilled in the art depending on the envisaged use. Various sources of cells comprising hematopoietic stem cells have been described in the art, including bone marrow, peripheral blood, neonatal umbilical cord blood, placenta or other sources such as liver, particularly fetal liver.

The cell population may first be subjected to enrichment or purification steps, including negative and/or positive selection of cells based on specific cellular markers in order to provide the starting cell population. Methods for isolating said starting cell population based on specific cellular markers may use fluorescent activated cell sorting (FACS) technology also called flow cytometry or solid or insoluble substrate to which is bound antibodies or ligands that interact with specific cell surface markers. For example, cells may be contacted with a solid substrate (e.g., column of beads, flasks, magnetic particles) containing the antibodies and any unbound cells are removed. When a solid substrate comprising magnetic or paramagnetic beads is used, cells bound to the beads can be readily isolated by a magnetic separator.

In one embodiment, said starting cell population is enriched in a desirable cell marker phenotype (e.g., CD34+, CD133+, CD90+) or based on efflux of dyes such as rhodamine, Hoechst or aldehyde dehydrogenase activity. In one specific embodiment, said starting cell population is enriched in CD34+ cells. Methods for enriching blood cell population in CD34+ cells include kits commercialized by Miltenyi Biotec (CD34+ direct isolation kit, Miltenyi Biotec, Bergisch, Gladbach, Germany) or by Baxter (Isolex 3000).

In some embodiments, the hematopoietic stem cells are CD34+ hematopoietic stem cells. In some embodiments, the hematopoietic stem cells are CD90+ hematopoietic stem cells. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells.

In some embodiments, the hematopoietic stem cells are CD90+CD45RA− hematopoietic stem cells. In some embodiments, the hematopoietic stem cells are CD34+CD90+CD45RA− hematopoietic stem cells.

In some embodiments, the hematopoietic stem cells are mammalian cells, such as human cells. In some embodiments, the human cells are CD34+ cells, such as CD34+ cells are CD34+, CD34+CD38−, CD34+CD38−CD90+, CD34+CD38−CD90+CD45RA−, CD34+CD38−CD90+CD45RA−CD49F+, or CD34+CD90+CD45RA− cells.

In some embodiments, the hematopoietic stem cells are obtained from human cord blood, mobilized human peripheral blood, or human bone marrow. The hematopoietic stem cells may, for example, be freshly isolated from the human or may have been previously cryopreserved.

The amount of cord blood from a single birth is often inadequate to treat an adult or an older child. One advantage of the expansion methods using the compounds of the invention, or an agent capable of down-regulating the activity and/or expression of aryl hydrocarbon receptor and/or a down-stream effector of aryl hydrocarbon receptor pathway, is that it enables the production of a sufficient amount of hematopoietic stem cells from only one cord blood unit.

Accordingly, in one embodiment, the starting cell population is derived from neonatal umbilical cord blood cells which have been enriched in CD34+ cells. In one related embodiment, said starting cell population is derived from one or two umbilical cord blood units.

In another embodiment, the starting cell population is derived from human mobilized peripheral blood cells which have been enriched in CD34+ cells. In one related embodiment, said starting cell population is derived from human mobilized peripheral blood cells isolated from only one patient.

Said starting cell population enriched in CD34+ cells may preferably contain at least about 50% CD34+ cells, in some embodiments, more than about 90% CD34+ cells, and may comprise between 10⁵ and 10⁹ nucleated cells.

The starting cell population may be used directly for expansion or frozen and stored for use at a later date.

Conditions for culturing the starting cell population for hematopoietic stem cell expansion will vary depending, inter alia, on the starting cell population, the desired final number of cells, and desired final proportion of HSCs.

In one embodiment, the culturing conditions comprises the use of other cytokines and growth factors, generally known in the art for hematopoietic stem cell expansion. Such cytokines and growth factors include without limitation IL-1, IL-3, IL-6, IL-11, G-CSF, GM-CSF, SCF, FIT3-L, thrombopoietin (TPO), erythropoeitin, and analogs thereof. As used herein, “analogs” include any structural variants of the cytokines and growth factors having the biological activity as the naturally occurring forms, including without limitation, variants with enhanced or decreased biological activity when compared to the naturally occurring forms or cytokine receptor agonists such as an agonist antibody against the TPO receptor (for example, VB22B sc(Fv)2 as detailed in patent publication WO 2007/145227, and the like). Cytokine and growth factor combinations are chosen to expand HSC and progenitor cells while limiting the production of terminally differentiated cells. In one specific embodiment, one or more cytokines and growth factors are selected from the group consisting of SCF, Flt3-L and TPO. In one specific embodiment, at least TPO is used in a serum-free medium under suitable conditions for HSC expansion. In one related embodiment, a mixture of IL6, SCF, Flt3-L and TPO is used in the method for expanding HSCs in combination with the compound of the present disclosure.

The expansion of HSCs may be carried out in a basal medium, which may be supplemented with mixtures of cytokines and growth factors. A basal medium typically comprises amino acids, carbon sources, vitamins, serum proteins (e.g. albumin), inorganic salts, divalent cations, buffers and any other element suitable for use in expansion of HSC. Examples of such basal medium appropriate for a method of expanding HSC include, without limitation, StemSpan® SFEM-Serum-Free Expansion Medium (StemCell Technologies, Vancouver, Canada), StemSpan® H3000-Defined Medium (StemCell Technologies, Vancouver, Canada), CellGro® SCGM (CellGenix, Freiburg Germany), StemPro®-34 SFM (Invitrogen).

In one aspect, the present disclosure further relates to a composition comprising any one of the compounds and/or AHR modulating agents of the present disclosure and a cell culture medium.

In certain embodiments, the cell culture medium is any such medium as described above.

In certain embodiments, the composition comprises any one of the compounds and/or AHR modulating agents of the present disclosure and a basal cell culture medium.

In certain embodiments, the composition comprises any one of the compounds and/or AHR modulating agents of the present disclosure and a serum free cell culture medium.

In certain embodiments, the composition comprises any one of the compounds and/or AHR modulating agents of the present disclosure and a cell culture medium comprising one or more cytokines or growth factors selected from the group consisting of IL-1, IL-3, IL-6, IL-11, G-CSF, GM-CSF, SCF, Flt3-L, thrombopoietin (TPO), erythropoietin, and analogs thereof.

In certain embodiments, the composition comprises any one of the compounds and/or AHR modulating agents of the present disclosure and a basal serum-free cell culture medium further comprising thrombopoietin (TPO), IL-6, SCF, and Flt3-L.

In one embodiment, the compound of the present disclosure is administered during the expansion method of said starting cell population under a concentration appropriate for HSC expansion. In one specific embodiment, said compound or AHR modulating agent is administered at a concentration comprised between 1 pM and 100 μM, for example between 10 pM and 10 μM, or between 100 pM and 1 μM.

In one embodiment where starting cell population essentially consists of CD34+ enriched cells from one or two cord blood units, the cells are grown under conditions for HSC expansion from about 3 days to about 90 days, for example between 7 and 2 days and/or until the indicated fold expansion and the characteristic cell populations are obtained. In one specific embodiment, the cells are grown under conditions for HSC expansion not more than 21 days, 14 days or 7 days.

In one embodiment, the starting cell population is cultured during a time sufficient to reach an absolute number of CD34+ cells of at least 10⁵, 10⁶, 10⁷, 10⁸ or 10⁹ cells. In another embodiment, said starting cell population is cultured during a time sufficient for a 10 to 50000 fold expansion of CD34+ cells, for example between 100 and 10000 fold expansion, for examples between 50 and 1000 fold expansion.

In some embodiments, the expanding amount, referring to a quantity or concentration of an agent, such as an aryl hydrocarbon receptor antagonist described herein, sufficient to induce the proliferation of a population of CD34+ cells (e.g., a CD34+CD90+ cells), for example, by from about 1.1-fold to about 1,000-fold, about 1.1-fold to about 5,000-fold, or more (e.g., about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 3.1-fold, 3.2-fold, 3.3-fold, 3.4-fold, 3.5-fold, 3.6-fold, 3.7-fold, 3.8-fold, 3.9-fold, 4-fold, 4.1-fold, 4.2-fold, 4.3-fold, 4.4-fold, 4.5-fold, 4.6-fold, 4.7-fold, 4.8-fold, 4.9-fold, 5-fold, 5.1-fold, 5.2-fold, 5.3-fold, 5.4-fold, 5.5-fold, 5.6-fold, 5.7-fold, 5.8-fold, 5.9-fold, 6-fold, 6.1-fold, 6.2-fold, 6.3-fold, 6.4-fold, 6.5-fold, 6.6-fold, 6.7-fold, 6.8-fold, 6.9-fold, 7-fold, 7.1-fold, 7.2-fold, 7.3-fold, 7.4-fold, 7.5-fold, 7.6-fold, 7.7-fold, 7.8-fold, 7.9-fold, 8-fold, 8.1-fold, 8.2-fold, 8.3-fold, 8.4-fold, 8.5-fold, 8.6-fold, 8.7-fold, 8.8-fold, 8.9-fold, 9-fold, 9.1-fold, 9.2-fold, 9.3-fold, 9.4-fold, 9.5-fold, 9.6-fold, 9.7-fold, 9.8-fold, 9.9-fold, 10-fold, 50-fold, 100-fold, 200-fold, 300-fold, 400-fold, 500-fold, 600-fold, 700-fold, 800-fold, 900-fold, 1,000-fold, or more).

In one embodiment, the expanding amount, referring to a quantity or concentration of an agent, such as an aryl hydrocarbon receptor antagonist described herein, sufficient to induce the proliferation of a population of CD34+ cells (e.g., a CD34+CD90+ cells), for example, by from about 60-fold to about 900-fold, from about 80-fold to about 800-fold, from about 100-fold to about 700-fold, from about 150-fold to about 600-fold, from about 200-fold to about 500-fold, from about 250-fold to about 400-fold, from about 275-fold to about 350-fold, or about 325-fold.

The cell population obtained after the expansion method may be used without further purification or may be subject to further purification or selection steps.

The cell population may then be washed to remove the compound of the present disclosure and/or any other components of the cell culture and resuspended in an appropriate cell suspension medium for short term use or in a long-term storage medium, for example a medium suitable for cryopreservation.

Cell Population with Expanded Hematopoietic Stem Cells as Obtained by the Expansion Method and Therapeutic Compositions

In another aspect, the disclosure features a composition comprising a population of hematopoietic stem cells, wherein the hematopoietic stem cells or progenitors thereof have been contacted with the compound of any one of the above aspects or embodiments, thereby expanding the hematopoietic stem cells or progenitors thereof.

The invention further provides a cell population with expanded hematopoietic stem cells obtainable or obtained by the expansion method described above. In one embodiment, such cell population is resuspended in a pharmaceutically acceptable medium suitable for administration to a mammalian host, thereby providing a therapeutic composition.

The compound as defined in the present disclosure enables the expansion of HSCs, for example from only one or two cord blood units, to provide a cell population quantitatively and qualitatively appropriate for efficient short and long term engraftment in a human patient in need thereof. In one embodiment, the present disclosure relates to a therapeutic composition comprising a cell population with expanded HSCs derived from not more than one or two cord blood units. In one embodiment, the present disclosure relates to a therapeutic composition containing a total amount of cells of at least about 10⁵, at least about 10⁶, at least about 10⁷, at least about 10⁸ or at least about 10⁹ cells with about 20% to about 100%, for example between about 43% to about 80%, of total cells being CD34+ cells. In certain embodiments, said composition contains between 20-100%, for example between 43-80%, of total cells being CD34+CD90+CD45RA−.

In some embodiments, the hematopoietic stem cells are CD34+ hematopoietic stem cells. In some embodiments, the hematopoietic stem cells are CD90+ hematopoietic stem cells. In some embodiments, the hematopoietic stem cells are CD45RA− hematopoietic stem cells. In some embodiments, the hematopoietic stem cells are CD34+CD90+ hematopoietic stem cells. In some embodiments, the hematopoietic stem cells are CD34+CD45RA− hematopoietic stem cells. In some embodiments, the hematopoietic stem cells are CD90+CD45RA− hematopoietic stem cells. In some embodiments, the hematopoietic stem cells are CD34+CD90+CD45RA− hematopoietic stem cells.

In some embodiments, the hematopoietic stem cells of the therapeutic composition are mammalian cells, such as human cells. In some embodiments, the human cells are CD34+ cells, such as CD34+ cells are CD34+, CD34+CD38−, CD34+CD38−CD90+, CD34+CD38−CD90+CD45RA−, CD34+CD38−CD90+CD45RA−CD49F+, or CD34+CD90+CD45RA− cells.

In some embodiments, the hematopoietic stem cells of the therapeutic composition are obtained from human cord blood, mobilized human peripheral blood, or human bone marrow. The hematopoietic stem cells may, for example, be freshly isolated from the human or may have been previously cryopreserved.

### Methods of Genetic Modification of Hematopoietic Stem and Progenitor Cells

The compositions and methods described herein provide strategies for disrupting a gene of interest and for promoting the expression of target genes in populations of hematopoietic stem and progenitor cells, as well as for expanding these cells. For instance, a population of hematopoietic stem cells may be expanded according to the methods described herein and may be genetically modified, e.g., so as to exhibit an altered gene expression pattern. Alternatively, a population of cells may be enriched with hematopoietic stem cells, or a population of hematopoietic stem cells may be maintained in a multi-potent state, and the cells may further be modified using established genome editing techniques known in the art. For instance, one may use a genome editing procedure to promote the expression of an exogenous gene or inhibit the expression of an endogenous gene within a hematopoietic stem cell. Populations of hematopoietic stem cells may be expanded, enriched, or maintained in a multi-potent state according to the methods described herein and subsequently genetically modified so as to express a desired target gene, or populations of these cells may be genetically modified first and then expanded, enriched, or maintained in a multi-potent state. A wide array of methods has been established for the incorporation of target genes into the genome of a cell (e.g., a mammalian cell, such as a murine or human cell) so as to facilitate the expression of such genes.

### Polynucleotides Encoding Target Genes

One example of a platform that can be used to facilitate the expression of a target gene in a hematopoietic stem cell is by the integration of the polynucleotide encoding a target gene into the nuclear genome of the cell. A variety of techniques have been developed for the introduction of exogenous genes into a eukaryotic genome. One such technique involves the insertion of a target gene into a vector, such as a viral vector. Vectors for use with the compositions and methods described herein can be introduced into a cell by a variety of methods, including transformation, transfection, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition, Cold Spring Harbor University Press, New York (2014); and Ausubel, et al., Current Protocols in Molecular Biology, John Wiley & Sons, New York (2015), the disclosures of each of which are incorporated herein by reference.

Exogenous genes can also be introduced into a mammalian cell through the use of a vector containing the gene of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Viral vectors containing the VSV-G protein are described in further detail, e.g., in U.S. Pat. No. 5,512,421; and in U.S. Pat. No. 5,670,354, the disclosures of each of which are incorporated by reference herein.

Recognition and binding of the polynucleotide encoding a target gene by mammalian RNA polymerase is an important molecular event for gene expression to occur. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Alternatively, promoters derived from viral genomes can be used for the stable expression of target genes in mammalian cells. Examples of functional viral promoters that can be used to promote mammalian expression of these enzymes include adenovirus late promoter, vaccinia virus 7.5K promoter, SV40 promoter, cytomegalovirus promoter, mouse mammary tumor virus (MMTV) promoter, LTR promoter of HIV, promoter of moloney virus, Epstein barr virus (EBV) promoter, Rous sarcoma virus (RSV) promoter, and the cytomegalovirus (CMV) promoter. Additional viral promoters include the SV40 late promoter from simian virus 40, the Baculovirus polyhedron enhancer/promoter element, Herpes Simplex Virus thymidine kinase (HSV tk) promoter, and the 35S promoter from Cauliflower Mosaic Virus. Suitable phage promoters for use with the compositions and methods described herein include, but are not limited to, the E. coli T7 and T3 phage promoters, the S. typhimurium phage SP6 promoter, B. subtilis SPO1 phage and B. subtilis phage phi 29 promoters, and N4 phage and K11 phage promoters as described in U.S. Pat. No. 5,547,892, the disclosure of which is incorporated herein by reference.

Upon incorporation of a polynucleotide encoding a target gene has been incorporated into the genome of a cell (e.g., the nuclear genome of a hematopoietic stem cell), the transcription of this polynucleotide can be induced by methods known in the art. For example expression can be induced by exposing the mammalian cell to an external chemical reagent, such as an agent that modulates the binding of a transcription factor and/or RNA polymerase to the mammalian promoter and thus regulate gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use with the compositions and methods described herein include enhancer sequences. Enhancers represent another class of regulatory elements that induce a conformational change in the polynucleotide comprising the gene of interest such that the DNA adopts a three-dimensional orientation that is favorable for binding of transcription factors and RNA polymerase at the transcription initiation site. Thus, polynucleotides for use with the compositions and methods described herein include those that encode a target gene and additionally include a mammalian enhancer sequence. Many enhancer sequences are now known from mammalian genes, and examples include enhancers from the genes that encode mammalian globin, elastase, albumin, α-fetoprotein, and insulin.

Enhancers for use with the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al. Nature 297:17 (1982), the disclosure of which is incorporated herein by reference. An enhancer may be spliced into a vector containing a polynucleotide encoding a target gene, for example, at a position 5′ or 3′ to this gene. In a preferred orientation, the enhancer is positioned at the 5′ side of the promoter, which in turn is located 5′ relative to the polynucleotide encoding the target gene.

In addition to promoting high rates of transcription and translation, stable expression of an exogenous gene in a hematopoietic stem cell can be achieved by integration of the polynucleotide comprising the gene into the nuclear DNA of the cell. A variety of vectors for the delivery and integration of polynucleotides encoding exogenous proteins into the nuclear DNA of a mammalian cell have been developed. Examples of expression vectors are disclosed in, e.g., WO94/11026, the disclosure of which is incorporated herein by reference. Expression vectors for use with the compositions and methods described herein contain a polynucleotide sequence that encodes a target gene, as well as, e.g., additional sequence elements used for the expression of these enzymes and/or the integration of these polynucleotide sequences into the genome of a mammalian cell. Certain vectors that can be used for the expression of target genes include plasmids that contain regulatory sequences, such as promoter and enhancer regions, which direct gene transcription. Other useful vectors for expression of target genes contain polynucleotide sequences that enhance the rate of translation of these genes or improve the stability or nuclear export of the mRNA that results from gene transcription. These sequence elements often encode features within RNA transcripts that enhance the nuclear export, cytosolic half-life, and ribosomal affinity of these molecules, e.g., 5′ and 3′ untranslated regions, an internal ribosomal entry site (IRES), and polyadenylation signal site in order to direct efficient transcription of the gene carried on the expression vector. Exemplary expression vectors may also contain a polynucleotide encoding a marker for selection of cells that contain such a vector. Non-limiting examples of a suitable marker include genes that encode resistance to antibiotics, such as ampicillin, chloramphenicol, kanamycin, or nourseothricin.

### Vectors for the Expression of Target Genes

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and often do not require added proteins or reagents in order to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication. In Fundamental Virology, Third Edition, B. N. Fields, et al., Eds., Lippincott-Raven Publishers, Philadelphia, 1996, the disclosure of which is incorporated herein by reference). Other examples of viral vectors include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described in, e.g., U.S. Pat. No. 5,801,030, the disclosure of which is incorporated herein by reference.

### Additional Transfection Methods

Other techniques that can be used to introduce a polynucleotide, such as DNA or RNA (e.g., mRNA, tRNA, siRNA, miRNA, shRNA, chemically modified RNA) into a mammalian cell are well known in the art. For instance, electroporation can be used to permeabilize mammalian cells by the application of an electrostatic potential. Mammalian cells, such as hematopoietic stem cells, subjected to an external electric field in this manner are subsequently predisposed to the uptake of exogenous nucleic acids. Electroporation of mammalian cells is described in detail, e.g., in Chu et al. Nucleic Acids Research 15:1311 (1987), the disclosure of which is incorporated herein by reference. A similar technique, Nucleofection™, utilizes an applied electric field in order to stimulate the update of exogenous polynucleotides into the nucleus of a eukaryotic cell. Nucleofection™ and protocols useful for performing this technique are described in detail, e.g., in Distler et al. Experimental Dermatology 14:315 (2005), as well as in US 2010/0317114, the disclosures of each of which are incorporated herein by reference.

Additional techniques useful for the transfection of hematopoietic stem cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a hematopoietic stem cell. Squeeze-poration is described in detail, e.g., in Sharei et al. Journal of Visualized Experiments 81:e50980 (2013), the disclosure of which is incorporated herein by reference.

Lipofection represents another technique useful for transfection of hematopoietic stem cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, e.g., by direct fusion of the liposome with the cell membrane or by endocytosis of the complex. Lipofection is described in detail, e.g., in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) and diethylaminoethyl (DEAE)-dextran, the use of which as a transfection agent is described in detail, e.g., in Gulick et al. Current Protocols in Molecular Biology 40:I:9.2:9.2.1 (1997), the disclosure of which is incorporated herein by reference. Magnetic beads are another tool that can be used to transfect hematopoietic stem cells in a mild and efficient manner, as this methodology utilizes an applied magnetic field in order to direct the uptake of nucleic acids. This technology is described in detail, e.g., in US 2010/0227406, the disclosure of which is incorporated herein by reference.

Another useful tool for inducing the uptake of exogenous nucleic acids by hematopoietic stem cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al. Methods in Cell Biology 82:309 (2007), the disclosure of which is incorporated herein by reference.

Microvesicles represent another potential vehicle that can be used to modify the genome of a hematopoietic stem cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al. Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Modulation of Gene Expression Using Gene Editing Techniques

In addition to viral vectors, a variety of additional tools have been developed that can be used for the incorporation of exogenous genes into hematopoietic stem cells. One such method that can be used for incorporating polynucleotides encoding target genes into hematopoietic stem cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Transposon systems include the piggybac transposon (described in detail in, e.g., WO 2010/085699) and the sleeping beauty transposon (described in detail in, e.g., US2005/0112764), the disclosures of each of which are incorporated herein by reference.

Another useful tool for the disruption and integration of target genes into the genome of a hematopoietic stem cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can theoretically design a CRISPR/Cas system to cleave any target DNA molecule of interest. This technique has been exploited in order to edit eukaryotic genomes (Hwang et al. Nature Biotechnology 31:227 (2013), the disclosure of which is incorporated herein by reference) and can be used as an efficient means of site-specifically editing hematopoietic stem cell genomes in order to cleave DNA prior to the incorporation of a gene encoding a target gene. The use of CRISPR/Cas to modulate gene expression has been described in, e.g., U.S. Pat. No. 8,697,359, the disclosure of which is incorporated herein by reference.

The CRISPR/Cas system can be used to create one or more double stranded breaks in a target DNA sequence, which can then be repaired by either the homologous recombination (HR) or non-homologous end joining (NHEJ) DNA repair pathways. The Cas9 enzyme, together with a guide RNA specific to the target DNA (gRNA), can be supplied to a cell to induce one or more double strand breask. The Cas9 enzyme can be supplied as a protein, as a ribonucleoprotein complexed with the guide RNA, or as an RNA or DNA encoding the Cas9 protein that is then used by the cell to synthesize the Cas9 protein. The gRNA may comprise both tracrRNA and crRNA sequences in a chimeric RNA. Alternatively, or in addition, the gRNA may comprise a scaffold region that binds to the Cas9 protein, and a complementary base pairing region, also sometimes called a spacer, that targets the gRNA Cas9 protein complex to a particular DNA sequence. In some cases, the complementary base pairing region can be about 20 nucletodes in length, and is complementary to target DNA sequence immediately adjacent to a protospacer adjacent motif (e.g., a PAM motif). In some cases, the PAM comprises a sequence of NGG, NGA or NAG. The complementary base pairing region of the gRNA hybridizes to the target DNA sequence, and guides the gRNA Cas9 protein complex to the target sequence where the Cas9 endonuclease domains then cut within the target sequence, generating a double strand break that may be 3-4 nucleotides upstream of the PAM. Thus, by altering the complementary base pairing region, almost any DNA sequence can be targeted for the generation of a double stranded break. Methods for selecting an appropriate complementary base pairing region will be known to those skilled in the art. For example, gRNAs can be selected to minimize the number of off-target binding sites of the gRNA in the target DNA sequence. In some cases, modified Cas9 genome editing systems may be used to, for example, increase DNA targeting specificity. An example of a modified Cas9 genome editing system comprises split Cas9 systems such as the Dimeric Cas9-Fokl genome editing system.

The double strand break or breaks generated by CRISPR/Cas9 genome editing system may be repaired by the non homologous end joining pathway (NHEJ), which ligates the ends of the double strand break together. NHEJ may result in deletions in the DNA around or near the site of the double strand break. Alternatively, the double strand break generated by CRISPR/Cas9 genome editing system may be repaired through a homology directed repair, also called homologous recombination (HR) repair pathway. In the HR pathway, the double strand break is repaired by exchanging sequences between two similar or identical DNA molecules. The HR repair pathway can therefore be used to introduce exogenous DNA sequences into the genome. In using the HR pathway for genome editing, a DNA template is supplied to the cell along with the Cas9 and gRNA. In some cases, the template may contain exogenous sequences to be introduced into the genome via genome editing flanked by homology arms that comprise DNA sequences on either side of the site of the Cas9 induced double strand break. These homology arms may be, for example, between about 50 or 1000 nucleotides, or in other cases up to several kilobases in length or longer. The template may be a linear DNA, or a circular DNA such as a plasmid, or may be supplied using a viral vector or other means of delivery. The template DNA may comprise double stranded or single stranded DNA. All manner of delivering the template DNA, the gRNA and the Cas9 protein to the cell to achieve the desired genome editing are envisaged as being within the scope of the invention.

The CRISPR/Cas9 and HR based genome editing systems of the disclosure provide not only methods of introducing exogenous DNA sequences into a genome or DNA sequence of interest, but also a platform for correcting mutations in genes. An altered or corrected version of a mutated sequence, for example a sequence changing one or more point mutations back to the wild type concensus sequence, inserting a deleted sequence, or deleting an inserted sequence, could be supplied to the cell as a template sequence, and that template sequence used by the cell to fix a CRISPR/Cas9 induced double strand break via the HR pathway. For example, in a patient with one or more disease causing mutations, hematopoietic stem and/or progenitor cells such as the hematopoietic stem and/or progenitor cells of the patient, can be removed from the body. The mutation can then corrected by CRISPR/Cas9 and HR mediated genome editing in the genome of one or more of these hematopoietic stem and/or progenitor cells, the corrected hematopoietic stem and/or progenitor cell(s) expanded with the methods of the disclosure, and then the edited cell population infused back into the patient, thereby supplying a source of the wild type version of the gene and curing the patient of the disease caused by the mutation or mutations in that gene. Mutations that can cause genetic diseases include not only point mutations, but also insertions, deletions and inversions. These mutations can be in protein coding sequence and affect the amino acid sequence of the protein, or they may be in non-coding sequences such as untranslated regions, promoters, cis regulatory elements required for gene expression, sequences required for splicing, or sequences required for DNA structure. All mutations are potentially editable by CRISPR/Cas9 mediated genome editing methods of the disclosure. In some cases, the patient may be conditioned to eliminate or reduce the native hematopoietic stem and/or progenitor cells that carry the mutant version of the gene, thus enriching for the exogenously supplied genome edited hematopoietic stem and/or progenitor cells. Both autologous and allogeneic genome edited hematopoietic stem and/or progenitor cells can be used to treat a genetic disease of a patient of the disclosure.

In addition to the CRISPR/Cas9 system, alternative methods for disruption of a target DNA by site-specifically cleaving genomic DNA prior to the incorporation of a gene of interest in a hematopoietic stem and/or progenitor cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (2010); and in Joung et al. Nature Reviews Molecular Cell Biology 14:49 (2013), the disclosure of both of which are incorporated herein by reference. As with the CRISPR/Cas9 genome editing systems, double strand breaks introduced by TALENS or ZFNs can also repaired via the HR pathway, and this pathway can be used to introduce exogenous DNA sequences or repair mutations in the DNA.

Additional genome editing techniques that can be used to disrupt or incorporate polynucleotides encoding target genes into the genome of a hematopoietic stem cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a hematopoietic stem cell. These single-chain nucleases have been described extensively in, e.g., U.S. Pat. Nos. 8,021,867 and 8,445,251, the disclosures of each of which are incorporated herein by reference.

### Methods of Treatment

As described herein, hematopoietic stem cell transplant therapy can be administered to s a subject in need of treatment so as to populate or repopulate one or more blood cell types, such as a blood cell lineage that is deficient or defective in a patient suffering from a stem cell disorder. Hematopoietic stem and progenitor cells exhibit multi-potency, and can thus differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Hematopoietic stem cells are additionally capable of self-renewal, and can thus give rise to daughter cells that have equivalent potential as the mother cell, and also feature the capacity to be reintroduced into a transplant recipient whereupon they home to the hematopoietic stem cell niche and re-establish productive and sustained hematopoiesis. Thus, hematopoietic stem and progenitor cells represent a useful therapeutic modality for the treatment of a wide array of disorders in which a patient has a deficiency or defect in a cell type of the hematopoietic lineage. The deficiency or defect may be caused, for example, by depletion of a population of endogenous cells of the hematopoietic system due to administration of a chemotherapeutic agent (e.g., in the case of a patient suffering from a cancer, such as a hematologic cancer described herein). The deficiency or defect may be caused, for example, by depletion of a population of endogenous hematopoietic cells due to the activity of self-reactive immune cells, such as T lymphocytes or B lymphocytes that cross-react with self antigens (e.g., in the case of a patient suffering from an autoimmune disorder, such as an autoimmune disorder described herein). Additionally or alternatively, the deficiency or defect in cellular activity may be caused by aberrant expression of an enzyme (e.g., in the case of a patient suffering from various metabolic disorders, such as a metabolic disorder described herein).

Thus, hematopoietic stem cells can be administered to a patient defective or deficient in one or more cell types of the hematopoietic lineage in order to re-constitute the defective or deficient population of cells in vivo, thereby treating the pathology associated with the defect or depletion in the endogenous blood cell population. Hematopoietic stem and progenitor cells can be used to treat, e.g., a non-malignant hemoglobinopathy (e.g., a hemoglobinopathy selected from the group consisting of sickle cell anemia, thalassemia, Fanconi anemia, aplastic anemia, and Wiskott-Aldrich syndrome). In these cases, for example, a population of hematopoietic stem cells may be expanded ex vivo by culturing the cells in the presence of an aryl hydrocarbon receptor antagonist described herein. The hematopoietic stem cells thus expanded may then be administered to a patient, where the cells may home to a hematopoietic stem cell niche and re-constitute a population of cells that are damaged or deficient in the patient.

Hematopoietic stem or progenitor cells mobilized to the peripheral blood of a subject may be withdrawn (e.g., harvested or collected) from the subject by any suitable technique. For example, the hematopoietic stem or progenitor cells may be withdrawn by a blood draw. In some embodiments, hematopoietic stem or progenitor cells mobilized to a subject's peripheral blood as contemplated herein may be harvested (i.e., collected) using apheresis. In some embodiments, apheresis may be used to enrich a donor's blood with mobilized hematopoietic stem or progenitor cells.

Additionally or alternatively, hematopoietic stem and progenitor cells can be used to treat an immunodeficiency, such as a congenital immunodeficiency. Additionally or alternatively, the compositions and methods described herein can be used to treat an acquired immunodeficiency (e.g., an acquired immunodeficiency selected from the group consisting of HIV and AIDS). In these cases, for example, a population of hematopoietic stem cells may be expanded ex vivo by culturing the cells in the presence of an aryl hydrocarbon receptor antagonist described herein. The hematopoietic stem cells thus expanded may then be administered to a patient, where the cells may home to a hematopoietic stem cell niche and re-constitute a population of immune cells (e.g., T lymphocytes, B lymphocytes, NK cells, or other immune cells) that are damaged or deficient in the patient.

Hematopoietic stem and progenitor cells can also be used to treat a metabolic disorder (e.g., a metabolic disorder selected from the group consisting of glycogen storage diseases, mucopolysaccharidoses, Gaucher's Disease, Hurlers Disease, sphingolipidoses, Sly Syndrome, alpha-Mannosidosis, X-ALD, Aspartylglucosaminuria, Wolman Disease, late infantile metachromatic leukodystrophy, Niemann Pick Type C disease, Niemann Pick Type B disease, Juvenile Tay Sachs, Infantile Tay Sachs, Juvenile Sandhoff, Infantile Sandhoff, GM1 gangliosidosis, MPSIV (Morquio), Presymptomatic or milder forms of globoid cell leukodystrophy, infantile Krabbe when newborn and asymptomatic, early diagnosis fucosidosis, Fabry, MPSIS, MPSIH/S, MPSII, MPSVI in conjunction with ERT or where alloantibodies attenuate efficacy of ERT, Pompe where alloantibodies attenuate efficacy of ERT, Mucolipidosis II, and metachromatic leukodystrophy). In these cases, for example, a population of hematopoietic stem cells may be expanded ex vivo by culturing the cells in the presence of an aryl hydrocarbon receptor antagonist described herein. The hematopoietic stem cells thus expanded may then be administered to a patient, where the cells may home to a hematopoietic stem cell niche and re-constitute a population of hematopoietic cells that are damaged or deficient in the patient.

Additionally or alternatively, hematopoietic stem or progenitor cells can be used to treat a malignancy or proliferative disorder, such as a hematologic cancer or myeloproliferative disease. In the case of cancer treatment, for example, a population of hematopoietic stem cells may be expanded ex vivo by culturing the cells in the presence of an aryl hydrocarbon receptor antagonist described herein. The hematopoietic stem cells thus expanded may then be administered to a patient, where the cells may home to a hematopoietic stem cell niche and re-constitute a population of cells that are damaged or deficient in the patient, such as a population of hematopoietic cells that is damaged or deficient due to the administration of one or more chemotherapeutic agents to the patient. In some embodiments, hematopoietic stem or progenitor cells may be infused into a patient in order to repopulate a population of cells depleted during cancer cell eradication, such as during systemic chemotherapy. Exemplary hematological cancers that can be treated by way of administration of hematopoietic stem and progenitor cells in accordance with the compositions and methods described herein are acute myeloid leukemia, acute lymphoid leukemia, chronic myeloid leukemia, chronic lymphoid leukemia, multiple myeloma, diffuse large B-cell lymphoma, and non-Hodgkin's lymphoma, as well as other cancerous conditions, including neuroblastoma.

Additional diseases that can be treated by the administration of hematopoietic stem and progenitor cells to a patient include, without limitation, adenosine deaminase deficiency and severe combined immunodeficiency, hyper immunoglobulin M syndrome, Chediak-Higashi disease, hereditary lymphohistiocytosis, osteopetrosis, osteogenesis imperfecta, storage diseases, thalassemia major, systemic sclerosis, systemic lupus erythematosus, multiple sclerosis, and juvenile rheumatoid arthritis.

In addition, administration of hematopoietic stem and progenitor cells can be used to treat autoimmune disorders. In some embodiments, upon infusion into a patient, transplanted hematopoietic stem and progenitor cells may home to a stem cell niche, such as the bone marrow, and establish productive hematopoiesis. This, in turn, can re-constitute a population of cells depleted during autoimmune cell eradication, which may occur due to the activity of self-reactive lymphocytes (e.g., self-reactive T lymphocytes and/or self-reactive B lymphocytes).

Autoimmune diseases that can be treated by way of administering hematopoietic stem and progenitor cells to a patient include, without limitation, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

Hematopoietic stem cell transplant therapy may additionally be used to treat neurological disorders, such as Parkinson's disease, Alzheimer's disease, multiple sclerosis, Amyotrophic lateral sclerosis, Huntington's disease, mild cognitive impairment, amyloidosis, AIDS-related dementia, encephalitis, stroke, head trauma, epilepsy, mood disorders, and dementia. As described herein, upon transplantation into a patient, hematopoietic stem cells may migrate to the central nervous system and differentiate into, for example, microglial cells, thereby re-constituting a population of cells that may be damaged or deficient in a patient suffering from a neurological disorder. In these cases, for example, a population of hematopoietic stem cells may be expanded ex vivo by culturing the cells in the presence of an aryl hydrocarbon receptor antagonist described herein. The hematopoietic stem cells thus expanded may then be administered to a patient suffering from a neurological disorder, where the cells may home to the central nervous system, such as the brain of the patient, and re-constitute a population of hematopoietic cells (e.g., microglial cells) that are damaged or deficient in the patient.

### Methods of Treating Inherited Metabolic Disorders—Administration of Expanded CD90+ Stem Cells for Microglial Engraftment in the Brain

As described herein, hematopoietic stem cell transplant therapy can be administered to a subject in need of treatment so as to populate or repopulate one or more blood cell types, such as a blood cell lineage that is deficient or defective in a patient suffering from a stem cell disorder. Hematopoietic stem and progenitor cells exhibit multi-potency, and can thus differentiate into multiple different blood lineages including, in one embodiment, microglia.

In one embodiment, hematopoietic stem cell transplant therapy or hematopoietic stem cell transplantation of inherited metabolic disorders may be accomplished using cross-correction. (Wynn, R. “Stem Cell Transplantation in Inherited Metabolic Disorders” Hematology 2011, pp. 285-291.) Cross correction involves engrafiment of expanded HSCs in the patient or host tissue, where the implanted cells secrete the deficient enzyme and said deficient enzyme is then taken up by cells in the patient which are deficient in that enzyme.

In one embodiment, the inherited metabolic disorder to be treated is selected from Hurler syndrome (Hurler's Disease), mucopolysaccharide disorders (e.g., Maroteaux Lamy syndrome), lysosomal storage disorders, and peroxisomal disorders (e.g., X-linked adrenoleukodystrophy), glycogen storage diseases, mucopolysaccharidoses, Mucolipidosis II, Gaucher's Disease, sphingolipidoses, and metachromatic leukodystrophy.

In certain embodiments, HSCs in the patient or in a healthy donor are mobilized using a CXCR2 agonist and/or CXCR4 antogonist of the disclosure. The CXCR4 antagonist may be plerixafor or a variant thereof, and a CXCR2 agonist may be Gro-β or a variant thereof, such as a truncation of Gro-β, for instance, Gro-β T. Mobilized HSCs are then isolated from a peripheral blood sample of the subject. Methods of isolating HSCs will be readily apparent to one of ordinary skill in the art. If the HSCs were isolated from the subject with the inherited metabolic disorder, the HSCs can then be genetically modified to correct the genetic defect leading to the disorder, expanded using the methods of the disclosure, and the corrected, expanded cells then transplanted back into the patient (an autologous transplantation). Optionally, HSCs may be expanded prior to genetic modification. Alternatively, HSCs may be mobilized using a CXCR2 agonist and/or CXCR4 antogonist of the disclosure in a healthy individual who (1) does not suffer from an inherited metabolic disorder and (2) is a compatible donor for the subject who does suffer from the inherited metabolic disorder. HSCs can be isolated from a blood sample taken from this healthy individual collected following mobilization, the HSCs can then be expanded using the expansion methods of the disclosure, and the expanded cells transplanted into the subject with the inherited metabolic disorder.

Expanded HSCs prepared according to the methods of the disclosure (i.e., contacting with a compound of the present disclosure) may advantageously lead to more microglia engraftment than fresh cells or cells cultured in the presence of cytokines. Without wishing to be bound by any theory, it is believed that this may be due to the presence of more CD90+ cells in expanded cell populations.

The methods disclosed herein for treating inherited metabolic disorders in a subject in need thereof comprise the administration of an expanded population of hematopoietic stem cells to a subject in need thereof. In one embodiment, the number of expanded hematopoietic stem cells administered to the subject is equal to or greater than the amount of hematopoietic stem cells needed to achieve a therapeutic benefit. In one embodiment, the number of expanded hematopoietic stem cells administered to the subject is greater than the amount of hematopoietic stem cells needed to achieve a therapeutic benefit. In one embodiment, the therapeutic benefit achieved is proportional to the number of expanded hematopoietic stem cells that are administered.

A dose of the expanded hematopoietic stem cell composition of the disclosure is deemed to have achieved a therapeutic benefit if it alleviates a sign or a symptom of the disease. The sign or symptom of the disease may comprise one or more biomarkers associated with the disease, or one or more clinical symptoms of the disease.

For example, administration of the expanded hematopoietic stem cell composition may result in the reduction of a biomarker that is elevated in individuals suffering from the disease, or elevate the level of a biomarker that is reduced in individuals suffering from the disease.

For example, administering the expanded hematopoietic stem cell composition of the disclosure may elevate the level of an enzyme that is reduced in an individual suffering from a metabolic disorder. This change in biomarker level may be partial, or the level of the biomarker may return to levels normally seen in healthy individuals.

In one embodiment, when the disease is, for example, an inherited metabolic disorder with a neurological component, the expanded hematopoietic stem cell composition may partly or fully reduce one or more clinical symptoms of the inherited metabolic disorder. Exemplary but non-limiting symptoms that may be affected by administration of the expanded hematopoietic stem cell composition of the disclosure comprise ataxias, dystonia, movement, disorders, epilepsies, and peripheral neuropathy.

In some cases, the sign or symptom of the inherited metabolic disorder with a neurological component comprises psychological signs or symptoms. For example, the sign or symptom of the disorder may comprise acute psychotic disorder, hallucinations, depressive syndrome, other symptoms or combinations of symptoms. Methods of evaluating psychological signs or symptoms associated with metabolic disorders with a neurological component will be known to one of ordinary skill in the art.

In some embodiments, the onset of the inherited metabolic disorder may be adult or pediatric.

In some embodiments, the inherited metabolic disorder may lead to degeneration of the nervous system.

In some embodiments, alleviating a sign or a symptom of the disorder may comprise slowing the rate of neurodegeneration or the rate of the progression of the disease.

In some embodiments, alleviating a sign or a symptom of the disorder may comprise reversing neurodegeneration or reversing the progression of the disease. Non-limiting exemplary symptoms of neurodegeneration comprise memory loss, apathy, anxiety, agitation, loss of inhibition and mood changes. Methods of evaluating neurodegeneration, and the progression thereof, will be known to one of ordinary skill in the art.

For example, in a patient suffering from Hurler syndrome, heparan and dermatan sulfate accumulation follows from α-L-iduronidase deficiency. Without wishing to be bound by any theory, it is believed that treatments that better clear these accumulated substrates will better correct the underlying disorder.

As described herein, hematopoietic stem cell transplant therapy can be administered to a subject in need of treatment so as to populate or repopulate one or more blood cell types, such as a blood cell lineage that is deficient or defective in a patient suffering from a stem cell disorder. Hematopoietic stem and progenitor cells exhibit multi-potency, and can thus differentiate into multiple different blood lineages.

The methods disclosed herein for treating disorders in a subject in need thereof comprise the administration of an expanded population of hematopoietic stem cells to a subject in need thereof. In one embodiment, the number of expanded hematopoietic stem cells administered to the subject is equal to or greater than the amount of hematopoietic stem cells needed to achieve a therapeutic benefit. In one embodiment, the number of expanded hematopoietic stem cells administered to the subject is greater than the amount of hematopoietic stem cells needed to achieve a therapeutic benefit. In one embodiment, the therapeutic benefit achieved is proportional to the number of expanded hematopoietic stem cells that are administered.

A dose of the expanded hematopoietic stem cell composition of the disclosure is deemed to have achieved a therapeutic benefit if it alleviates a sign or a symptom of the disease. The sign or symptom of the disease may comprise one or more biomarkers associated with the disease, or one or more clinical symptoms of the disease.

For example, administration of the expanded hematopoietic stem cell composition may result in the reduction of a biomarker that is elevated in individuals suffering from the disease, or elevate the level of a biomarker that is reduced in individuals suffering from the disease.

For example, administering the expanded hematopoietic stem cell composition of the disclosure may elevate the level of an enzyme that is reduced in an individual suffering from a metabolic disorder. This change in biomarker level may be partial, or the level of the biomarker may return to levels normally seen in healthy individuals.

### Selection of Donors and Patients

In some embodiments, the patient is the donor. In such cases, withdrawn hematopoietic stem or progenitor cells may be re-infused into the patient, such that the cells may subsequently home hematopoietic tissue and establish productive hematopoiesis, thereby populating or repopulating a line of cells that is defective or deficient in the patient (e.g., a population of megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes). In this scenario, the transplanted hematopoietic stem or progenitor cells are least likely to undergo graft rejection, as the infused cells are derived from the patient and express the same HLA class I and class II antigens as expressed by the patient.

Alternatively, the patient and the donor may be distinct. In some embodiments, the patient and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs).

In some embodiments, the patient and the donor are HLA-mismatched, which occurs when at least one HLA antigen, in particular with respect to HLA-A, HLA-B, and HLA-DR, is mismatched between the donor and recipient. To reduce the likelihood of graft rejection, for example, one haplotype may be matched between the donor and recipient, and the other may be mismatched.

Administration and Dosing of Hematopoietic Stem or Progenitor Cells Hematopoietic stem and progenitor cells described herein may be administered to a subject, such as a mammalian subject (e.g., a human subject) suffering from a disease, condition, or disorder described herein, by one or more routes of administration. For instance, hematopoietic stem cells described herein may be administered to a subject by intravenous infusion. Hematopoietic stem cells may be administered at any suitable dosage. Non-limiting examples of dosages included about 1×10⁵ CD34+ cells/kg of recipient to about 1×10⁸ CD34+ cells/kg (e.g., from about 2×10⁵ CD34+ cells/kg to about 9×10⁷ CD34+ cells/kg, from about 3×10⁵ CD34+ cells/kg to about 8×10⁷ CD34+ cells/kg, from about 4×10⁵ CD34+ cells/kg to about 7×10⁷ CD34+ cells/kg, from about 5×10⁵ CD34+ cells/kg to about 6×10⁷ CD34+ cells/kg, from about 5×10⁵ CD34+ cells/kg to about 1×10⁸ CD34+ cells/kg, from about 6×10⁵ CD34+ cells/kg to about 1×10⁸ CD34+ cells/kg, from about 7×10⁵ CD34+ cells/kg to about 1×10⁸ CD34+ cells/kg, from about 8×10⁵ CD34+ cells/kg to about 1×10⁸ CD34+ cells/kg, from about 9×10⁵ CD34+ cells/kg to about 1×10⁸ CD34+ cells/kg, from about 1×10⁷ CD34+ cells/kg to about 1×10⁸ CD34+ cells/kg, or from about 1×10⁶ CD34+ cells/kg to about 1×10⁷ CD34+ cells/kg, among others).

Hematopoietic stem or progenitor cells and pharmaceutical compositions described herein may be administered to a subject in one or more doses. When multiple doses are administered, subsequent doses may be provided one or more days, weeks, months, or years following the initial dose.

### Methods of Modulating Aryl Hydrocarbon Receptor Activity and Treating Aryl Hydrocarbon Receptor Related Diseases

In another aspect, the disclosure features a method of modulating the activity of an aryl hydrocarbon receptor, comprising administering to a subject in need thereof an effective amount of a compound of any one of the above aspects, or a pharmaceutically acceptable salt, hydrate, or solvate thereof.

In another aspect, the disclosure features a method of treating or preventing a disease or disorder, comprising administering to a subject in need thereof an effective amount of a compound of any one of the above aspects, or a pharmaceutically acceptable salt, hydrate, or solvate thereof.

In some embodiments, the disease or disorder is characterized by the production of an aryl hydrocarbon receptor agonist.

In some embodiments, the disease or disorder is a cancer, a cancerous condition, or a tumor.

In some embodiments, the tumor is an invasive tumor.

In some embodiments, the tumor is a solid tumor. Exemplary solid tumors include, but are not limited to, sarcomas (such as Ewing sarcoma, osteosarcoma, rhabdomyosarcoma) and carcinomas (such as adrenocortical carcinoma).

In some embodiments, the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

In some embodiments, the method further comprises administering one or more additional anti-cancer therapies.

In some embodiments, the methods of the present disclosure may comprise contacting a compound or aryl hydrocarbon receptor antagonist as described herein with a tumor activity system, wherein said tumor activity system may comprise (i) a tumor cell and/or (ii) a mixture comprising one or more extracellular matrix components. In some embodiments, the tumor activity being measured in the tumor activity assay system may be tumor cell proliferation or tumor cell invasiveness.

Without wishing to be bound by theory, it has been suggested that the enzymes IDO1 and TDO2 may be involved in a pathway that produces aryl hydrocarbon agonists, which suppress the immune system and enable a tumor to evade eradication by the immunes system. Anti-cancer and/or anti-tumor activity is thus suggested by an immune-oncology mechanism whereby administering aryl hydrocarbon receptor antagonists, such as those disclosed herein, may counteract the immunosuppressive effects of aryl hydrocarbon agonist, thereby allowing a patient's immune system to recognize and/or eradicate a tumor.

In some embodiments, the anticancer activity of the small molecule aryl hydrocarbon receptor antagonists disclosed herein, compositions thereof, methods and uses thereof described herein may be established in a cell line model, tumor cell line model, and/or an animal model. Exemplary cell lines include, but are not limited to, human breast cancer cells (MCF-7, MDA-468, and SK-Br-3), human liver carcinoma cells (Hep-G2), human colon adinocarcinoma cells (Colo320 D-M), human acute promylocytic leukemia cells (HL-60), mouse sarcoma cells (Sarcoma 180), mouse melanoma cells (C₅₇/B1/6J). Cells may be maintained or grown in suitable media and contacted and/or incubated with various concentrations of the small molecule aryl hydrocarbon receptor antagonists disclosed herein and compositions thereof as described herein. Morphological changes in the cells and cell proliferation activity may be observed and demonstrate the anti-cancer activity of the aryl hydrocarbon receptor antagonists of the present disclosure.

In some embodiments the small molecule aryl hydrocarbon receptor antagonists disclosed herein, compositions thereof, methods and uses thereof described herein may produce marked anti-cancer effects in a human subject without causing significant toxicities or adverse effects. The efficacy of the treatments described herein can be measured by various parameters commonly used in evaluating cancer treatments, including but not limited to, tumor regression, tumor weight or size shrinkage, reduction in rate of tumor growth, the presence or the size of a dormant tumor, the presence or size of metastases or micrometastases, degree of tumor or cancer invasiveness, size or number of the blood vessels, time to progression, duration of survival, progression free survival, overall response rate, duration of response, and quality of life. For example, tumor shrinkage of greater than 50% in a 2-dimensional analysis may be a cut-off for declaring a response.

In some embodiments, the small molecule aryl hydrocarbon receptor antagonists disclosed herein, compositions thereof, methods and uses thereof described herein may be used to cause inhibition of metastatic spread without shrinkage of the primary tumor, or may simply exert a tumoristatic effect. In the case of cancers, the small molecule aryl hydrocarbon receptor antagonists disclosed herein, compositions thereof, methods and uses thereof described herein can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the small molecule aryl hydrocarbon receptor antagonists disclosed herein, compositions thereof, methods and uses thereof described herein may prevent growth and/or kill existing cancer cells, it can be cytostatic and/or cytotoxic. For cancer therapy, efficacy in vivo can, for example, be measured by assessing the duration of survival, duration of progression free survival (PFS), the response rates (RR), duration of response, and/or quality of life.

One aspect of this application provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast; ovary; cervix: prostate; testis, genitourinary tract; esophagus larynx, glioblastoma; neuroblastoma; stomach; skin, keratoacanthoma; lung, epidermoid carcinoma, large cell carcinoma, small cell carcinoma, lung adenocarcinoma; bone; colon: colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colonrectum, large intestine, rectum, brain and central nervous system; chronic myeloid leukemia (CML), and leukemia. The term “cancer” includes, but is not limited to, the following cancers: myeloma, lymphoma, or a cancer selected from gastric, renal, or and the following cancers: head and neck, oropharangeal, non-small cell lung cancer (NSCLC), endometrial, hepatocarcinoma, Non-Hodgkins lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the application, the present application provides for the use of one or more compounds of the application in the manufacture of a medicament for the treatment of cancer, including without limitation the various types of cancer disclosed herein.

In some embodiments, the compounds of this application are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this application are useful for treating hematopoietic disorders, in particular, acute-myelogenous leukemia (AML), chronic-myelogenous leukemia (CML), acute-promyelocytic leukemia, and acute lymphocytic leukemia (ALL).

This application further embraces the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

In accordance with the foregoing, the present application further provides a method for preventing or treating any of the diseases or disorders described above in a subject in need of such treatment, which method comprises administering to said subject a therapeutically effective amount of a compound or aryl hydrocarbon receptor antagonist of the application, or a pharmaceutically acceptable salt, hydrate, or solvate thereof, and optionally a second agent or anti-cancer therapy. For any of the above uses, the required dosage will vary depending on the mode of administration, the particular condition to be treated and the effect desired.

In other embodiments, the compound and the one or more additional anti-cancer therapies are administered simultaneously or sequentially.

### Pharmaceutical Compositions

In another aspect, the disclosure features a pharmaceutical composition comprising a compound of any one of the above aspects, or a pharmaceutically acceptable salt, hydrate, or solvate thereof, and a pharmaceutically acceptable carrier.

Compounds of the application can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present application in free form or in a pharmaceutically acceptable salt form with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present application with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

The pharmaceutical compositions of the present application comprise a therapeutically effective amount of a compound of the present application formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions of this application can be administered to humans and other animals orally, rectally, parenterally, intracisternally, intravaginally, intraperitoneally, topically (as by powders, ointments, or drops), buccally, or as an oral or nasal spray.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-iritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this application, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present application, disorders are treated or prevented in a subject, such as a human or other animal, by administering to the subject a therapeutically effective amount of a compound of the application, in such amounts and for such time as is necessary to achieve the desired result. The term “therapeutically effective amount” of a compound of the application, as used herein, means a sufficient amount of the compound so as to decrease the symptoms of a disorder in a subject. As is well understood in the medical arts a therapeutically effective amount of a compound of this application will be at a reasonable benefit/risk ratio applicable to any medical treatment.

In general, compounds of the application will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present application may range from about 0.1 mg/Kg to about 500 mg/Kg, alternatively from about 1 to about 50 mg/Kg. In general, treatment regimens according to the present application comprise administration to a patient in need of such treatment from about 10 mg to about 1000 mg of the compound(s) of this application per day in single or multiple doses. Therapeutic amounts or doses will also vary depending on route of administration, as well as the possibility of co-usage with other agents.

Upon improvement of a subject's condition, a maintenance dose of a compound, composition or combination of this application may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present application will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The application also provides for a pharmaceutical combinations, e.g., a kit, comprising a) a first agent which is a compound of the application as disclosed herein, in free form or in pharmaceutically acceptable salt form, and optionally b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the application and a co-agent, are both administered to a patient simultaneously in the form of a single entity or dosage. The term “non-fixed combination” means that the active ingredients, e.g., a compound of the application and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In certain embodiments, these compositions optionally further comprise one or more additional therapeutic agents. For example, an agent that modulates aryl hydrocarbon receptor activity, chemotherapeutic agents or other antiproliferative agents may be combined with the compounds or aryl hydrocarbon receptor antagonists of the present disclosure to treat proliferative diseases and cancers.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase modulators or pharmaceutical salts thereof may be formulated into pharmaceutical compositions for administration to animals or humans. These pharmaceutical compositions, which comprise an amount of the protein modulator effective to treat or prevent a protein kinase-mediated condition and a pharmaceutically acceptable carrier, are other embodiments of the present application.

The application is further illustrated by the following examples and synthesis schemes, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### EXAMPLES

The following examples are put forth so as to provide those of ordinary skill in the art with a description of how the compositions and methods described herein may be used, made, and evaluated, and are intended to be purely exemplary and are not intended to limit the scope of what the inventors regard as the invention.

### Example 1. Identification of Novel Aryl Hydrocarbon Receptor (AHR) Antagonists by High-throughput Screening

### Screen Rationale

HepG2 hepatocellular carcinoma cells were transfected with a dioxin-response element (DRE) Firefly luciferase reporter construct. Luciferase transcription was activated with the aryl hydrocarbon receptor (AHR) agonist, VAF347. Addition of an AHR antagonist results in inhibition of the luciferase transcription and a loss of signal in the assay. The objective of this study was to discover novel AHR antagonists.

### HepG2 Cell Line Transfection

HepG2, a hepatocellular carcinoma cell line, was transfected with the dioxin-response element Firefly luciferase reporter construct using the TransIT-transfection system. Prior to transfection, HepG2 cells (Sigma) were cultured at 90% confluency in Eagle's Modified Essential Medium (Sigma)+2 mM Glutamine (Sigma), +1^{%} Non Essential Amino Acids (Sigma)+10% Fetal Bovine Serum (Hyclone) and harvested and counted (Biorad TC20 cell counter). OptilMEM serum-free media and TransIT (Mirus) were mixed for transfection in 150 mm dishes by adding 240 μL Transit to 3.0 mL OptiMEM, vortexing briefly to mix, and incubating at room temperature for 20 minutes. The DNA mixture was prepared by adding 80 μg plasmid (pGudLuc6.1 plasmid, Garrison et al., (1996) Fundam. Appl. Toxicol., 30, 194-203), inverting to mix, and incubating at room temperature for 30 minutes. 30×10⁶ HepG2 cells were seeded in 30 mL media and 3.3 mL of the DNA, OptiMEM, and TransIT mixture was added dropwise to the plate. The plate was mixed by rocking and incubated at 37° C./5% CO₂ overnight. The next day, cells were harvested by aspirating media, washing once with sterile phosphate buffered saline (PBS, Gibco), trypsinzing with 0.05% Trypsin (Corning) and harvesting cells with culture medium, and freezing with Cell Freezing Media (Gibco) at 5 50×10⁶ cells/vial in 1 mL/vial.

### DRE-Luciferase Reporter High-Throughput Screen

Transiently-transfected frozen HepG2 cells with DRE-Luciferase, were thawed in a 37° C. water bath and resuspended in complete media. 4 μL cells at 15,000 cells per well was transferred into the wells of a 1536-well Alphaplate assay plate (PerkinElmer) using TEMPEST liquid dispenser. The plate was centrifuged at 1000 rpm for 1 minute. 30 nL of the controls (1 μM (S)-2-(6-((2-(1H-indol-3-yl)ethyl)amino)-2-(5-fluoropyridin-3-yl)-9H-purin-9-yl)propan-1-ol or 100% DMSO) were transferred to the assay plate using the Pin Tool on BRAVO. 1 μL of VAF347 (15 nM final concentration) in complete media was added to the wells using TEMPEST. The plate was centrifuged at 1000 rpm for 1 minute and incubated at 37° C./5% CO2 overnight. After incubation, the plate was removed from the incubator and brought to room temperature. 2 μL of room temperature Steady-Glo luciferase detection reagent (Promega) was added to the wells with TEMPEST. The plate was centrifuged at 2000 rpm for 1 minute, sealed with clear plastic seal and kept at room temperature for 15 minutes prior to measuring luminescence on ENVISION.

Approximately 166,000 compounds were screened in 132 1536-well plates in the luciferase reporter gene assay at a final concentration of 12 μM. The average Z′ for the screen was 0.52 and the average S/B ratio was 138-fold. 5,120 compounds were selected for confirmation assay based on having greater than approximately 49% inhibition in the screen and either having passed all chemistry filters or having failed one or more chemistry filters, clustered for diversity, and confirmed good starting material. Each compound was tested at 0.1 μM, 1.0 μM, and 10 μM in the DRE luciferase assay and Cell Titer Glo cytotoxicity assay (Promega). For the cytotoxicity assay, an equal volume of CellTiter-Glo reagent was added to the cell culture medium present in each well. Contents were mixed for 2 minutes on an orbital shaker to induce lysis and the plate was incubated at room temperature for 10 minutes to stabilize luminescent signal prior to recording luminescence. 512 compounds were selected for 10-point dose-response in the AHR luciferase reporter assay and the Cell Titer Glo cytotoxicity assay.

### IRF-Luciferase Report Assay Counter-Screen

HepG2 cells stably transfected with IRF-Luciferase (Gibco) were resuspended in IRF media containing sterile-filtered Dulbecco's Modified Eagle's Medium (Corning/Gibco), 10% Fetal Bovine Serum (Hyclone), and 1% Penicillin/Streptomycin (Gibco/Life Technologies). 4 μL of cells at 15,000 cells/well was transferred into the wells of the Alphaplate assay plate using TEMPEST liquid dispenser. The plate was centrifuged at 1000 rpm for 1 minute and 30 nL of 100% DMSO was transferred to the assay plate using the Pin Tool on BRAVO. 1 μL of IFN-γ (5 nM (85 ng/mL) final concentration, Peprotech) in IRF media was added to the wells using TEMPEST. The plate was centrifuged at 1000 rpm for 1 minute and incubated at 37° C./5% CO₂ for 24 hours. After incubation, the plate was removed from the incubator and brought to room temperature. 2 μL of room temperature Steady-Glo luciferase detection reagent was added to the wells with TEMPEST. The plate was centrifuged at 2000 rpm for 1 minute, sealed with clear plastic seal and kept at room temperature for 15 minutes prior to measuring luminescence on ENVISION.

### Selection of Compounds

For confirmatory studies in primary hematopoietic stem cells (HSCs), compounds were selected based on the following criteria: (1) no luciferase activity in the counter screen, (2) greater than 90% inhibition in the dose-response AHR luciferase assay with (3) an effective concentration that inhibits 50% of the luciferase signal (EC₅₀) of less than 1 nM.

### Compound Dilutions

Stock dilutions of compounds were prepared at 10 mM DMSO and aliquots were stored at −20° C.

### Confirmatory AHR Antagonist Screen

To confirm the AHR antagonist assay from screen results, vials containing 1 mL 50×10⁶ HepG2 transfected cells were rapidly thawed in a 37° C. water bath. 10 mL Complete Media was added dropwise to the cells. Cells were spun at 500×g for 5 minutes at room temperature to wash the cells. Cells were resuspended at an appropriate volume for 25,000 cells per well in a 384-well plate. 32 μL of cells per well were plated in a white 384-well plate (Corning). VAF347 agonist (EMD Millipore, 1 mM stock in DMSO) was diluted to 10× (800 nM) in complete media. DMSO was prepared as done above for VAF347. 4 μL of the VAF347 agonist at 80 nM final or DMSO control was added to the appropriate wells. 4 μL of additional assay compound was added on top of agonist. (S)-2-(6-((2-(H-indol-3-yl)ethyl)amino)-2-(5-fluoropyridin-3-yl)-9H-purin-9-yl)propan-1-ol serves as a positive control for the assay, with a top concentration of 10 μM. Compounds were mixed by gently tapping the plate. Cells were placed in 37° C./5% CO₂ incubator overnight. After incubation, 40 μL Bright-Glo Luciferase reagent (Promega) was added to each well, the plate was gently tapped to mix, and incubated at room temperature for 2 minutes. Luminescence was measured on a luminometer (Biotek).

### CD34+ Expansion Assay

Approximately 3,000 mobilized peripheral blood CD34+ cells were plated per well in 384-well plate at a final volume of 45 μL in HSC growth media (SFEM supplemented with Pen/Strep, 50 ng/mL FLT3L, TPO, SCF and IL-6). Serial dilutions were made in HSC growth media with a top final concentration of 10 μM. 5 μL 10× stock was added to each well. The plate was gently tapped on all sides and incubated for 7 days at 37° C./5% CO₂. On day 7, the media was aspirated on a Biotek plate washer and 30 μL staining solution was added to the cells with antibodies against CD34-PE, CD90-APC, CD45RA-PE-CF₅₉₄ in PBS. Cells were washed once in PBS and resuspended in 80 μL for final volume. 40 μL was acquired by flow cytometry (BD Celesta).

TABLE 7
Summary of AHR Antagonist High-throughput
Screen Results (DRE-Luc IC₅₀: 0 < A < 0.2 μM; 0.2 ≤ B < 0.5 μM;
0.5 ≤ C < 1.0 μM; 1.0 ≤ D < 2.0 μM; 2.0 μM ≤ E and CD34
Frequency EC₅₀: 0 < A < 500 nM; 500 ≤ B < 1000 nM;
1000 ≤ C < 5000 nM; 5000 nM ≤ D; NT = Not Tested).

| Cmpd. | DRE-Luc | CD34 Frequency |
|---|---|---|
| # | IC50 (μM) | EC50 (nM) |
| 1 | B | B |
| 2 | B | B |
| 3 | B | B |
| 4 | C | B |
| 5 | C | C |
| 6 | C | C |
| 7 | C | C |
| 8 | C | C |
| 9 | C | D |
| 10 | C | NT |
| 11 | C | C |
| 12 | C | B |
| 13 | C | A |
| 14 | A | A |
| 15 | A | A |
| 16 | B | A |
| 17 | C | A |
| 18 | B | A |
| 19 | A | A |
| 20 | C | B |
| 21 | B | B |
| 22 | C | B |
| 23 | B | B |
| 24 | B | B |
| 25 | B | B |
| 26 | B | C |
| 27 | C | C |
| 28 | B | C |
| 29 | C | C |
| 30 | C | D |
| 31 | B | NT |
| 32 | B | NT |
| 33 | B | NT |
| 34 | B | NT |
| 35 | C | NT |
| 36 | C | NT |
| 37 | C | NT |
| 38 | C | C |
| 39 | C | NT |
| 40 | A | NT |
| 41 | A | D |
| 42 | C | C |
| 43 | B | B |
| 44 | C | NT |
| 45 | A | B |
| 46 | B | B |
| 47 | C | C |
| 48 | A | NT |
| 49 | A | NT |
| 50 | A | NT |
| 51 | B | NT |
| 52 | C | C |
| 53 | C | B |
| 54 | C | NT |
| 55 | C | NT |
| 56 | B | NT |
| 57 | B | B |
| 58 | C | D |
| 59 | C | A |
| 60 | C | C |
| 61 | B | NT |
| 62 | C | NT |
| 63 | C | NT |
| 64 | C | NT |
| 126 | C | C |
| 65 | C | C |
| 66 | B | NT |
| 67 | C | NT |
| 68 | B | B |
| 69 | B | B |
| 70 | B | B |
| 71 | C | B |
| 72 | B | B |
| 73 | C | C |
| 74 | C | C |
| 75 | B | C |
| 76 | B | D |
| 77 | C | D |
| 78 | C | D |
| 79 | C | D |
| 80 | A | NT |
| 81 | B | NT |
| 111 | C | NT |
| 82 | A | B |
| 83 | A | A |
| 84 | B | NT |
| 85 | C | NT |
| 86 | B | NT |
| 87 | C | NT |
| 88 | B | C |
| 89 | C | A |
| 90 | A | NT |
| 91 | B | A |
| 92 | C | D |
| 93 | B | NT |
| 94 | C | A |
| 95 | C | C |
| 96 | C | C |
| 97 | B | NT |
| 112 | C | A |
| 98 | C | C |
| 113 | B | C |
| 114 | C | C |
| 99 | C | C |
| 115 | B | C |
| 100 | A | A |
| 101 | C | B |
| 102 | B | NT |
| 103 | C | NT |
| 104 | C | C |
| 105 | B | C |
| 106 | C | B |
| 107 | C | C |
| 108 | C | NT |
| 109 | B | B |
| 110 | C | NT |
| 116 | A | A |
| 117 | A | A |
| 118 | A | A |
| 119 | B | A |
| 120 | B | B |
| 121 | C | C |
| 122 | C | C |
| 123 | C | C |
| 124 | B | C |
| 125 | C | C |
| 127 | C | C |
| 128 | C | C |
| 129 | B | C |
| 130 | C | C |
| 131 | C | C |
| 132 | C | C |
| 133 | C | C |
| 134 | C | C |
| 135 | C | C |
| 136 | C | D |
| 137 | C | D |
| 138 | C | D |
| 139 | C | D |
| 140 | C | D |
| 141 | B | NT |
| 142 | B | NT |
| 143 | B | NT |
| 144 | C | NT |
| 145 | C | NT |
| 146 | C | NT |
| 147 | C | NT |
| 148 | C | NT |
| 149 | C | NT |
| 150 | C | NT |
| 151 | C | NT |
| 152 | C | NT |
| 153 | C | NT |
| 154 | C | NT |
| 155 | C | NT |
| 156 | C | NT |
| 157 | C | NT |

| Cmpd. | DRE-Luc |
|---|---|
| # | IC50 (μm) |
| 1A | E |
| 2A | E |
| 3A | D |
| 4A | D |
| 5A | D |
| 6A | D |
| 7A | D |
| 8A | E |
| 9A | E |
| 10A | D |
| 11A | D |
| 12A | D |
| 13A | E |
| 14A | E |
| 15A | D |
| 16A | D |
| 17A | D |
| 18A | E |
| 19A | D |
| 20A | D |
| 21A | D |
| 22A | D |
| 23A | D |
| 24A | E |
| 25A | E |
| 26A | D |
| 27A | D |
| 28A | D |
| 29A | D |
| 30A | D |
| 31A | E |
| 32A | D |
| 33A | E |
| 34A | E |
| 35A | D |
| 36A | D |
| 37A | D |
| 38A | E |
| 39A | D |
| 40A | D |
| 41A | E |
| 42A | D |
| 43A | D |
| 44A | D |
| 45A | E |
| 46A | D |
| 47A | E |
| 48A | E |
| 49A | D |
| 50A | E |
| 51A | D |
| 52A | D |
| 53A | D |
| 54A | D |
| 55A | D |
| 56A | E |
| 57A | D |
| 58A | D |
| 59A | E |
| 60A | E |
| 61A | D |
| 62A | E |
| 63A | E |
| 64A | D |
| 65A | D |
| 66A | D |
| 67A | D |
| 68A | D |
| 69A | D |
| 70A | E |
| 71A | E |
| 72A | D |
| 73A | E |
| 74A | E |
| 75A | D |
| 76A | D |
| 77A | D |
| 78A | D |
| 79A | D |
| 80A | D |
| 81A | D |
| 82A | D |
| 83A | E |
| 84A | D |
| 85A | D |
| 86A | D |
| 87A | D |
| 88A | E |
| 89A | E |
| 90A | E |
| 91A | D |
| 92A | D |
| 93A | D |
| 94A | D |
| 95A | E |
| 964 | D |
| 97A | D |
| 98A | D |
| 99A | D |
| 100A | E |
| 101A | D |
| 102A | D |
| 103A | D |
| 104A | E |
| 105A | E |
| 106A | D |
| 107A | D |
| 108A | E |
| 109A | D |
| 110A | D |
| 111A | D |
| 112A | D |
| 113A | D |
| 114A | D |
| 115A | D |
| 116A | E |
| 117A | E |
| 118A | D |
| 119A | D |
| 120A | D |
| 121A | E |
| 122A | D |
| 123A | D |
| 124A | E |
| 125A | D |
| 126A | D |
| 127A | D |
| 128A | D |
| 129A | D |
| 130A | D |
| 131A | D |
| 132A | D |
| 133A | D |
| 134A | D |
| 135A | E |
| 136A | E |
| 137A | E |
| 138A | D |
| 139A | D |
| 140A | D |
| 141A | D |
| 142A | E |
| 143A | E |
| 144A | D |
| 145A | D |
| 146A | D |
| 147A | E |
| 148A | D |
| 149A | E |
| 150A | E |
| 151A | D |
| 152A | D |
| 153A | E |
| 154A | E |
| 155A | D |
| 156A | D |
| 157A | D |
| 158A | D |
| 159A | D |
| 160A | D |
| 161A | E |
| 162A | D |
| 163A | D |
| 164A | D |
| 165A | E |
| 166A | D |
| 167A | E |
| 168A | D |
| 169A | D |
| 170A | D |
| 171A | D |
| 172A | D |
| 173A | D |
| 174A | E |
| 175A | E |
| 176A | D |
| 177A | D |
| 178A | E |
| 179A | E |
| 180A | E |
| 181A | D |
| 182A | D |
| 183A | E |
| 184A | D |
| 185A | E |
| 186A | E |
| 187A | E |
| 188A | D |
| 189A | E |
| 190A | D |
| 191A | E |
| 192A | D |
| 193A | D |
| 194A | E |
| 195A | D |
| 196A | E |
| 197A | D |
| 1B | D |
| 2B | D |
| 3B | B |
| 4B | E |
| 5B | C |
| 6B | C |
| 7B | D |
| 8B | C |
| 9B | D |
| 10B | B |
| 11B | C |
| 12B | D |
| 13B | C |
| 14B | C |
| 15B | C |
| 16B | D |
| 17B | C |
| 18B | E |
| 19B | E |
| 20B | C |
| 21B | D |
| 22B | D |
| 23B | D |
| 24B | C |
| 25B | B |
| 26B | C |
| 27B | D |
| 28B | B |
| 29B | D |
| 30B | D |
| 31B | D |
| 32B | D |
| 33B | B |
| 34B | D |
| 35B | E |
| 36B | D |
| 37B | B |
| 38B | C |
| 39B | D |
| 40B | D |
| 41B | D |
| 42B | B |
| 43B | E |
| 44B | D |
| 45B | D |
| 46B | B |
| 47B | D |
| 48B | C |
| 49B | D |
| 50B | C |
| 51B | D |
| 52B | B |
| 53B | D |
| 54B | C |
| 55B | C |
| 56B | C |
| 57B | D |
| 58B | E |
| 59B | C |
| 60B | D |
| 61B | D |
| 62B | C |
| 63B | D |
| 64B | C |
| 65B | D |
| 66B | D |
| 67B | D |
| 68B | D |
| 69B | A |
| 70B | C |
| 71B | D |
| 72B | A |
| 73B | C |
| 74B | D |
| 75B | D |
| 76B | B |
| 77B | E |
| 78B | C |
| 79B | D |
| 80B | C |
| 81B | E |
| 82B | C |
| 83B | C |
| 84B | C |
| 85B | C |
| 86B | E |
| 87B | B |
| 88B | B |
| 89B | D |
| 90B | D |
| 91B | D |
| 92B | D |
| 93B | B |
| 92B | C |
| 95B | C |
| 96B | E |
| 97B | D |
| 98B | D |
| 99B | E |
| 100B | E |
| 101B | B |
| 102B | D |
| 103B | C |
| 104B | C |
| 105B | D |
| 106B | A |
| 107B | B |
| 108B | D |
| 109B | D |
| 110B | B |
| 111B | B |
| 112B | B |
| 113B | D |
| 114B | E |
| 115B | C |
| 116B | D |
| 117B | D |
| 118B | E |
| 119B | B |
| 120B | C |
| 121B | C |
| 122B | D |
| 123B | D |
| 124B | C |
| 125B | B |
| 126B | D |
| 127B | E |

### OTHER EMBODIMENTS

All publications, patents and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

What is claimed is:

1. An aryl hydrocarbon receptor (AHR) modulator compound of Formula (I) or a salt thereof 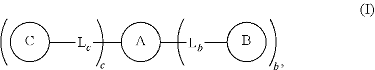
wherein:
A is an optionally substituted monocyclic, bicyclic, or tricyclic ring selected from 6- to 14-membered aryl and 5 to 14-membered saturated or unsaturated heterocyclyl comprising 1-5 heteroatoms selected from N, O and S;
b is 0 or 1;
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
L_{b} is a covalent bond, *—O—**, *—NR_{bb}—**, *—NR_{bb}C(O)NR_{bb}—**, *—C(O)— *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and A and ** denotes the linkage between L_{b} and B;
each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;
each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;
c is 0 or 1;
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;
each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl;
when c is 1, b is 1; and
when b is 0 and c is 0, A is an optionally substituted tricyclic ring selected from 14-membered aryl and 12- to 14-membered saturated or unsaturated heterocycle comprising 1-3 heteroatoms selected from N, O and S.

2. The compound of claim 1, wherein b is 1 and c is 0.

3. The compound of any one of the preceding claims, wherein A is an optionally substituted monocyclic ring selected from the group consisting of benzene, pyridine, thiazole, piperazine, pyrimidine, 1,2,3-triazole, pyrazole, furan, isoxazole, 4H-pyridazine, thiophene, oxazole, and 2H-pyridine.

4. The compound of any one of the preceding claims, wherein A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, and [1,2,4]triazolo[1,5-a]pyrimidine.

5. The compound of any one of the preceding claims, wherein A is an optionally substituted tricyclic ring selected from the group consisting of 4H-pyrido[1,2-a]thieno[2,3-d]pyrimidine, 2,4-dihydrothiochromeno[4,3-c]pyrazole, 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-c]quinolone.

6. The compound of any one of the preceding claims, wherein A is an optionally substituted tricyclic 13-membered ring comprising 2 heteroatoms selected from the group consisting of nitrogen and sulfur.

7. The compound of any one of the preceding claims, wherein B is an optionally substituted monocyclic ring selected from the group consisting of benzene, pyridine, pyrazole, thiophene, 1,2,3-triazole, pyrimidine, pyrrole, imidazole, pyrazine, pyrrolidine, 2,3-dihydropyrrole, 2,3-dihydrothiazole, 1,2,3,4-tetrahydropyridine, 1,2,3,6-tetrahydropyridine, isoxazole, and 1,3,4-oxadiazole.

8. The compound of any one of the preceding claims, wherein B is an optionally substituted bicyclic ring selected from the group consisting of quinolone, benzo[d]imidazole, benzo[d]oxazole, indoline, thieno[2,3-d]pyrimidine, benzo[d]isothiazole, indole, naphthalene, and benzofuran.

9. The compound of any one of the preceding claims, wherein B is an optionally substituted tricyclic dibenzo[b,d]furan.

10. The compound of any one of the preceding claims, wherein C is an optionally substituted monocyclic ring selected from the group consisting of benzene, isoxazole, pyridazine, thiazole, 1,3,4-oxadiazole, pyridine, pyrazole, pyrrole, thiophene, pyrimidine, morpholine, furan, and piperidine.

11. The compound of any one of the preceding claims, wherein C is an optionally substituted benzene.

12. The compound of any one of the preceding claims, wherein C is an optionally substituted bicyclic ring selected from the group consisting of benzo[d]oxazole, imidazo[1,2-a]pyridine, quinazoline, indole, 1,2,3,4-tetrahydronaphthalene, benzo[d] imidazole and benzo[d] thiazole.

13. The compound of any one of preceding claims, wherein L_{b} is a covalent bond, *—O—**, *—NH—**, *—NHC(O)NH—**, *—C(O)—**, *—SO₂—**, *=N—**, *—C(O)—N=**, *—OCH₂—**, *—C(O)NH—**, *—NR_{bb}C(O)—**, *—NH(CH₂)₂O—**, *—NH—R_{ba}—**, *—R_{ba}—NR_{bb}—**, *—SCH₂—**, *—SO₂CH₂—**, *—NH—N═CR_{bb}—**, *—C(O)NH—N═CH—**, *—OCH₂C(O)NH—**, *—NHC(O)CH₂NH—**, *—NHC(O)OCH₂—**, or *—CH₂N(CH₃)CH₂C(O)NHC(O)NH—**.

14. The compound of any one of preceding claims, wherein L_{b} is a covalent bond or *—C(O)NH—**.

15. The compound of any one of preceding claims, wherein L_{b} is a covalent bond.

16. The compound of any one of preceding claims, wherein L_{b} is *—C(O)NH—**.

17. The compound of any one of preceding claims, wherein L_{c} is a covalent bond, *—NH—**, C₁-C₃ alkyl, *—C(O)—**, *—N═CH₂—**, *—C(O)NH—**, *—SO₂—**, *—SCH₂—**, or *—OCH₂—**.

18. The compound of any one of the preceding claims, wherein L_{C} is a covalent bond.

19. The compound of any one of the preceding claims, wherein A is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

20. The compound of any one of the preceding claims, wherein B is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

21. The compound of any one of the preceding claims, wherein C is optionally substituted with one or more of: —CF₃, —OCF₃, —CN, —NO₂, —N(R)₂, —OR, —SR, —C(O)N(R)₂, —S(O)₂N(R)₂, —NRS(O)₂R, halo, oxo, ═NOR, —NROH, C₃-C₆ cycloalkyl, —S(CH₂)ₙF, —S(O)₂R, —C(O)R, —C(O)OR, —N(R)C(O)R, —OC(O)N(R)₂, —(CH₂)ₙN(R)C(O)R, 5- to 10-membered saturated or unsaturated heterocyclyl comprising 1-5 heteroatoms selected from N, O and S, phenyl optionally substituted with halogen or NO₂, and C₁-C₆ alkyl optionally substituted with C₂-C₆ alkynyl, halogen, or —OR in which each R is independently selected from the group consisting of H, —C(O)C₁-C₆ alkyl, C₃-C₆ cycloalkyl, an optionally substituted monocyclic or bicyclic ring selected from 6- to 10-membered aryl and 5- to 10-membered saturated or unsaturated heterocyclyl comprising 1-5 heteroatoms selected from N, O and S and C₁-C₆ alkyl optionally substituted with halogen; and each n is independently an integer from 1 to 4.

22. The compound of any one of the preceding claims, wherein the compound is represented by Formula (Ia) 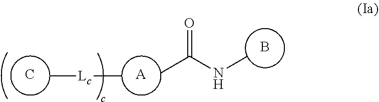
wherein
A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
c is 0 or 1,
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;
each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

23. The compound of any one of the preceding claims, wherein the compound is represented by Formula (Ia) and A is an optionally substituted monocyclic ring selected from the group consisting of benzene, pyridine, thiazole, 1,2,3-triazole, pyrazole, furan, isoxazole, 4H-pyridazine, thiophene, oxazole, 2H-pyridine, thizaole, pyrrole, and pyridinone.

24. The compound of any one of the preceding claims, wherein the compound is represented by Formula (Ia) and A is an optionally substituted monocyclic ring selected from the group consisting of benzene, pyridine, thiazole, 1,2,3-triazole, pyrazole, furan, isoxazole, 4H-pyridazine, thiophene, oxazole, and 2H-pyridine.

25. The compound of any one of the preceding claims, wherein the compound is represented by Formula (Ia) and A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, [1,2,4]triazolo[1,5-a]pyrimidine, naphthalene, thieno[3,2-d]imidazole, imidazo[1,5-a]pyridine, thieneo[3,2-d]pyrazole, indole, 2,3-dihydro-1H-indene, 5,6-dihydro-4H-cyclopenta[b]thiophene, and 2,3-dihydrobenzofuran.

26. The compound of any one of the preceding claims, wherein the compound is represented by Formula (Ia) and A is an optionally substituted bicyclic ring selected from the group consisting of benzo[d][1,2,3]triazole, thieno[2,3-b]pyridine, imidazo[1,2-a]pyridine, quinolone, pyrido[1,2-a]pyrimidine, 6,7-dihydro-5H-thiazolo[4,5-b]pyridine, benzo[d]imidazole, isoindoline, benzo[d]isothiazole, benzo[d]thiazole, benzo[b]thiophene, indoline, and [1,2,4]triazolo[1,5-a]pyrimidine.

27. The compound of any one of the preceding claims, wherein the compound is represented by Formula (Ia) and A is an optionally substituted tricyclic ring selected from the group consisting of 4H-pyrido[1,2-a]thieno[2,3-d]pyrimidine, 4H-pyrido[1,2-a]pyrrolo[2,3-d]pyrimidine, 2,4-dihydrothiochromeno[4,3-c]pyrazole, 3H-benz[e]indole, and 6,7,8,9=tetrahydrothieno[2,3-c]isoquinoline.

28. The compound of any one of the preceding claims, wherein the compound is represented by Formula (Ia) and A is an optionally substituted tricyclic ring selected from the group consisting of 4H-pyrido[1,2-a]thieno[2,3-d]pyrimidine, 4H-pyrido[1,2-a]pyrrolo[2,3-d]pyrimidine, and 2,4-dihydrothiochromeno[4,3-c]pyrazole.

29. The compound of any one of the preceding claims, wherein the compound is represented by Formula (Ia) and B is an optionally substituted monocyclic ring selected from the group consisting of benzene, pyridine, pyrazole, thiophene, pyrimidine, thiazole, isoxazole, imidazole, 1,2,4-triazole, 1,3,4-triazole, pyridine-2-one, and pyran-2-one.

30. The compound of any one of the preceding claims, wherein the compound is represented by Formula (Ia) and B is an optionally substituted monocyclic ring selected from the group consisting of benzene, pyridine, pyrazole, and thiophene.

31. The compound of any one of the preceding claims, wherein the compound is represented by Formula (Ia) and B is an optionally substituted bicyclic ring selected from the group consisting of indoline, quinolone, benzo[d]imidazole, benzo[d]oxazole, benzo[b]thiophene, benzo[d]thiazole, naphthalene, quinolone, 4H-chromen-4-one, 5,6-dihydro-4H-cyclopenta[b]thiophene, 4,5,6,7-tetrahydrobenzo[b]thiophene, and 7,8-2H-1-quinoline-2,5(6H)-dione.

32. The compound of any one of the preceding claims, wherein the compound is represented by Formula (Ia) and B is an optionally substituted bicyclic ring selected from the group consisting of indoline, quinolone, benzo[d]imidazole, and benzo[d]oxazole.

33. The compound of any one of the preceding claims, wherein the compound is represented by Formula (Ia) and B is an optionally substituted dibenzo[b,d]furan.

34. The compound of any of the preceding claims, wherein the compound is represented by Formula (Ia) and L_{c} is selected from the group consisting of a covalent bond, *—NH—**, and C₁-C₃ alkyl.

35. The compound of any one of the preceding claims, wherein the compound is represented by Formula (Ia) and C is an optionally substituted monocyclic ring selected from the group consisting of benzene, isoxazole, pyridazine, thiazole, pyrazole, imidazole, pyrimidine, pyridine, morpholine, and imidazolidine-2,4-dione.

36. The compound of any one of the preceding claims, wherein the compound is represented by Formula (Ia) and C is an optionally substituted monocyclic ring selected from the group consisting of benzene, isoxazole, pyridazine, and thiazole.

37. The compound of any one of the preceding claims, wherein the compound is represented by Formula (Ia) and C is an optionally substituted benzo[d]oxazole.

38. The compound of any one of the preceding claims, wherein the compound is represented by Formula (Ia) and A, B, or both A and B is an optionally substituted benzene.

39. The compound of any one of the preceding claims, wherein the compound is represented by Formula (Ia) and A or B is an optionally substituted thiophene.

40. The compound of any one of the preceding claims, wherein the compound is represented by Formula (Ia) and c is 0.

41. The compound of any one of the preceding claims, wherein the compound is selected from the group consisting of

| Compd. | |
|---|---|
| No. | Structure |
| 1 | 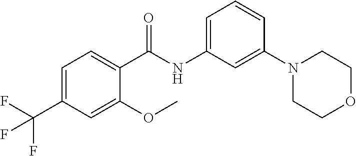 [SMILES: COc1cc(C(F)(F)F)ccc1C(=O)Nc1cccc(N2CCOCC2)c1] |
| 2 | 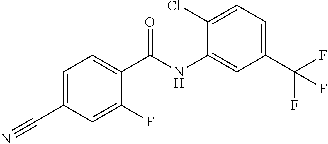 [SMILES: N#Cc1ccc(C(=O)Nc2cc(C(F)(F)F)ccc2Cl)c(F)c1] |
| 3 | 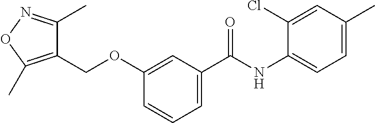 [SMILES: Cc1ccc(NC(=O)c2cccc(OCc3c(C)noc3C)c2)c(Cl)c1] |
| 4 | 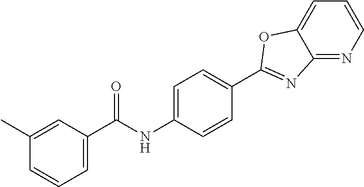 [SMILES: Cc1cccc(C(=O)Nc2ccc(-c3nc4ncccc4o3)cc2)c1] |
| 5 | 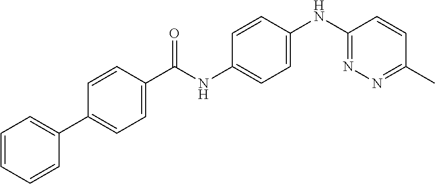 [SMILES: Cc1ccc(Nc2ccc(NC(=O)c3ccc(-c4ccccc4)cc3)cc2)nn1] |
| 6 | 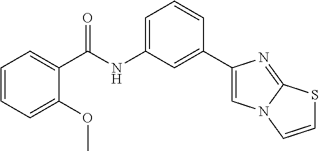 [SMILES: COc1ccccc1C(=O)Nc1cccc(-c2cn3ccsc3n2)c1] |
| 7 | 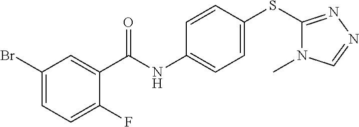 [SMILES: Cn1cnnc1Sc1ccc(NC(=O)c2cc(Br)ccc2F)cc1] |
| 8 | 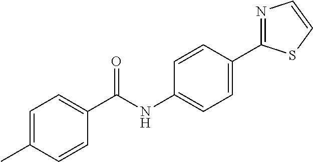 [SMILES: Cc1ccc(C(=O)Nc2ccc(-c3nccs3)cc2)cc1] |
| 9 | 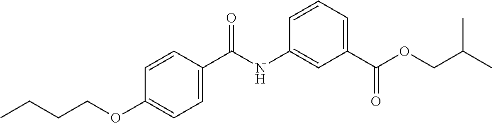 [SMILES: CCCCOc1ccc(C(=O)Nc2cccc(C(=O)OCC(C)C)c2)cc1] |
| 10 | 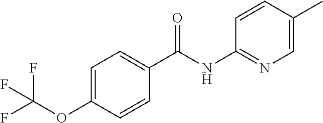 [SMILES: Cc1ccc(NC(=O)c2ccc(OC(F)(F)F)cc2)nc1] |
| 11 | 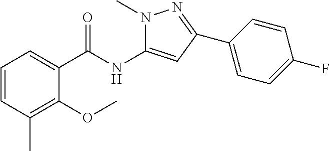 [SMILES: COc1c(C)cccc1C(=O)Nc1cc(-c2ccc(F)cc2)nn1C] |
| 12 | 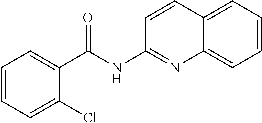 [SMILES: O=C(Nc1ccc2ccccc2n1)c1ccccc1Cl] |
| 13 | 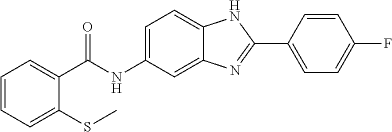 [SMILES: CSc1ccccc1C(=O)Nc1ccc2[nH]c(-c3ccc(F)cc3)nc2c1] |
| 14 | 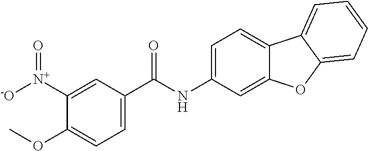 |
| 15 | 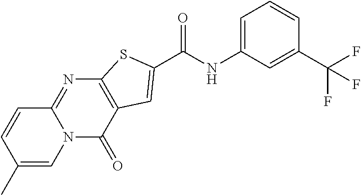 [SMILES: Cc1ccc2nc3sc(C(=O)Nc4cccc(C(F)(F)F)c4)cc3c(=O)n2c1] |
| 16 | 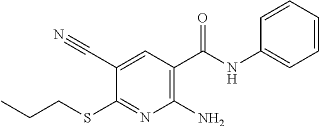 [SMILES: CCCSc1nc(N)c(C(=O)Nc2ccccc2)cc1C#N] |
| 17 | 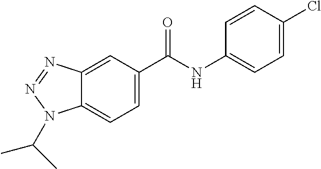 [SMILES: CC(C)n1nnc2cc(C(=O)Nc3ccc(Cl)cc3)ccc21] |
| 18 | 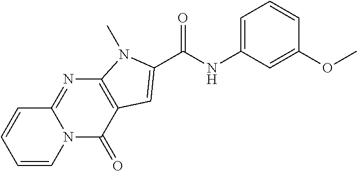 [SMILES: COc1cccc(NC(=O)c2cc3c(=O)n4ccccc4nc3n2C)c1] |
| 19 | 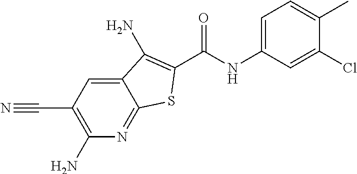 [SMILES: Cc1ccc(NC(=O)c2sc3nc(N)c(C#N)cc3c2N)cc1Cl] |
| 20 | 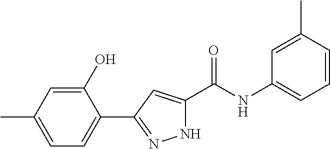 [SMILES: Cc1cccc(NC(=O)c2cc(-c3ccc(C)cc3O)n[nH]2)c1] |
| 21 | 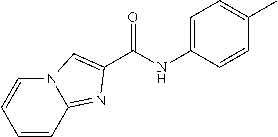 [SMILES: Cc1ccc(NC(=O)c2cn3ccccc3n2)cc1] |
| 22 | 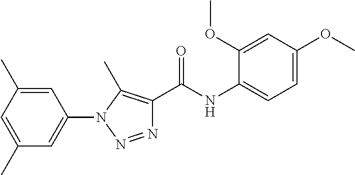 [SMILES: COc1ccc(NC(=O)c2nnn(-c3cc(C)cc(C)c3)c2C)c(OC)c1] |
| 23 | 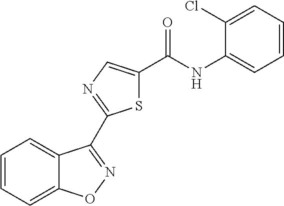 [SMILES: O=C(Nc1ccccc1Cl)c1cnc(-c2noc3ccccc23)s1] |
| 24 | 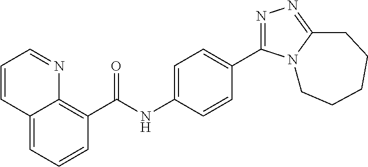 [SMILES: O=C(Nc1ccc(-c2nnc3n2CCCCC3)cc1)c1cccc2cccnc12] |
| 25 | 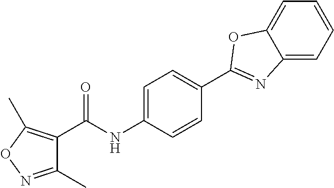 [SMILES: Cc1noc(C)c1C(=O)Nc1ccc(-c2nc3ccccc3o2)cc1] |
| 26 | 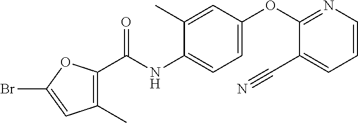 [SMILES: Cc1cc(Oc2ncccc2C#N)ccc1NC(=O)c1oc(Br)cc1C] |
| 27 | 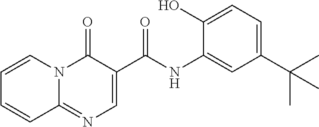 [SMILES: CC(C)(C)c1ccc(O)c(NC(=O)c2cnc3ccccn3c2=O)c1] |
| 28 | 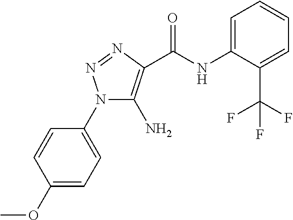 [SMILES: COc1ccc(-n2nnc(C(=O)Nc3ccccc3C(F)(F)F)c2N)cc1] |
| 29 | 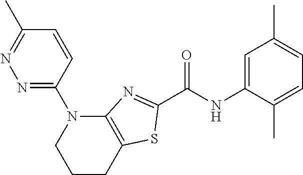 [SMILES: Cc1ccc(C)c(NC(=O)c2nc3c(s2)CCCN3c2ccc(C)nn2)c1] |
| 30 | 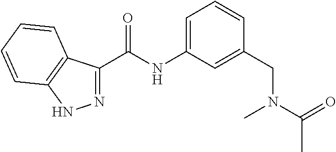 [SMILES: CC(=O)N(C)Cc1cccc(NC(=O)c2n[nH]c3ccccc23)c1] |
| 31 | 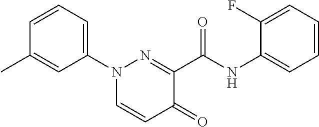 [SMILES: Cc1cccc(-n2ccc(=O)c(C(=O)Nc3ccccc3F)n2)c1] |
| 32 | 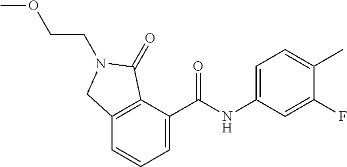 |
| 33 | 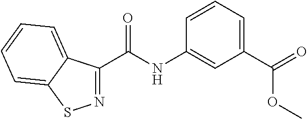 [SMILES: COC(=O)c1cccc(NC(=O)c2nsc3ccccc23)c1] |
| 34 | 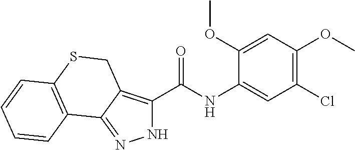 [SMILES: COc1cc(OC)c(NC(=O)c2[nH]nc3c2CSc2ccccc2-3)cc1Cl] |
| 35 | 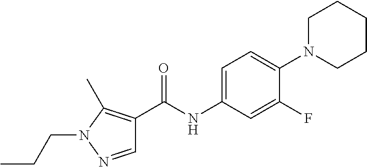 [SMILES: CCCn1ncc(C(=O)Nc2ccc(N3CCCCC3)c(F)c2)c1C] |
| 36 | 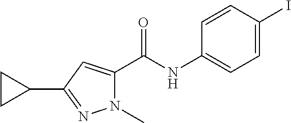 [SMILES: Cn1nc(C2CC2)cc1C(=O)Nc1ccc(I)cc1] |
| 37 | 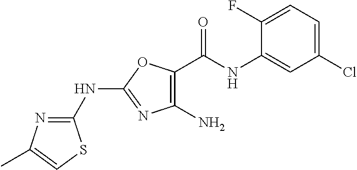 [SMILES: Cc1csc(Nc2nc(N)c(C(=O)Nc3cc(Cl)ccc3F)o2)n1] |
| 38 | 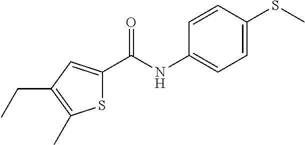 [SMILES: CCc1cc(C(=O)Nc2ccc(SC)cc2)sc1C] |
| 39 | 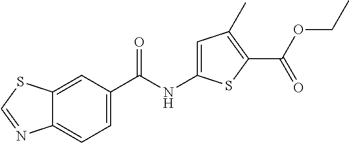 [SMILES: CCOC(=O)c1sc(NC(=O)c2ccc3ncsc3c2)cc1C] |
| 40 | 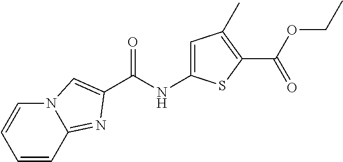 [SMILES: CCOC(=O)c1sc(NC(=O)c2cn3ccccc3n2)cc1C] |
| 41 | 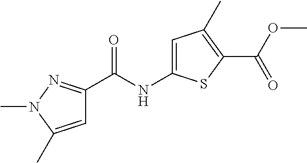 |
| 42 | 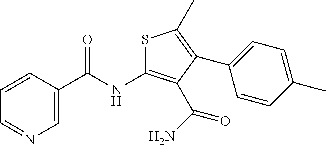 [SMILES: Cc1ccc(-c2c(C)sc(NC(=O)c3cccnc3)c2C(N)=O)cc1] |
| 43 | 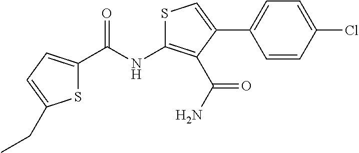 [SMILES: CCc1ccc(C(=O)Nc2scc(-c3ccc(Cl)cc3)c2C(N)=O)s1] |
| 44 | 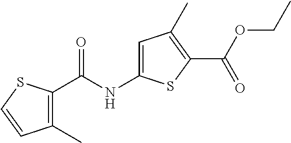 [SMILES: CCOC(=O)c1sc(NC(=O)c2sccc2C)cc1C] |
| 45 | 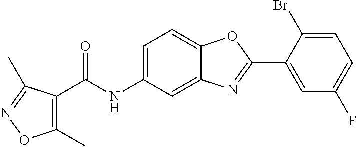 [SMILES: Cc1noc(C)c1C(=O)Nc1ccc2oc(-c3cc(F)ccc3Br)nc2c1] |
| 46 | 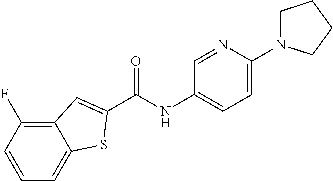 [SMILES: O=C(Nc1ccc(N2CCCC2)nc1)c1cc2c(F)cccc2s1] |
| 47 | 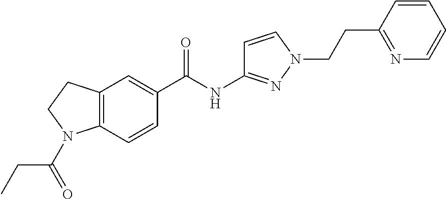 [SMILES: CCC(=O)N1CCc2cc(C(=O)Nc3ccn(CCc4ccccn4)n3)ccc21] |
| 48 | 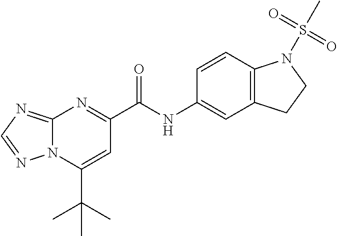 [SMILES: CC(C)(C)c1cc(C(=O)Nc2ccc3c(c2)CCN3S(C)(=O)=O)nc2ncnn12] |
| | and |
| 49 | 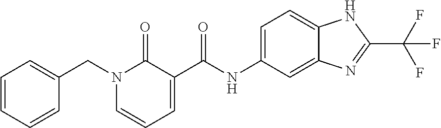 [SMILES: O=C(Nc1ccc2[nH]c(C(F)(F)F)nc2c1)c1cccn(Cc2ccccc2)c1=O] |

42. The compound of any one of the preceding claims, wherein the compound is selected from the group consisting of

| Compd. | |
|---|---|
| No. | Structure |
| 1A | 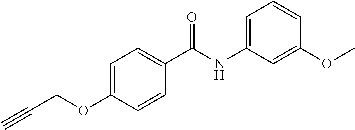 [SMILES: C#CCOc1ccc(C(=O)Nc2cccc(OC)c2)cc1] |
| 2A | 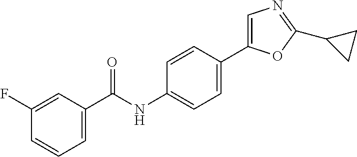 [SMILES: O=C(Nc1ccc(-c2cnc(C3CC3)o2)cc1)c1cccc(F)c1] |
| 3A | 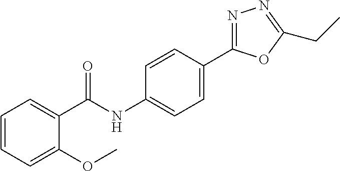 [SMILES: CCc1nnc(-c2ccc(NC(=O)c3ccccc3OC)cc2)o1] |
| 4A | 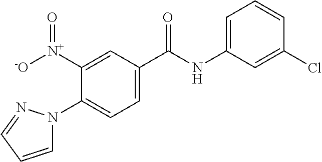 |
| 5A | 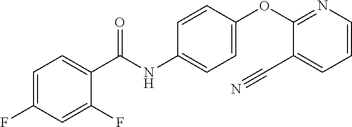 [SMILES: N#Cc1cccnc1Oc1ccc(NC(=O)c2ccc(F)cc2F)cc1] |
| 6A | 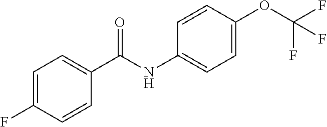 [SMILES: O=C(Nc1ccc(OC(F)(F)F)cc1)c1ccc(F)cc1] |
| 7A | 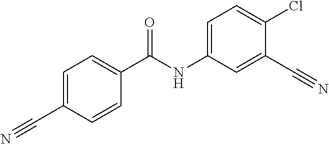 [SMILES: N#Cc1ccc(C(=O)Nc2ccc(Cl)c(C#N)c2)cc1] |
| 8A | 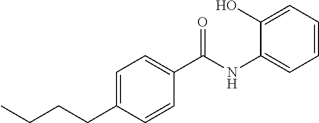 [SMILES: CCCCc1ccc(C(=O)Nc2ccccc2O)cc1] |
| 9A | 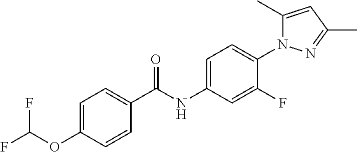 [SMILES: Cc1cc(C)n(-c2ccc(NC(=O)c3ccc(OC(F)F)cc3)cc2F)n1] |
| 10A | 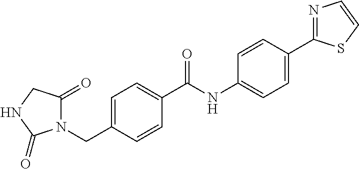 [SMILES: O=C(Nc1ccc(-c2nccs2)cc1)c1ccc(CN2C(=O)CNC2=O)cc1] |
| 11A | 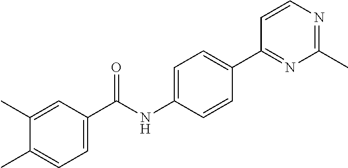 |
| 12A | 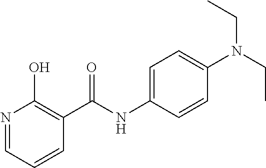 [SMILES: CCN(CC)c1ccc(NC(=O)c2cccnc2O)cc1] |
| 13A | 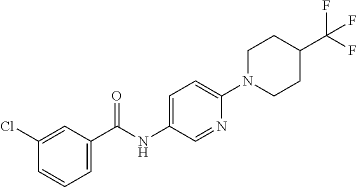 [SMILES: O=C(Nc1ccc(N2CCC(C(F)(F)F)CC2)nc1)c1cccc(Cl)c1] |
| 14A | 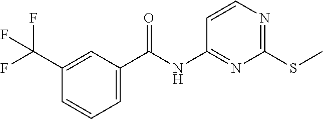 [SMILES: CSc1nccc(NC(=O)c2cccc(C(F)(F)F)c2)n1] |
| 15A | 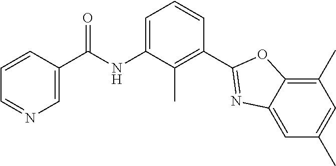 [SMILES: Cc1cc(C)c2oc(-c3cccc(NC(=O)c4cccnc4)c3C)nc2c1] |
| 16A | 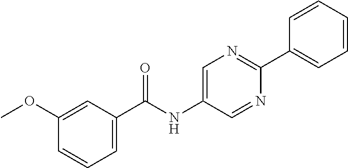 [SMILES: COc1cccc(C(=O)Nc2cnc(-c3ccccc3)nc2)c1] |
| 17A | 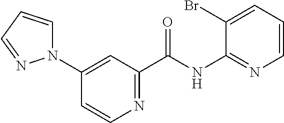 [SMILES: O=C(Nc1ncccc1Br)c1cc(-n2cccn2)ccn1] |
| 18A | 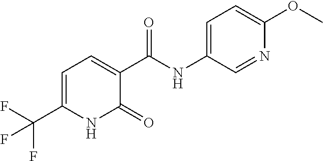 [SMILES: COc1ccc(NC(=O)c2ccc(C(F)(F)F)[nH]c2=O)cn1] |
| 19A | 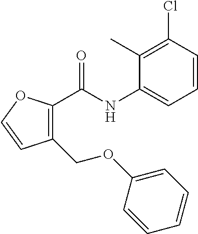 [SMILES: Cc1c(Cl)cccc1NC(=O)c1occc1COc1ccccc1] |
| 20A | 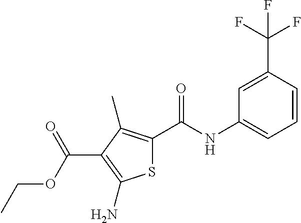 [SMILES: CCOC(=O)c1c(N)sc(C(=O)Nc2cccc(C(F)(F)F)c2)c1C] |
| 21A | 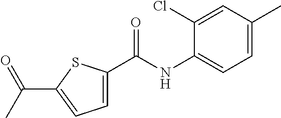 [SMILES: CC(=O)c1ccc(C(=O)Nc2ccc(C)cc2Cl)s1] |
| 22A | 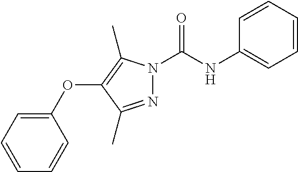 [SMILES: Cc1nn(C(=O)Nc2ccccc2)c(C)c1Oc1ccccc1] |
| 23A | 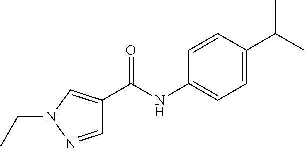 [SMILES: CCn1cc(C(=O)Nc2ccc(C(C)C)cc2)cn1] |
| 24A | 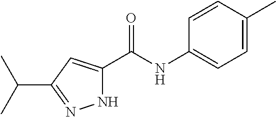 [SMILES: Cc1ccc(NC(=O)c2cc(C(C)C)n[nH]2)cc1] |
| 25A | 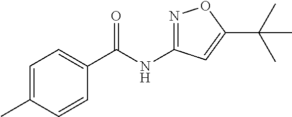 [SMILES: Cc1ccc(C(=O)Nc2cc(C(C)(C)C)on2)cc1] |
| 26A | 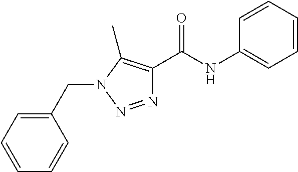 [SMILES: Cc1c(C(=O)Nc2ccccc2)nnn1Cc1ccccc1] |
| 27A | 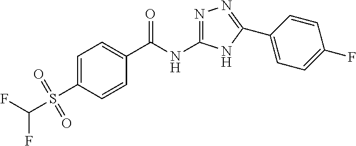 [SMILES: O=C(Nc1nnc(-c2ccc(F)cc2)[nH]1)c1ccc(S(=O)(=O)C(F)F)cc1] |
| 28A | 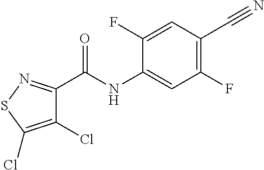 [SMILES: N#Cc1cc(F)c(NC(=O)c2nsc(Cl)c2Cl)cc1F] |
| 29A | 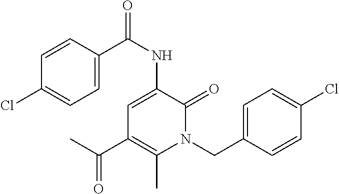 [SMILES: CC(=O)c1cc(NC(=O)c2ccc(Cl)cc2)c(=O)n(Cc2ccc(Cl)cc2)c1C] |
| 30A | 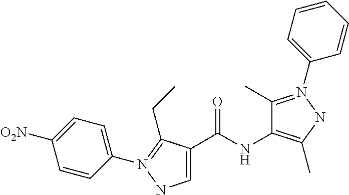 |
| 31A | 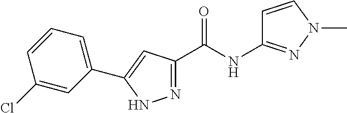 [SMILES: Cn1ccc(NC(=O)c2cc(-c3cccc(Cl)c3)[nH]n2)n1] |
| 32A | 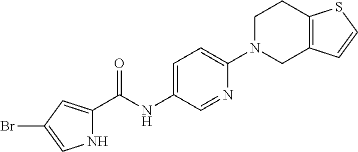 [SMILES: O=C(Nc1ccc(N2CCc3sccc3C2)nc1)c1cc(Br)c[nH]1] |
| 33A | 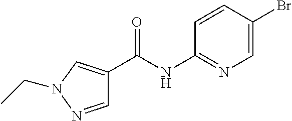 [SMILES: CCn1cc(C(=O)Nc2ccc(Br)cn2)cn1] |
| 34A | 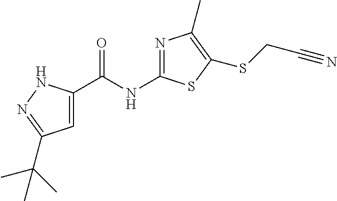 [SMILES: Cc1nc(NC(=O)c2cc(C(C)(C)C)n[nH]2)sc1SCC#N] |
| 35A | 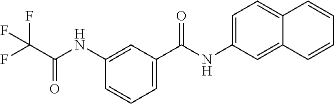 [SMILES: O=C(Nc1ccc2ccccc2c1)c1cccc(NC(=O)C(F)(F)F)c1] |
| 36A | 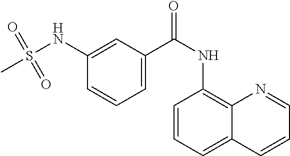 [SMILES: CS(=O)(=O)Nc1cccc(C(=O)Nc2cccc3cccnc23)c1] |
| 37A | 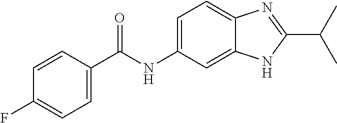 [SMILES: CC(C)c1nc2ccc(NC(=O)c3ccc(F)cc3)cc2[nH]1] |
| 38A | 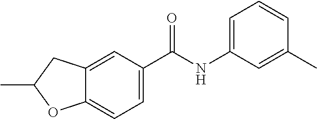 |
| 39A | 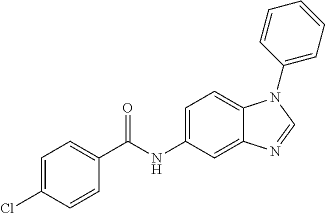 [SMILES: O=C(Nc1ccc2c(c1)ncn2-c1ccccc1)c1ccc(Cl)cc1] |
| 40A | 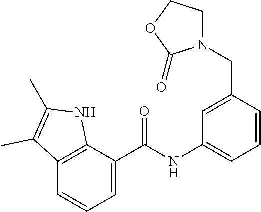 [SMILES: Cc1[nH]c2c(C(=O)Nc3cccc(CN4CCOC4=O)c3)cccc2c1C] |
| 41A | 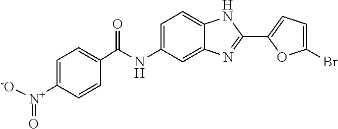 |
| 42A | 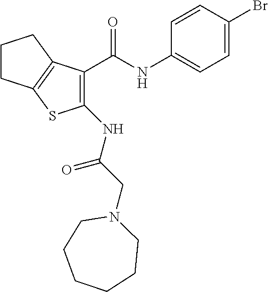 [SMILES: O=C(CN1CCCCCC1)Nc1sc2c(c1C(=O)Nc1ccc(Br)cc1)CCC2] |
| 43A | 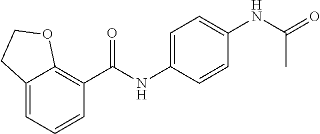 [SMILES: CC(=O)Nc1ccc(NC(=O)c2cccc3c2OCC3)cc1] |
| 44A | 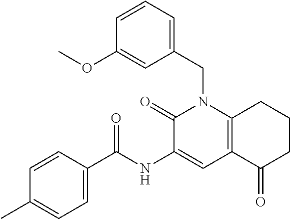 [SMILES: COc1cccc(Cn2c3c(cc(NC(=O)c4ccc(C)cc4)c2=O)C(=O)CCC3)c1] |
| 45A | 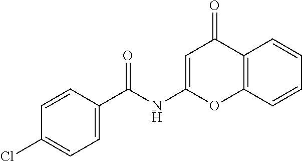 [SMILES: O=C(Nc1cc(=O)c2ccccc2o1)c1ccc(Cl)cc1] |
| 46A | 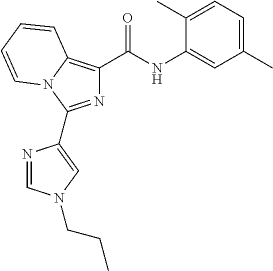 [SMILES: CCCn1cnc(-c2nc(C(=O)Nc3cc(C)ccc3C)c3ccccn23)c1] |
| 47A | 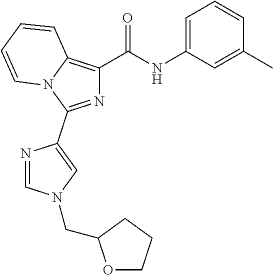 [SMILES: Cc1cccc(NC(=O)c2nc(-c3cn(CC4CCCO4)cn3)n3ccccc23)c1] |
| 48A | 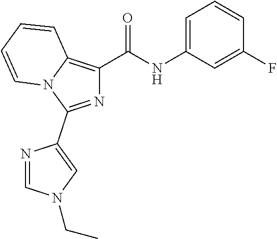 [SMILES: CCn1cnc(-c2nc(C(=O)Nc3cccc(F)c3)c3ccccn23)c1] |
| 49A | 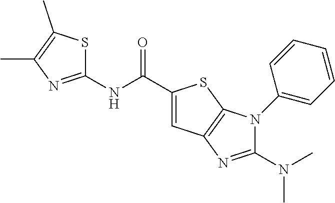 [SMILES: Cc1nc(NC(=O)c2cc3nc(N(C)C)n(-c4ccccc4)c3s2)sc1C] |
| 50A | 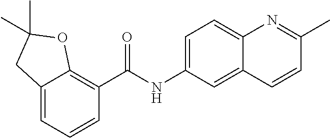 |
| 51A | 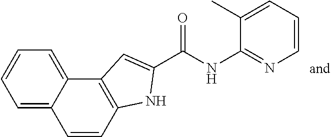 [SMILES: Cc1cccnc1NC(=O)c1cc2c(ccc3ccccc32)[nH]1] |
| 52A | 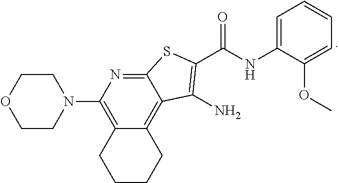 [SMILES: COc1ccccc1NC(=O)c1sc2nc(N3CCOCC3)c3c(c2c1N)CCCC3] |

43. The compound of any one of the preceding claims, wherein the compound is selected from the group consisting of

| Compd. | |
|---|---|
| No. | Structure |
| 1B | 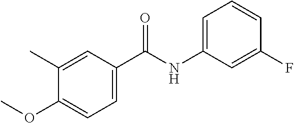 [SMILES: COc1ccc(C(=O)Nc2cccc(F)c2)cc1C] |
| 2B | 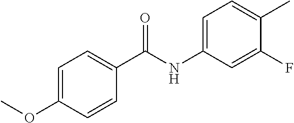 [SMILES: COc1ccc(C(=O)Nc2ccc(C)c(F)c2)cc1] |
| 3B | 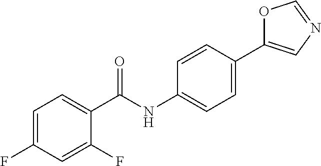 [SMILES: O=C(Nc1ccc(-c2cnco2)cc1)c1ccc(F)cc1F] |
| 4B | 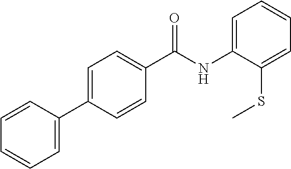 [SMILES: CSc1ccccc1NC(=O)c1ccc(-c2ccccc2)cc1] |
| 5B | 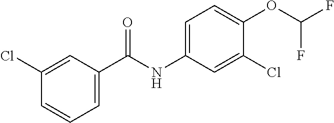 [SMILES: O=C(Nc1ccc(OC(F)F)c(Cl)c1)c1cccc(Cl)c1] |
| 6B | 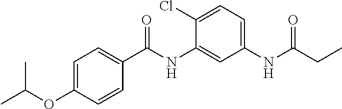 [SMILES: CCC(=O)Nc1ccc(Cl)c(NC(=O)c2ccc(OC(C)C)cc2)c1] |
| 7B | 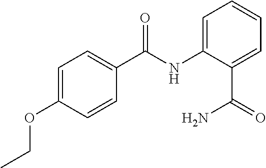 [SMILES: CCOc1ccc(C(=O)Nc2ccccc2C(N)=O)cc1] |
| 8B | 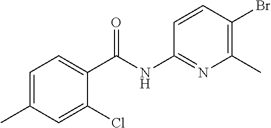 [SMILES: Cc1ccc(C(=O)Nc2ccc(Br)c(C)n2)c(Cl)c1] |
| 9B | 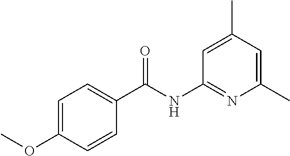 |
| 10B | 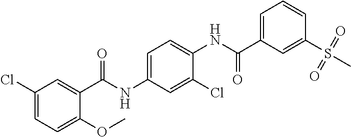 [SMILES: COc1ccc(Cl)cc1C(=O)Nc1ccc(NC(=O)c2cccc(S(C)(=O)=O)c2)c(Cl)c1] |
| 11B | 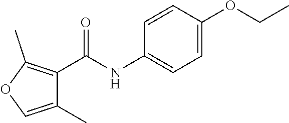 [SMILES: CCOc1ccc(NC(=O)c2c(C)coc2C)cc1] |
| 12B | 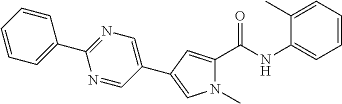 [SMILES: Cc1ccccc1NC(=O)c1cc(-c2cnc(-c3ccccc3)nc2)cn1C] |
| 13B | 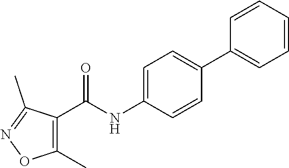 [SMILES: Cc1noc(C)c1C(=O)Nc1ccc(-c2ccccc2)cc1] |
| 14B | 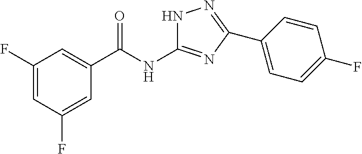 [SMILES: O=C(Nc1nc(-c2ccc(F)cc2)n[nH]1)c1cc(F)cc(F)c1] |
| 15B | 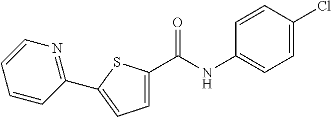 [SMILES: O=C(Nc1ccc(Cl)cc1)c1ccc(-c2ccccn2)s1] |
| 16B | 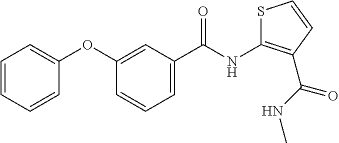 [SMILES: CNC(=O)c1ccsc1NC(=O)c1cccc(Oc2ccccc2)c1] |
| 17B | 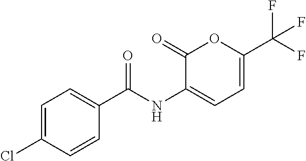 [SMILES: O=C(Nc1ccc(C(F)(F)F)oc1=O)c1ccc(Cl)cc1] |
| 18B | 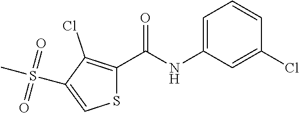 [SMILES: CS(=O)(=O)c1csc(C(=O)Nc2cccc(Cl)c2)c1Cl] |
| 19B | 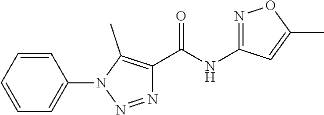 [SMILES: Cc1cc(NC(=O)c2nnn(-c3ccccc3)c2C)no1] |
| 20B | 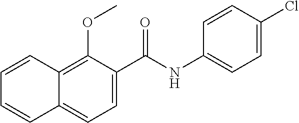 [SMILES: COc1c(C(=O)Nc2ccc(Cl)cc2)ccc2ccccc12] |
| 21B | 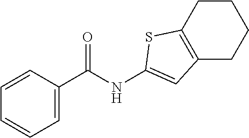 [SMILES: O=C(Nc1cc2c(s1)CCCC2)c1ccccc1] |
| 22B | 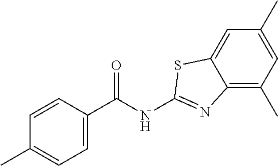 |
| 23B | 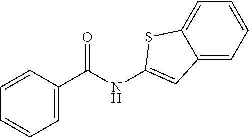 [SMILES: O=C(Nc1cc2ccccc2s1)c1ccccc1] |
| 24B | 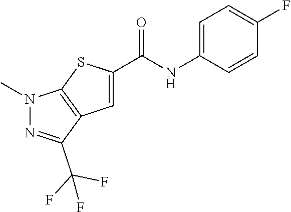 [SMILES: Cn1nc(C(F)(F)F)c2cc(C(=O)Nc3ccc(F)cc3)sc21] |
| 25B | 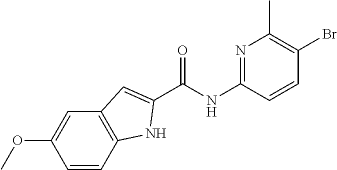 [SMILES: COc1ccc2[nH]c(C(=O)Nc3ccc(Br)c(C)n3)cc2c1] |
| 26B | 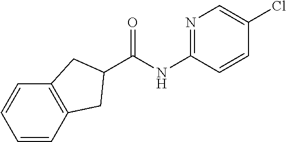 [SMILES: O=C(Nc1ccc(Cl)cn1)C1Cc2ccccc2C1] |
| 27B | 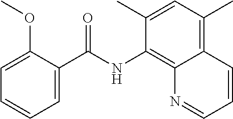 [SMILES: COc1ccccc1C(=O)Nc1c(C)cc(C)c2cccnc12] |
| 28B | 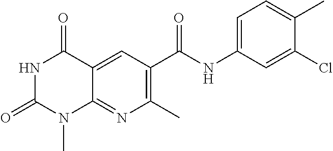 [SMILES: Cc1ccc(NC(=O)c2cc3c(=O)[nH]c(=O)n(C)c3nc2C)cc1Cl] |
| 29B | 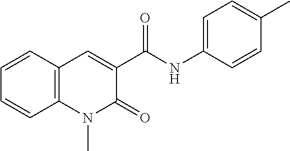 [SMILES: Cc1ccc(NC(=O)c2cc3ccccc3n(C)c2=O)cc1] |
| 30B | 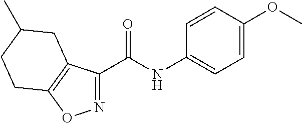 [SMILES: COc1ccc(NC(=O)c2noc3c2CC(C)CC3)cc1] |
| 31B | 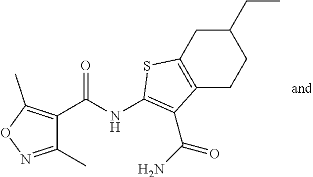 [SMILES: CCC1CCc2c(sc(NC(=O)c3c(C)noc3C)c2C(N)=O)C1] |
| 32B | 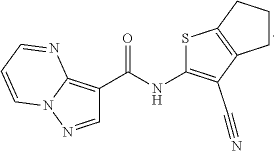 [SMILES: N#Cc1c(NC(=O)c2cnn3cccnc23)sc2c1CCC2] |

44. The compound of any one of the preceding claims, wherein A is 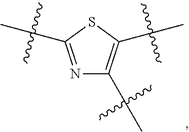 [SMILES: *c1nc(*)c(*)s1]
in which each [symbol] independently denotes the linkage between A and hydrogen, -L_{b}-B, -L_{c}-C, or a substituent.

45. The compound of any one of the preceding claims, wherein the compound is represented by Formula (Ib) 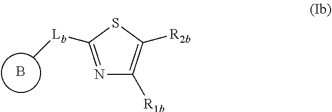
wherein
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a thiazole carbon and ** denotes the linkage between L_{b} and B;
each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;
each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;
R_{1b} is hydrogen or -L_{c}-C;
R_{2b} is hydrogen, an optionally substituted pyrazole ring, or CONR_{3b}R_{4b}, wherein each R_{3b} and R_{4b} is independently hydrogen or C₁-C₆ alkyl;
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—(O)NR_{cb}NR_{cb}C(O)—**, in which * denotes the linkage between L_{c} and a thiazole carbon and ** denotes the linkage between L_{c} and C;
each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl; and
R_{1b} and R_{2b} are not both hydrogen.

46. The compound of any one of the preceding claims, wherein the compound is represented by Formula (Ib) and R_{1b} is hydrogen.

47. The compound of any one of the preceding claims, wherein the compound is represented by Formula (Ib) and B is an optionally substituted monocyclic ring selected from the group consisting of benzene, pyridine, 2,3-dihydropyrrole, 1,2,3-triazole, pyrrolidine, thiophene, piperazine, imidazole, tetrazole, pyrrolidin-2-one, and 1,2-dihydro-3H-pyrrol-3-one.

48. The compound of any one of the preceding claims, wherein the compound is represented by Formula (Ib) and B is an optionally substituted monocyclic ring selected from the group consisting of benzene, pyridine, 2,3-dihydropyrrole, 1,2,3-triazole, pyrrolidine, and thiophene.

49. The compound of any one of the preceding claims, wherein the compound is represented by Formula (Ib) and B is an optionally substituted bicyclic ring selected from the group consisting of benzo[d]isooxazole, 2,3-dihydrobenzofuran, and imidazo[1,2-a]pyridine.

50. The compound of any one of the preceding claims, wherein the compound is represented by Formula (Ib) and L_{b} is selected from the group consisting of a covalent bond, *—NH—**, and *—NR_{bb}C(O)—**.

51. The compound of any one of the preceding claims, wherein the compound is represented by Formula (Ib) and L_{b} is a covalent bond.

52. The compound of any one of the preceding claims, wherein the compound is represented by Formula (Ib), R_{1b} is -L_{c}-C and L_{c} is a covalent bond.

53. The compound of any one of the preceding claims, wherein the compound is represented by Formula (Ib), R_{1b} is -L_{c}-C and C is an optionally substituted monocyclic ring selected from the group consisting of benzene, pyridine, pyrrole, pyrazole, 1,3,4-oxadiazole, 4H-1,2,4-triazole, thiophene, 1H-1,2,4-triazole, 1,2,3,4-tetrahydropyrimidine, and pyrimidine-2,4(1H,3H)-dione.

54. The compound of any one of the preceding claims, wherein the compound is represented by Formula (Ib), R_{1b} is -L_{c}-C and C is an optionally substituted monocyclic ring selected from the group consisting of benzene, pyridine, pyrrole, pyrazole, and 1,3,4-oxadiazole.

55. The compound of any one of the preceding claims, wherein the compound is represented by Formula (Ib), R_{1b} is -L_{c}-C and C is an optionally substituted bicyclic ring selected from the group consisting of imidazo[1,2-a]pyridine, benzo[d]imidazole, indoline, 1,2,3,4-tetrahydroquinoline, octahydro-1H-benzo[d]imidazole, and octahydro-2h-benzo[d]imidazole-2-one.

56. The compound of any one of the preceding claims, wherein the compound is represented by Formula (Ib), R_{1b} is -L_{c}-C and C is an optionally substituted bicyclic ring selected from the group consisting of imidazo[1,2-a]pyridine and benzo[d]imidazole.

57. The compound of any one of the preceding claims, wherein the compound is represented by Formula (Ib), R_{1b} is -L_{c}-C and both B and C are an optionally substituted monocyclic ring selected from benzene and pyridine.

58. The compound of any one of the preceding claims, wherein the compound is selected from the group consisting of

| Compd. | |
|---|---|
| No. | Structure |
| 50 | 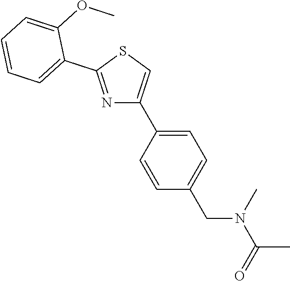 [SMILES: COc1ccccc1-c1nc(-c2ccc(CN(C)C(C)=O)cc2)cs1] |
| 51 | 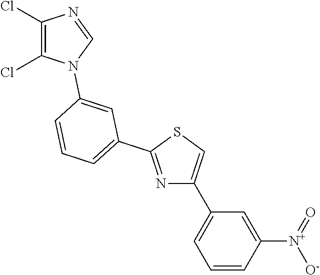 [SMILES: O=[N+]([O-])c1cccc(-c2csc(-c3cccc(-n4cnc(Cl)c4Cl)c3)n2)c1] |
| 52 | 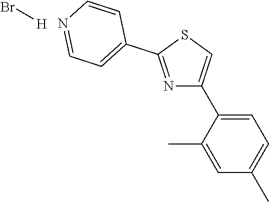 |
| 53 | 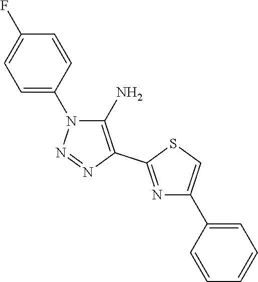 [SMILES: Nc1c(-c2nc(-c3ccccc3)cs2)nnn1-c1ccc(F)cc1] |
| 54 | 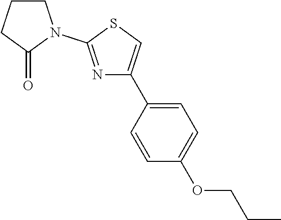 [SMILES: CCCOc1ccc(-c2csc(N3CCCC3=O)n2)cc1] |
| 55 | 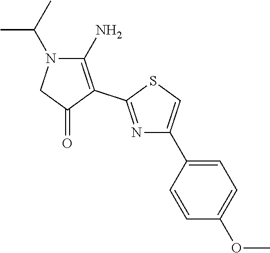 [SMILES: COc1ccc(-c2csc(C3=C(N)N(C(C)C)CC3=O)n2)cc1] |
| 56 | 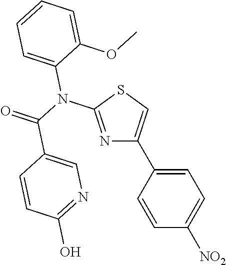 [SMILES: COc1ccccc1N(C(=O)c1ccc(O)nc1)c1nc(-c2ccc([N+](=O)[O-])cc2)cs1] |
| 57 | 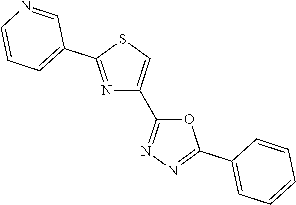 [SMILES: c1ccc(-c2nnc(-c3csc(-c4cccnc4)n3)o2)cc1] |
| 58 | 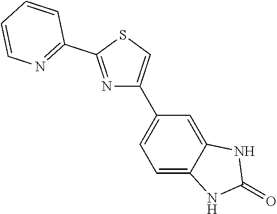 [SMILES: O=c1[nH]c2ccc(-c3csc(-c4ccccn4)n3)cc2[nH]1] |
| 59 | 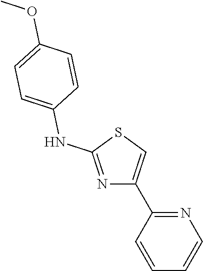 [SMILES: COc1ccc(Nc2nc(-c3ccccn3)cs2)cc1] |
| 60 | 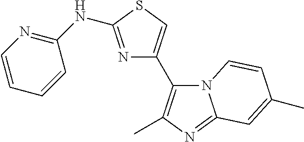 [SMILES: Cc1ccn2c(-c3csc(Nc4ccccn4)n3)c(C)nc2c1] |
| 61 | 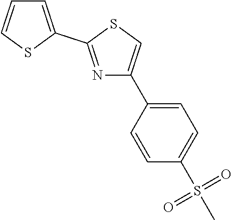 [SMILES: CS(=O)(=O)c1ccc(-c2csc(-c3cccs3)n2)cc1] |
| 62 | 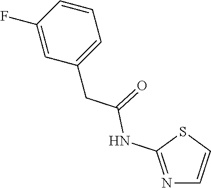 [SMILES: O=C(Cc1cccc(F)c1)Nc1nccs1] |
| 63 | 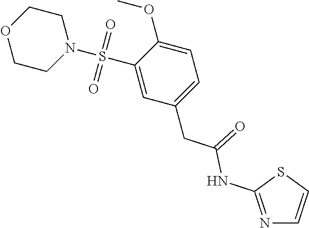 [SMILES: COc1ccc(CC(=O)Nc2nccs2)cc1S(=O)(=O)N1CCOCC1] |
| 64 | 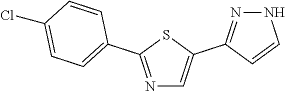 [SMILES: Clc1ccc(-c2ncc(-c3cc[nH]n3)s2)cc1] |
| 126 | 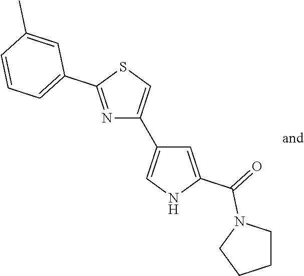 [SMILES: Cc1cccc(-c2nc(-c3c[nH]c(C(=O)N4CCCC4)c3)cs2)c1] |
| 65 | 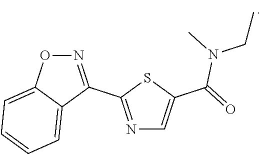 [SMILES: CCN(C)C(=O)c1cnc(-c2noc3ccccc23)s1] |

59. The compound of any one of the preceding claims, wherein the compound is selected from the group consisting of

| Compd. | |
|---|---|
| No. | Structure |
| 53A | 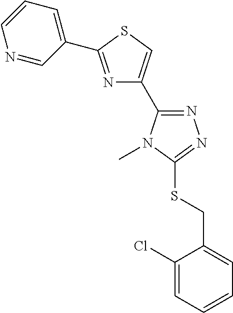 [SMILES: Cn1c(SCc2ccccc2Cl)nnc1-c1csc(-c2cccnc2)n1] |
| 54A | 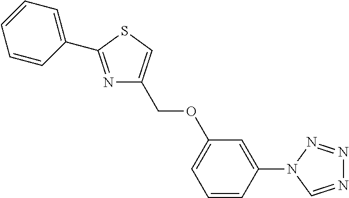 [SMILES: c1ccc(-c2nc(COc3cccc(-n4cnnn4)c3)cs2)cc1] |
| 55A | 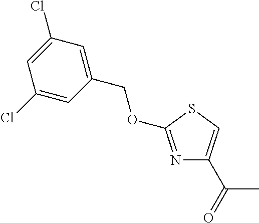 [SMILES: CC(=O)c1csc(OCc2cc(Cl)cc(Cl)c2)n1] |
| 56A | 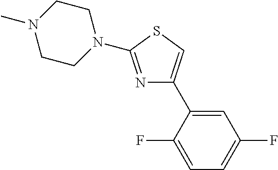 [SMILES: CN1CCN(c2nc(-c3cc(F)ccc3F)cs2)CC1] |
| 57A | 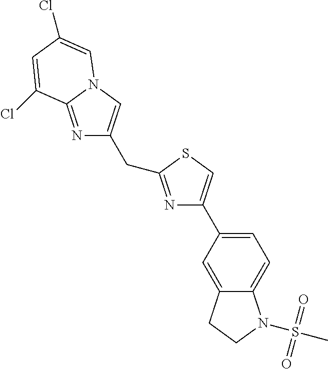 [SMILES: CS(=O)(=O)N1CCc2cc(-c3csc(Cc4cn5cc(Cl)cc(Cl)c5n4)n3)ccc21] |
| 58A | 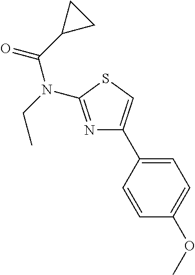 [SMILES: CCN(C(=O)C1CC1)c1nc(-c2ccc(OC)cc2)cs1] |
| 59A | 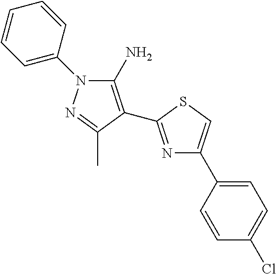 [SMILES: Cc1nn(-c2ccccc2)c(N)c1-c1nc(-c2ccc(Cl)cc2)cs1] |
| 60A | 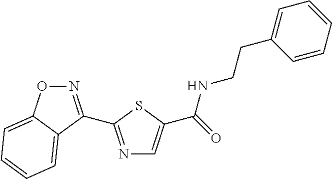 [SMILES: O=C(NCCc1ccccc1)c1cnc(-c2noc3ccccc23)s1] |
| 61A | 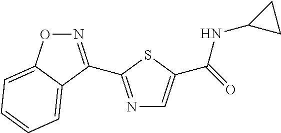 [SMILES: O=C(NC1CC1)c1cnc(-c2noc3ccccc23)s1] |
| 62A | 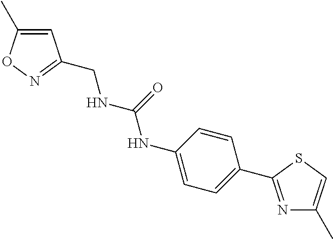 |
| 63A | 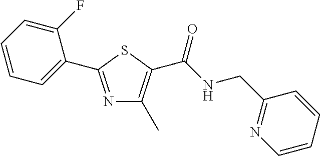 [SMILES: Cc1nc(-c2ccccc2F)sc1C(=O)NCc1ccccn1] |
| 64A | 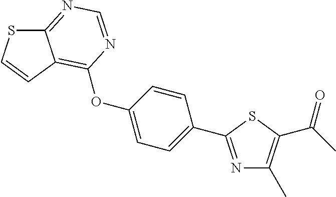 [SMILES: CC(=O)c1sc(-c2ccc(Oc3ncnc4sccc34)cc2)nc1C] |
| 65A | 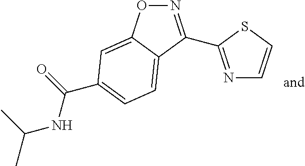 [SMILES: CC(C)NC(=O)c1ccc2c(-c3nccs3)noc2c1] |
| 66A | 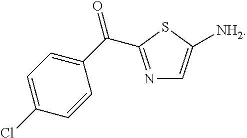 [SMILES: Nc1cnc(C(=O)c2ccc(Cl)cc2)s1] |

60. The compound of any one of the preceding claims, wherein the compound is selected from the group consisting of

| Compd. | |
|---|---|
| No. | Structure |
| 33B | 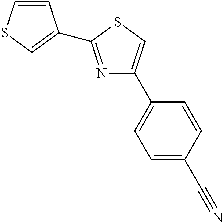 [SMILES: N#Cc1ccc(-c2csc(-c3ccsc3)n2)cc1] |
| 34B | 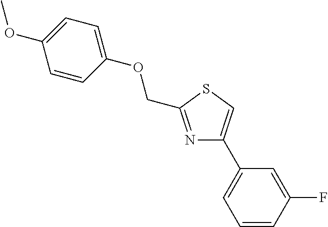 [SMILES: COc1ccc(OCc2nc(-c3cccc(F)c3)cs2)cc1] |
| 35B | 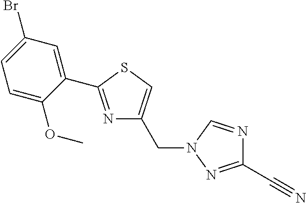 [SMILES: COc1ccc(Br)cc1-c1nc(Cn2cnc(C#N)n2)cs1] |
| 36B | 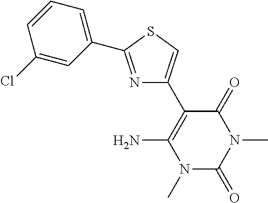 [SMILES: Cn1c(N)c(-c2csc(-c3cccc(Cl)c3)n2)c(=O)n(C)c1=O] |
| 37B | 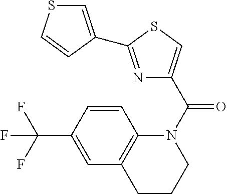 [SMILES: O=C(c1csc(-c2ccsc2)n1)N1CCCc2cc(C(F)(F)F)ccc21] |
| 38B | 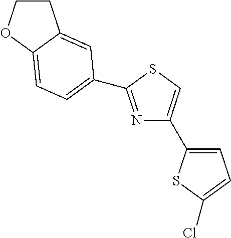 [SMILES: Clc1ccc(-c2csc(-c3ccc4c(c3)CCO4)n2)s1] |
| 39B | 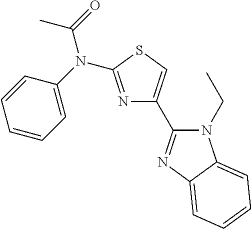 [SMILES: CCn1c(-c2csc(N(C(C)=O)c3ccccc3)n2)nc2ccccc21] |
| 40B | 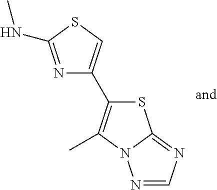 [SMILES: CNc1nc(-c2sc3ncnn3c2C)cs1] |
| 41B | 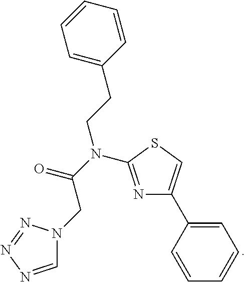 [SMILES: O=C(Cn1cnnn1)N(CCc1ccccc1)c1nc(-c2ccccc2)cs1] |

61. The compound of any one of the preceding claims, wherein A is 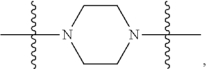 [SMILES: *N1CCN(*)CC1]
in which each [symbol] independently denotes the linkage between A and hydrogen, -L_{b}-B, -L_{c}-C, or a substituent.

62. The compound of any one of the preceding claims, wherein the compound is represented by Formula (Ic) 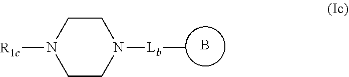
wherein
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S— **, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a piperazine nitrogen and ** denotes the linkage between L_{b} and B;
each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;
each R_{bb} independently is H, —C(O)R_{bba}, or a 6- to 10-membered aryl optionally substituted with one or more halogen, —CF₃, —CN, —OR_{bba}, or —NR_{bba}R_{bba}, in which each R_{bba} is independently H or C₁-C₆ alkyl;
R_{1c} is -L_{c}-C, C(O)R₂ₐ, or C(O)OR₂ₐ, wherein each R₂ₐ is C₁-C₆ alkyl;
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{bb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a piperazine nitrogen and ** denotes the linkage between L_{c} and C;
each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

63. The compound of any one of the preceding claims, wherein the compound is represented by Formula (Ic) and R_{1c} is selected from the group consisting of C(O)CH₃ and C(O)OCH₂CH₃.

64. The compound of any one of the preceding claims, wherein the compound is represented by Formula (Ic) and B is an optionally substituted monocyclic ring selected from the group consisting of benzene, pyrimidine, pyridine, thiophene, 1,3,5-triazine, 1,3,4-thiadiazole, 4,5-dihydrothiazole, and thiazol-4(5H)-one.

65. The compound of any one of the preceding claims, wherein the compound is represented by Formula (Ic) and B is an optionally substituted monocyclic ring selected from the group consisting of benzene, pyrimidine, pyridine, and thiophene.

66. The compound of any one of the preceding claims, wherein the compound is represented by Formula (Ic) and B is an optionally substituted bicyclic ring selected from the group consisting of benzo[d]isothiazaole, thieno[2,3-d]pyrimidine, pteridine, [1,2,4]triazolo[4,3-b]pyridazine, 5,6,7,8-tetrahydroquinazoline, 7,8-dihydroquinazolin-5(6H)-one, and 4a,6,7,7a-tetrahydro-5H-cyclopenta[b]pyridine.

67. The compound of any one of the preceding claims, wherein the compound is represented by Formula (Ic) and B is an optionally substituted bicyclic ring selected from the group consisting of benzo[d]isothiazaole and thieno[2,3-d]pyrimidine.

68. The compound of any one of the preceding claims, wherein the compound is represented by Formula (Ic) and L_{b} is selected from the group consisting of a covalent bond and *—SO₂—**.

69. The compound of any one of the preceding claims, wherein the compound is represented by Formula (Ic) and L_{b} is a covalent bond.

70. The compound of any one of the preceding claims, wherein the compound is represented by Formula (Ic), R_{1c} is -L_{c}-C and L_{c} is selected from the group consisting of a covalent bond, *—C(O)—**, *—N═CH₂—**, *—C(O)NH—**.

71. The compound of any one of the preceding claims, wherein the compound is represented by Formula (Ic), R₁ₐ is -L_{c}-C and C is an optionally substituted monocyclic ring selected from the group consisting of benzene, pyrimidine, and thiazole.

72. The compound of any one of the preceding claims, wherein the compound is represented by Formula (Ic), R₁ₐ is -L_{c}-C and C is an optionally substituted bicyclic ring selected from the group consisting of quinazoline and indole.

73. The compound of any one of the preceding claims, wherein the compound is represented by Formula (Ic), R₁ₐ is -L_{c}-C and C is an optionally substituted bicyclic ring selected from the group consisting of quinazoline and indole.

74. The compound of any one of the preceding claims, wherein the compound is selected from the group consisting of

| Compd. | |
|---|---|
| No. | Structure |
| 74 | 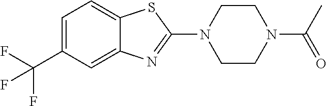 [SMILES: CC(=O)N1CCN(c2nc3cc(C(F)(F)F)ccc3s2)CC1] |
| 75 | 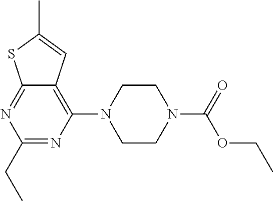 [SMILES: CCOC(=O)N1CCN(c2nc(CC)nc3sc(C)cc23)CC1] |
| 76 | 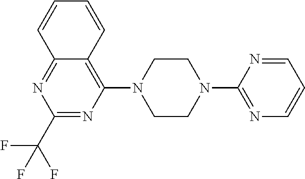 [SMILES: FC(F)(F)c1nc(N2CCN(c3ncccn3)CC2)c2ccccc2n1] |
| 77 | 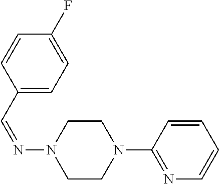 [SMILES: Fc1ccc(/C=N\N2CCN(c3ccccn3)CC2)cc1] |
| 78 | 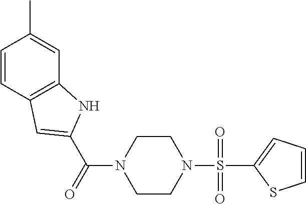 [SMILES: Cc1ccc2cc(C(=O)N3CCN(S(=O)(=O)c4cccs4)CC3)[nH]c2c1] |
| 79 | 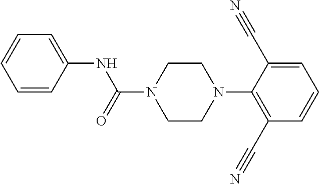 [SMILES: N#Cc1cccc(C#N)c1N1CCN(C(=O)Nc2ccccc2)CC1] |
| 80 | 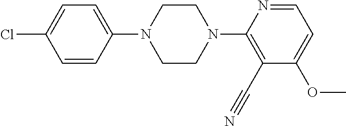 [SMILES: COc1ccnc(N2CCN(c3ccc(Cl)cc3)CC2)c1C#N] |
| 81 | 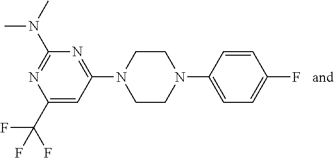 [SMILES: CN(C)c1nc(N2CCN(c3ccc(F)cc3)CC2)cc(C(F)(F)F)n1] |
| 111 | 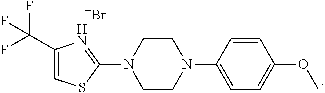 |

75. The compound of any one of the preceding claims, wherein the compound is selected from the group consisting of

| Compd. | |
|---|---|
| No. | Structure |
| 67A | 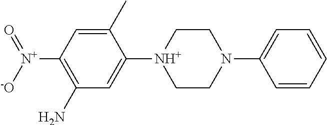 |
| 68A | 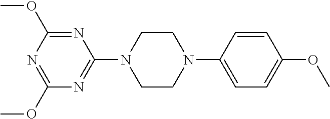 |
| 69A | 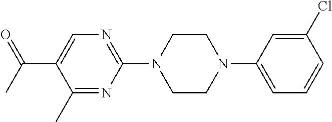 [SMILES: CC(=O)c1cnc(N2CCN(c3cccc(Cl)c3)CC2)nc1C] |
| 70A | 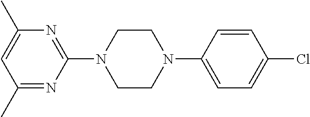 [SMILES: Cc1cc(C)nc(N2CCN(c3ccc(Cl)cc3)CC2)n1] |
| 71A | 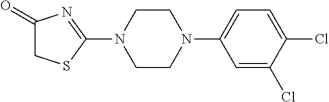 [SMILES: O=C1CSC(N2CCN(c3ccc(Cl)c(Cl)c3)CC2)=N1] |
| 72A | 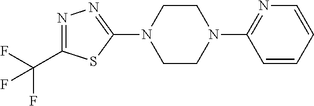 [SMILES: FC(F)(F)c1nnc(N2CCN(c3ccccn3)CC2)s1] |
| 73A | 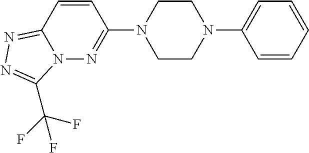 [SMILES: FC(F)(F)c1nnc2ccc(N3CCN(c4ccccc4)CC3)nn12] |
| 74A | 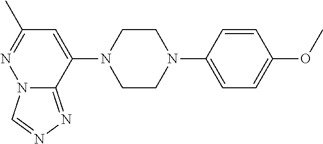 [SMILES: COc1ccc(N2CCN(c3cc(C)nn4cnnc34)CC2)cc1] |
| 75A | 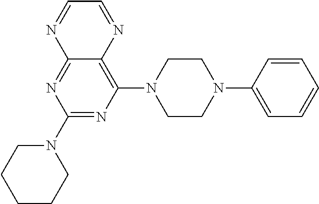 [SMILES: c1ccc(N2CCN(c3nc(N4CCCCC4)nc4nccnc34)CC2)cc1] |
| 76A | 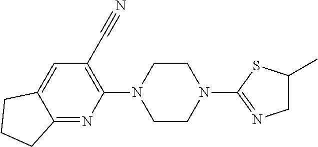 [SMILES: CC1CN=C(N2CCN(c3nc4c(cc3C#N)CCC4)CC2)S1] |
| 77A | 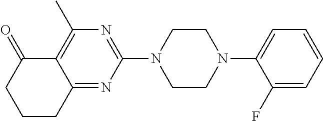 [SMILES: Cc1nc(N2CCN(c3ccccc3F)CC2)nc2c1C(=O)CCC2] |
| 78A | 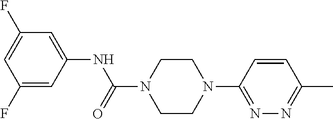 [SMILES: Cc1ccc(N2CCN(C(=O)Nc3cc(F)cc(F)c3)CC2)nn1] |
| 79A | 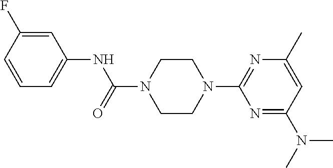 [SMILES: Cc1cc(N(C)C)nc(N2CCN(C(=O)Nc3cccc(F)c3)CC2)n1] |
| 80A | 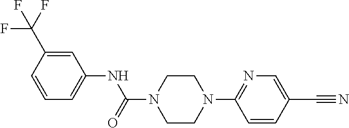 [SMILES: N#Cc1ccc(N2CCN(C(=O)Nc3cccc(C(F)(F)F)c3)CC2)nc1] |
| 81A | 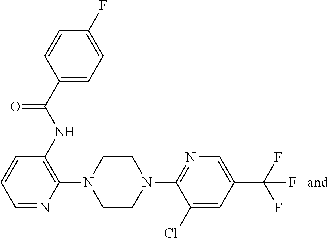 [SMILES: O=C(Nc1cccnc1N1CCN(c2ncc(C(F)(F)F)cc2Cl)CC1)c1ccc(F)cc1] |
| 82A | 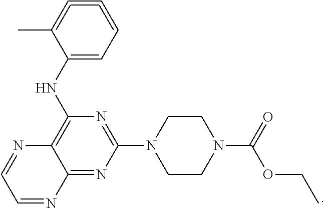 [SMILES: CCOC(=O)N1CCN(c2nc(NC3=CC=C=C=C3C)c3nccnc3n2)CC1] |

76. The compound of any one of the preceding claims, wherein the compound is

| Compd. | |
|---|---|
| No. | Structure |
| 42B | 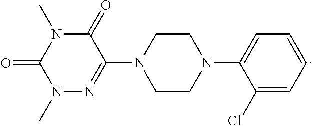 [SMILES: Cn1nc(N2CCN(c3ccccc3Cl)CC2)c(=O)n(C)c1=O] |

77. The compound of any one of the preceding claims, wherein the compound is represented by Formula (Id1) or Formula (Id2) 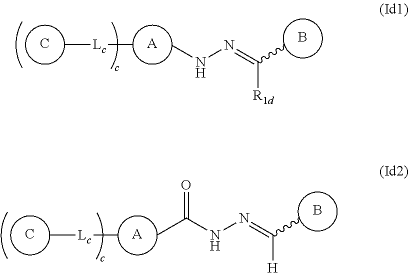
wherein
A is an optionally substituted monocyclic, bicyclic, or tricyclic ring selected from 6- to 14-membered aryl and 5- to 14-membered saturated or unsaturated heterocycle comprising 1-5 heteroatoms selected from N, O and S;
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
c is 0 or 1;
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and A and ** denotes the linkage between L_{c} and C;
each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl;
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl; and
R_{1d} is hydrogen or C₁-C₃ alkyl.

78. The compound of any one of the preceding claims, wherein the compound is represented by Formula (Id1) or Formula (Id2), c is 1, L_{c} is a covalent bond and C is an optionally substituted monocyclic ring selected from the group consisting of benzene and pyridine.

79. The compound of any one of the preceding claims, wherein the compound is represented by Formula (Id1) or Formula (Id2) and B is an optionally substituted benzene.

80. The compound of any one of the preceding claims, wherein the compound is represented by Formula (Id1) or Formula (Id2) and B is an optionally substituted benzofuran.

81. The compound of any one of the preceding claims, wherein the compound is represented by Formula (Id1) or Formula (Id2) and A is an optionally substituted monocyclic ring selected from the group consisting of pyrimidine, benzene, and thiazole.

82. The compound of any one of the preceding claims, wherein the compound is represented by Formula (Id1) or Formula (Id2) and A is an optionally substituted 4,5-dihydro-1H-benzo[g]indazole.

83. The compound of any one of the preceding claims, wherein the compound is selected from the group consisting of

| Compd. | |
|---|---|
| No. | Structure |
| 112 | 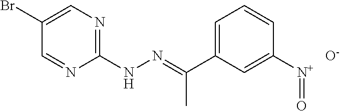 |
| 98 | 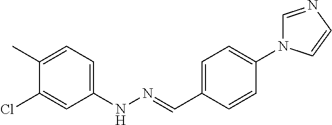 [SMILES: Cc1ccc(N/N=C/c2ccc(-n3ccnc3)cc2)cc1Cl] |
| 113 | 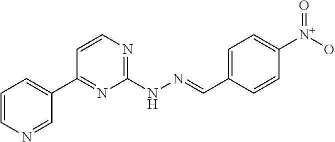 |
| 114 | 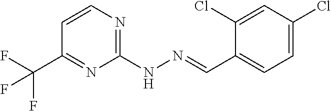 [SMILES: FC(F)(F)c1ccnc(N/N=C/c2ccc(Cl)cc2Cl)n1] |
| 99 | 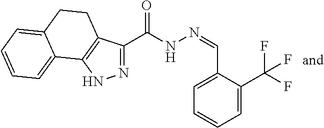 [SMILES: O=C(N/N=C\c1ccccc1C(F)(F)F)c1n[nH]c2c1CCc1ccccc1-2] |
| 115 | 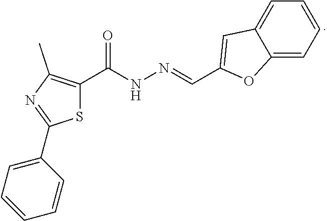 [SMILES: Cc1nc(-c2ccccc2)sc1C(=O)N/N=C/c1cc2ccccc2o1] |

84. The compound of any one of the preceding claims, wherein the compound is

| Compd. | |
|---|---|
| No. | Structure |
| 83A | 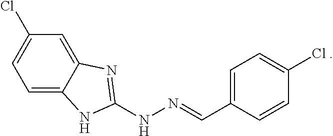 [SMILES: Clc1ccc(/C=N/Nc2nc3cc(Cl)ccc3[nH]2)cc1 ; "."] |

85. The compound of any one of the preceding claims, wherein the compound is selected from the group consisting of

| Compd. | |
|---|---|
| No. | Structure |
| 43B | 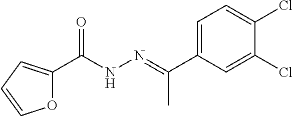 [SMILES: C/C(=N\NC(=O)c1ccco1)c1ccc(Cl)c(Cl)c1] |
| 44B | 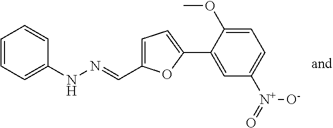 [SMILES: COc1ccc([N+](=O)[O-])cc1-c1ccc(/C=N/Nc2ccccc2)o1] |
| 45B | 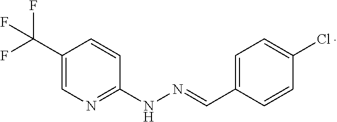 [SMILES: FC(F)(F)c1ccc(N/N=C/c2ccc(Cl)cc2)nc1] |

86. The compound of any one of the preceding claims, wherein A is 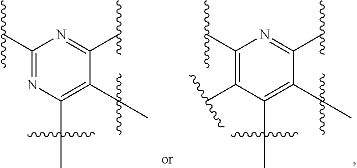 [SMILES: *c1nc(*)c(*)c(*)n1 ; *c1nc(*)c(*)c(*)c1*]
in which each [symbol] independently denotes the linkage between A and hydrogen, -L_{b}-B, -L_{c}-C, or a substituent.

87. The compound of any one of the preceding claims, wherein A is 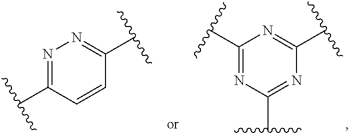 [SMILES: *c1ccc(*)nn1 ; *c1nc(*)nc(*)n1]
in which each [symbol] independently denotes the linkage between A and hydrogen, -L_{b}-B, -L_{c}-C, or a substituent.

88. The compound of any one of the preceding claims, wherein the compound is represented by Formula (Ie1) or Formula (Ie2) 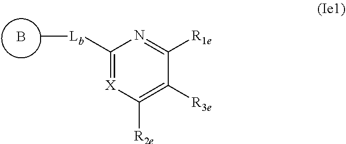 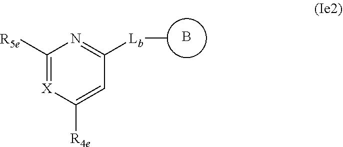
wherein
X is N or CR₆ₑ in which R₆ₑ is hydrogen, halogen, or —CN;
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)—**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR_{bb}—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a pyridine or pyrimidine carbon and ** denotes the linkage between L_{b} and B;
each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;
each R_{bb} independently is H, —C(O)R_{bba}, or a 6- to 10-membered aryl optionally substituted with one or more halogen, —CF₃, —CN, —OR_{ba}, or —NR_{bba}R_{bba}, in which each R_{bba} is independently H or C₁-C₆ alkyl;
R₁ₑ is hydrogen, —CF₃, or -L_{c}-C;
R₂ₑ is hydrogen, —CF₃, L_{c}-C, or 6-membered aryl optionally substituted with one or more halogen, —CF₃, or —CN;
R₃ₑ is hydrogen or when R₁ₑ is hydrogen and R₂ₑ is hydrogen R₃ₑ is L-C;
R₄ₑ is hydrogen or L_{c}-C;
R₅ₑ is hydrogen or L_{c}-C;
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N—**, *—C(O)NR_{cb}—**, *—NR_{cb}C(O)—**, *—S—R_{ca}—**, *—R_{ca}—S—**, *—O—R_{ca}—**, *—R_{ca}—O—**, *—C(O)NR_{cb}NR_{bb}C(O)—**, in which * denotes the linkage between L_{c} and a pyridine or pyrimidine carbon and ** denotes the linkage between L_{c} and C;
each R_{ca} independently is H or C₁-C₃ alkyl optionally substituted with one or more halogen, —CF₃, —CN, —OR_{caa}, or —NR_{caa}═R_{caa}, in which each R_{caa} is independently H or C₁-C₆ alkyl; and
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

89. The compound of any one of the preceding claims, wherein the compound is represented by Formula (Ie1) wherein X is N.

90. The compound of any one of the preceding claims, wherein the compound is represented by Formula (Ie1) or Formula (Ie2) and B is an optionally substituted monocyclic ring selected from the group consisting of pyrazole, benzene, and pyridine.

91. The compound of any one of the preceding claims, wherein the compound is represented by Formula (Ie1) or Formula (Ie2) and B is an optionally substituted indole.

92. The compound of any one of the preceding claims, wherein the compound is represented by Formula (Ie1) or Formula (Ie2) and C is an optionally substituted monocyclic ring selected from the group consisting of benzene and pyridine.

93. The compound of any one of the preceding claims, wherein the compound is represented by Formula (Ie1) or Formula (Ie2) and L_{b} is selected from the group consisting of a covalent bond, *—NH—**, and *—NHCH₂CH(OH)—**.

94. The compound of any one of the preceding claims, wherein the compound is represented by Formula (Ie1) or Formula (Ie2) and L_{b} is a covalent bond.

95. The compound of any one of the preceding claims, wherein the compound is represented by Formula (Ie1) or Formula (Ie2) wherein at least one of R₁ₑ, R₂ₑ, R₃ₑ, R₄ₑ and R₅ₑ is L_{c}-C and L is selected from the group consisting of a covalent bond, *—NH—**, and *—SCH₂—**.

96. The compound of any one of the preceding claims, wherein the compound is represented by Formula (le 1) or Formula (Ie2) wherein at least one of R₁ₑ, R₂ₑ, R₃ₑ, R₄ₑ and R₅ₑ is L_{c}-C and L_{c} is a covalent bond.

97. The compound of any one of the preceding claims, wherein the compound is selected from the group consisting of

| Compd. | |
|---|---|
| No. | Structure |
| 82 | 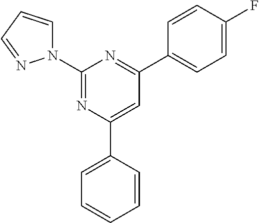 [SMILES: Fc1ccc(-c2cc(-c3ccccc3)nc(-n3cccn3)n2)cc1] |
| 83 | 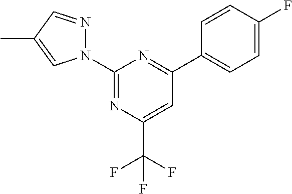 [SMILES: Cc1cnn(-c2nc(-c3ccc(F)cc3)cc(C(F)(F)F)n2)c1] |
| 84 | 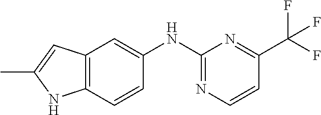 [SMILES: Cc1cc2cc(Nc3nccc(C(F)(F)F)n3)ccc2[nH]1] |
| 85 | 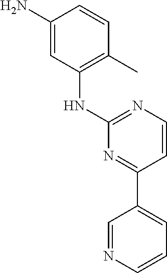 [SMILES: Cc1ccc(N)cc1Nc1nccc(-c2cccnc2)n1] |
| 86 | 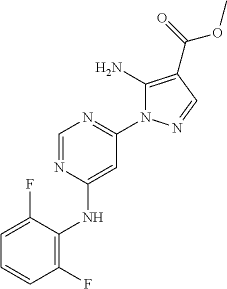 [SMILES: COC(=O)c1cnn(-c2cc(Nc3c(F)cccc3F)ncn2)c1N] |
| 87 | 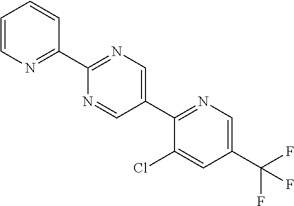 [SMILES: FC(F)(F)c1cnc(-c2cnc(-c3ccccn3)nc2)c(Cl)c1] |
| 88 | 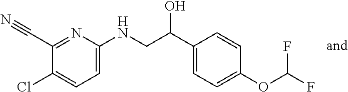 [SMILES: N#Cc1nc(NCC(O)c2ccc(OC(F)F)cc2)ccc1Cl] |
| 89 | 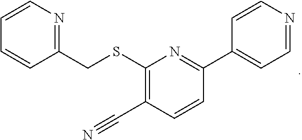 [SMILES: N#Cc1ccc(-c2ccncc2)nc1SCc1ccccn1] |

98. The compound of any one of the preceding claims, wherein the compound is selected from the group consisting of

| Compd. | |
|---|---|
| No. | Structure |
| 84A | 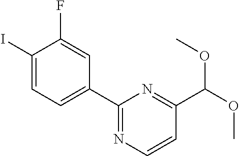 [SMILES: COC(OC)c1ccnc(-c2ccc(I)c(F)c2)n1] |
| 85A | 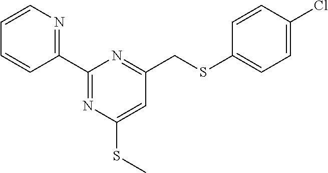 [SMILES: CSc1cc(CSc2ccc(Cl)cc2)nc(-c2ccccn2)n1] |
| 86A | 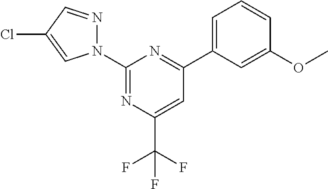 [SMILES: COc1cccc(-c2cc(C(F)(F)F)nc(-n3cc(Cl)cn3)n2)c1] |
| 87A | 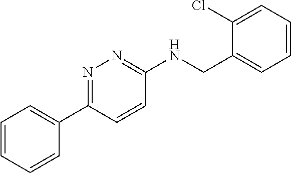 [SMILES: Clc1ccccc1CNc1ccc(-c2ccccc2)nn1] |
| 88A | 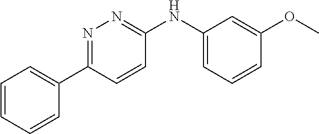 [SMILES: COc1cccc(Nc2ccc(-c3ccccc3)nn2)c1] |
| 89A | 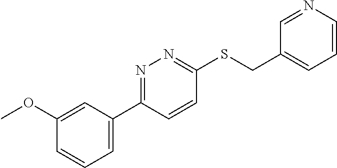 [SMILES: COc1cccc(-c2ccc(SCc3cccnc3)nn2)c1] |
| 90A | 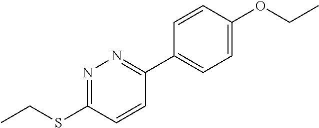 [SMILES: CCOc1ccc(-c2ccc(SCC)nn2)cc1] |
| 91A | 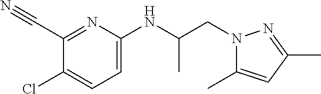 [SMILES: Cc1cc(C)n(CC(C)Nc2ccc(Cl)c(C#N)n2)n1] |
| 92A | 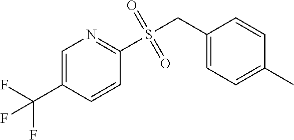 [SMILES: Cc1ccc(CS(=O)(=O)c2ccc(C(F)(F)F)cn2)cc1] |
| 93A | 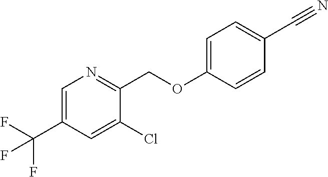 [SMILES: N#Cc1ccc(OCc2ncc(C(F)(F)F)cc2Cl)cc1] |
| 94A | 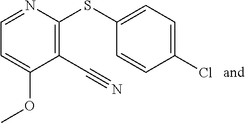 [SMILES: COc1ccnc(Sc2ccc(Cl)cc2)c1C#N] |
| 95A | 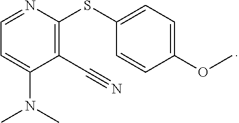 [SMILES: COc1ccc(Sc2nccc(N(C)C)c2C#N)cc1] |

99. The compound of any one of the preceding claims, wherein the compound is selected from the group consisting of

| Compd | |
|---|---|
| No. | Structure |
| 46B | 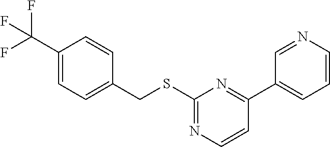 [SMILES: FC(F)(F)c1ccc(CSc2nccc(-c3cccnc3)n2)cc1] |
| 47B | 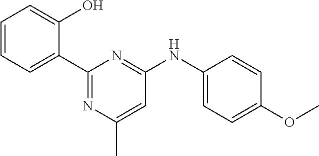 [SMILES: COc1ccc(Nc2cc(C)nc(-c3ccccc3O)n2)cc1] |
| 48B | 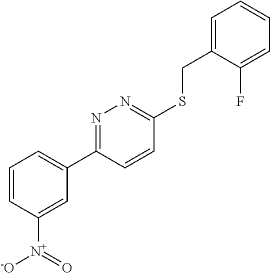 |
| 49B | 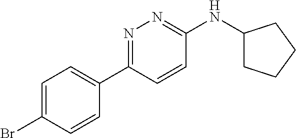 [SMILES: Brc1ccc(-c2ccc(NC3CCCC3)nn2)cc1] |
| 50B | 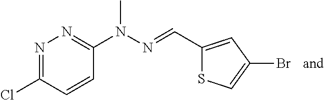 [SMILES: CN(/N=C/c1cc(Br)cs1)c1ccc(Cl)nn1] |
| 51B | 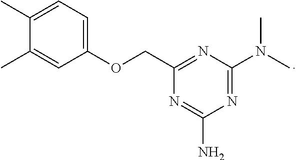 |

100. The compound of any one of the preceding claims, wherein the compound is represented by Formula (If) 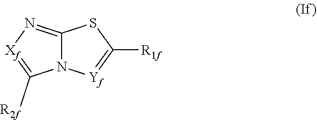
wherein
X_{f} is N or CR_{3f} in which R_{3f} is hydrogen, C₁-C₆ alkyl, or -L_{b}-B;
Y_{f} is N or CR_{4f} in which R_{4f} is hydrogen or C₁-C₆ alkyl;
B is an optionally substituted monocyclic, bicyclic, or tricyclic ring selected from 6- to 14-membered aryl and 5- to 14-membered saturated or unsaturated heterocyclyl comprising 1-5 heteroatoms selected from N, O and S;
L_{b} is a covalent bond, *—O—**, *—NR_{bb}—**, *—NR_{bb}C(O)NR_{bb}—**, *—C(O)—**, *—SO₂—**, *=N—**, *—N=**, *=N—C(O)**, *—C(O)—N=**, *—O—R_{ba}—**, *—R_{ba}—O—**, *—C(O)NR_{bb}—**, *—NR_{bb}C(O)—**, *—NR_{bb}—R_{ba}—(O)—**, *—O—R_{ba}—NR_{bb}—**, *—NR_{bb}—R_{ba}—**, *—R_{ba}—NR_{bb}—**, *—S—R_{ba}—**, *—R_{ba}—S—**, *—SO₂—R_{ba}—**, *—R_{ba}—SO₂—**, *—NR_{bb}—N═CR_{bb}—**, *—CR_{bb}═N—NR_{bb}—**, *—C(O)NR—N═CR_{bb}—**, *—CR_{bb}═N—NR_{bb}C(O)—**, *—O—R_{ba}—C(O)NR_{bb}—**, *NR_{bb}C(O)—R_{ba}—O—**, *—NR_{bb}—R_{ba}—C(O)NR_{bb}—**, *—NR_{bb}C(O)—R_{ba}—NR_{bb}—**, *—NR_{bb}C(O)O—R_{ba}—**, *—R_{ba}—OC(O)NR_{bb}—**, *—R_{ba}—NR_{bb}—R_{ba}—C(O)NR_{bb}—C(O)NR_{bb}—**, *—NR_{bb}C(O)—NR_{bb}C(O)—R_{ba}—NR_{bb}—R_{ba}—**, in which * denotes the linkage between L_{b} and a imidazo[2,1-b]thiazole or imidazo[2,1-b][1,3,4]thiadiazole carbon and ** denotes the linkage between L_{b} and B;
each R_{ba} independently is H or C₁-C₃ alkyl optionally substituted with one or more halogen, —CF₃, —CN, —OR_{baa}, —NR_{baa}R_{baa} in which each R_{baa} is independently H or C₁-C₆ alkyl;
each R_{bb} is independently H, —C(O)R_{bba}, or a 6- to 10-membered aryl optionally substituted with one or more halogen, —CF₃, —CN, —OR_{ba}, or —NR_{bba}R_{bba}, in which each R_{bba} is independently H or C₁-C₆ alkyl;
R_{1f} is CF₃, C₁-C₆ alkyl, -L_{b}-B, or C(O)NHR_{5f} in which R_{5f} is C₁-C₃ alkyl;
R_{2f} is hydrogen or -L_{b}-B when X_{f} is CR_{3f};
R_{2f} is hydrogen or -L_{c}-C when X_{f} is N;
C is an optionally substituted monocyclic or bicyclic ring selected from 6- to 10-membered aryl and 5- to 10-membered saturated or unsaturated heterocyclyl comprising 1-5 heteroatoms selected from N, O and S;
L_{c} is a covalent bond, *—NR_{cb}—**, *—R_{ca}—**, *—C(O)—**, *—SO₂—**, *—N═CR_{cb}—**, *—CR_{cb}═N-b-**C(O)N *, *—NR_{cb}C(O)—**, *—S—R_{ca}—**, *—R_{ca}—S—**, *—O—R_{ca}—**, *—R_{ca}—O—**, *—C(O)NR_{cb}NR_{cb}C(O)—**, in which * denotes the linkage between L_{c} and a [1,2,4]triazolo[3,4-b][1,3,4]thiadiazole carbon and ** denotes the linkage between L_{c} and C;
each R_{ca} independently is H or C₁-C₃ alkyl optionally substituted with one or more halogen, —CF₃, —CN, —OR_{caa}, or —NR_{caa}R_{caa}, in which each R_{caa} is independently H or C₁-C₆ alkyl; and
each R_{cb} independently is H, —C(O)R_{cba}, or a 6- to 10-membered aryl optionally substituted with one or more halogen, —CF₃, —CN, —OR_{cba}, or —NR_{cba}R_{cba}, in which each R_{cba} is independently H or C₁-C₆ alkyl.

101. The compound of any one of the preceding claims, wherein the compound is represented by Formula (If) and B is an optionally substituted monocyclic ring selected from the group consisting of benzene, pyridine, thiazole and pyrazole.

102. The compound of any one of the preceding claims, wherein the compound is represented by Formula (If) and B is an optionally substituted monocyclic ring selected from the group consisting of benzene, pyridine, and pyrazole.

103. The compound of any one of the preceding claims, wherein the compound is represented by Formula (If) and C is an optionally substituted monocyclic ring selected from the group consisting of pyrazole and thiophene.

104. The compound of any one of the preceding claims, wherein the compound is represented by Formula (If) and B is an optionally substituted bicyclic ring selected from the group consisting of 4,5,6,7-tetrahydrobenz[b]thiophene and 2-azabicyclo[2.2.1]heptane.

105. The compound of any one of the preceding claims, wherein the compound is represented by Formula (If) wherein Y_{f} is N and X_{f} is CR_{3f}.

106. The compound of any one of the preceding claims, wherein the compound is represented by Formula (If) wherein Y_{f} is N, X_{f} is —CCH₃ and R_{1f} is -L_{b}-B.

107. The compound of any one of the preceding claims, wherein the compound is represented by Formula (If) wherein L_{b} is a covalent bond.

108. The compound of any one of the preceding claims, wherein the compound is represented by Formula (If) wherein Y_{f} is N, X_{f} is —CCH₃ and R_{1f} is -L_{b}-B in which L_{b} is *—NHCH₂CH₂O—**.

109. The compound of any one of the preceding claims, wherein the compound is represented by Formula (If) wherein X_{f} is N and Y_{f} is N.

110. The compound of any one of the preceding claims, wherein the compound is represented by Formula (If) and L_{c} is a covalent bond.

111. The compound of any one of the preceding claims, wherein the compound is represented by Formula (If) wherein X_{f} is N, Y_{f} is N, and L_{c} is a covalent bond.

112. The compound of any one of the preceding claims, wherein the compound is selected from the group consisting of

| Compd. | |
|---|---|
| No. | Structure |
| 66 | 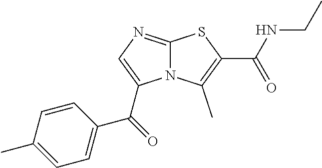 [SMILES: CCNC(=O)c1sc2ncc(C(=O)c3ccc(C)cc3)n2c1C] |
| 67 | 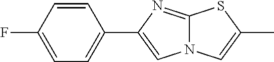 [SMILES: Cc1cn2cc(-c3ccc(F)cc3)nc2s1] |
| 68 | 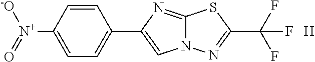 |
| 69 | 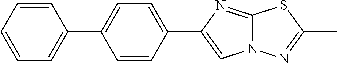 [SMILES: Cc1nn2cc(-c3ccc(-c4ccccc4)cc3)nc2s1] |
| 70 | 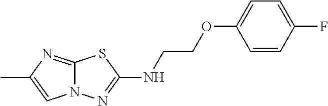 [SMILES: Cc1cn2nc(NCCOc3ccc(F)cc3)sc2n1] |
| 71 | 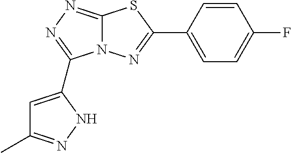 [SMILES: Cc1cc(-c2nnc3sc(-c4ccc(F)cc4)nn23)[nH]n1] |
| 72 | 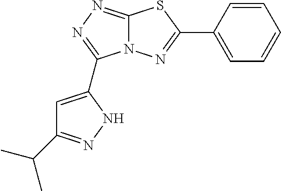 [SMILES: CC(C)c1cc(-c2nnc3sc(-c4ccccc4)nn23)[nH]n1] |
| 73 | 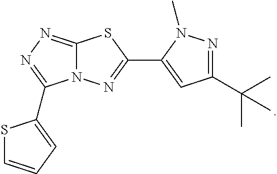 [SMILES: Cn1nc(C(C)(C)C)cc1-c1nn2c(-c3cccs3)nnc2s1] |

113. The compound of any one of the preceding claims, wherein the compound is selected from the group consisting of

| Compd. | |
|---|---|
| No. | Structure |
| 96A | 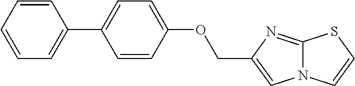 [SMILES: c1ccc(-c2ccc(OCc3cn4ccsc4n3)cc2)cc1] |
| 97A | 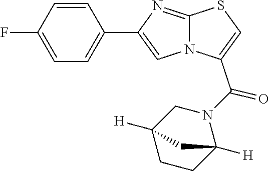 [SMILES: O=C(c1csc2nc(-c3ccc(F)cc3)cn12)N1C[C@@H]2CC[C@@H]1C2] |
| 97A1 | 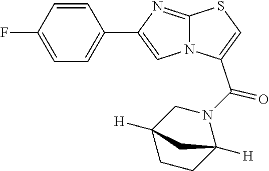 [SMILES: O=C(c1csc2nc(-c3ccc(F)cc3)cn12)N1C[C@H]2CC[C@@H]1C2] |
| 98A | 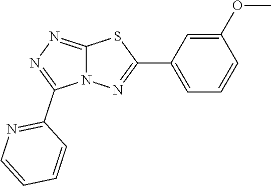 [SMILES: COc1cccc(-c2nn3c(-c4ccccn4)nnc3s2)c1] |
| 99A | 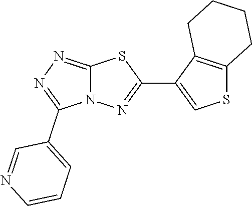 [SMILES: c1cncc(-c2nnc3sc(-c4csc5c4CCCC5)nn23)c1] |
| 97A2 | 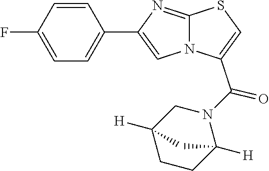 [SMILES: O=C(c1csc2nc(-c3ccc(F)cc3)cn12)N1C[C@@H]2CC[C@H]1C2] |

114. The compound of any one of the preceding claims, wherein the compound is

| Compd. | |
|---|---|
| No. | Structure |
| 40B | 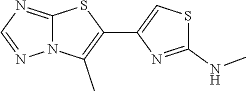 [SMILES: CNc1nc(-c2sc3ncnn3c2C)cs1] |

115. The compound of any one of the preceding claims, wherein the compound is represented by at least one formula selected from the group consisting of Ia, Ib, Ic, Id1, Id2, Ie1, Ie2, and If.

116. The compound of any one of the preceding claims, wherein A is an optionally substituted monocyclic 5-membered heterocycle comprising 2-4 nitrogen heteroatoms.

117. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole.

118. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted benzene.

119. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole and B is an optionally substituted bicyclic ring selected from the group consisting of benzo[d]isothiazole and naphthalene.

120. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted monocyclic ring selected from the group consisting of benzene, thiophene, and furan.

121. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole and C is an optionally substituted 1,2,3,4-tetrahydronaphthalene.

122. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole and L_{b} is selected from the group consisting of a covalent bond, *—SCH₂—**, and *—R_{ba}—NR_{bb}—**.

123. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole, L_{b} is a covalent bond and B is an optionally substituted benzene.

124. The compound of any one of the preceding claims, wherein A is an optionally substituted monocyclic 5-membered heterocycle comprising 2-4 nitrogen heteroatoms selected from the group consisting of pyrazole, 1,2,3-triazole, 1,2,4-triazole, and tetrazole, L_{C} is a covalent bond, *—C(O)—**, or *—C(O)NHNHC(O)—**.

125. The compound of any one of the preceding claims, wherein the compound is selected from the group consisting of

| Compd. | |
|---|---|
| No. | Structure |
| 90 | 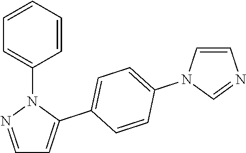 [SMILES: c1ccc(-n2nccc2-c2ccc(-n3ccnc3)cc2)cc1] |
| 91 | 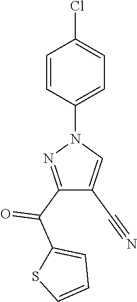 [SMILES: N#Cc1cn(-c2ccc(Cl)cc2)nc1C(=O)c1cccs1] |
| 92 | 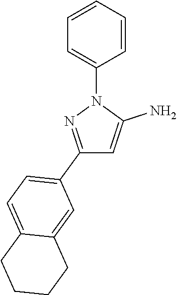 [SMILES: Nc1cc(-c2ccc3c(c2)CCCC3)nn1-c1ccccc1] |
| 93 | 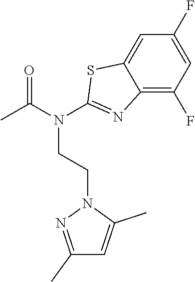 [SMILES: CC(=O)N(CCn1nc(C)cc1C)c1nc2c(F)cc(F)cc2s1] |
| 94 | 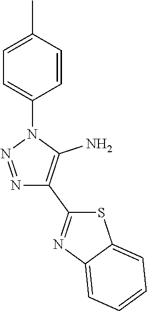 [SMILES: Cc1ccc(-n2nnc(-c3nc4ccccc4s3)c2N)cc1] |
| 95 | 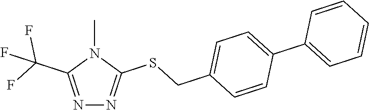 [SMILES: Cn1c(SCc2ccc(-c3ccccc3)cc2)nnc1C(F)(F)F] |
| 96 | 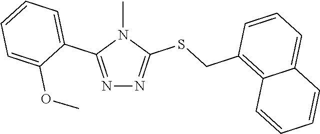 [SMILES: COc1ccccc1-c1nnc(SCc2cccc3ccccc23)n1C] |
| 97 | 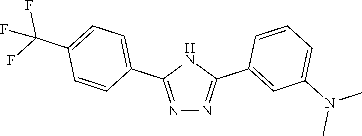 [SMILES: CN(C)c1cccc(-c2nnc(-c3ccc(C(F)(F)F)cc3)[nH]2)c1] |
| 139 | 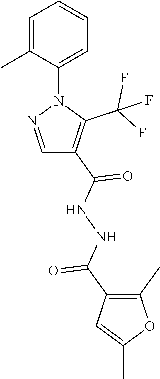 |
| | and |
| 154 | 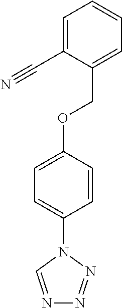 [SMILES: N#Cc1ccccc1COc1ccc(-n2cnnn2)cc1] |

126. The compound of any one of the preceding claims, wherein the compound is selected from the group consisting of

| Compd. | |
|---|---|
| No. | Structure |
| 100A | 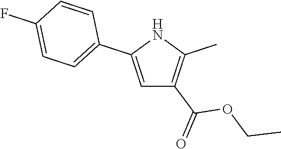 [SMILES: CCOC(=O)c1cc(-c2ccc(F)cc2)[nH]c1C] |
| 101A | 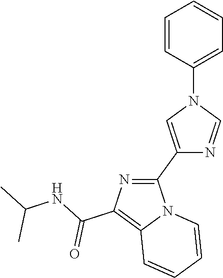 [SMILES: CC(C)NC(=O)c1nc(-c2cn(-c3ccccc3)cn2)n2ccccc12] |
| 102A | 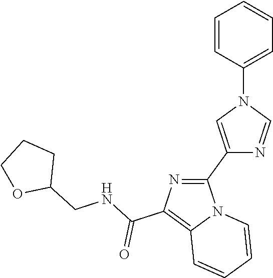 [SMILES: O=C(NCC1CCCO1)c1nc(-c2cn(-c3ccccc3)cn2)n2ccccc12] |
| 103A | 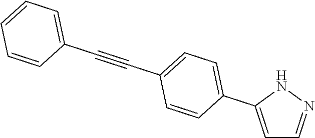 [SMILES: C(#Cc1ccc(-c2ccn[nH]2)cc1)c1ccccc1] |
| 104A | 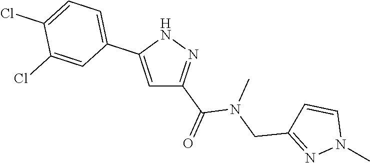 [SMILES: CN(Cc1ccn(C)n1)C(=O)c1cc(-c2ccc(Cl)c(Cl)c2)[nH]n1] |
| 105A | 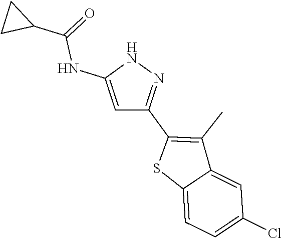 [SMILES: Cc1c(-c2cc(NC(=O)C3CC3)[nH]n2)sc2ccc(Cl)cc12] |
| 106A | 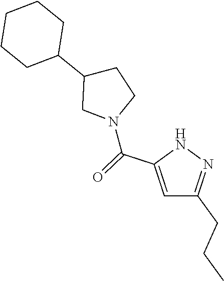 [SMILES: CCCc1cc(C(=O)N2CCC(C3CCCCC3)C2)[nH]n1] |
| 107A | 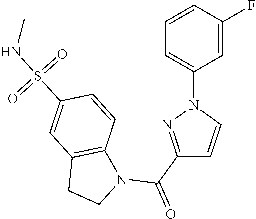 [SMILES: CNS(=O)(=O)c1ccc2c(c1)CCN2C(=O)c1ccn(-c2cccc(F)c2)n1] |
| 108A | 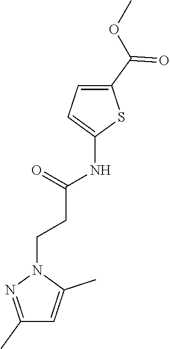 [SMILES: COC(=O)c1ccc(NC(=O)CCn2nc(C)cc2C)s1] |
| 109A | 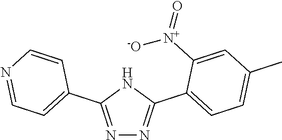 |
| | and |
| 110A | 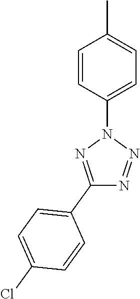 [SMILES: Cc1ccc(-n2nnc(-c3ccc(Cl)cc3)n2)cc1] |

127. The compound of any one of the preceding claims, wherein the compound is selected from the group consisting of

| Compd. | |
|---|---|
| No. | Structure |
| 52B | 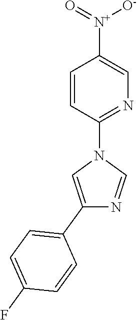 [SMILES: O=[N+]([O-])c1ccc(-n2cnc(-c3ccc(F)cc3)c2)nc1] |
| 53B | 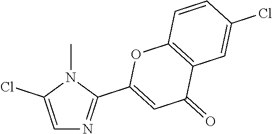 [SMILES: Cn1c(Cl)cnc1-c1cc(=O)c2cc(Cl)ccc2o1] |
| 54B | 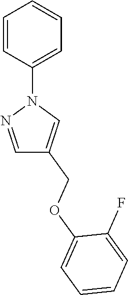 [SMILES: Fc1ccccc1OCc1cnn(-c2ccccc2)c1] |
| 55B | 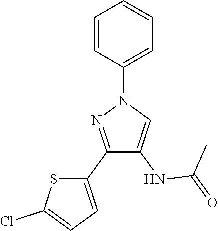 [SMILES: CC(=O)Nc1cn(-c2ccccc2)nc1-c1ccc(Cl)s1] |
| 56B | 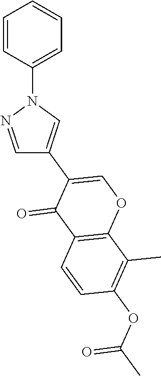 [SMILES: CC(=O)Oc1ccc2c(=O)c(-c3cnn(-c4ccccc4)c3)coc2c1C] |
| 57B | 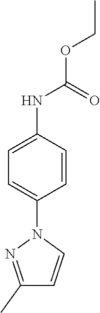 [SMILES: CCOC(=O)Nc1ccc(-n2ccc(C)n2)cc1] |
| 58B | 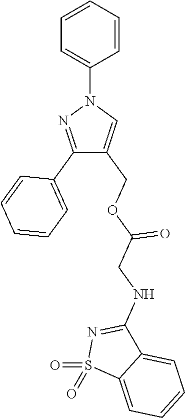 [SMILES: O=C(CNC1=NS(=O)(=O)c2ccccc21)OCc1cn(-c2ccccc2)nc1-c1ccccc1] |
| 59B | 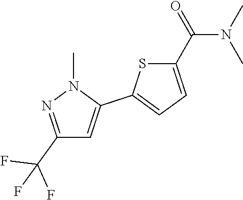 [SMILES: CN(C)C(=O)c1ccc(-c2cc(C(F)(F)F)nn2C)s1] |
| 60B | 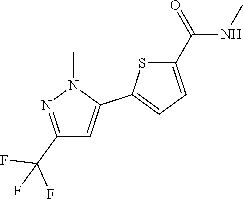 [SMILES: CNC(=O)c1ccc(-c2cc(C(F)(F)F)nn2C)s1] |
| | and |
| 61B | 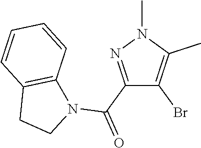 [SMILES: Cc1c(Br)c(C(=O)N2CCc3ccccc32)nn1C] |

128. The compound of any one of the preceding claims, wherein A is an optionally substituted bicyclic 9-membered heterocycle comprising 2-5 nitrogen heteroatoms.

129. The compound of any one of the preceding claims, wherein A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, and tetrazolo[1,5-b]pyridazine.

130. The compound of any one of the preceding claims, wherein A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted monocyclic ring selected from thiophene, pyrrole, benzene, pyridine, imidazole, and 1,2,3,4-tetrahydropyridine.

131. The compound of any one of the preceding claims, wherein A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and B is an optionally substituted indole.

132. The compound of any one of the preceding claims, wherein A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and C is an optionally substituted benzene.

133. The compound of any one of the preceding claims, wherein A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and Lt is selected from the group consisting of a covalent bond, *—NH—**, and *—SCH₂—**.

134. The compound of any one of the preceding claims, wherein A is an optionally substituted bicyclic 9-membered heterocycle comprising 2-5 nitrogen heteroatoms selected from the group consisting of imidazo[1,2-a]pyridine, pyrazolo[1,5-a]pyrimidine, pyrazolo[5,4-b]pyridine, pyrazolo[5,1-c][1,2,4]triazine, [1,2,4]triazolo[1,5-a]pyrimidine, [1,2,4]triazolo[4,3-b]pyridazine, tetrazolo[1,5-b]pyridazine, and 7H-[1,2,4]triazolo[5,1-b]pyrimidine and L_{c} is a covalent bond.

135. The compound of any one of the preceding claims, wherein the compound is selected from the group consisting of

| Compd. | |
|---|---|
| No. | Structure |
| 100 | 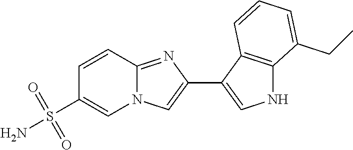 [SMILES: CCc1cccc2c(-c3cn4cc(S(N)(=O)=O)ccc4n3)c[nH]c12] |
| 101 | 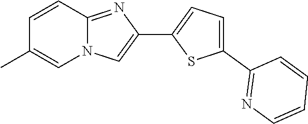 [SMILES: Cc1ccc2nc(-c3ccc(-c4ccccn4)s3)cn2c1] |
| 102 | 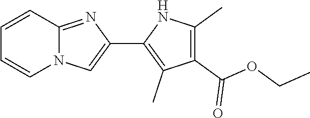 [SMILES: CCOC(=O)c1c(C)[nH]c(-c2cn3ccccc3n2)c1C] |
| 103 | 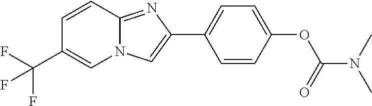 [SMILES: CN(C)C(=O)Oc1ccc(-c2cn3cc(C(F)(F)F)ccc3n2)cc1] |
| 104 | 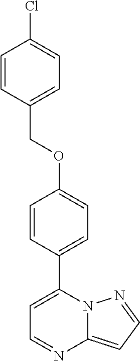 [SMILES: Clc1ccc(COc2ccc(-c3ccnc4ccnn34)cc2)cc1] |
| 105 | 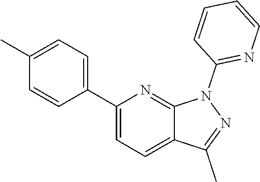 [SMILES: Cc1ccc(-c2ccc3c(C)nn(-c4ccccn4)c3n2)cc1] |
| 106 | 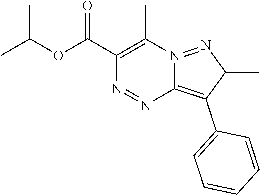 |
| 107 | 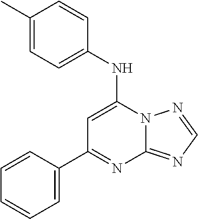 [SMILES: Cc1ccc(Nc2cc(-c3ccccc3)nc3ncnn23)cc1] |
| 108 | 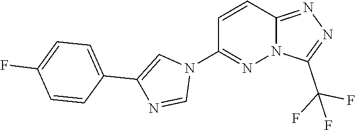 [SMILES: Fc1ccc(-c2cn(-c3ccc4nnc(C(F)(F)F)n4n3)cn2)cc1] |
| 110 | 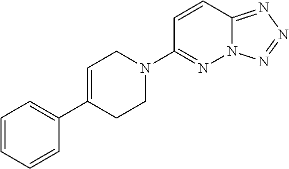 [SMILES: C1=C(c2ccccc2)CCN(c2ccc3nnnn3n2)C1] |
| | and |
| 125 | 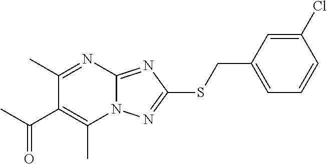 [SMILES: CC(=O)c1c(C)nc2nc(SCc3cccc(Cl)c3)nn2c1C] |

136. The compound of any one of the preceding claims, wherein the compound is selected from the group consisting of

| Compd. | |
|---|---|
| No. | Structure |
| 111A | 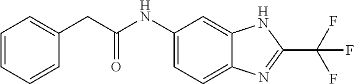 [SMILES: O=C(Cc1ccccc1)Nc1ccc2nc(C(F)(F)F)[nH]c2c1] |
| 112A | 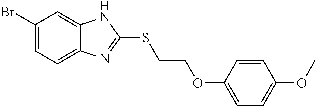 [SMILES: COc1ccc(OCCSc2nc3ccc(Br)cc3[nH]2)cc1] |
| 113A | 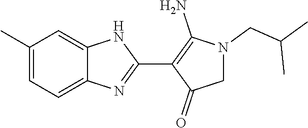 [SMILES: Cc1ccc2nc(C3=C(N)N(CC(C)C)CC3=O)[nH]c2c1] |
| 114A | 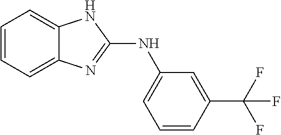 [SMILES: FC(F)(F)c1cccc(Nc2nc3ccccc3[nH]2)c1] |
| 115A | 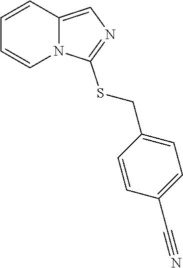 [SMILES: N#Cc1ccc(CSc2ncc3ccccn23)cc1] |
| 116A | 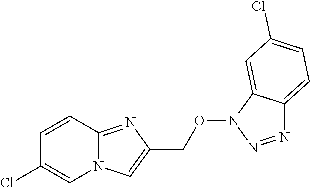 [SMILES: Clc1ccc2nnn(OCc3cn4cc(Cl)ccc4n3)c2c1] |
| 117A | 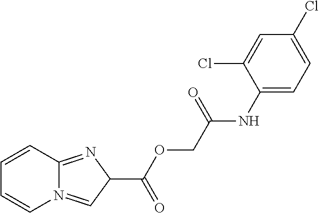 |
| 118A | 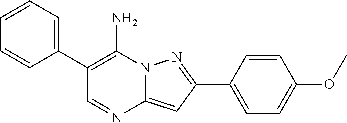 [SMILES: COc1ccc(-c2cc3ncc(-c4ccccc4)c(N)n3n2)cc1] |
| 119A | 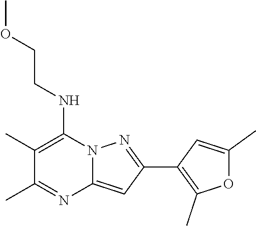 |
| 120A | 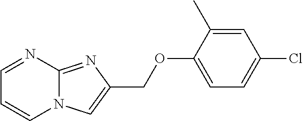 [SMILES: Cc1cc(Cl)ccc1OCc1cn2cccnc2n1] |
| 121A | 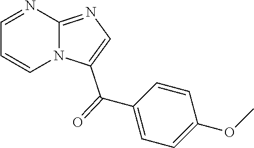 [SMILES: COc1ccc(C(=O)c2cnc3ncccn23)cc1] |
| 122A | 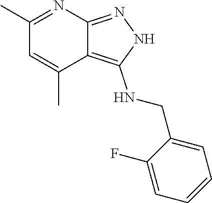 [SMILES: Cc1cc(C)c2c(NCc3ccccc3F)[nH]nc2n1] |
| 123A | 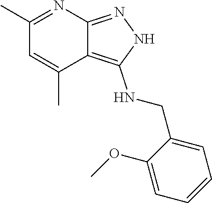 [SMILES: COc1ccccc1CNc1[nH]nc2nc(C)cc(C)c12] |
| 124A | 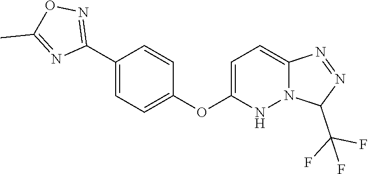 [SMILES: Cc1nc(-c2ccc(OC3=CC=C4N=NC(C(F)(F)F)N4N3)cc2)no1] |
| 125A | 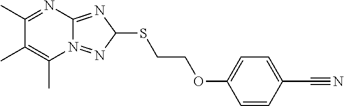 |
| | and |
| 126A | 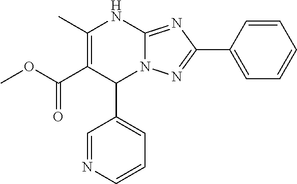 [SMILES: COC(=O)C1=C(C)Nc2nc(-c3ccccc3)nn2C1c1cccnc1] |

137. The compound of any one of the preceding claims, wherein the compound is selected from the group consisting of

| Compd. | |
|---|---|
| No. | Structure |
| 62B | 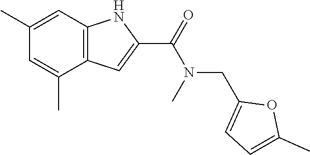 |
| 63B | 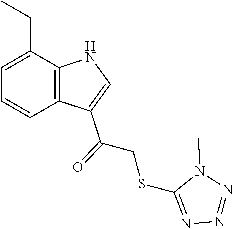 [SMILES: CCc1cccc2c(C(=O)CSc3nnnn3C)c[nH]c12] |
| 64B | 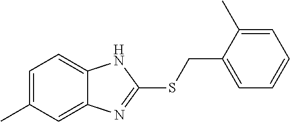 [SMILES: Cc1ccc2[nH]c(SCc3ccccc3C)nc2c1] |
| 65B | 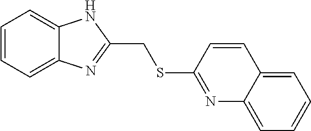 [SMILES: c1ccc2nc(SCc3nc4ccccc4[nH]3)ccc2c1] |
| 66B | 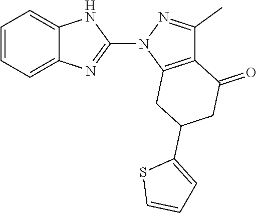 [SMILES: Cc1nn(-c2nc3ccccc3[nH]2)c2c1C(=O)CC(c1cccs1)C2] |
| 67B | 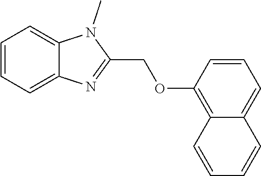 [SMILES: Cn1c(COc2cccc3ccccc23)nc2ccccc21] |
| 68B | 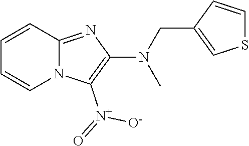 [SMILES: CN(Cc1ccsc1)c1nc2ccccn2c1[N+](=O)[O-]] |
| 69B | 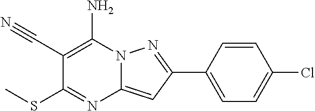 [SMILES: CSc1nc2cc(-c3ccc(Cl)cc3)nn2c(N)c1C#N] |
| 70B | 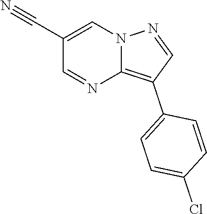 [SMILES: N#Cc1cnc2c(-c3ccc(Cl)cc3)cnn2c1] |
| 71B | 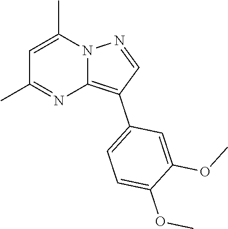 |
| 72B | 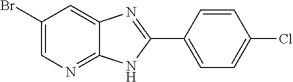 [SMILES: Clc1ccc(-c2nc3cc(Br)cnc3[nH]2)cc1] |
| 73B | 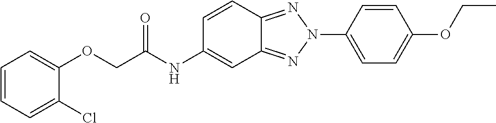 [SMILES: CCOc1ccc(-n2nc3ccc(NC(=O)COc4ccccc4Cl)cc3n2)cc1] |
| 74B | 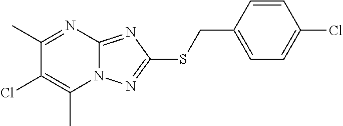 [SMILES: Cc1nc2nc(SCc3ccc(Cl)cc3)nn2c(C)c1Cl] |
| | and |
| 75B | 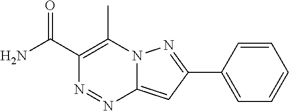 [SMILES: Cc1c(C(N)=O)nnc2cc(-c3ccccc3)nn12] |

138. The compound of any one of the preceding claims, wherein A is an optionally substituted monocyclic 5-membered heterocycle comprising 1 oxygen heteroatom and 1-2 nitrogen heteroatoms.

139. The compound of any one of the preceding claims, wherein A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole.

140. The compound of any one of the preceding claims, wherein A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and B is an optionally substituted monocyclic ring selected from isoxazole, pyridine, pyrazine, thiophene, and benzene.

141. The compound of any one of the preceding claims, wherein A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and C is an optionally substituted monocyclic ring selected from pyrazole and benzene.

142. The compound of any one of the preceding claims, wherein A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and L is selected from the group consisting of a covalent bond and *—CH₂NH—**.

143. The compound of any one of the preceding claims, wherein A is an optionally substituted monocyclic 5-membered heterocycle comprising 1 oxygen heteroatom and 1-2 nitrogen heteroatoms selected from the group consisting of oxazole, 1,3,4-oxadiazole, and 1,2,4-oxadiazole and L_{c} is a covalent bond.

144. The compound of any one of the preceding claims, wherein the compound is selected from the group consisting of

| Compd. | |
|---|---|
| No. | Structure |
| 122 | 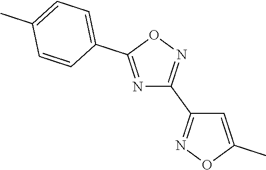 |
| 123 | 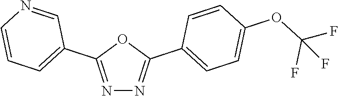 [SMILES: FC(F)(F)Oc1ccc(-c2nnc(-c3cccnc3)o2)cc1] |
| 140 | 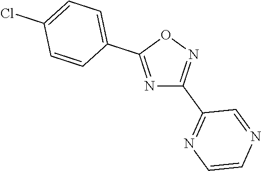 [SMILES: Clc1ccc(-c2nc(-c3cnccn3)no2)cc1] |
| 141 | 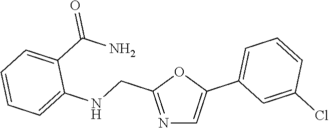 [SMILES: NC(=O)c1ccccc1NCc1ncc(-c2cccc(Cl)c2)o1] |
| 144 | 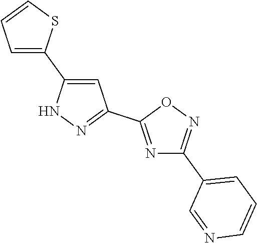 [SMILES: c1cncc(-c2noc(-c3cc(-c4cccs4)[nH]n3)n2)c1] |
| 149 | 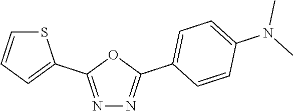 [SMILES: CN(C)c1ccc(-c2nnc(-c3cccs3)o2)cc1] |
| | and |
| 157 | 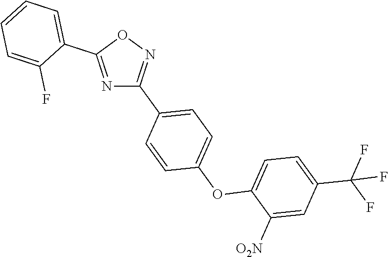 [SMILES: O=[N+]([O-])c1cc(C(F)(F)F)ccc1Oc1ccc(-c2noc(-c3ccccc3F)n2)cc1] |

145. The compound of any one of the preceding claims, wherein the compound is selected from the group consisting of

| Compd. | |
|---|---|
| No. | Structure |
| 127A | 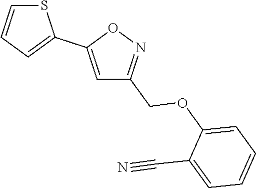 [SMILES: N#Cc1ccccc1OCc1cc(-c2cccs2)on1] |
| 128A | 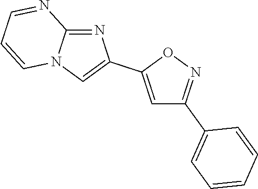 [SMILES: c1ccc(-c2cc(-c3cn4cccnc4n3)on2)cc1] |
| 129A | 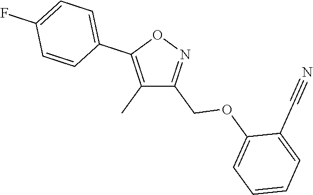 [SMILES: Cc1c(COc2ccccc2C#N)noc1-c1ccc(F)cc1] |
| 130A | 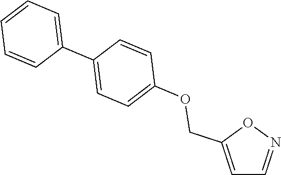 [SMILES: c1ccc(-c2ccc(OCc3ccno3)cc2)cc1] |
| 131A | 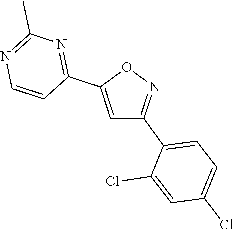 [SMILES: Cc1nccc(-c2cc(-c3ccc(Cl)cc3Cl)no2)n1] |
| 132A | 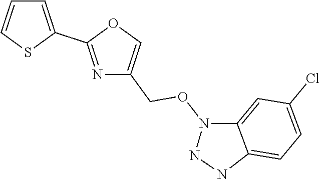 [SMILES: Clc1ccc2c(c1)N(OCc1coc(-c3cccs3)n1)NN2] |
| 133A | 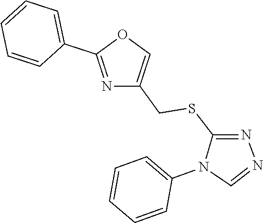 [SMILES: c1ccc(-c2nc(CSc3nncn3-c3ccccc3)co2)cc1] |
| 134A | 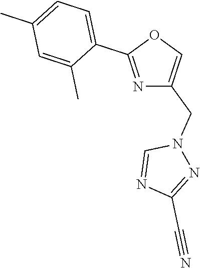 [SMILES: Cc1ccc(-c2nc(Cn3cnc(C#N)n3)co2)c(C)c1] |
| 135A | 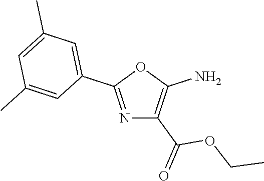 [SMILES: CCOC(=O)c1nc(-c2cc(C)cc(C)c2)oc1N] |
| 136A | 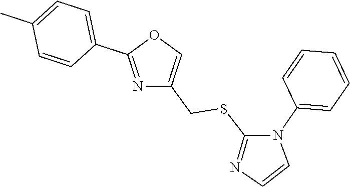 [SMILES: Cc1ccc(-c2nc(CSc3nccn3-c3ccccc3)co2)cc1] |
| 137A | 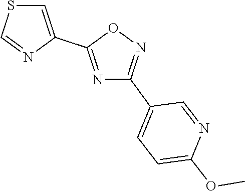 [SMILES: COc1ccc(-c2noc(-c3cscn3)n2)cn1] |
| 138A | 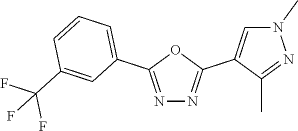 [SMILES: Cc1nn(C)cc1-c1nnc(-c2cccc(C(F)(F)F)c2)o1] |
| 139A | 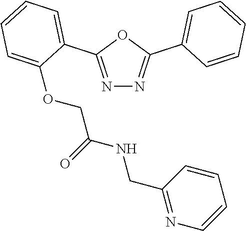 [SMILES: O=C(COc1ccccc1-c1nnc(-c2ccccc2)o1)NCc1ccccn1] |
| | and |
| 140A | 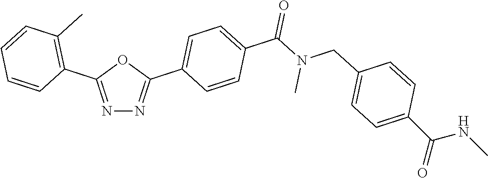 [SMILES: CNC(=O)c1ccc(CN(C)C(=O)c2ccc(-c3nnc(-c4ccccc4C)o3)cc2)cc1] |

146. The compound of any one of the preceding claims, wherein the compound is selected from the group consisting of

| Compd. | |
|---|---|
| No. | Structure |
| 76B | 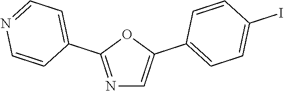 [SMILES: Ic1ccc(-c2cnc(-c3ccncc3)o2)cc1] |
| 77B | 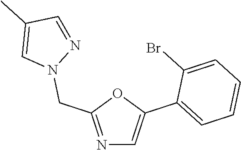 [SMILES: Cc1cnn(Cc2ncc(-c3ccccc3Br)o2)c1] |
| 78B | 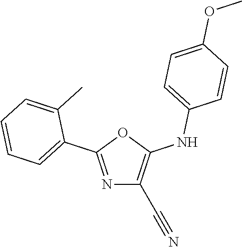 [SMILES: COc1ccc(Nc2oc(-c3ccccc3C)nc2C#N)cc1] |
| 79B | 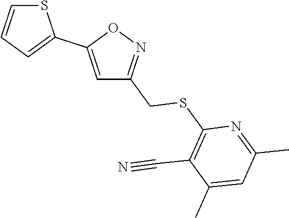 [SMILES: Cc1cc(C)c(C#N)c(SCc2cc(-c3cccs3)on2)n1] |
| 80B | 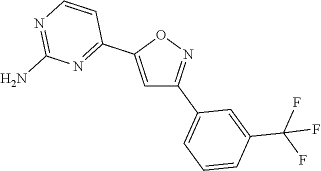 [SMILES: Nc1nccc(-c2cc(-c3cccc(C(F)(F)F)c3)no2)n1] |
| 81B | 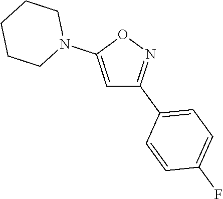 [SMILES: Fc1ccc(-c2cc(N3CCCCC3)on2)cc1] |
| 82B | 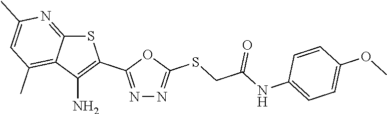 |
| 83B | 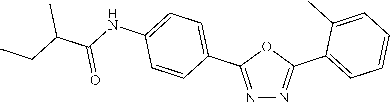 [SMILES: CCC(C)C(=O)Nc1ccc(-c2nnc(-c3ccccc3C)o2)cc1] |
| 84B | 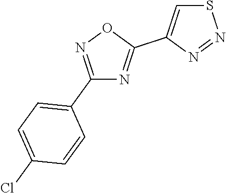 [SMILES: Clc1ccc(-c2noc(-c3csnn3)n2)cc1] |
| 85B | 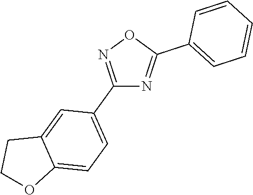 [SMILES: c1ccc(-c2nc(-c3ccc4c(c3)CCO4)no2)cc1] |
| 86B | 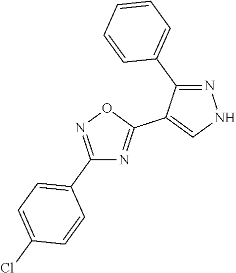 [SMILES: Clc1ccc(-c2noc(-c3c[nH]nc3-c3ccccc3)n2)cc1] |
| | and |
| 87B | 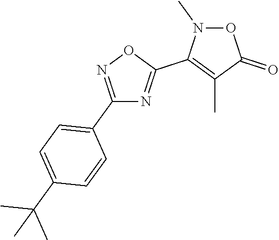 [SMILES: Cc1c(-c2nc(-c3ccc(C(C)(C)C)cc3)no2)n(C)oc1=O] |

147. The compound of any one of the preceding claims, wherein A is an optionally substituted benzene.

148. The compound of any one of the preceding claims, wherein A is an optionally substituted benzene and B is an optionally substituted monocyclic ring selected from the group consisting of benzene, thiophene, 2,3-dihydrothiazole, and 1,2,3,6-tetrahydropyridine.

149. The compound of any one of the preceding claims, wherein A is an optionally substituted benzene and C is an optionally substituted monocyclic ring selected from the group consisting of benzene and isoxazole.

150. The compound of any one of the preceding claims, wherein A is an optionally substituted benzene and L_{b} is selected from the group consisting of a covalent bond, *—C(O)—N=**, *—OCH₂C(O)NH—**, and *—NHC(O)CH₂NH—**.

151. The compound of any one of the preceding claims, wherein A is an optionally substituted benzene and L_{c} is *OCH₂—**.

152. The compound of any one of the preceding claims, wherein the compound is selected from the group consisting of

| Compd. | |
|---|---|
| No. | Structure |
| 118 | 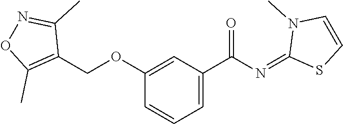 [SMILES: Cc1noc(C)c1COc1cccc(C(=O)/N=c2/sccn2C)c1] |
| 119 | 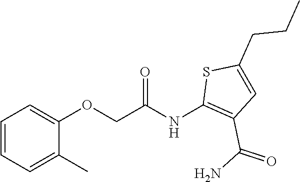 [SMILES: CCCc1cc(C(N)=O)c(NC(=O)COc2ccccc2C)s1] |
| 127 | 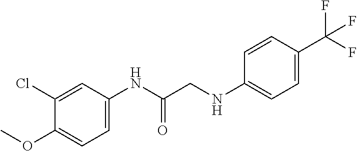 [SMILES: COc1ccc(NC(=O)CNc2ccc(C(F)(F)F)cc2)cc1Cl] |
| 143 | 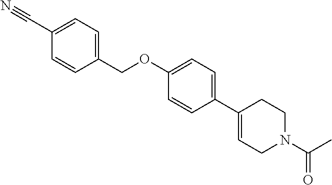 [SMILES: CC(=O)N1CC=C(c2ccc(OCc3ccc(C#N)cc3)cc2)CC1] |

153. The compound of any one of the preceding claims, wherein the compound is selected from the group consisting of

| Compd. | |
|---|---|
| No. | Structure |
| 141A | 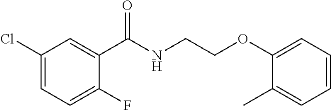 [SMILES: Cc1ccccc1OCCNC(=O)c1cc(Cl)ccc1F] |
| 142A | 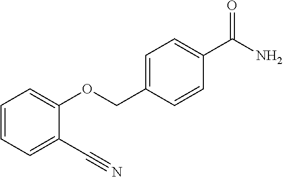 [SMILES: N#Cc1ccccc1OCc1ccc(C(N)=O)cc1] |
| | and |
| 143A | 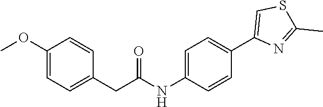 |

154. The compound of any one of the preceding claims, wherein the compound is selected from the group consisting of

| Compd. | |
|---|---|
| No. | Structure |
| 88B | 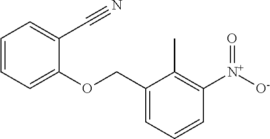 [SMILES: Cc1c(COc2ccccc2C#N)cccc1[N+](=O)[O-]] |
| 89B | 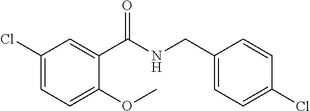 [SMILES: COc1ccc(Cl)cc1C(=O)NCc1ccc(Cl)cc1] |
| 90B | 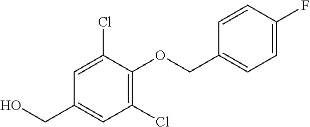 [SMILES: OCc1cc(Cl)c(OCc2ccc(F)cc2)c(Cl)c1] |
| 91B | 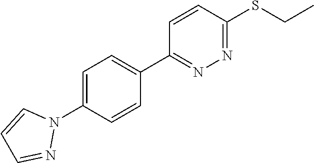 [SMILES: CCSc1ccc(-c2ccc(-n3cccn3)cc2)nn1] |
| 92B | 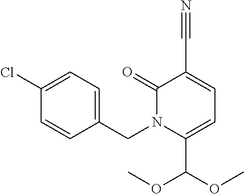 [SMILES: COC(OC)c1ccc(C#N)c(=O)n1Cc1ccc(Cl)cc1] |
| 93B | 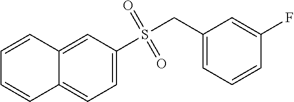 [SMILES: O=S(=O)(Cc1cccc(F)c1)c1ccc2ccccc2c1] |
| 94B | 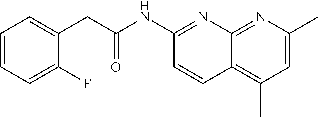 [SMILES: Cc1cc(C)c2ccc(NC(=O)Cc3ccccc3F)nc2n1] |
| 95B | 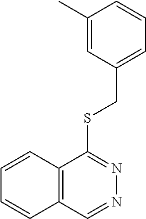 [SMILES: Cc1cccc(CSc2nncc3ccccc23)c1] |
| 96B | 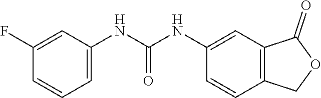 [SMILES: O=C(Nc1cccc(F)c1)Nc1ccc2c(c1)C(=O)OC2] |
| 97B | 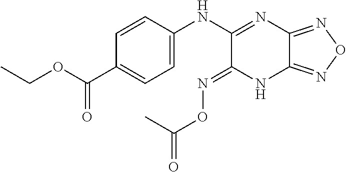 [SMILES: CCOC(=O)c1ccc(Nc2nc3nonc3[nH]/c2=N\OC(C)=O)cc1] |

155. The compound of any one of the preceding claims, wherein A is an optionally substituted monocyclic 5-membered heterocycle comprising 1-3 heteroatoms selected from nitrogen, oxygen, and sulfur.

156. The compound of any one of the preceding claims, wherein the compound is selected from the group consisting of

| Compd. | |
|---|---|
| No. | Structure |
| 144A | 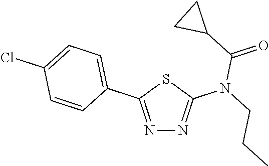 [SMILES: CCCN(C(=O)C1CC1)c1nnc(-c2ccc(Cl)cc2)s1] |
| 145A | 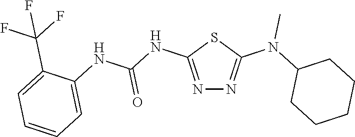 [SMILES: CN(c1nnc(NC(=O)Nc2ccccc2C(F)(F)F)s1)C1CCCCC1] |
| 146A | 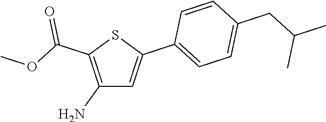 [SMILES: COC(=O)c1sc(-c2ccc(CC(C)C)cc2)cc1N] |
| 147A | 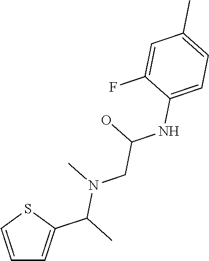 [SMILES: Cc1ccc(NC(O)CN(C)C(C)c2cccs2)c(F)c1] |
| 148A | 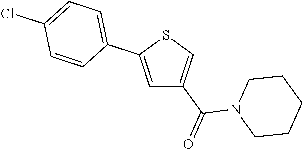 [SMILES: O=C(c1csc(-c2ccc(Cl)cc2)c1)N1CCCCC1] |
| 149A | 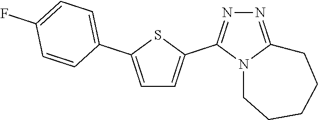 [SMILES: Fc1ccc(-c2ccc(-c3nnc4n3CCCCC4)s2)cc1] |
| 150A | 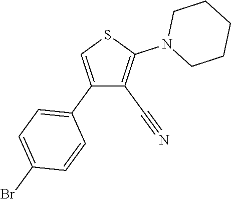 [SMILES: N#Cc1c(-c2ccc(Br)cc2)csc1N1CCCCC1] |
| 151A | 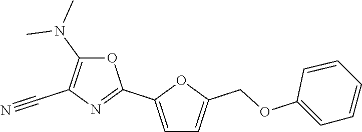 [SMILES: CN(C)c1oc(-c2ccc(COc3ccccc3)o2)nc1C#N] |
| 152A | 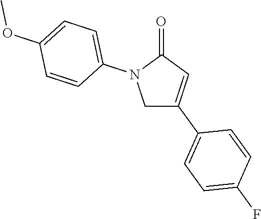 [SMILES: COc1ccc(N2CC(c3ccc(F)cc3)=CC2=O)cc1] |
| 153A | 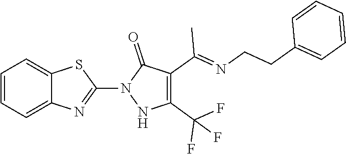 [SMILES: C/C(=N\CCc1ccccc1)c1c(C(F)(F)F)[nH]n(-c2nc3ccccc3s2)c1=O] |

157. The compound of any one of the preceding claims, wherein the compound is selected from the group consisting of

| Compd. | |
|---|---|
| No. | Structure |
| 98B | 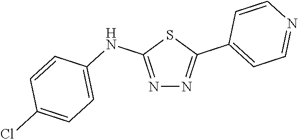 [SMILES: Clc1ccc(Nc2nnc(-c3ccncc3)s2)cc1] |
| 99B | 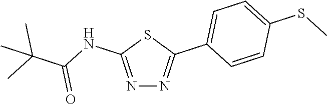 [SMILES: CSc1ccc(-c2nnc(NC(=O)C(C)(C)C)s2)cc1] |
| 100B | 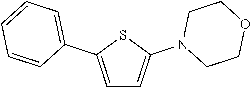 [SMILES: c1ccc(-c2ccc(N3CCOCC3)s2)cc1] |
| 101B | 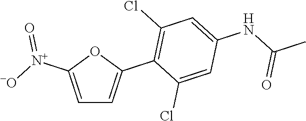 |
| 102B | 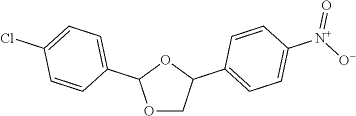 [SMILES: O=[N+]([O-])c1ccc(C2COC(c3ccc(Cl)cc3)O2)cc1] |
| 103B | 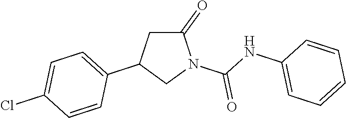 [SMILES: O=C1CC(c2ccc(Cl)cc2)CN1C(=O)Nc1ccccc1] |

158. The compound of any one of the preceding claims, wherein A is an optionally substituted bicyclic 10-membered heterocycle comprising 1-2 nitrogen heteroatoms.

159. The compound of any one of the preceding claims, wherein A is an optionally substituted bicyclic 10-membered heterocycle comprising 1-2 nitrogen heteroatoms selected from the group consisting of quinolone, quinoxaline, and pthalazine.

160. The compound of any one of the preceding claims, wherein A is an optionally substituted bicyclic 10-membered heterocycle comprising 1-2 nitrogen heteroatoms selected from the group consisting of quinolone, quinoxaline, and pthalazine and B is an optionally substituted monocyclic ring selected from benzene and pyrimidine.

161. The compound of any one of the preceding claims, wherein A is an optionally substituted bicyclic 10-membered heterocycle comprising 1-2 nitrogen heteroatoms selected from the group consisting of quinolone, quinoxaline, and pthalazine and C is an optionally substituted benzene.

162. The compound of any one of the preceding claims, wherein A is an optionally substituted bicyclic 10-membered heterocycle comprising 1-2 nitrogen heteroatoms selected from the group consisting of quinolone, quinoxaline, and pthalazine and L_{b} is selected from the group consisting of a covalent bond and *—NH—**.

163. The compound of any one of the preceding claims, wherein A is an optionally substituted bicyclic 10-membered heterocycle comprising 1-2 nitrogen heteroatoms selected from the group consisting of quinolone, quinoxaline, and pthalazine and L_{c} is a covalent bond.

164. The compound of any one of the preceding claims, wherein the compound is selected from the group consisting of

| Compd. | |
|---|---|
| No. | Structure |
| 116 | 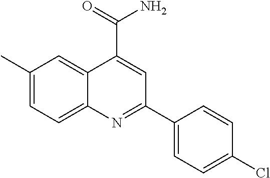 [SMILES: Cc1ccc2nc(-c3ccc(Cl)cc3)cc(C(N)=O)c2c1] |
| 124 | 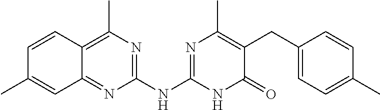 |
| 130 | 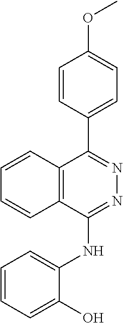 [SMILES: COc1ccc(-c2nnc(Nc3ccccc3O)c3ccccc23)cc1] |
| 131 | 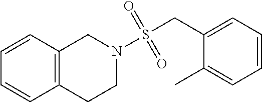 [SMILES: Cc1ccccc1CS(=O)(=O)N1CCc2ccccc2C1] |
| 132 | 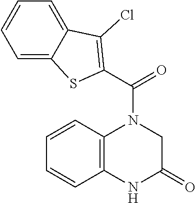 [SMILES: O=C1CN(C(=O)c2sc3ccccc3c2Cl)c2ccccc2N1] |
| 134 | 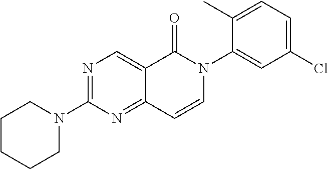 [SMILES: Cc1ccc(Cl)cc1-n1ccc2nc(N3CCCCC3)ncc2c1=O] |
| 128 | 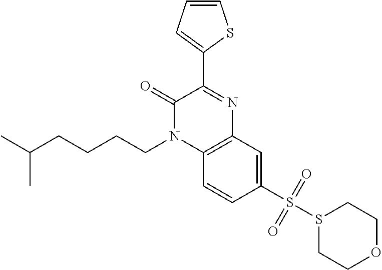 |
| 129 | 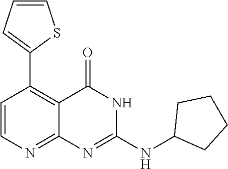 [SMILES: O=c1[nH]c(NC2CCCC2)nc2nccc(-c3cccs3)c12] |
| 151 | 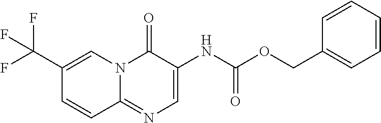 [SMILES: O=C(Nc1cnc2ccc(C(F)(F)F)cn2c1=O)OCc1ccccc1] |
| | and |
| 156 | 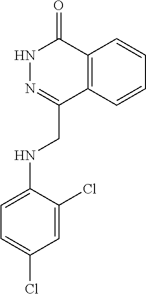 [SMILES: O=c1[nH]nc(CNc2ccc(Cl)cc2Cl)c2ccccc12] |

165. The compound of any one of the preceding claims, wherein the compound is selected from the group consisting of

| Compd. | |
|---|---|
| No. | Structure |
| 154A | 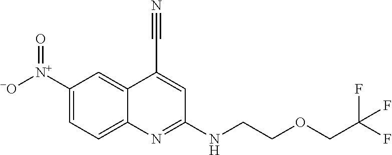 |
| 155A | 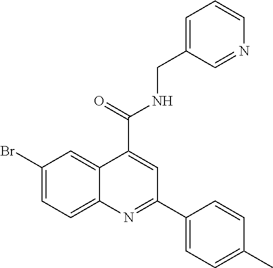 [SMILES: Cc1ccc(-c2cc(C(=O)NCc3cccnc3)c3cc(Br)ccc3n2)cc1] |
| 156A | 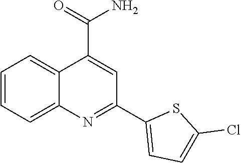 [SMILES: NC(=O)c1cc(-c2ccc(Cl)s2)nc2ccccc12] |
| 157A | 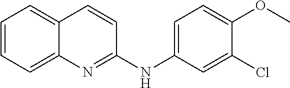 [SMILES: COc1ccc(Nc2ccc3ccccc3n2)cc1Cl] |
| 158A | 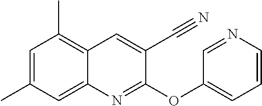 [SMILES: Cc1cc(C)c2cc(C#N)c(Oc3cccnc3)nc2c1] |
| 159A | 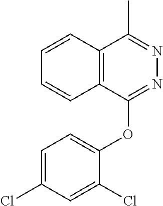 [SMILES: Cc1nnc(Oc2ccc(Cl)cc2Cl)c2ccccc12] |
| 160A | 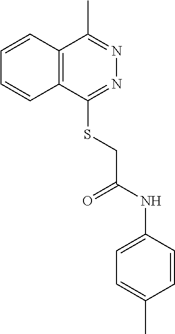 [SMILES: Cc1ccc(NC(=O)CSc2nnc(C)c3ccccc23)cc1] |
| 161A | 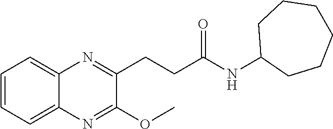 [SMILES: COc1nc2ccccc2nc1CCC(=O)NC1CCCCCC1] |
| 162A | 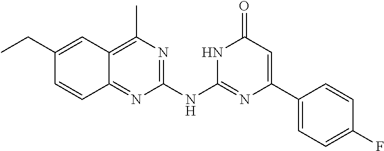 [SMILES: CCc1ccc2nc(Nc3nc(-c4ccc(F)cc4)cc(=O)[nH]3)nc(C)c2c1] |
| 163A | 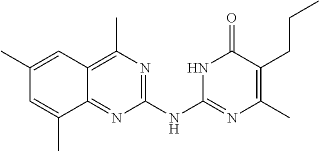 [SMILES: CCCc1c(C)nc(Nc2nc(C)c3cc(C)cc(C)c3n2)[nH]c1=O] |
| 164A | 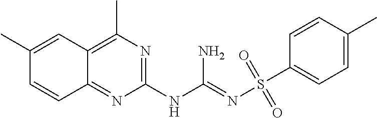 |
| 165A | 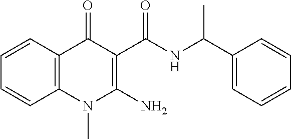 [SMILES: CC(NC(=O)c1c(N)n(C)c2ccccc2c1=O)c1ccccc1] |
| 166A | 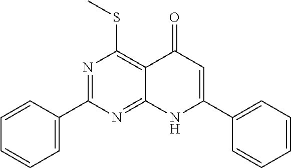 [SMILES: CSc1nc(-c2ccccc2)nc2[nH]c(-c3ccccc3)cc(=O)c12] |
| 167A | 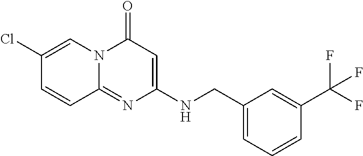 [SMILES: O=c1cc(NCc2cccc(C(F)(F)F)c2)nc2ccc(Cl)cn12] |
| | and |
| 168A | 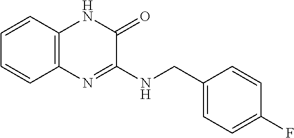 [SMILES: O=c1[nH]c2ccccc2nc1NCc1ccc(F)cc1] |

166. The compound of any one of the preceding claims, wherein the compound is selected from the group consisting of

| Compd. | |
|---|---|
| No. | Structure |
| 104B | 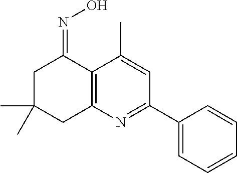 [SMILES: Cc1cc(-c2ccccc2)nc2c1/C(=N\O)CC(C)(C)C2] |
| | and |
| 105B | 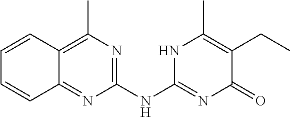 [SMILES: CCc1c(C)[nH]c(Nc2nc(C)c3ccccc3n2)nc1=O] |

167. The compound of any one of the preceding claims, wherein A is an optionally substituted bicyclic 10-membered heterocycle comprising 1-3 nitrogen heteroatoms and B is an optionally substituted monocyclic ring selected from the group consisting of benzene and thiophene.

168. The compound of any one of the preceding claims, wherein A is an optionally substituted bicyclic 10-membered heterocycle comprising 1-3 nitrogen heteroatoms and B is an optionally substituted benzo[b]thiophene.

169. The compound of any one of the preceding claims, wherein A is an optionally substituted bicyclic 10-membered heterocycle comprising 1-3 nitrogen heteroatoms and C is an optionally substituted monocyclic ring selected from the group consisting of piperidine and morpholine.

170. The compound of any one of the preceding claims, wherein A is an optionally substituted bicyclic 10-membered heterocycle comprising 1-3 nitrogen heteroatoms and L_{b} is selected from the group consisting of a covalent bond, *—NHC(O)OCH₂—**, *—CH₂NH—**, *—SO₂CH₂—**, and *—C(O)—**.

171. The compound of any one of the preceding claims, wherein A is an optionally substituted bicyclic 10-membered heterocycle comprising 1-3 nitrogen heteroatoms and L_{c} is selected from the group consisting of a covalent bond and *—SO₂—**.

172. The compound of any one of the preceding claims, wherein A is an optionally substituted bicyclic 9-membered heterocycle comprising 1-2 nitrogen heteroatoms and 1 sulfur heteroatom.

173. The compound of any one of the preceding claims, wherein A is an optionally substituted bicyclic 9-membered heterocycle comprising 1-2 nitrogen heteroatoms and 1 sulfur heteroatom and B is an optionally substituted benzene.

174. The compound of any one of the preceding claims, wherein A is an optionally substituted bicyclic 9-membered heterocycle comprising 1-2 nitrogen heteroatoms and 1 sulfur heteroatom and c is 0.

175. The compound of any one of the preceding claims, wherein A is an optionally substituted bicyclic 9-membered heterocycle comprising 1-2 nitrogen heteroatoms and 1 sulfur heteroatom and L_{b} is selected from the group consisting of a covalent bond, *—O—**, and *—NHC(O)NH—**.

176. The compound of any one of the preceding claims, wherein the compound is selected from the group consisting of

| Compd. | |
|---|---|
| No, | Structure |
| 121 | 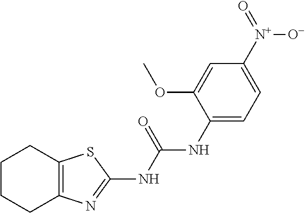 [SMILES: COc1cc([N+](=O)[O-])ccc1NC(=O)Nc1nc2c(s1)CCCC2] |
| 136 | 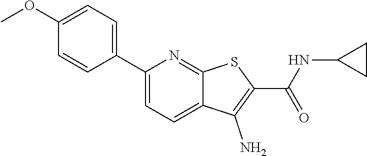 [SMILES: COc1ccc(-c2ccc3c(N)c(C(=O)NC4CC4)sc3n2)cc1] |
| 138 | 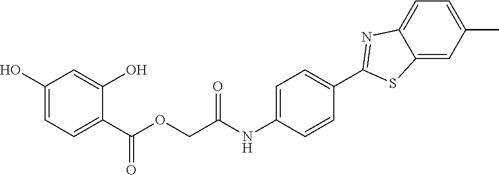 [SMILES: Cc1ccc2nc(-c3ccc(NC(=O)COC(=O)c4ccc(O)cc4O)cc3)sc2c1] |
| 147 | 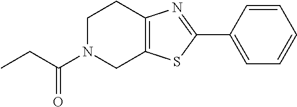 [SMILES: CCC(=O)N1CCc2nc(-c3ccccc3)sc2C1] |
| | and |
| 150 | 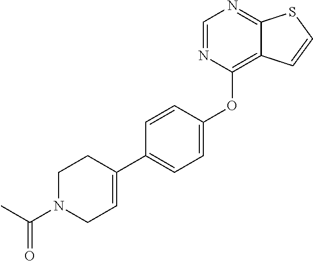 [SMILES: CC(=O)N1CC=C(c2ccc(Oc3ncnc4sccc34)cc2)CC1] |

177. The compound of any one of the preceding claims, wherein the compound is selected from the group consisting of

| Compd. | |
|---|---|
| No. | Structure |
| 169A | 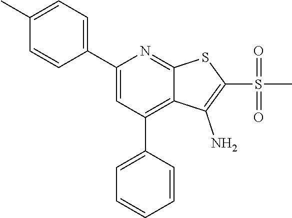 |
| 170A | 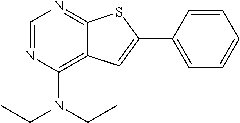 [SMILES: CCN(CC)c1ncnc2sc(-c3ccccc3)cc12] |
| | and |
| 171A | 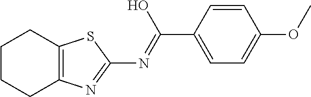 [SMILES: COc1ccc(/C(O)=N/c2nc3c(s2)CCCC3)cc1] |

178. The compound of any one of the preceding claims, wherein the compound is selected from the group consisting of

| Compd. | |
|---|---|
| No. | Structure |
| 106B | 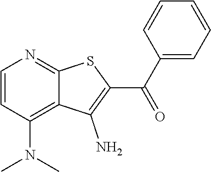 [SMILES: CN(C)c1ccnc2sc(C(=O)c3ccccc3)c(N)c12] |
| 107B | 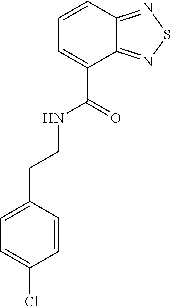 [SMILES: O=C(NCCc1ccc(Cl)cc1)c1cccc2nsnc12] |
| | and |
| 108B | 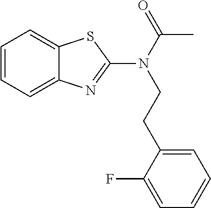 [SMILES: CC(=O)N(CCc1ccccc1F)c1nc2ccccc2s1] |

179. The compound of any one of the preceding claims, wherein A is an optionally substituted bicyclic 8- to 10-membered heterocycle comprising 1-4 heteroatoms selected from N, O, and S.

180. The compound of any one of the preceding claims, wherein A is an optionally substituted bicyclic 9-membered heterocycle comprising 1-4 nitrogen heteroatoms.

181. The compound of any one of the preceding claims, wherein A is an optionally substituted bicyclic 9-membered heterocycle comprising 1-4 nitrogen heteroatoms and B is an optionally substituted benzene.

182. The compound of any one of the preceding claims, wherein A is an optionally substituted bicyclic 9-membered heterocycle comprising 1-4 nitrogen heteroatoms and C is an optionally substituted benzene.

183. The compound of any one of the preceding claims, wherein A is an optionally substituted bicyclic 9-membered heterocycle comprising 1-4 nitrogen heteroatoms and L_{b} is covalent bond.

184. The compound of any one of the preceding claims, wherein A is an optionally substituted bicyclic 9-membered heterocycle comprising 1-4 nitrogen heteroatoms and L_{c} is covalent bond.

185. The compound of any one of the preceding claims, wherein the compound is selected from the group consisting of

| Compd. | |
|---|---|
| No. | Structure |
| 109 | 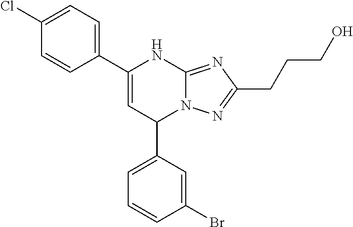 [SMILES: OCCCc1nc2n(n1)C(c1cccc(Br)c1)C=C(c1ccc(Cl)cc1)N2] |
| 117 | 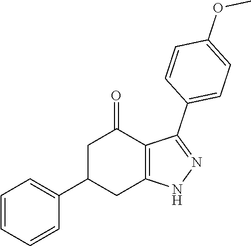 [SMILES: COc1ccc(-c2n[nH]c3c2C(=O)CC(c2ccccc2)C3)cc1] |
| 135 | 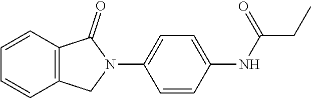 [SMILES: CCC(=O)Nc1ccc(N2Cc3ccccc3C2=O)cc1] |
| | and |
| 137 | 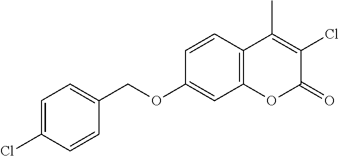 [SMILES: Cc1c(Cl)c(=O)oc2cc(OCc3ccc(Cl)cc3)ccc12] |

186. The compound of any one of the preceding claims, wherein the compound is selected from the group consisting of

| Compd. | |
|---|---|
| No. | Structure |
| 172A | 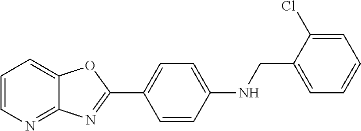 [SMILES: Clc1ccccc1CNc1ccc(-c2nc3ncccc3o2)cc1] |
| 173A | 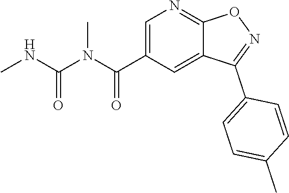 [SMILES: CNC(=O)N(C)C(=O)c1cnc2onc(-c3ccc(C)cc3)c2c1] |
| 174A | 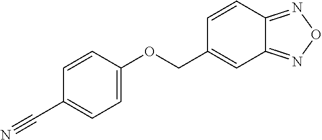 [SMILES: N#Cc1ccc(OCc2ccc3nonc3c2)cc1] |
| 175A | 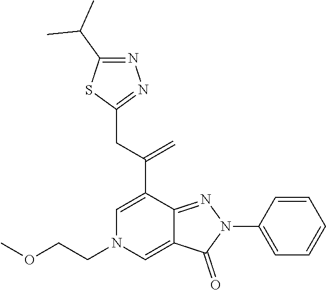 [SMILES: C=C(Cc1nnc(C(C)C)s1)c1cn(CCOC)cc2c(=O)n(-c3ccccc3)nc1-2] |
| 176A | 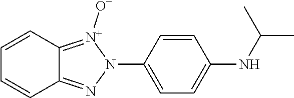 [SMILES: CC(C)Nc1ccc(-n2nc3ccccc3[n+]2[O-])cc1] |
| 177A | 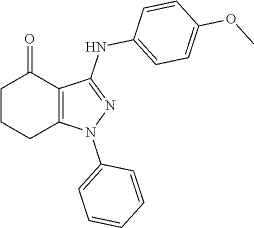 [SMILES: COc1ccc(Nc2nn(-c3ccccc3)c3c2C(=O)CCC3)cc1] |
| 178A | 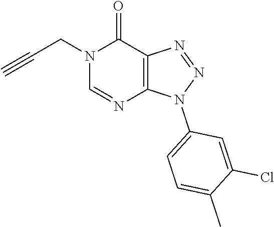 [SMILES: C#CCn1cnc2c(nnn2-c2ccc(C)c(Cl)c2)c1=O] |
| 179A | 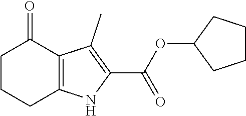 [SMILES: Cc1c(C(=O)OC2CCCC2)[nH]c2c1C(=O)CCC2] |
| 180A | 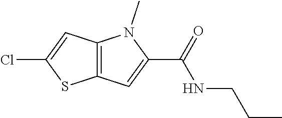 [SMILES: CCCNC(=O)c1cc2sc(Cl)cc2n1C] |
| 181A | 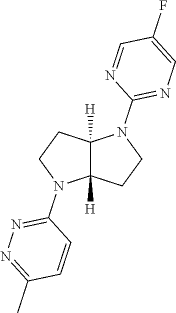 [SMILES: Cc1ccc(N2CC[C@@H]3[C@@H]2CCN3c2ncc(F)cn2)nn1] |
| | and |
| 182A | 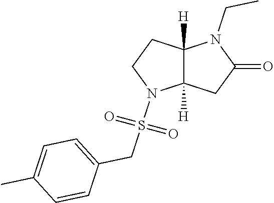 [SMILES: CCN1C(=O)C[C@@H]2[C@@H]1CCN2S(=O)(=O)Cc1ccc(C)cc1] |

187. The compound of any one of the preceding claims, wherein the compound is selected from the group consisting of

| Compd. | |
|---|---|
| No. | Structure |
| 109B | 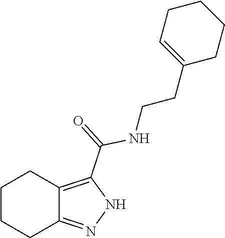 [SMILES: O=C(NCCC1=CCCCC1)c1[nH]nc2c1CCCC2] |
| 110B | 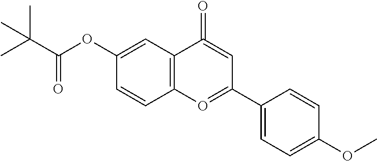 |
| 111B | 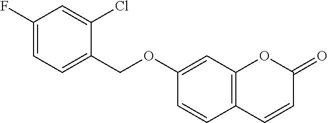 [SMILES: O=c1ccc2ccc(OCc3ccc(F)cc3Cl)cc2o1] |
| 112B | 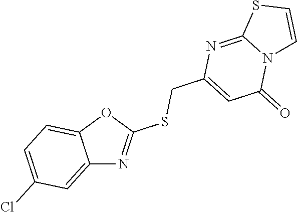 [SMILES: O=c1cc(CSc2nc3cc(Cl)ccc3o2)nc2sccn12] |
| 113B | 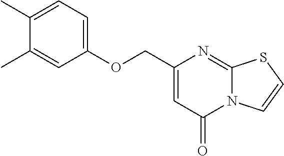 [SMILES: Cc1ccc(OCc2cc(=O)n3ccsc3n2)cc1C] |
| 114B | 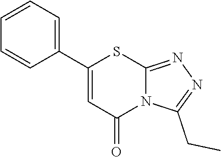 [SMILES: CCc1nnc2sc(-c3ccccc3)cc(=O)n12] |
| 115B | 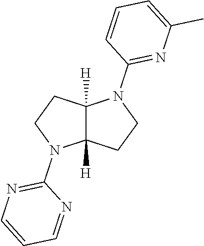 [SMILES: Cc1cccc(N2CC[C@H]3[C@H]2CCN3c2ncccn2)n1] |
| | and |
| 116B | 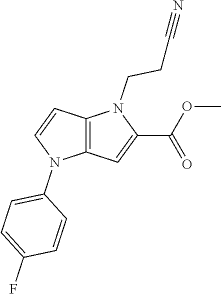 [SMILES: COC(=O)c1cc2c(ccn2-c2ccc(F)cc2)n1CCC#N] |

188. The compound of any one of the preceding claims, wherein A is an optionally substituted tricyclic 11- to 15-membered ring comprising 1-4 heteroatoms selected from the group consisting of nitrogen, oxygen and sulfur.

189. The compound of any one of the preceding claims, wherein A is an optionally substituted tricyclic 13-membered ring comprising 2 heteroatoms selected from the group consisting of nitrogen and sulfur.

190. The compound of any one of the preceding claims, wherein A is an optionally substituted tricyclic 13-membered ring comprising 2 heteroatoms selected from the group consisting of nitrogen and sulfur and B is an optionally substituted monocyclic ring selected from the group consisting of benzene and 1,3,4-oxadiazole.

191. The compound of any one of the preceding claims, wherein A is an optionally substituted tricyclic 13-membered ring comprising 2 heteroatoms selected from the group consisting of nitrogen and sulfur and L_{b} is a covalent bond.

192. The compound of any one of the preceding claims, wherein A is an optionally substituted tricyclic 13-membered ring comprising 2 heteroatoms selected from the group consisting of nitrogen and sulfur and c is 0.

193. The compound of any one of the preceding claims, wherein A is an optionally substituted bicyclic 10-membered heterocycle comprising 1 oxygen heteroatom.

194. The compound of any one of the preceding claims, wherein A is an optionally substituted 2H-chromene and B is an optionally substituted benzene.

195. The compound of any one of the preceding claims, wherein A is an optionally substituted 2H-chromene, B is an optionally substituted benzene and L_{b} is *—OCH₂—**.

196. The compound of any one of the preceding claims, wherein b is 0, c is 0 and A is an optionally substituted tricyclic ring selected from the group consisting of 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-c]quinolone.

197. The compound of any one of the preceding claims, wherein the compound is selected from the group consisting of

| Compd. | |
|---|---|
| No. | Structure |
| 120 | 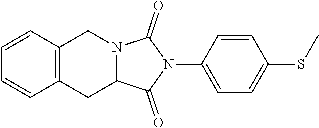 [SMILES: CSc1ccc(N2C(=O)C3Cc4ccccc4CN3C2=O)cc1] |
| 155 | 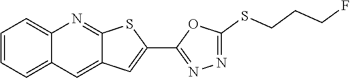 [SMILES: FCCCSc1nnc(-c2cc3cc4ccccc4nc3s2)o1] |
| 152 | 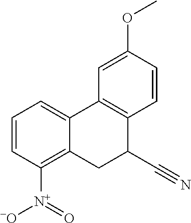 |
| 148 | 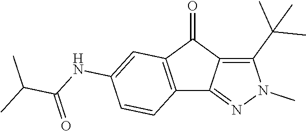 [SMILES: CC(C)C(=O)Nc1ccc2c(c1)C(=O)c1c-2nn(C)c1C(C)(C)C] |
| 146 | 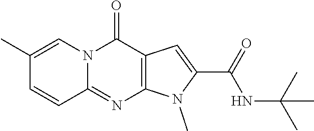 [SMILES: Cc1ccc2nc3c(cc(C(=O)NC(C)(C)C)n3C)c(=O)n2c1] |
| | and |
| 145 | 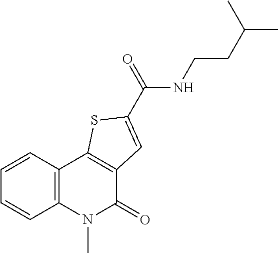 [SMILES: CC(C)CCNC(=O)c1cc2c(=O)n(C)c3ccccc3c2s1] |

198. The compound of any one of the preceding claims, wherein the compound is selected from the group consisting of

| Compd. | |
|---|---|
| No. | Structure |
| 183A | 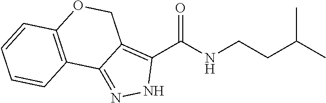 [SMILES: CC(C)CCNC(=O)c1[nH]nc2c1COc1ccccc1-2] |
| 184A | 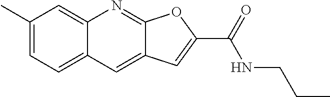 [SMILES: CCCNC(=O)c1cc2cc3ccc(C)cc3nc2o1] |
| 185A | 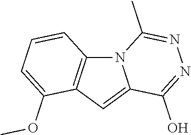 [SMILES: COc1cccc2c1cc1c(O)nnc(C)n12] |
| 186A | 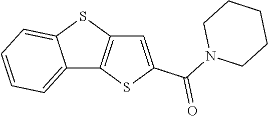 [SMILES: O=C(c1cc2sc3ccccc3c2s1)N1CCCCC1] |
| 187A | 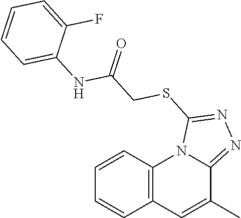 [SMILES: Cc1cc2ccccc2n2c(SCC(=O)Nc3ccccc3F)nnc12] |
| 188A | 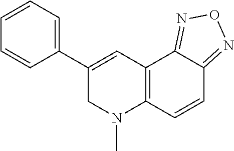 [SMILES: CN1CC(c2ccccc2)=Cc2c1ccc1nonc21] |
| 189A | 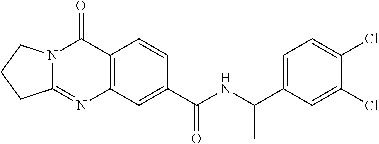 [SMILES: CC(NC(=O)c1ccc2c(=O)n3c(nc2c1)CCC3)c1ccc(Cl)c(Cl)c1] |
| 190A | 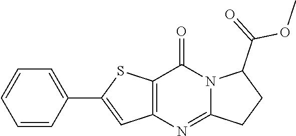 [SMILES: COC(=O)C1CCc2nc3cc(-c4ccccc4)sc3c(=O)n21] |
| 191A | 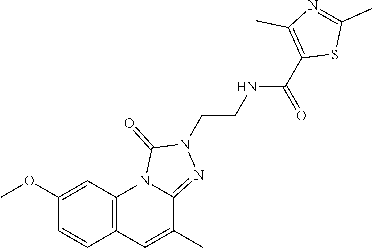 |
| 192A | 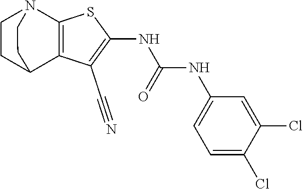 [SMILES: N#Cc1c(NC(=O)Nc2ccc(Cl)c(Cl)c2)sc2c1C1CCN2CC1] |
| | and |
| 193A | 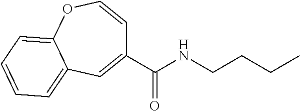 [SMILES: CCCCNC(=O)C1=Cc2ccccc2OC=C1] |

199. The compound of any one of the preceding claims, wherein the compound is selected from the group consisting of

| Compd. | |
|---|---|
| No. | Structure |
| 118B | 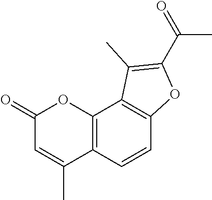 [SMILES: CC(=O)c1oc2ccc3c(C)cc(=O)oc3c2c1C] |
| 119B | 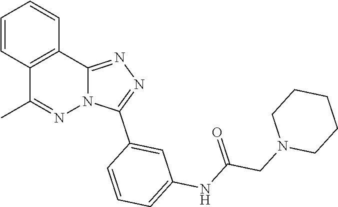 [SMILES: Cc1nn2c(-c3cccc(NC(=O)CN4CCCCC4)c3)nnc2c2ccccc12] |
| 120B | 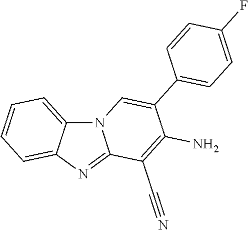 [SMILES: N#Cc1c(N)c(-c2ccc(F)cc2)cn2c1nc1ccccc12] |
| 121B | 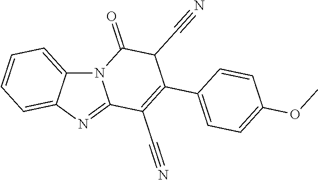 [SMILES: COc1ccc(C2=C(C#N)c3nc4ccccc4n3C(=O)C2C#N)cc1] |
| 122B | 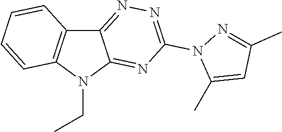 [SMILES: CCn1c2ccccc2c2nnc(-n3nc(C)cc3C)nc21] |
| 123B | 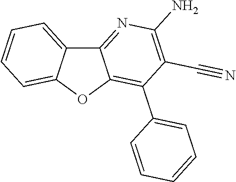 [SMILES: N#Cc1c(N)nc2c(oc3ccccc32)c1-c1ccccc1] |
| 124B | 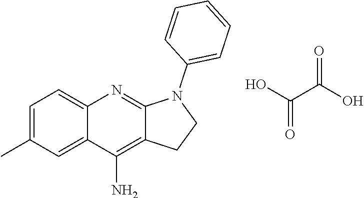 [SMILES: Cc1ccc2nc3c(c(N)c2c1)CCN3c1ccccc1 ; O=C(O)C(=O)O] |
| 125B | 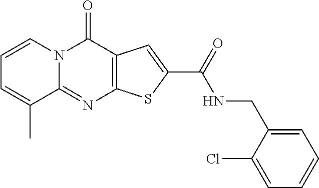 [SMILES: Cc1cccn2c(=O)c3cc(C(=O)NCc4ccccc4Cl)sc3nc12] |
| 126B | 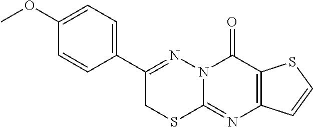 [SMILES: COc1ccc(C2=Nn3c(nc4ccsc4c3=O)SC2)cc1] |
| 127B | 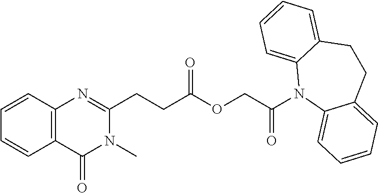 [SMILES: Cn1c(CCC(=O)OCC(=O)N2c3ccccc3CCc3ccccc32)nc2ccccc2c1=O] |
| | and |
| 117B | 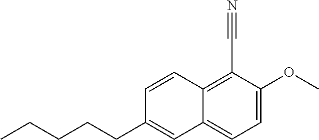 [SMILES: CCCCCc1ccc2c(C#N)c(OC)ccc2c1] |

200. The compound of any one of the preceding claims, wherein A is an optionally substituted bicyclic 10-membered heterocycle comprising 1 oxygen heteroatom.

201. The compound of any one of the preceding claims, wherein A is an optionally substituted 2H-chromene and B is an optionally substituted benzene.

202. The compound of any one of the preceding claims, wherein A is an optionally substituted 2H-chromene, B is an optionally substituted benzene and L_{b} is *—OCH₂—**.

203. The compound of any one of the preceding claims, wherein b is 0, c is 0 and A is an optionally substituted tricyclic ring selected from the group consisting of 9,10-dihydrophenanthrene, 2,4-dihydroindeno[1,2-c]pyrazole, 1,4-dihydropyrido[1,2-a]pyrrolo[2,3-d]pyrimidine, and 4,5-dihydrothieno[3,2-c]quinolone.

204. The compound of any one of the preceding claims, wherein the compound is selected from the group consisting of

| Compd. | |
|---|---|
| No. | Structure |
| 133 | 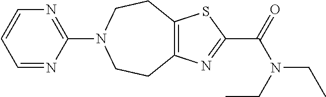 [SMILES: CCN(CC)C(=O)c1nc2c(s1)CCN(c1ncccn1)CC2] |
| 142 | 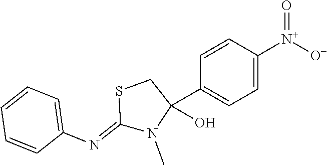 [SMILES: CN1/C(=N/c2ccccc2)SCC1(O)c1ccc([N+](=O)[O-])cc1] |
| | and |
| 153 | 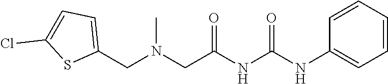 [SMILES: CN(CC(=O)NC(=O)Nc1ccccc1)Cc1ccc(Cl)s1] |

205. The compound of any one of the preceding claims, wherein the compound is selected from the group consisting of

| Compd. | |
|---|---|
| No. | Structure |
| 194A | 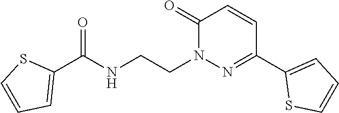 [SMILES: O=C(NCCn1nc(-c2cccs2)ccc1=O)c1cccs1] |
| 195A | 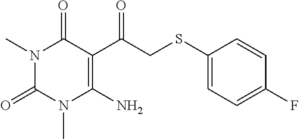 [SMILES: Cn1c(N)c(C(=O)CSc2ccc(F)cc2)c(=O)n(C)c1=O] |
| 196A | 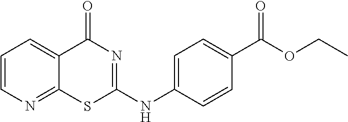 [SMILES: CCOC(=O)c1ccc(Nc2nc(=O)c3cccnc3s2)cc1] |
| | and |
| 197A | 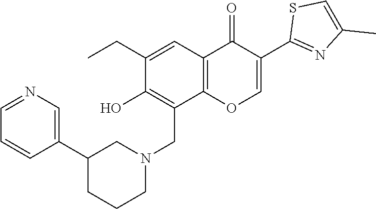 [SMILES: CCc1cc2c(=O)c(-c3nc(C)cs3)coc2c(CN2CCCC(c3cccnc3)C2)c1O] |

206. A method of producing an expanded population of hematopoietic stem cells ex vivo, the method comprising contacting a population of hematopoietic stem cells with the compound of any one of the preceding claims in an amount sufficient to produce an expanded population of hematopoietic stem cells.

207. A method of enriching a population of cells with hematopoietic stem cells ex vivo, said method comprising contacting a population of hematopoietic stem cells with the compound of any one of the preceding claims.

208. A method of maintaining the hematopoietic stem cell functional potential of a population of hematopoietic stem cells ex vivo for two or more days, said method comprising contacting a first population of hematopoietic stem cells with the compound of any one of the preceding claims, wherein the first population of hematopoietic stem cells exhibits a hematopoietic stem cell functional potential after two or more days that is greater than that of a control population of hematopoietic stem cells cultured under the same conditions and for the same time as said first population of hematopoietic stem cells but not contacted with said compound.

209. The method of any one of the preceding claims, wherein said first population of hematopoietic stem cells exhibits a hematopoietic stem cell functional potential after three or more days of culture that is greater than that of said control population of hematopoietic stem cells.

210. The method of any one of the preceding claims, wherein said first population of hematopoietic stem cells exhibits a hematopoietic stem cell functional potential after ten or more days of culture that is greater than that of said control population of hematopoietic stem cells.

211. The method of any one of the preceding claims, wherein said first population of hematopoietic stem cells exhibits a hematopoietic stem cell functional potential after thirty or more days of culture that is greater than that of said control population of hematopoietic stem cells.

212. The method of any one of the preceding claims, wherein said first population of hematopoietic stem cells exhibits a hematopoietic stem cell functional potential after sixty or more days of culture that is greater than that of said control population of hematopoietic stem cells.

213. The method of any one of the preceding claims, wherein said hematopoietic stem cells are mammalian cells.

214. The method of any one of the preceding claims, wherein said mammalian cells are human cells.

215. The method of any one of the preceding claims, wherein said human cells are CD34+ cells.

216. The method of any one of the preceding claims, wherein said CD34+ cells are CD34+, CD34+CD38−, CD34+CD38−CD90+, CD34+CD38−CD90+CD45RA−, CD34+CD38−CD90+CD45RA−CD49F+, or CD34+CD90+CD45RA− cells.

217. The method of any one of the preceding claims, wherein said hematopoietic stem cells are obtained from human cord blood.

218. The method of any one of the preceding claims, wherein said hematopoietic stem cells are obtained from mobilized human peripheral blood.

219. The method of any one of the preceding claims, wherein said hematopoietic stem cells are obtained from human bone marrow.

220. The method of any one of the preceding claims, wherein said hematopoietic stem cells are freshly isolated from said human.

221. The method of any one of the preceding claims, wherein said hematopoietic stem cells have been previously cryopreserved.

222. The method of any one of the preceding claims, wherein said hematopoietic stem cells or progeny thereof maintain hematopoietic stem cell functional potential after two or more days upon transplantation of said hematopoietic stem cells into a human subject.

223. The method of any one of the preceding claims, wherein said hematopoietic stem cells or progeny thereof are capable of localizing to hematopoietic tissue and reestablishing hematopoiesis upon transplantation of said hematopoietic stem cells into a human subject.

224. The method of any one of the preceding claims, wherein upon transplantation into a human subject, said hematopoietic stem cells give rise to a population of cells selected from the group consisting of megakaryocytes, thrombocytes, platelets, erythrocytes, mast cells, myoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

225. A method of treating a human patient suffering from a stem cell disorder, said method comprising administering to said patient a population of hematopoietic stem cells, wherein said hematopoietic stem cells were produced by contacting said hematopoietic stem cells or progenitors thereof with the compound of any one of the preceding claims.

226. A method of preparing an expanded population of hematopoietic stem cells for transplantation into a human patient suffering from a stem cell disorder, said method comprising contacting a first population of hematopoietic stem cells with the compound of any one of the preceding claims for a time sufficient to produce said expanded population of hematopoietic stem cells.

227. A method of treating a human patient suffering from a stem cell disorder, said method comprising:
a. preparing an expanded population of hematopoietic stem cells by contacting a first population of hematopoietic stem cells with the compound of any one of the preceding claims for a time sufficient to produce said expanded population of hematopoietic stem cells; and
b. administering said expanded population of hematopoietic stem cells to said patient.

228. The method of any one of the preceding claims, wherein said stem cell disorder is a hemoglobinopathy.

229. The method of any one of the preceding claims, wherein said stem cell disorder is selected from the group consisting of sickle cell anemia, thalassemia, Fanconi anemia, and Wiskott-Aldrich syndrome.

230. The method of any one of the preceding claims, wherein said stem cell disorder is Fanconi anemia.

231. The method of any one of the preceding claims, wherein said stem cell disorder is an immunodeficiency disorder.

232. The method of any one of the preceding claims, wherein said immunodeficiency disorder is a congenital immunodeficiency.

233. The method of any one of the preceding claims, wherein said immunodeficiency disorder is an acquired immunodeficiency.

234. The method of any one of the preceding claims, wherein said acquired immunodeficiency is human immunodeficiency virus or acquired immune deficiency syndrome.

235. The method of any one of the preceding claims, wherein said stem cell disorder is a metabolic disorder.

236. The method of any one of the preceding claims, wherein said metabolic disorder is selected from the group consisting of glycogen storage diseases, mucopolysaccharidoses, Gaucher's Disease, Hurlers Disease, sphingolipidoses, and metachromatic leukodystrophy.

237. A method of producing microglia in the central nervous system of a patient (e.g., a human patient) in need thereof, comprising administering an expanded population of hematopoietic stem cells to the patient, wherein the expanded population of hematopoietic stem cells is prepared by contacting a first population of hematopoietic stem cells with a compound of any one of the preceding claims for a time sufficient to produce the expanded population of hematopoietic stem cells, and wherein administration of the expanded population of hematopoietic stem cells results in formation of microglia in the central nervous system of the patient.

238. A method of producing an expanded population comprising genetically modified hematopoietic stem or progenitor cells ex vivo, the method comprising contacting the population comprising genetically modified hematopoietic stem or progenitor cells with an expanding amount of a compound of any one of the preceding claims.

239. The method of claim 325, further comprising disrupting an endogenous gene in a plurality of hematopoietic stem or progenitor cells, thereby producing a population comprising genetically modified hematopoietic stem or progenitor cells.

240. The method of claim 325, further comprising introducing a polynucleotide into a plurality of hematopoietic stem or progenitor cells, thereby producing a population comprising genetically modified hematopoietic stem or progenitor cells that express the polynucleotide.

241. A composition comprising a population hematopoietic stem cells, wherein said hematopoietic stem cells or progenitors thereof have been contacted with the compound of any one of the preceding claims, thereby expanding said hematopoietic stem cells or progenitors thereof.

242. A composition comprising:
the compound of any one of the preceding claims; and
a cell culture medium.

243. The composition of claim 242, wherein the cell culture medium is a basal medium.

244. The composition of claim 242, wherein the cell culture medium is a serum free medium.

245. The composition of claim 242, wherein the cell culture medium comprises one or more cytokines or growth factors selected from the group consisting of IL-1, IL-3, IL-6, IL-11, G-CSF, GM-CSF, SCF, Fl3-L, thrombopoietin (TPO), erythropoietin, and analogs thereof.

246. The composition of claim 242, wherein the cell culture medium is a basal serum-free medium further comprising thrombopoietin (TPO), IL-6, SCF, and Flt3-L.

247. A kit comprising the compound of any one of the preceding claims and a package insert, wherein said package insert instructs a user of said kit to contact a population of hematopoietic stem cells with said compound for a time sufficient to produce an expanded population of hematopoietic stem cells.

248. A kit comprising the compound of any one of the preceding claims and a package insert, wherein said package insert instructs a user of said kit to contact a population of cells comprising hematopoietic stem cells with said compound for a time sufficient to produce a population of cells enriched with hematopoietic stem cells.

249. A kit comprising the compound of any one of the preceding claims and a package insert, wherein said package insert instructs a user of said kit to contact a population of hematopoietic stem cells with said compound for a time sufficient to maintain the hematopoietic stem cell functional potential of said population of hematopoietic stem cells ex vivo for two or more days.

250. The kit of any one of the previous claims, wherein said kit further comprises a population of cells comprising hematopoietic stem cells.

251. A pharmaceutical composition comprising a compound of any one of the preceding claims, or a pharmaceutically acceptable salt, hydrate, or solvate thereof, and a pharmaceutically acceptable carrier.

252. A method of modulating the activity of an aryl hydrocarbon receptor, comprising administering to a subject in need thereof an effective amount of a compound of any one the preceding claims, or a pharmaceutically acceptable salt, hydrate, or solvate thereof.

253. A method of treating or preventing a disease or disorder, comprising administering to a subject in need thereof an effective amount of a compound of any one of the preceding claims, or a pharmaceutically acceptable salt, hydrate, or solvate thereof.

254. The method of claim 253, wherein the disease or disorder is characterized by the production of an aryl hydrocarbon receptor agonist.

255. The method of claim 253 or 254, wherein the disease or disorder is a cancer, a cancerous condition, or a tumor.

256. The method of claim 255, wherein the tumor is an invasive tumor.

257. The method of claim 255, wherein the tumor is a solid tumor.

258. The method of claim 255, wherein the cancer is a breast cancer, squamous cell cancer, lung cancer, a cancer of the peritoneum, a hepatocellular cancer, a gastric cancer, a pancreatic cancer, a glioblastoma, a cervical cancer, an ovarian cancer, a liver cancer, a bladder cancer, a hepatoma, a colon cancer, a colorectal cancer, an endometrial or uterine carcinoma, a salivary gland carcinoma, a kidney or renal cancer, a prostate cancer, a vulval cancer, a thyroid cancer, a head and neck cancer, a B-cell lymphoma, a chronic lymphocytic leukemia (CLL); an acute lymphoblastic leukemia (ALL), a Hairy cell leukemia, or a chronic myeloblastic leukemia.

259. The method of claim 255, further comprising administering one or more additional anti-cancer therapies.

260. A method of identifying a compound as an aryl hydrocarbon receptor antagonist, the method comprising:
activating luciferase transcription in a cell line transfected with a dioxin-response element luciferase reporter construct with an aryl hydrocarbon receptor agonist and measuring a first level of luciferase transcription;
contacting the cell line with the compound; and
measuring a second level of luciferase transcription;
wherein when the first level of luciferase transcription is greater than the second level of luciferase transcription the compound is identified as an aryl hydrocarbon receptor antagonist.
